(19) United States
(12) Patent Application Publication
(10) Pub. No.: US 2023/0212565 A1
(43) Pub. Date: Jul. 6, 2023

(54) NUCLEIC ACID MOLECULE FOR TREATING THROMBOCYTOPENIA AND APPLICATION THEREOF

(71) Applicant: Ractigen Therapeutics, Nantong City, Jiangsu (CN)

(72) Inventors: Longcheng LI, Nantong City (CN); Moorim KANG, Nantong City (CN)

(21) Appl. No.: 17/753,927

(22) PCT Filed: Sep. 18, 2020
(86) PCT No.: PCT/CN2020/116227

§ 371 Date: Mar. 18, 2022

(30) Foreign Application Priority Data
Sep. 20, 2019 (CN) 201910891508.8

(51) Int. Cl. C12N 15/113; A61P 7/04

(52) U.S. Cl. CPC C12N 15/113; A61P 7/04; C12N 2310/11; C12N 2310/53; C12N 2320/11; C12N 2830/00

(57) ABSTRACT

The present invention relates to a small activating nucleic acid molecules and uses thereof for treating diseases and conditions, such as thrombocytopenia, related to THPO protein deficiency or insufficiency. As described herein, small activating nucleic acid molecules can be double-stranded or single-stranded RNA molecules targeting the promoter region of the Thpo/THPO gene through an RNA activation mechanism and comprise a first nucleic acid strand and a second nucleic acid strand. The double-stranded RNA molecule targeting the promoter region of the Thpo/THPO gene comprises two nucleic acid strands of 16 to 35 nucleotides in length, wherein one nucleic acid strand has at least 75% homology or complementarity to a target selected from the promoter region of the Thpo/THPO gene. The present invention also relates to pharmaceutical compositions and formulations comprising the small activating nucleic acid molecules and methods for up-regulating the expression of the Thpo/THPO gene in a cell and treating diseases and conditions, related to THPO protein deficiency or insufficiency, by administering small activating nucleic acid molecules, pharmaceutical compositions, and formulations thereof.

### TECHNICAL FIELD

The present invention belongs to the technical field of nucleic acids, particularly as it relates to double-stranded nucleic acid molecules associated with gene activation, e.g., small activating nucleic acid molecules, uses of the small activating nucleic acid molecules in activating/up-regulating the transcription of the Thrombopoietin (THPO, TPO) gene, and uses thereof for treating diseases and conditions, such as thrombocytopenia, related to THPO protein deficiency or insufficiency.

### BACKGROUND

Thrombocyte is one of the blood components in peripheral blood derived from nucleus-free megakaryocytes in the bone marrow (Machlus, Thon, and Italiano 2014). The main function of the thrombocyte is to promote hemostasis, accelerate coagulation, and maintain the integrity of capillary walls, while playing an important role in physiological hemostasis and pathological thrombosis (Laki 1972).

Thrombocytopenia is a disease in which the number of platelets in peripheral blood is abnormally reduced, and characterized clinically by ecchymosis, mucosal bleeding, or intracranial bleeding, all of which endanger the lives of patients with this disease. Thrombocytopenia is considered to be present when platelet concentrations are lower than 100×10⁹/L by direct blood platelet count (Kuter 2009). Spontaneous bleeding, manifesting as subcutaneous purpuraa, occurs frequently when platelet concentrations are lower than 50×10⁹/L. When the platelet concentrations are lower than 20×10⁹/L, a patient's condition can be life-threatening, especially due to trauma or sudden intracranial bleeding, gastrointestinal bleeding, and the like.

Thrombocytopenia can be classified into 3 categories based on the pathogenesis: myelo-thrombocytopenia, increased peripheral platelet destruction, and splenic sequestration (Seo et al. 2017). Factors causing a reduction in platelets comprise congenital (hereditary) and acquired thrombocytopenia. It has been found currently that hereditary thrombocytopenia can be caused by at least mutations in genes such as MPL, GP1BA, GP1BB, GP9, MYH9, FLI1, NBEAL2, WAS, GATA1, PRKACG, GFI1b, STIM1, FYB, SLFN14, ETV6, DIAPH1 and SRC. The thrombocytopenia caused by acquired factors include thrombocytopenia caused by aplastic anemia, myelodysplastic syndromes, leukemia, drugs, infection, tumor diseases, radiotherapy, bone marrow transplantation, and chronic liver diseases, and immune thrombocytopenia (ITP). Factors causing increased platelet destruction comprise thrombotic thrombocytopenia, heparin-induced thrombocytopenia, drug-induced thrombocytopenia, ITP, and thrombotic thrombocytopenia purpura (TPP) (Hassan and Waller 2015). Thrombocytopenia may also be caused by splenomegaly sequestration, transfusion of banked blood with low platelet content, and the like.

Thrombopoietin (THPO), also known as Megakaryocyte Growth and Development Factor (MGDF) or C-MPL ligand, is a hormone-like glycoprotein encoded by the human THPO gene and mainly produced and secreted by the liver and kidney (de Sauvage et al. 1994). The human THPO gene is located on the long arm of chromosome 3 (3q26.3-3q27.2) and encodes a precursor protein containing 353 amino acids and has a molecular weight of 36 kDa. After the removal of the signal peptide containing 21 amino acids, the remaining 332 amino acids are glycosylated to form a 95 kDa glycoprotein which is then released into circulation to bind the C-MPL receptor on megakaryocytes and platelets stimulating the formation and differentiation of megakaryocytes, while promoting thrombopoiesis (Bartley et al. 1994). The mouse Thpo gene (also known as Ml, Tpo, Mgdf Mpllg) is homologous to the human THPO gene and encodes mouse thrombopoietin. C-MPL was first discovered in mice infected with Myeloproliferative Leukemia Virus (MPLV), and then the human C-MPL gene was cloned (Mignotte et al. 1994).

The first-line clinical treatment for thrombocytopenia is primarily intravenous injections of glucocorticoids (high doses of dexamethasone or prednisone) and gamma globulin (IVIg). If first-line therapy fails or cannot be maintained, the second-line therapy such as drug therapy or splenectomy would then be chosen. Drug therapy is the commonly used second-line treatment method at present, because splenectomy is susceptible to infection, long-term relapse, prolonged hospitalization and high death risk (Provan et al. 2010).

The drugs currently used to treat thrombocytopenia are mainly thrombopoietic agents that stimulate megakaryocytes to grow, maturate, and produce platelet by supplementing THPO or simulating the function thereof. Such thrombopoietic agents include two major classes, recombinant human thrombopoietin (rhTHPO) and C-MPL receptor agonist (C-MPL-RA). Initially, peptides with homology to endogenous THPO (first generation thrombopoietic agents) had shown good results in early clinical studies in patients with thrombocytopenia. However, later clinical studies have found that the body can produce neutralizing antibodies that cross-react with endogenous THPO, resulting in persistent thrombocytopenia (Basser 2002; Solberg 2005). In addition, since recombinant THPO produced in vitro cannot be completely glycosylated, its thrombopoietic function is limited. Second generation thrombopoietic agents are primarily C-MMPL-RAs, which include Eltrombopag and Romiplostim approved by the FDA in 2008 for the ITP treatment, and Avatrombopag approved by the FDA and marketed for the treatment of ITP in 2018. C-MPL agonist drugs have some curative effects, however, they have been associated with various complications and withdrawal reactions.

Although the aforementioned drugs have some curative effects by supplementing or mimicking the function of THPO to promote thrombopoiesis or simulate the function thereof, these drugs have certain defects, and all belong to second-line treatments and are all mainly used for treating ITP. In order to effectively treat thrombocytopenia caused by various reasons, there is a need to develop treatment methods based on a novel mechanism.

### SUMMARY

One objective of the present invention is to provide a small activating nucleic acid molecule based on an RNA activation mechanism, which promotes thrombopoiesis or treats a disease and condition related to THPO protein deficiency or insufficiency or thrombocytopenia caused by various reasons, such as drug-induced thrombocytopenia and immune thrombocytopenia, by activating/up-regulating THPO gene transcription to increase protein expression of THPO.

Another objective of the present invention is to provide a composition or formulation comprising the small activating nucleic acid molecule.

Another objective of the present invention is to provide a use of a small activating nucleic acid molecule or a composition or formulation comprising same in the preparation of a drug for activating/up-regulating the expression of the THPO gene in a cell.

Yet another objective of the present invention is to provide a method for activating/up-regulating the expression of the Thpo/THPO gene in a cell.

Still another objective of the present invention is to provide a use of a small activating nucleic acid molecule or a composition or formulation thereof in the preparation of a drug for treating thrombocytopenia and/or a disease or a condition related to THPO protein deficiency or insufficiency, or in a method of treating thrombocytopenia and/or a disease or a condition related to THPO protein deficiency or insufficiency.

Still yet another objective of the present invention is to provide an isolated small activating nucleic acid molecule target site of the Thpo/THPO gene, wherein the target site comprises or is selected from any continuous sequence of 16 to 35 nucleotides in length in any sequence set forth in SEQ ID NO: 2 to SEQ ID NO: 4 and SEQ ID NO: 601 to SEQ ID NO: 605, or a sequence having at least 75%, e.g., at least about 79%, about 80%, about 85%, about 90%, about 95%, about 99% or 100% homology to a sequence consisting of any of the aforementioned continuous 16 to 35 nucleotides in length.

### Technical Solution

In one aspect of the present invention, a small activating nucleic acid (e.g., a small activating RNA molecule, saRNA) molecule activating/up-regulating the expression of the Thpo gene in a mouse cell is provided, wherein one strand of the small activating nucleic acid molecule has at least 75% homology or complementarity to any nucleic acid sequence of 16 to 35 nucleotides in length in a promoter region of a mouse Thpo gene, thereby activating or up-regulating the expression of the gene, wherein the promoter region comprises 500 nucleotides upstream of a transcription start site. Specifically, one strand of the small activating nucleic acid molecule comprises or is selected from a nucleic acid sequence having at least 75% (e.g., at least about 79%, about 80%, about 85%, about 90%, about 95%, about 99% or 100%) homology or complementarity to a continuous sequence of 16 to 35 nucleotides in length at positions −493 to −356 (hotspot 1, SEQ ID NO: 2), positions −273 to −183 (hotspot 2, SEQ ID NO: 3), or positions −164 to −80 (hotspot 3, SEQ ID NO: 4) upstream of the transcription start site of the Thpo gene promoter. More specifically, one strand of the small activating nucleic acid molecule of the present invention has at least 75% (e.g., at least about 79%, about 80, about 85%, about 90%, about 95%, about 99% or about 100%) homology or complementarity to any nucleotide sequence selected from SEQ ID NO: 200 to SEQ ID NO: 296. In one specific embodiment, one strand of the small activating nucleic acid molecule of the present invention comprises a nucleic acid sequence having at least 75% (e.g., at least about 79%, about 80%, about 85%, about 90%, about 95%, about 99% or about 100%) homology or complementarity to any nucleotide sequence selected from SEQ ID NO: 200 to SEQ ID NO: 296. In another embodiment, one strand of the small activating nucleic acid molecule of the present invention consists of a nucleic acid sequence having at least 75% (e.g., at least about 79%, about 80%, about 85%, about 90%, about 95%, about 99% or about 100%) homology or complementarity to any nucleotide sequence selected from SEQ ID NO: 200 to SEQ ID NO: 296. In yet another embodiment, one strand of the small activating nucleic acid molecule of the present invention is a nucleic acid sequence having at least 75% (e.g., at least about 79%, about 80%, about 85%, about 90%, about 95%, about 99% or about 100%) homology or complementarity to any nucleotide sequence selected from SEQ ID NO: 200 to SEQ ID NO: 296.

The small activating nucleic acid molecule of the present invention comprises a double-stranded small activating nucleic acid molecule, such as the small activating RNA (saRNA) molecule, targeting the promoter region of a mouse Thpo gene comprising a first nucleic acid strand and a second nucleic acid strand, wherein the first nucleic acid strand has at least 75% (e.g., at least about 79%, about 80%, about 85%, about 90%, about 95%, about 99% or about 100%) homology or complementarity to any continuous sequence of 16 to 35 nucleotides in length in positions −493 to −356 (hotspot 1, SEQ ID NO: 2), positions −273 to −183 (hotspot 2, SEQ ID NO: 3) or positions −164 to −80 (hotspot 3, SEQ ID NO: 4) away from the transcription start site of the Thpo gene promoter, and the first nucleic acid strand and the second nucleic acid strand can complementarily form a double-stranded nucleic acid structure capable of activating the expression of the Thpo gene in a cell.

In another aspect of the present invention, a small activating nucleic acid (such as a small activating RNA molecule, saRNA) molecule activating/up-regulating the expression of the THPO gene in a mouse cell is provided, wherein one strand of the small activating nucleic acid molecule has at least 75% homology or complementarity to any nucleic acid sequence of 16 to 35 nucleotides in length in a promoter region of the human THPO gene, thereby activating or up-regulating the expression of the gene, wherein the promoter region comprises 500 nucleotides upstream of a transcription start site. Specifically, one strand of the small activating nucleic acid molecule comprises or is selected from a nucleic acid sequence having at least 75% (e.g., at least about 79%, about 80%, about 85%, about 90%, about 95%, about 99% or 100%) homology or complementarity to a continuous sequence of 16 to 35 nucleotides in length at positions −1339 to −1044 (hotspot 1, SEQ ID NO: 601), positions −1027 to −903 (hotspot 2, SEQ ID NO: 602), positions −861 to −754 (hotspot 3, SEQ ID NO: 603), positions −728 to −611 (hotspot 4, SEQ ID NO: 604), or positions −593 to −1 (hotspot 5, SEQ ID NO: 605) upstream of the transcription start site of the THPO gene promoter. More specifically, one strand of the small activating nucleic acid molecule of the present invention has at least 75% (e.g., at least about 79%, about 80, about 85%, about 90%, about 95%, about 99% or about 100%) homology or complementarity to any nucleotide sequence selected from SEQ ID NO: 606 to SEQ ID NO: 1047. In one specific embodiment, one strand of the small activating nucleic acid molecule of the present invention comprises a nucleic acid sequence having at least 75% (e.g., at least about 79%, about 80%, about 85%, about 90%, about 95%, about 99% or about 100%) homology or complementarity to any nucleotide sequence selected from SEQ ID NO: 606 to SEQ ID NO: 1047. In another embodiment, one strand of the small activating nucleic acid molecule of the present invention consists of a nucleic acid sequence having at least 75% (e.g., at least about 79%, about 80%, about 85%, about 90%, about 95%, about 99% or about 100%) homology or complementarity to any nucleotide sequence selected from SEQ ID NO: 606 to SEQ ID NO: 1047. In yet another embodiment, one strand of the small activating nucleic acid molecule of the present invention is a nucleic acid sequence having at least 75% (e.g., at least about 79%, about 80%, about 85%, about 90%, about 95%, about 99% or about 100%) homology or complementarity to any nucleotide sequence selected from SEQ ID NO: 606 to SEQ ID NO: 1047.

A small activating nucleic acid molecule of the present invention comprises a double-stranded small activating nucleic acid molecule, such as the small activating RNA (saRNA) molecule, targeting the promoter region of the human THPO gene comprising a first nucleic acid strand and a second nucleic acid strand, wherein the first nucleic acid strand has at least 75% (e.g., at least about 79%, about 80%, about 85%, about 90%, about 95%, about 99% or about 100%) homology or complementarity to any continuous sequence of 16 to 35 nucleotides in length at positions −1339 to −1044 (hotspot 1, SEQ ID NO: 601), positions −1027 to −903 (hotspot 2, SEQ ID NO: 602), positions −861 to −754 (hotspot 3, SEQ ID NO: 603), positions −728 to −611 (hotspot 4, SEQ ID NO: 604) or positions −593 to −1 (hotspot 5, SEQ ID NO: 605) upstream of the transcription start site of the THPO gene promoter, and the first nucleic acid strand and the second nucleic acid strand can complementarily form a double-stranded nucleic acid structure capable of activating the expression of the human THPO gene in a cell.

The first nucleic acid strand and the second nucleic acid strand of a small activating nucleic acid molecule of the present invention may be present on either two different nucleic acid strands or on the same nucleic acid strand. When the first nucleic acid strand and the second nucleic acid strand are located on two different strands, at least one strand of the small activating nucleic acid molecule may have overhangs at the 5′ terminus and/or the 3′ terminus, e.g. overhangs of 0 to 6 nucleotides in length at 3′ terminus, such that the overhangs are of 0, 1, 2, 3, 4, 5 or 6 nucleotides in length. Preferably, both strands of the small activating nucleic acid molecule of the present invention have overhangs; more preferably, the 3′ terminus of both strands of the small activating nucleic acid molecule can have overhangs of 0 to 6 nucleotides in length, e.g., overhangs of 0, 1, 2, 3, 4, 5 or 6 nucleotides in length; most preferably overhangs of 2 or 3 nucleotides in length. Preferably, the nucleotide of the overhang is dT.

A small activating nucleic acid molecule of the present invention can also comprise a small activating nucleic acid molecule capable of forming a double-stranded region hairpin structure, e.g., a single-stranded small activating RNA molecule. In one embodiment, a small activating nucleic acid molecule of the present invention comprises a single-stranded small activating RNA molecule targeting the promoter region of the THPO gene, wherein the single-stranded small activating nucleic acid molecule can form a double-stranded region hairpin structure. Preferably, when the first nucleic acid strand and the second nucleic acid strand are present on the same nucleic acid strand, a small activating nucleic acid molecule of the present invention can be a hairpin single-stranded nucleic acid molecule, wherein the first nucleic acid strand and the second nucleic acid strand have complementary regions capable of forming a double-stranded nucleic acid structure, and the double-stranded nucleic acid structure can promote the expression of the THPO gene in a cell with, for example, a RNA activation mechanism.

In the aforementioned small activating nucleic acid molecules, the first nucleic acid strand and the second nucleic acid strand can have 16 to 35 nucleotides (e.g., 16, 17, 18, 19, 20, 21, 22, 23, 24, 25, 26, 27, 28, 29, 30, 31, 32, 33, 34 or 35 nucleotides) in length.

In one embodiment, the first nucleic acid strand of a small activating nucleic acid molecule of the present invention has at least 75% (e.g., at least about 79%, about 80%, about 85%, about 90%, about 95%, about 99% or about 100%) identity or homology to any nucleotide sequence selected from SEQ ID NOs: 6-102, and the second nucleic acid strand of the small activating nucleic acid molecule has at least 75% (e.g., at least about 79%, about 80%, about 85%, about 90%, about 95%, about 99% or about 100%) identity or homology to any nucleotide sequence selected from SEQ ID NOs: 103-199. In one embodiment, the first nucleic acid strand of a small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NOs: 6-102, or consists of a nucleic acid sequence having at least 75% (e.g., at least about 79%, about 80%, about 85%, about 90%, about 95%, about 99% or about 100%) identity or homology to any nucleotide sequence selected from SEQ ID NOs: 6-142; the second nucleic acid strand of the small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NOs: 103-199, or consists of a nucleic acid sequence having at least 75% (e.g., at least about 79%, about 80%, about 85%, about 90%, about 95%, about 99% or about 100%) identity or homology to any nucleotide sequence selected from SEQ ID NOs: 103-199. In a specific embodiment, the first nucleic acid strand of a small activating nucleic acid molecule of the present invention can comprise or be selected from any nucleotide sequence set forth in SEQ ID NOs: 6-102, and the second strand can comprise or be selected from any nucleotide sequence set forth in SEQ ID NOs: 103-199. In one embodiment, the small activating nucleic acid molecule described herein can be synthesized, transcribed in vitro, or expressed by a vector.

In another embodiment, the first nucleic acid strand of a small activating nucleic acid molecule of the present invention has at least 75% (e.g., at least about 79%, about 80%, about 85%, about 90%, about 95%, about 99% or about 100%) identity or homology to any nucleotide sequence selected from SEQ ID NO: 1048 to SEQ ID NO: 1489, and the second nucleic acid strand of a small activating nucleic acid molecule has at least 75% (e.g., at least about 79%, about 80%, about 85%, about 90%, about 95%, about 99% or about 100%) identity or homology to any nucleotide sequence selected from SEQ ID NO: 1490 to SEQ ID NO: 1931. In one embodiment, the first nucleic acid strand of a small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NO: 1048 to SEQ ID NO: 1489, or consists of a nucleic acid sequence having at least 75% (e.g., at least about 79%, about 80%, about 85%, about 90%, about 95%, about 99% or about 100%) identity or homology to any nucleotide sequence selected from SEQ ID NO: 1048 to SEQ ID NO: 1489; the second nucleic acid strand of a small activating nucleic acid molecule of the present invention comprises a nucleic acid sequence having at least 75% (e.g., at least about 79%, about 80%, about 85%, about 90%, about 95%, about 99% or about 100%) identity or homology to any nucleotide sequence selected from SEQ ID NO: 1490 to SEQ ID NO: 1931, or consists of a nucleic acid sequence having at least 75% (e.g., at least about 79%, about 80%, about 85%, about 90%, about 95%, about 99% or about 100%) identity or homology to any nucleotide sequence selected from SEQ ID NO: 1490 to SEQ ID NO: 1931. In a specific embodiment, the first nucleic acid strand of a small activating nucleic acid molecule of the present invention can comprise or be selected from any nucleotide sequence set forth in SEQ ID NO: 1048 to SEQ ID NO: 1489, and the second strand can comprise or be selected from any nucleotide sequence set forth in SEQ ID NO: 1490 to SEQ ID NO: 1931. In one embodiment, the small activating nucleic acid molecule described herein can be synthesized, transcribed in vitro, or expressed by a vector.

All the nucleotides in the small activating nucleic acid molecule described herein can be natural non-chemically modified nucleotides or can comprise at least one modification. In one embodiment, the modification in the small activating nucleic acid molecule described herein can comprise chemical modification, for example, at least one nucleotide can have a chemical modification, and the chemical modification used in the present invention can comprise or be selected from one or more combinations of the following modifications:

(1) modification of a phosphodiester bond of nucleotides in the nucleotide sequence of the small activating nucleic acid molecule;

(2) modification of 2′-OH of a ribose in the nucleotide sequence of the small activating nucleic acid molecule;

(3) modification of a base in the nucleotide sequence of the small activating nucleic acid molecule; and

(4) at least one nucleotide in the nucleotide sequence of the small activating nucleic acid molecule being a locked nucleic acid.

The chemical modifications described herein is well-known to those skilled in the art, and the modification of the phosphodiester bond refers to the modification of oxygen in the phosphodiester bond, including, but not limited to, phosphorothioate modification and boranophosphate modification. Both modifications can stabilize an saRNA structure and maintain high specificity and high affinity for base pairing.

The ribose modification refers to the modification of 2′-OH in pentose of a nucleotide, i.e., the introduction of some substituents into hydroxyl positions of the ribose, for example, including, but not limited to, 2′-fluoro modification, 2′-oxymethyl modification, 2′-oxyethylidene methoxy modification, 2,4′-dinitrophenol modification, locked nucleic acid (LNA), 2′-amino modification, 2′-deoxy modification, and the like.

The base modification refers to the modification of the base of a nucleotide, for example, including, but not limited to, 5′-bromouracil modification, 5′-iodouracil modification, N-methyluracil modification, 2,6-diaminopurine modification, and the like.

These modifications can increase the bioavailability of the small activating nucleic acid molecule, improve affinity to a target sequence, and enhance resistance to nuclease hydrolysis in a cell.

In addition, in order to promote the access of a small activating nucleic acid molecule into a cell, on the basis of the aforementioned modifications, a lipophilic group, such as cholesterol,) can be introduced on the terminus of the first nucleic acid strand and/or the second nucleic acid strand of the small activating nucleic acid molecule to facilitate the interaction with the promoter region of a gene in the cell nucleus, as the cell membrane and nuclear membrane are composed of lipid bilayers.

After contacting and entering a cell, the small activating nucleic acid molecule of the present invention can effectively activate or up-regulate the expression of the Thpo/THPO gene in the cell, preferably by at least 10%.

Another aspect of the present invention also relates to a nucleic acid encoding a small activating nucleic acid molecule of the invention. In one embodiment, the nucleic acid is a DNA molecule.

Another aspect of the present invention provides a cell comprising the aforementioned small activating nucleic acid molecule or the nucleic acid encoding the small activating nucleic acid molecule described herein. In one embodiment, a small activating nucleic acid molecule of the present invention can be a double-stranded small activating nucleic acid molecule, such as a double-stranded small activating RNA (saRNA) molecule, which targets the promoter region of the Thpo/THPO gene comprising a first nucleic acid strand and a second nucleic acid strand. In another embodiment, a small activating nucleic acid molecule of the present invention can be a single-stranded small activating nucleic acid molecule, such as a hairpin-structured single-stranded nucleic acid, targeting the promoter region of the Thpo/THPO gene, wherein the hairpin-structured single-stranded nucleic acid produces a double-stranded small activating RNA (saRNA) molecule in a cell after being introduced into the cell.

Another aspect of the present invention provides a composition (e.g., a pharmaceutical composition). The composition comprises a small activating nucleic acid molecule of the present invention or a nucleic acid encoding the small activating nucleic acid molecule described herein, and optionally, a pharmaceutically acceptable carrier. In one embodiment, the pharmaceutically acceptable carrier can comprise or be selected from a liposome, a high-molecular polymer, and a polypeptide.

In another aspect of the present invention, a formulation is provided which comprises a small activating nucleic acid molecule described in the present invention, a nucleic acid encoding the small activating nucleic acid molecule described in the present invention, or a cell comprising a small activating nucleic acid molecule of the present invention or a nucleic acid encoding a small activating nucleic acid molecule of the present invention, or a composition comprising a small activating nucleic acid molecule of the present invention.

In another aspect of the present invention, a kit is provided which comprises a small activating nucleic acid molecule of the present invention, a nucleic acid encoding a small activating nucleic acid molecule described herein, or a cell comprising a small activating nucleic acid molecule of the present invention or a nucleic acid encoding a small activating nucleic acid molecule of the present invention, or a composition comprising a small activating nucleic acid molecule of the present invention.

Another aspect of the present invention relates to uses of the small activating nucleic acid molecule described in the present invention, a nucleic acid encoding the small activating nucleic acid molecule of the present invention, a cell comprising the small activating nucleic acid molecule of the present invention, or the nucleic acid encoding the small activating nucleic acid molecule of the present invention, or a composition comprising the small activating nucleic acid molecule of the present invention in the preparation of a drug or formulation for activating/up-regulating the expression of the Thpo/THPO gene in a cell.

Another aspect of the present invention also relates to a method for activating/up-regulating the expression of the Thpo/THPO gene in a cell. The method comprises administering the small activating nucleic acid molecule described in the present invention, a nucleic acid encoding the small activating nucleic acid molecule of the present invention or a composition or formulation comprising the small activating nucleic acid molecule of the present invention to a cell. In one embodiment, the method for activating/up-regulating the expression of the Thpo/THPO gene in the cell comprises administering the small activating nucleic acid molecule described in the present invention, the nucleic acid encoding the small activating nucleic acid molecule of the present invention, or the composition or formulation comprising the small activating nucleic acid molecule of the present invention to the cell. The aforementioned cell comprises a mammal cell, e.g., a cell from a human body, such as a human embryo liver cell, a human hepatoma cell, such as an HepG2 cell, and a human liver cell, or a cell from a mouse, such as a mouse embryonic liver cell, such as embryonic cell line BNL·CL2, a mouse liver cancer cell, such as the liver cancer cell line LPC-H12, and a mouse primary liver cell. The aforementioned cell can be in vitro, or can be present in a mammalian body, such as a mouse or human body.

The small activating nucleic acid molecule of the present invention can be directly introduced into a cell, or can be produced in the cell after a nucleotide sequence encoding the small activating nucleic acid molecule of the present invention is introduced into the cell. The cell is preferably a mammalian cell, more preferably a mouse or human cell. The aforementioned cell may be in vitro, such as a cell line or a cell strain, or may present in a mammalian body, such as a mouse or human body, and more specifically, such as a liver cell from a mouse or human body. The human body may be a patient suffering from a disease or condition related to insufficient or decreased expression of the THPO protein, or a patient with thrombocytopenia due to various causes. The small activating nucleic acid molecule of the present invention can be administered at a sufficient dose to treat the disease or condition related to a deficiency in the amount of THPO protein or insufficient or decreased expression of the THPO protein. Specifically, the disease or condition related to a deficiency in the amount of THPO protein or insufficient or decreased expression of the THPO protein may be thrombocytopenia.

Another aspect of the present invention provides an isolated small activating nucleic acid molecule acting site of the Thpo/THPO gene, which has any continuous sequence of 16 to 35 nucleotides in length in the promoter region of the Thpo/THPO gene, and preferably, the acting site comprises or is selected from any continuous sequence of 16 to 35 nucleotides in length in any of the sequences set forth in SEQ ID NOs: 2-4 and SEQ ID NOs: 601-605. Specifically, the acting site may comprise or be selected from any nucleotide sequence set forth in SEQ ID NOs: 200-296 and SEQ ID NOs: 606-1047.

Another aspect of the present invention relates to a method for treating a disease or condition related to insufficient or decreased expression of the THPO protein in a subject such as a human or mouse or thrombocytopenia, which comprises administering a small activating nucleic acid molecule of the present invention, a nucleic acid encoding a small activating nucleic acid molecule of the present invention, a cell comprising a small activating nucleic acid molecule of the present invention, or a nucleic acid encoding a small activating nucleic acid molecule of the present invention, or a composition comprising a small activating nucleic acid molecule of the present invention at a curative dose to the subject. In one embodiment, a method for treating thrombocytopenia and/or a disease or condition related to insufficient or decreased expression of THPO protein in a subject in the present invention can comprise administering a small activating nucleic acid molecule of the present invention, a nucleic acid encoding the small activating nucleic acid molecule of the present invention, a cell comprising the small activating nucleic acid molecule of the present invention, or a nucleic acid encoding a small activating nucleic acid molecule of the present invention, or a composition comprising the small activating nucleic acid molecule of the present invention, and other formulations at a curative dose to the subject, wherein the other formulations comprises, such as a low-molecular-weight compound, antibody, polypeptide or protein, etc. The subject can be a mammal, e.g., a mouse or a human. In the present invention, thrombocytopenia is caused by various factors, including, but not limited to, myelo-thrombocytopenia, increased peripheral platelet destruction, and splenic sequestration. Factors causing a reduction in platelets comprise congenital (hereditary) and acquired thrombocytopenia. In one embodiment, factors causing acquired thrombocytopenia include, but are not limited to, thrombocytopenia caused by aplastic anemia, myelodysplastic syndromes, leukemia, drugs, infection, tumor diseases, radiotherapy, bone marrow transplantation, and chronic liver diseases, and immune thrombocytopenia (ITP). Factors causing increased platelet destruction include, but are not limited to, thrombotic thrombocytopenia, heparin-induced thrombocytopenia, drug-induced thrombocytopenia, ITP, and thrombotic thrombocytopenia purpura. In one embodiment, thrombocytopenia is related to a disease or condition related to insufficient or decreased expression of the THPO protein. In another embodiment, thrombocytopenia is related to insufficient or decreased expression of the THPO protein. Thrombocytopenia can comprise drug-induced thrombocytopenia, immune thrombocytopenia, thrombotic thrombocytopenia purpura, and the like. The thrombocytopenia described herein can be caused by various factors, such as myelo-thrombocytopenia, increased peripheral platelet destruction and splenomegaly sequestration, such as tumor radiotherapy, tumor chemotherapy, bone marrow transplantation, chronic liver disease, drug-induced thrombocytopenia, immune thrombocytopenia, thrombotic thrombocytopenic purpura, and hereditary thrombocytopenia. Thrombocytopenia is generally characterized clinically by ecchymosis, changes in megakaryocytes in the bone marrow, mucosal bleeding or intracranial bleeding, all of which will endanger the lives of patients with severe thrombocytopenia. When the platelet concentrations are lower than 100×10⁹/L by direct blood platelet count, thrombocytopenia is considered to be present (Kuter 2009). When the platelet concentrations are lower than 50×10⁹/L, spontaneous bleeding, commonly manifesting as subcutaneous purpuraa, occurs frequently. When the platelet concentrations are lower than 20×10⁹/L, a patient's condition can be life-threatening, caused by trauma or sudden intracranial bleeding, gastrointestinal bleeding, and the like. In the method of the present invention, the thrombocytopenia in the subject can be characterized by a platelet concentration of less than 100×10⁹/L, less than 50×10⁹/L, or less than 20×10⁹/L as indicated by direct blood platelet count. In one embodiment, a disease or condition related to insufficient or decreased expression of the THPO protein in a subject is alleviated, relieved, or cured after administering a small activating nucleic acid molecule of the present invention, a nucleic acid encoding the small activating nucleic acid molecule of the present invention, the cell comprising the small activating nucleic acid molecule of the present invention, or the nucleic acid encoding the small activating nucleic acid molecule of the present invention, or the composition comprising the small activating nucleic acid molecule of the present invention at a curative dose.

Another aspect of the present invention relates to a method for treating thrombocytopenia in a subject, which comprises administering a small activating nucleic acid molecule of the present invention, a nucleic acid encoding the small activating nucleic acid molecule of the present invention, a cell comprising the small activating nucleic acid molecule of the present invention, a nucleic acid encoding the small activating nucleic acid molecule of the present invention, or a composition comprising the small activating nucleic acid molecule of the present invention at a curative dose to the subject. In the method of the present invention, the thrombocytopenia in the subject can be characterized by a platelet concentration of less than 100×10⁹/L, less than 50×10⁹/L, or less than 20×10⁹/L as indicated by direct blood platelet count. In a method of the present invention, the thrombocytopenia can be related to insufficient or decreased expression of the THPO protein, drug-induced thrombocytopenia, immune thrombocytopenia, thrombotic thrombocytopenic purpuraa, and the like. The thrombocytopenia as described herein can be caused due to various factors such as myelo-thrombocytopenia, increased peripheral platelet destruction and splenomegaly sequestration, such as tumor radiotherapy, tumor chemotherapy, bone marrow transplantation, chronic liver disease, drug-induced thrombocytopenia, immune thrombocytopenia, thrombotic thrombocytopenic purpura, and hereditary thrombocytopenia. In one embodiment, the method for treating thrombocytopenia of the present invention comprises administering a small activating nucleic acid molecule of the present invention, a nucleic acid encoding a small activating nucleic acid molecule of the present invention, a cell comprising a small activating nucleic acid molecule of the present invention or a nucleic acid encoding the small activating nucleic acid molecule of the present invention, or a composition comprising a small activating nucleic acid molecule of the present invention and/or the aforementioned the small activating nucleic acid molecule, the nucleic acid encoding the aforementioned small activating nucleic acid molecule, the cell comprising the small activating nucleic acid molecule of the present invention or the nucleic acid encoding the small activating nucleic acid molecule of the present invention, or a composition consisting of the composition comprising the small activating nucleic acid molecule of the present invention and one or more other formulations at a curative dose to the subject, wherein the other formulation comprises, such as a low-molecular-weight compound, antibody, polypeptide and protein. The subject can be a mammal, such as a mouse or human. In the aforementioned method, the thrombocytopenia can comprise drug-induced thrombocytopenia, immune thrombocytopenia, thrombotic thrombocytopenia purpura, and the like. In the aforementioned method, the subject comprises a mammal, such as a mouse or human. In one embodiment, the thrombocytopenia in a subject is alleviated, relieved, or cured after administering the small activating nucleic acid molecule of the present invention, the nucleic acid encoding the small activating nucleic acid molecule of the present invention, the cell comprising the small activating nucleic acid molecule of the present invention, or the nucleic acid encoding the small activating nucleic acid molecule of the present invention, or the composition comprising the small activating nucleic acid molecule of the present invention at a curative dose. Preferably, the alleviation, relief, or cure may be an increase or recovery of the platelet concentration to near normal physiological levels, such as in the range of 100×10⁹/L to 300×10⁹/L, as indicated by direct blood platelet count.

Another aspect of the present invention relates to a use of a small activating nucleic acid molecule of the present invention, a nucleic acid encoding the small activating nucleic acid molecule of the present invention, a cell comprising the small activating nucleic acid molecule of the present invention or the nucleic acid encoding the small activating nucleic acid molecule of the present invention, or the composition comprising the small activating nucleic acid molecule of the present invention in the preparation of a drug for treating a disease or condition related to thrombocytopenia and/or insufficient or decreased expression of the THPO protein. The subject can be a mammal, such as a human. In uses of present invention, thrombocytopenia is caused by various factors, including, but not limited to, myelo-thrombocytopenia, increased peripheral platelet destruction, and splenic sequestration. Factors causing a reduction in platelets comprise congenital (hereditary) and acquired thrombocytopenia. In one embodiment, thrombocytopenia caused by acquired factors include, but are not limited to, thrombocytopenia caused by aplastic anemia, myelodysplastic syndromes, leukemia, drugs, infection, tumor diseases, radiotherapy, bone marrow transplantation, and chronic liver diseases, and immune thrombocytopenia (ITP). Factors causing increased platelet destruction include, but are not limited to, thrombotic thrombocytopenia, heparin-induced thrombocytopenia, drug-induced thrombocytopenia, ITP, and thrombotic thrombocytopenia purpura. In one embodiment, the thrombocytopenia can comprise, for example, thrombocytopenia related to insufficient or decreased expression of the THPO protein. Preferably, the thrombocytopenia can comprises or be selected from thrombocytopenia related to insufficient or decreased expression of the THPO protein, drug-induced thrombocytopenia, immune thrombocytopenia, thrombotic thrombocytopenic purpuraa, and the like. Thrombocytopenia as described herein can be caused by various factors such as tumor radiotherapy, tumor chemotherapy, bone marrow transplantation, chronic liver disease, splenomegaly sequestration, transfusion of banked blood with low platelet content, and an increase of platelet consumption due to drugs. In one embodiment, thrombocytopenia can be manifested as a platelet concentration of less than 100×10⁹/L, less than 50×10⁹/L, or less than 20×10⁹/L as indicated by direct blood platelet counts.

In one embodiment, a use of a small activating nucleic acid molecule described in the present invention, a nucleic acid encoding the small activating nucleic acid molecule of the present invention, a cell comprising the small activating nucleic acid molecule of the present invention or the nucleic acid encoding the small activating nucleic acid molecule of the present invention, or the composition comprising the small activating nucleic acid molecule of the present invention in the preparation of a drug for treating thrombocytopenia or a disease related to insufficient or decreased expression of the THPO protein is provided. In one embodiment, thrombocytopenia can comprise thrombocytopenia related to insufficient or decreased expression of THPO protein, and preferably, thrombocytopenia can comprise drug-induced thrombocytopenia, immune thrombocytopenia, thrombotic thrombocytopenic purpuraa, and the like. In one embodiment, thrombocytopenia can be manifested as a platelet concentration of less than 100×10⁹/L, less than 50×10⁹/L, or less than 20×10⁹/L as indicated by direct blood platelet count.

Another aspect of the present invention relates to a use of the small activating nucleic acid molecule described in the present invention, the nucleic acid encoding the small activating nucleic acid molecule of the present invention, the cell comprising the small activating nucleic acid molecule of the present invention or the nucleic acid encoding the small activating nucleic acid molecule of the present invention, or the composition or formulation comprising the small activating nucleic acid molecule of the present invention in the preparation of a drug or pharmaceutical composition for treating thrombocytopenia. Preferably, the thrombocytopenia can comprise or be selected from thrombocytopenia related to insufficient or decreased expression of the THPO protein or thrombocytopenia due to other various causes, such as drug-induced thrombocytopenia, immune thrombocytopenia, thrombotic thrombocytopenic purpuraa, and the like. The thrombocytopenia described herein can be caused by various factors such as tumor radiotherapy, tumor chemotherapy, bone marrow transplantation, chronic liver disease, splenomegaly sequestration, transfusion of banked blood with low platelet content and an increase of platelet consumption due to drugs. In one embodiment, thrombocytopenia can be manifested as a platelet concentration of less than 100×10⁹/L, less than 50×10⁹/L, or less than 20×10⁹/L as indicated by direct blood platelet count.

In one embodiment, a use of s small activating nucleic acid molecule described in the present invention, a nucleic acid encoding the small activating nucleic acid molecule of the present invention, a cell comprising the small activating nucleic acid molecule of the present invention or the nucleic acid encoding the small activating nucleic acid molecule of the present invention, or a composition or formulation comprising the small activating nucleic acid molecule of the present invention in the preparation of a drug or pharmaceutical composition for treating a disease related to insufficient or decreased expression of the THPO protein or thrombocytopenia.

### Advantages of the Present Invention

The small activating nucleic acid molecule, such as small activating RNA (saRNA), is capable of activating/up-regulating endogenous Thpo/THPO gene expression provided by the present invention and can specifically activate Thpo/THPO gene, thereby up-regulating or restoring the expression of Thpo/THPO gene and protein with lower toxic and side effects, which can be used for treating thrombocytopenia caused by insufficient platelet production or excessive destruction due to insufficient Thpo/THPO protein expression and various causes, or in preparing a drug or formulation for treating thrombocytopenia.

### BRIEF DESCRIPTION OF THE DRAWINGS

FIG. 1 shows changes in the expression of mouse Thpo mRNA mediated by mouse Thpo saRNA. BNL·CL2 cells were transfected with 322 saRNAs targeting mouse Thpo promoter at a final concentration of 10 nM, and Thpo mRNA expression was analyzed 72 h after transfection using one-step RT-qPCR. The figure shows the descending order of mRNA expression changes (log₂) of Thpo relative to control treatment (Mock). The ordinate values represent the mean of 2 repeat treatment±SD.

FIG. 2 shows the hotspot region of mouse Thpo saRNA on the mouse Thpo promoter. BNL·CL2 cells were transfected with 322 saRNAs targeting mouse Thpo promoter at a final concentration of 10 nM for 72 h, and Thpo mRNA expression was analyzed after transfection using one-step RT-qPCR. The figure shows changes in the expression of Thpo relative to control treatment (Mock) ranked from −500 to −0 according to the target positions of the saRNA in the Thpo promoter. Black solid dots represent functional saRNAs, white open dots represent non-functional saRNAs, and dotted lines frame the hotspot regions where 3 functional saRNAs gather (H1 to H3). The ordinate values represent the mean of 2 repeat treatment.

FIG. 3 shows the screening results of two-step RT-qPCR validated mouse Thpo saRNA HTS. As shown, the BNL·CL2 cells were transfected with saRNA at a final concentration of 10 nM for 72 h. After transfection, RNA was extracted with Qiagen RNeasy kit and qPCR amplification was performed with ABI 7500 rapid real-time PCR system after reverse transcription. The Tbp gene was amplified as internal control. The relative expression values of Thpo mRNA are shown after treatment of cells with a single saRNA. Mock, dsCon2, and DS03-432i are shown respectively as blank transfection, sequence-independent double-stranded RNA transfection, and small interference RNA control transfection. The ordinate values represent the mean of 2 repeat treatment±SD.

FIG. 4 shows the screening results of two-step RT-qPCR validated mouse Thpo saRNA HTS. As shown, the LPC-H12 cells were transfected with saRNA at a final concentration of 10 nM for 72 h. After transfection, RNA was extracted with Qiagen RNeasy kit after transfection and qPCR amplification was performed with ABI 7500 rapid real-time PCR system after reverse transcription. The Tbp gene was amplified as internal control. The relative expression values of Thpo mRNA are shown after treatment of cells with a single saRNA. Mock, dsCon2, and DS03-432i are shown respectively as blank transfection, sequence-independent double-stranded RNA transfection, and small interference RNA control transfection. The ordinate values represent the mean of 2 repeat treatment±SD.

FIG. 5 shows the screening results of two-step RT-qPCR validated mouse Thpo saRNA HTS in a primary mouse liver cell. As shown, the primary mouse liver cell were transfected with saRNA at a final concentration of 10 nM for 72 h. After transfection, RNA was extracted with Qiagen RNeasy kit after transfection and qPCR amplification was performed with ABI 7500 rapid real-time PCR system after reverse transcription. The Tbp gene was amplified as internal control. The relative expression values of Thpo mRNA are shown after treatment of cells with a single saRNA. Mock, dsCon2, and DS03-432i are shown respectively as blank transfection, sequence-independent double-stranded RNA transfection, and small interference RNA control transfection. The ordinate values represent the mean of 2 repeat treatment±SD.

FIG. 6 shows that mouse Thpo saRNA increases concentration of Thpo in mice serum. Fifteen male Balb/c mice were randomly divided into 3 groups with 5 mice in each group, and received PBS, LNP-entrapped Thpo-siRNA (DS03-432i, 5 mg/kg) and LNP-entrapped Thpo-saRNA (DS03-0024, 5 mg/kg) by tail vein injection. Blood samples were collected after 72 h and serum was separated. A mouse Thpo ELISA kit was used to determine Thpo protein levels in mice serum. PBS is a blank control and DS03-432i is a small interfering RNA control. (***p<0.001).

FIG. 7 shows that mouse Thpo saRNA increases concentration of Thpo in mice serum. Thirty two male Balb/c mice were randomly divided into 4 groups with 8 mice in each group, and received PBS and LNP-entrapped DS03-0024 (1 mg/kg, 3 mg/kg and 6 mg/kg) by tail vein injection. Blood samples were collected and serum was isolated 1 day prior to administration and 2 days after administration. A mouse Thpo ELISA kit was used to determine the Thpo protein levels in mice serum (****p<0.0001).

FIG. 8 shows that mouse Thpo saRNA increases the number of platelets of mouse. Thirty two male Balb/c mice were randomly divided into 4 groups with 8 mice in each group, and received PBS and LNP-entrapped DS03-0024 (1 mg/kg, 3 mg/kg and 6 mg/kg) by tail vein injection. Blood samples were collected 6 days after administration, and platelets were collected using a mouse peripheral platelet isolation kit, which utilized a labeled CD41a-FITC antibody to facilitate counting using a flow cytometer. (*p<0.05).

FIG. 9 shows that changes in the number of platelets of mouse caused by mouse Thpo saRNA 9 days after administration. Thirty two male Balb/c mice were randomly divided into 4 groups with 8 mice in each group, and received PBS and LNP-entrapped DS03-0024 (1 mg/kg, 3 mg/kg and 6 mg/kg) by tail vein injection. Blood samples were collected 17 days after administration and platelets were collected using a mouse peripheral platelet isolation kit, which utilized a labeled CD41a-FITC antibody to facilitate counting using a flow cytometer. (*p<0.05).

FIG. 10 shows changes in the expression of human THPO mRNA mediated by human THPO saRNA. HepG2 cells were transfected with candidate saRNAs targeting the human THPO promoter at a final concentration of 25 nM. Human THPO mRNA expression was analyzed 72 h after transfection using one-step RT-qPCR. The figure shows the descending order of mRNA expression changes (log₂) of THPO relative to control treatment (Mock). The ordinate values represent the mean of 2 repeat treatment±SD.

FIG. 11 shows the hotspot region of human THPO saRNA on the human THPO promoter. HepG2 cells were transfected with candidate saRNAs targeting the human THPO promoter at a final concentration of 25 nM for 72 h. Human THPO mRNA expression was analyzed after transfection using one-step RT-qPCR. The figure shows changes in the expression of THPO relative to control treatment (Mock) ranked according to the target positions of the saRNA in the THPO promoter. Black solid dots represent functional saRNAs, white open dots represent non-functional saRNAs, and dotted lines frame the hotspot regions where functional saRNAs gather. The ordinate values represent the mean of 2 repeat treatment±SD.

FIG. 12 shows the screening results of two-step RT-qPCR validated human THPO saRNA HTS. HepG2 cells were transfected with saRNA at a final concentration of 25 nM for 72 h. After transfection, RNA was extracted with Qiagen RNeasy kit. After reverse transcription, qPCR amplification was performed with ABI 7500 rapid real-time PCR system. TBP and HPRT1 genes were amplified as internal controls. Shown are the relative mRNA expression values of THPO after treating cells with a single saRNA. Shown are Mock, dsCon2, and siTHPO (DS3A-365i) which are blank transfection, sequence-independent double-stranded RNA transfection, and small interference RNA control transfection, respectively. The ordinate values represent the mean of 2 repeat treatment±SD.

FIG. 13 shows the effect of human THPO saRNA on the expression of human THPO protein by ELISA method. HepG2 cells were transfected with saRNA at a final concentration of 25 nM for 72 h. After transfection, culture medium was taken and the supernatant was centrifuged to remove dead cell debris. The level of THPO protein secreted into the culture medium was determined by ELISA kit (R & D Systems, DTP00B). Shown are the expression values of THPO protein after treating the cells with a single saRNA. Shown are Mock, dsCon2, and siTHPO (DS3A-365i) which are blank transfection, sequence-independent double-stranded RNA transfection, and small interference RNA control transfection, respectively. The ordinate values represent the mean of 2 repeat treatment±SD.

### DETAILED DESCRIPTION

In the present invention, the related terms are defined as follows.

The term “complementarity” as used herein refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity comprises complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the double-stranded oligonucleotide molecule without “mispairing”. “Incomplete complementarity” means that not all the nucleotide units of the two strands are bonded with each other by hydrogen bonds. For example, for two oligonucleotide strands each of 20 nucleotides in length in the double-stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95%, about 99% or about 100% complementarity.

The term “oligonucleotide” as used herein refers to polymers of nucleotides, and includes, but is not limited to, single-stranded or double-stranded molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. The oligonucleotide for activating target gene transcription described herein is a small activating nucleic acid molecule.

The terms “oligonucleotide strand” and “oligonucleotide sequence” as used herein can be used interchangeably, referring to a generic term for short nucleotide sequences having less than 35 bases (including nucleotides in deoxyribonucleic acid (DNA) or ribonucleic acid (RNA)). In the present invention, an oligonucleotide strand may have any of 16 to 35 nucleotides in length.

As used herein, the term “first nucleic acid strand” can be a sense strand or an antisense strand. The sense strand of a small activating RNA refers to a nucleic acid strand contained in a small activating RNA duplex which has identity to the coding strand of the promoter DNA sequence of a target gene, and the antisense strand refers to a nucleic acid strand in the small activating RNA duplex which is complementary to the sense strand.

As used herein, the term “second nucleic acid strand” can also be a sense strand or an antisense strand. If the first oligonucleotide strand is a sense strand, the second oligonucleotide strand is an antisense strand; if the first oligonucleotide strand is an antisense strand, the second oligonucleotide strand is a sense strand.

The term “gene” as used herein refers to all nucleotide sequences required to encode a polypeptide chain or to transcribe a functional RNA. “Gene” can be an endogenous or fully or partially recombinant gene for a host cell (for example, because an exogenous oligonucleotide and a coding sequence for encoding a promoter are introduced into a host cell, or a heterogeneous promoter adjacent to an endogenous encoding sequence is introduced into a host cell). For example, the term “gene” comprises a nucleic acid sequence consisting of exons and introns. Protein-encoding sequences are, for example, sequences contained within exons in an open reading frame between an initiation codon and a termination codon, and as used herein, “gene” can comprise such as a gene regulatory sequence, such as a promoter, an enhancer, and all other sequences known in the art for controlling the transcription, expression or activity of another gene, no matter whether the gene comprises a coding sequence or a non-coding sequence. In one case, for example, “gene” can be used to describe a functional nucleic acid comprising a regulatory sequence such as a promoter or an enhancer. The expression of a recombinant gene can be controlled by one or more types of heterogeneous regulatory sequences.

The term “target gene” as used herein can refer to nucleic acid sequences naturally present in organisms, transgenes, viral or bacterial sequences, can be chromosomes or extrachromosomal genes, and/or can be transiently or stably transfected or incorporated into cells and/or chromatins thereof. The target gene can be a protein-encoding gene or a non-protein-encoding gene (such as a microRNA gene and a long non-coding RNA gene). The target gene generally contains a promoter sequence, and the positive regulation for the target gene can be achieved by designing a small activating nucleic acid molecule having sequence identity (also called homology) to the promoter sequence, characterized as the up-regulation of expression of the target gene. “Sequence of a target gene promoter” refers to a non-coding sequence of the target gene, and the reference of the sequence of a target gene promoter in the phrase “complementary to the sequence of a target gene promoter” of the present invention refers to a coding strand of the sequence, also known as a non-template strand, i.e., a nucleic acid sequence having the same sequence as the coding sequence of the gene. “Target” or “target sequence” refers to a sequence fragment in the sequence of a target gene promoter which is homologous or complementary with a sense oligonucleotide strand or an antisense oligonucleotide strand of a small activating nucleic acid molecule.

As used herein, the terms “sense strand” and “sense nucleic acid strand” can be used interchangeably, and the sense oligonucleotide strand of a small activating nucleic acid molecule refers to the first nucleic acid strand having identity to the coding strand of the sequence of a target gene promoter in the small activating nucleic acid molecule duplex.

As used herein, the terms “antisense strand” and “antisense nucleic acid strand” can be used interchangeably, and the antisense oligonucleotide strand of a small activating nucleic acid molecule refers to the second nucleic acid strand complementary with the sense oligonucleotide strand in the small activating nucleic acid molecule duplex.

The term “coding strand” as used herein refers to a DNA strand in the target gene which cannot be used for transcription, and the nucleotide sequence of this strand is the same as that of an RNA produced from transcription (in the RNA, T in DNA is replaced by U). The coding strand of the double-stranded DNA sequence of the target gene promoter described herein refers to a promoter sequence on the same DNA strand as the DNA coding strand of the target gene.

The term “template strand” as used herein refers to the other strand complementary with the coding strand in the double-stranded DNA of the target gene, i.e., the strand that, as a template, can be transcribed into RNA, and this strand is complementary with the transcribed RNA (A to U and G to C). In the process of transcription, RNA polymerase binds to the template strand, moves along the 3′→5′ direction of the template strand, and catalyzes the synthesis of the RNA along the 5′→3′ direction. The template strand of the double-stranded DNA sequence of the target gene promoter described herein refers to a promoter sequence on the same DNA strand as the DNA template strand of the target gene.

The term “promoter” as used herein refers to a sequence which plays a regulatory role for the transcription of a protein-coding or RNA-coding nucleic acid sequence by associating with them spatially. Generally, a eukaryotic gene promoter contains 100 to 5000 base pairs, although this length range is not intended to limit the term “promoter” as used herein. Although the promoter sequence is generally located at the 5′ terminus of a protein-coding or RNA-coding sequence, the promoter sequence may also exist in exon and intron sequences.

The term “transcription start site” as used herein refers to a nucleotide marking the transcription start on the template strand of a gene. The transcription start site may appear on the template strand of the promoter region. A gene can have more than one transcription start site.

The term “identity” or “homology” as used herein means that one oligonucleotide strand (a sense or an antisense strand) of a small activating RNA has similarity to a coding strand or a template strand in a region of the promoter sequence of a target gene. As used herein, the “identity” or “homology” may be at least about 75%, about 79%, about 80%, about 85%, about 90%, about 95%, about 99% or about 100%.

The term “overhang” as used herein refers to non-base-paired nucleotides at the terminus (5′ or 3′) of an oligonucleotide strand, which is formed by one strand extending out of the other strand in a double-stranded oligonucleotide. A single-stranded region extending out of the 3′ terminus and/or 5′ terminus of a duplex is referred to as an overhang.

As used herein, the terms “gene activation”, “activating gene expression”, “gene up-regulation” and “up-regulating gene expression” can be used interchangeably, and mean an increase in transcription, translation, expression or activity of a certain nucleic acid as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “gene activation”, “activating gene expression”, “gene up-regulation” or “up-regulating gene expression” refers to an increase in activity associated with a nucleic acid sequence, regardless of the mechanism of such activation. For example, the nucleic acid sequence plays a regulatory role as a regulatory sequence, the nucleic acid sequence is transcribed into RNA and the RNA is translated into a protein, thereby increasing the expression of the protein.

As used herein, the terms “small activating RNA”, “saRNA”, and “small activating nucleic acid molecule” can be used interchangeably, and refer to a nucleic acid molecule that can up-regulate target gene expression and can be composed of the first nucleic acid fragment (antisense nucleic acid strand, also referred to as antisense oligonucleotide strand) containing a nucleotide sequence having sequence identity or homology with the non-coding nucleic acid sequence (e.g., a promoter and an enhancer) of a target gene and the second nucleic acid fragment (sense nucleic acid strand, also referred to as sense oligonucleotide strand) containing a nucleotide sequence complementary with the first nucleic acid fragment, wherein the first nucleic acid fragment and the second nucleic acid fragment form a duplex. The small activating nucleic acid molecule can also be composed of a synthesized or vector-expressed single-stranded RNA molecule that can form a hairpin structure by two complementary regions within the molecule, wherein the first region comprises a nucleotide sequence having sequence identity to the target sequence of a promoter of a gene, and the second region comprises a nucleotide sequence which is complementary to the first region. The length of the duplex region of the small activating nucleic acid molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45, or about 50 base pairs. In addition, the terms “saRNA”, “small activating RNA”, and “small activating nucleic acid molecule” also comprise nucleic acids other than the ribonucleotide, including, but not limited to, modified nucleotides or analogues.

As used herein, the term “hotspot” refers to a gene promoter region of at least 30 bp in length. The gathering of targets of functional small activating nucleic acid molecules appears in these hotspot regions, wherein at least 30% of the small activating nucleic acid molecules targeting these hotspot regions can induce a 1.1-fold to 2.89-fold in the mRNA expression of a target gene.

As used herein, the term “synthesis” refers to a method for synthesis of an oligonucleotide, including any method allowing RNA synthesis, such as chemical synthesis, in vitro transcription, and/or vector-based expression.

According to the present invention, the expression of the Thpo/THPO gene is up-regulated by RNA activation, and a related disease (particularly thrombocytopenia) is treated by increasing the expression of the THPO protein. The Thpo/THPO gene in the present invention is sometimes also called a target gene.

The method for preparing the small activating nucleic acid molecule provided by the present invention comprises sequence design and sequence synthesis.

The synthesis of the sequence of the small activating nucleic acid molecule of the present invention can be performed by adopting a chemical synthesis or can be entrusted to a biotechnology company specialized in nucleic acid synthesis.

Generally speaking, the chemical synthesis comprises the following four steps: (1) synthesis of oligomeric ribonucleotides, (2) deprotection, (3) purification and isolation; and (4) desalination and annealing.

For example, the specific steps for chemically synthesizing the saRNA described herein are as follows:

(1) Synthesis of Oligomeric Ribonucleotides

Synthesis of 1 μM RNA was set in an automatic DNA/RNA synthesizer (e.g., Applied Biosystems EXPEDITE8909), and the coupling time of each cycle was also set as 10 to 15 min. With a solid phase-bonded 5′-O-p-dimethoxytriphenylmethyl-thymidine substrate as an initiator, one base was bonded to the solid phase substrate in the first cycle, and then, in the n^{th} (19≥n≥2) cycle, one base was bonded to the base bonded in the n−1^{th} cycle. This process was repeated until the synthesis of the whole nucleic acid sequence was completed.

(2) Deprotection

The solid phase substrate bonded with the saRNA was put into a test tube, and 1 mL of a mixed solution of ethanol and ammonium hydroxide (volume ratio: 1:3) was added to the test tube. The test tube was then sealed and placed in an incubator, and the mixture was incubated at 25-70° C. for 2 to 30 h. The solution containing the solid phase substrate bonded with the saRNA was filtered, and the filtrate was collected. The solid phase substrate was rinsed with double distilled water twice (1 mL each time), and the filtrate was collected. The collected eluent was combined and dried under vacuum for 1 to 12 h. Then the solution was added with 1 mL of a solution of tetrabutylammonium fluoride in tetrahydrofuran (1 M), let stand at room temperature for 4 to 12 h, followed by addition of 2 mL of n-butanol. Precipitate was collected to give a single-stranded crude product of saRNA by high-speed centrifugation.

(3) Purification and Isolation

The resulting crude product of saRNA was dissolved in 2 mL of triethylamine acetate solution with a concentration of 1 mol/L, and the solution was separated by a reversed-phase C18 column of high pressure liquid chromatography to give a purified single-stranded product of saRNA.

(4) Desalination and Annealing

Salts were removed by gel filtration (size exclusion chromatography). A single sense oligomeric ribonucleic acid strand and a single antisense oligomeric ribonucleic acid strand were mixed in a 1 to 2 mL of buffer (10 mM Tris, pH=7.5-8.0, 50 mM NaCl) at a molar ratio of 1:1. The solution was heated to 95° C., and was then slowly cooled to room temperature to give a solution containing saRNA.

It was discovered in this study that after being introduced into a cell, the aforementioned saRNA could effectively increase Thpo/THPO mRNA and expression of THPO protein.

The present invention will be further illustrated with reference to specific examples and drawings below. It should be understood that these examples are merely intended to illustrate the present invention rather than limit the scope of the present invention. In the following examples, study methods without specific conditions were generally in accordance with conventional conditions, such as conditions described in Sambrook, et al., Molecular Cloning: Laboratory Manual (New York: Cold Spring Harbor Laboratory Press, 1989), or conditions recommended by the manufacturer.

### EXAMPLES

### Example 1: Design and Synthesis of saRNA Targeting Mouse Thpo Gene Promoter

To screen functional saRNAs capable of activating Thpo gene expression, a Thpo promoter sequence with a length of 500 bp was used as a template, a target with a length of 19 bp was selected from −500 bp upstream of TSS. Target sequences were filtered and the criteria for retaining target sequences were: (1) GC content between 35% and 65%, (2) with less than 5 continuous identical nucleotides, (3) with 3 or less dinucleotide repeat sequences; and (4) with 3 or less trinucleotide repeat sequences. There were 322 target sequences remaining after filtration and these entered the screening process as candidates. Corresponding double-stranded saRNAs were chemically synthesized based on these candidate sequences.

Each sense strand and antisense strand in the double-stranded saRNA used in the study had 21 nucleotides in length. The 19 nucleotides in the 5′ region of the first ribonucleic acid strand (sense strand) of the double-stranded saRNA had 100% identity to the target sequence of the promoter, and the 3′ terminus of the first ribonucleic acid strand contained a TT sequence. The 19 nucleotides in the 5′ region of the second ribonucleic acid strand were complementary to the first ribonucleic acid strand sequence, and the 3′ terminus of the second ribonucleic acid strand contained a TT sequence. The aforementioned two strands of the double-stranded saRNA were mixed at a molar ratio of 1:1, and after annealing, a double-stranded saRNA was formed.

The sequence of the mouse Thpo promoter region is shown as follows, which corresponds to position 1 to position 500 from 5′ to 3′ of SEQ ID No:1 in the sequence listing:

| | |
|---|---|
| −500 | taatataggc aatacctact ttgccagttg gaaaggggaa atgccaggaa |
| −450 | agacactcct ctggagcagg ggaatataga aggaaaagca agcaagccag |
| −400 | catgctgtca gaattcagca gcaggtatac tgttcaggga aaagaaattt |
| −350 | ggagagccag gaactgagac ttcatccatc taaaacaaga cctctctttc |
| −300 | ccagagagaa ccgtattctg ccagatttct cctggactaa ccctcttctc |
| −250 | tctctcactt aggtcccagg ttctcatgtc gccaggacat gagacagttc |
| −200 | ccgctataca aattctctct agagtgtgtg tgggtggagg agctgggctc |
| −150 | tattcatgga ccatggtcca gccatgggct ctagtcccag tagacaggaa |
| −100 | aagaagtcgt catttccggg ggagccttca cctgggcttg gtggctctct |
| −50 | gctctctgat tgggcggaag tggcctgggc aggcttgtga ccctactac |

### Example 2: High-throughput Screening of saRNAs Targeting Mouse Thpo Promoter

Cell Culture and Transfection

Mouse embryonic liver cells (BNL·CL2) (Shanghai Institutes for Biological Sciences, GNM22) were cultured in DMEM media (Gibco), containing 10% of calf serum (Sigma-Aldrich) and 1% of penicillin/streptomycin (Gibco). The cells were cultured at 37° C. under 5% CO₂. The BNL·CL2 cells were plated into a 96-well plate at 3500 cells/well. Following the instructions provided by the manufacturer, RNAiMax (Invitrogen, Carlsbad, Calif.) was used to transfect small activating RNAs at concentrations of 10 nM (unless otherwise specified), and the duration of transfection was 72 h with 2 replicate wells for each treatment.

(2) One-Step RT-qPCR

The media were discarded after transfection and each well was washed once with 150 μL of PBS. After discarding PBS, 50 μL of cell lysis buffer was added to each well and samples were incubated at room temperature for 5 min. From each well, 1 μL of cell lysis buffer was subjected to qPCR analysis on an ABI 7500 fast real-time PCR system (Applied Biosystems) using a one-step TB Green™ PrimeScrip™ RT-PCR kit II (Takara, RR086A) and each sample was amplified in 3 replicate wells. The PCR reaction conditions are shown in Table 1.

TABLE 1
PCR reaction preparation

| Reagent | Volume |
|---|---|
| 2 × One-step TB Green RT-PCR buffer 4 | 2.5 μL |
| PrimeScript 1 step enzyme mixture 2 | 0.2 μL |
| Mixture of forward and reverse primers (5 μM) | 0.4 μL |
| No RNase dH₂O | 1.4 μL |
| Crude lysate (RNA) | 0.5 μL |
| Sum | 5 μL |

The reaction conditions are as follows: Stage 1: reverse transcription reaction: 5 min at 42° C., 10 s at 95° C.; Stage 2: PCR reaction: 5 s at 95° C., 20 s at 60° C., 45 cycles of amplification. Sdha and Tbp were used as internal control genes. The PCR primers used by Thpo, Sdha and Tbp are shown in Table 2 and Thpo was amplified using Thpo F1/R1 primer pair.

TABLE 2
Primer sequences for RT-qPCR analysis

| Primer | Sequence No. | Sequence (5′-3′) |
|---|---|---|
| Thpo F1 | SEQ ID NO: 297 | TGGAGCTGACTGATTTGCTCCTG |
| Thpo R1 | SEQ ID NO: 298 | AACAGGGATAGACAAAGGGTCG |
| Sdha F | SEQ ID NO: 299 | ATGGTCACTAGGGCTGGTTTG |
| Sdha R | SEQ ID NO: 300 | ACGACACCCTTCTGTGATGAG |
| Tbp F | SEQ ID NO: 301 | CCGTGAATCTTGGCTGTAAACT |
| Tbp R | SEQ ID NO: 302 | TGTCCGTGGCTCTCTTATTC |

In order to calculate the expression value (Eᵣₑₗ) of a Thpo gene (target) in an saRNA-transfected sample relative to control treatment (Mock), the Ct values of the target gene and two internal control genes were inserted into Formula 1.

Eᵣₑₗ=2^{(CtTm-CtTs)}/((2^{CtR1m-CtR1s)}*2^{(CtR2-CtR2s)})^{(1/2)}) Formula 1

wherein CtTm is the Ct value of the target gene from the control treatment (Mock) sample, CtTs is the Ct value of the target gene from the saRNA-treated sample. CtR1m is the Ct value of the internal control gene 1 from the Mock-treated sample, CtR1s is the Ct value of the internal control gene 1 from the saRNA-treated sample, CtR2m is the Ct value of the internal control gene 2 from the control treatment sample, and CtR2s is the Ct value of the internal control gene 2 from the saRNA-treated sample.

(3) Screening of Mouse Functional saRNAs

To obtain saRNAs capable of activating Thpo transcription, BNL·CL2 cells were transfected by the aforementioned 322 saRNAs at a concentration of 10 nM. According to methods described herein, cells were lysed 72h later and subjected to one-step RT-qPCR analysis to obtain the relative expression value of the Thpo gene for each saRNA-treated sample when compared with the control treatment. As shown in Table 3, 18 (5.6%) saRNAs showed high activation, 79 (24.5%) saRNAs showed mild activation, and 142 (69.9%) saRNAs did not affect Thpo expression. The maximum activation was 2.89-fold, and the maximum inhibition was 0.26-fold. The saRNAs with activation activity are called activating saRNAs.

TABLE 3
High-throughput screening results of mouse Thpo saRNA

| | log₂ value (fold) of | Number of | |
|---|---|---|---|
| saRNA activity | changes in Thpo mRNA | saRNAs | Percentage |
| High activation | ≥0.58 (1.50)~≤1.53 (2.89) | 18 | 5.6% |
| Mild activation | ≥0.13 (1.10)~<0.58 (1.50) | 79 | 24.5% |
| No activating | <0.13 (1.10) | 225 | 69.9% |
| effect | | | |
| Sum | | 322 | 100% |

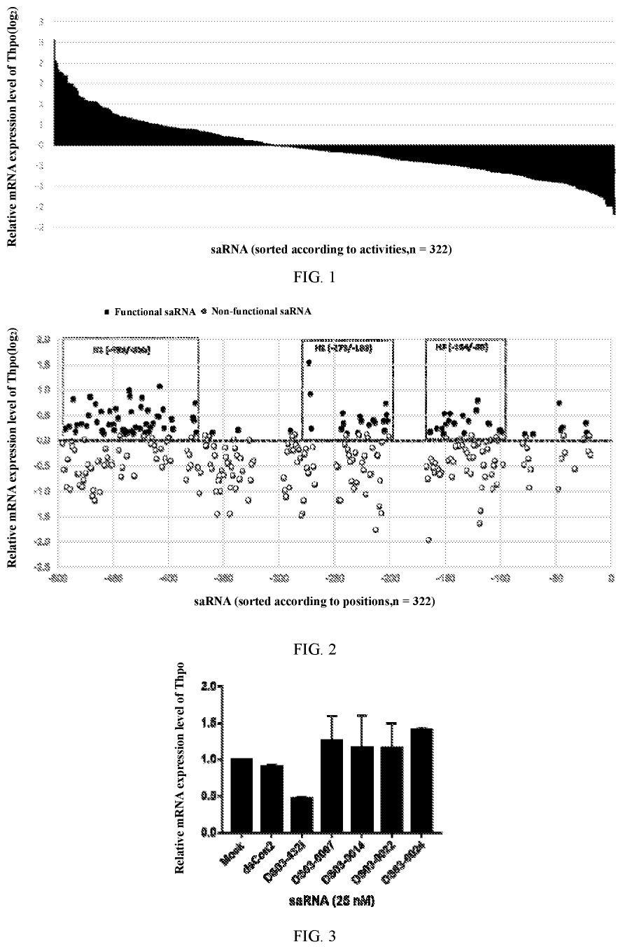

Shown in FIG. 1, are the changes in Thpo expression of mouse Thpo saRNA in descending order. Thpo active saRNA sequences, active target sequences, and fold of changes in relative expression of Thpo mRNA shown in Table 4.

TABLE 4
Mouse active saRNA sequences, active target sequences
thereof and changes in expression of mouse Thpo mRNA

| | | | | Fold of |
|---|---|---|---|---|
| | | | | changes in |
| | | | | relative |
| | | | | expression |
| | Target sequence | Sense sequence | Antisense sequence | of Thpo |
| saRNA | (5′-3′) | (5′-3′) of saRNA | (5′-3′) of saRNA | mRNA |
| RAG3-493 | GGCAATACCTACTTTGCCA | GGCAAUACCUACUUUGCCATT | UGGCAAAGUAGGUAUUGCCTT | 1.17 |
| | (SEQ ID NO: 200) | (SEQ ID NO: 6) | (SEQ ID NO: 103) | |
| RAG3-490 | AATACCTACTTTGCCAGTT | AAUACCUACUUUGCCAGUUTT | AACUGGCAAAGUAGGUAUUTT | 1.21 |
| | (SEQ ID NO: 201) | (SEQ ID NO: 7) | (SEQ ID NO: 104) | |
| RAG3-486 | CCTACTTTGCCAGTTGGAA | CCUACUUUGCCAGUUGGAATT | UUCCAACUGGCAAAGUAGGTT | 1.76 |
| | (SEQ ID NO: 202) | (SEQ ID NO: 8) | (SEQ ID NO: 105) | |
| RAG3-484 | TACTTTGCCAGTTGGAAAG | UACUUUGCCAGUUGGAAAGTT | CUUUCCAACUGGCAAAGUATT | 1.13 |
| | (SEQ ID NO: 203) | (SEQ ID NO: 9) | (SEQ ID NO: 106) | |
| RAG3-483 | ACTTTGCCAGTTGGAAAGG | ACUUUGCCAGUUGGAAAGGTT | CCUUUCCAACUGGCAAAGUTT | 1.24 |
| | (SEQ ID NO: 204) | (SEQ ID NO: 10) | (SEQ ID NO: 107) | |
| RAG3-479 | TGCCAGTTGGAAAGGGGAA | UGCCAGUUGGAAAGGGGAATT | UUCCCCUUUCCAACUGGCATT | 1.25 |
| | (SEQ ID NO: 205) | (SEQ ID NO: 11) | (SEQ ID NO: 108) | |
| RAG3-474 | GTTGGAAAGGGGAAATGCC | GUUGGAAAGGGGAAAUGCCTT | GGCAUUUCCCCUUUCCAACTT | 1.22 |
| | (SEQ ID NO: 206) | (SEQ ID NO: 12) | (SEQ ID NO: 109) | |
| RAG3-472 | TGGAAAGGGGAAATGCCAG | UGGAAAGGGGAAAUGCCAGTT | CUGGCAUUUCCCCUUUCCATT | 1.41 |
| | (SEQ ID NO: 207) | (SEQ ID NO: 13) | (SEQ ID NO: 110) | |
| RAG3-471 | GGAAAGGGGAAATGCCAGG | GGAAAGGGGAAAUGCCAGGTT | CCUGGCAUUUCCCCUUUCCTT | 1.81 |
| | (SEQ ID NO: 208) | (SEQ ID NO: 14) | (SEQ ID NO: 111) | |
| RAG3-466 | GGGGAAATGCCAGGAAAGA | GGGGAAAUGCCAGGAAAGATT | UCUUUCCUGGCAUUUCCCCTT | 1.64 |
| | (SEQ ID NO: 209) | (SEQ ID NO: 15) | (SEQ ID NO: 112) | |
| RAG3-465 | GGGAAATGCCAGGAAAGAC | GGGAAAUGCCAGGAAAGACTT | GUCUUUCCUGGCAUUUCCCTT | 1.29 |
| | (SEQ ID NO: 210) | (SEQ ID NO: 16) | (SEQ ID NO: 113) | |
| RAG3-461 | AATGCCAGGAAAGACACTC | AAUGCCAGGAAAGACACUCTT | GAGUGUCUUUCCUGGCAUUTT | 1.48 |
| | (SEQ ID NO: 211) | (SEQ ID NO: 17) | (SEQ ID NO: 114) | |
| RAG3-460 | ATGCCAGGAAAGACACTCC | AUGCCAGGAAAGACACUCCTT | GGAGUGUCUUUCCUGGCAUTT | 1.14 |
| | (SEQ ID NO: 212) | (SEQ ID NO: 18) | (SEQ ID NO: 115) | |
| RAG3-459 | TGCCAGGAAAGACACTCCT | UGCCAGGAAAGACACUCCUTT | AGGAGUGUCUUUCCUGGCATT | 1.10 |
| | (SEQ ID NO: 213) | (SEQ ID NO: 19) | (SEQ ID NO: 116) | |
| RAG3-454 | GGAAAGACACTCCTCTGGA | GGAAAGACACUCCUCUGGATT | UCCAGAGGAGUGUCUUUCCTT | 1.24 |
| | (SEQ ID NO: 214) | (SEQ ID NO: 20) | (SEQ ID NO: 117) | |
| RAG3-453 | GAAAGACACTCCTCTGGAG | GAAAGACACUCCUCUGGAGTT | CUCCAGAGGAGUGUCUUUCTT | 1.49 |
| | (SEQ ID NO: 215) | (SEQ ID NO: 21) | (SEQ ID NO: 118) | |
| RAG3-450 | AGACACTCCTCTGGAGCAG | AGACACUCCUCUGGAGCAGTT | CUGCUCCAGAGGAGUGUCUTT | 1.24 |
| | (SEQ ID NO: 216) | (SEQ ID NO: 22) | (SEQ ID NO: 119) | |
| RAG3-449 | GACACTCCTCTGGAGCAGG | GACACUCCUCUGGAGCAGGTT | CCUGCUCCAGAGGAGUGUCTT | 1.14 |
| | (SEQ ID NO: 217) | (SEQ ID NO: 23) | (SEQ ID NO: 120) | |
| RAG3-448 | ACACTCCTCTGGAGCAGGG | ACACUCCUCUGGAGCAGGGTT | CCCUGCUCCAGAGGAGUGUTT | 1.54 |
| | (SEQ ID NO: 218) | (SEQ ID NO: 24) | (SEQ ID NO: 121) | |
| RAG3-446 | ACTCCTCTGGAGCAGGGGA | ACUCCUCUGGAGCAGGGGATT | UCCCCUGCUCCAGAGGAGUTT | 1.36 |
| | (SEQ ID NO: 219) | (SEQ ID NO: 25) | (SEQ ID NO: 122) | |
| RAG3-441 | TCTGGAGCAGGGGAATATA | UCUGGAGCAGGGGAAUAUATT | UAUAUUCCCCUGCUCCAGATT | 1.15 |
| | (SEQ ID NO: 220) | (SEQ ID NO: 26) | (SEQ ID NO: 123) | |
| RAG3-440 | CTGGAGCAGGGGAATATAG | CUGGAGCAGGGGAAUAUAGTT | CUAUAUUCCCCUGCUCCAGTT | 1.14 |
| | (SEQ ID NO: 221) | (SEQ ID NO: 27) | (SEQ ID NO: 124) | |
| RAG3-439 | TGGAGCAGGGGAATATAGA | UGGAGCAGGGGAAUAUAGATT | UCUAUAUUCCCCUGCUCCATT | 1.10 |
| | (SEQ ID NO: 222) | (SEQ ID NO: 28) | (SEQ ID NO: 125) | |
| RAG3-437 | GAGCAGGGGAATATAGAAG | GAGCAGGGGAAUAUAGAAGTT | CUUCUAUAUUCCCCUGCUCTT | 1.12 |
| | (SEQ ID NO: 223) | (SEQ ID NO: 29) | (SEQ ID NO: 126) | |
| RAG3-436 | AGCAGGGGAATATAGAAGG | AGCAGGGGAAUAUAGAAGGTT | CCUUCUAUAUUCCCCUGCUTT | 1.99 |
| | (SEQ ID NO: 224) | (SEQ ID NO: 30) | (SEQ ID NO: 127) | |
| RAG3-435 | GCAGGGGAATATAGAAGGA | GCAGGGGAAUAUAGAAGGATT | UCCUUCUAUAUUCCCCUGCTT | 1.83 |
| | (SEQ ID NO: 225) | (SEQ ID NO: 31) | (SEQ ID NO: 128) | |
| RAG3-434 | CAGGGGAATATAGAAGGAA | CAGGGGAAUAUAGAAGGAATT | UUCCUUCUAUAUUCCCCUGTT | 1.47 |
| | (SEQ ID NO: 226) | (SEQ ID NO: 32) | (SEQ ID NO: 129) | |
| RAG3-433 | AGGGGAATATAGAAGGAAA | AGGGGAAUAUAGAAGGAAATT | UUUCCUUCUAUAUUCCCCUTT | 1.13 |
| | (SEQ ID NO: 227) | (SEQ ID NO: 33) | (SEQ ID NO: 130) | |
| RAG3-432 | GGGGAATATAGAAGGAAAA | GGGGAAUAUAGAAGGAAAATT | UUUUCCUUCUAUAUUCCCCTT | 1.10 |
| | (SEQ ID NO: 228) | (SEQ ID NO: 34) | (SEQ ID NO: 131) | |
| RAG3-430 | GGAATATAGAAGGAAAAGC | GGAAUAUAGAAGGAAAAGCTT | GCUUUUCCUUCUAUAUUCCTT | 1.20 |
| | (SEQ ID NO: 229) | (SEQ ID NO: 35) | (SEQ ID NO: 132) | |
| RAG3-426 | TATAGAAGGAAAAGCAAGC | UAUAGAAGGAAAAGCAAGCTT | GCUUGCUUUUCCUUCUAUATT | 1.18 |
| | (SEQ ID NO: 230) | (SEQ ID NO: 36) | (SEQ ID NO: 133) | |
| RAG3-425 | ATAGAAGGAAAAGCAAGCA | AUAGAAGGAAAAGCAAGCATT | UGCUUGCUUUUCCUUCUAUTT | 1.57 |
| | (SEQ ID NO: 231) | (SEQ ID NO: 37) | (SEQ ID NO: 134) | |
| RAG3-424 | TAGAAGGAAAAGCAAGCAA | UAGAAGGAAAAGCAAGCAATT | UUGCUUGCUUUUCCUUCUATT | 1.79 |
| | (SEQ ID NO: 232) | (SEQ ID NO: 38) | (SEQ ID NO: 135) | |
| RAG3-421 | AAGGAAAAGCAAGCAAGCC | AAGGAAAAGCAAGCAAGCCTT | GGCUUGCUUGCUUUUCCUUTT | 1.11 |
| | (SEQ ID NO: 233) | (SEQ ID NO: 39) | (SEQ ID NO: 136) | |
| RAG3-420 | AGGAAAAGCAAGCAAGCCA | AGGAAAAGCAAGCAAGCCATT | UGGCUUGCUUGCUUUUCCUTT | 1.25 |
| | (SEQ ID NO: 234) | (SEQ ID NO: 40) | (SEQ ID NO: 137) | |
| RAG3-419 | GGAAAAGCAAGCAAGCCAG | GGAAAAGCAAGCAAGCCAGTT | CUGGCUUGCUUGCUUUUCCTT | 1.51 |
| | (SEQ ID NO: 235) | (SEQ ID NO: 41) | (SEQ ID NO: 138) | |
| RAG3-418 | GAAAAGCAAGCAAGCCAGC | GAAAAGCAAGCAAGCCAGCTT | GCUGGCUUGCUUGCUUUUCTT | 1.12 |
| | (SEQ ID NO: 236) | (SEQ ID NO: 42) | (SEQ ID NO: 139) | |
| RAG3-417 | AAAAGCAAGCAAGCCAGCA | AAAAGCAAGCAAGCCAGCATT | UGCUGGCUUGCUUGCUUUUTT | 1.19 |
| | (SEQ ID NO: 237) | (SEQ ID NO: 43) | (SEQ ID NO: 140) | |
| RAG3-414 | AGCAAGCAAGCCAGCATGC | AGCAAGCAAGCCAGCAUGCTT | GCAUGCUGGCUUGCUUGCUTT | 1.27 |
| | (SEQ ID NO: 238) | (SEQ ID NO: 44) | (SEQ ID NO: 141) | |
| RAG3-410 | AGCAAGCCAGCATGCTGTC | AGCAAGCCAGCAUGCUGUCTT | GACAGCAUGCUGGCUUGCUTT | 1.39 |
| | (SEQ ID NO: 239) | (SEQ ID NO: 45) | (SEQ ID NO: 142) | |
| RAG3-409 | GCAAGCCAGCATGCTGTCA | GCAAGCCAGCAUGCUGUCATT | UGACAGCAUGCUGGCUUGCTT | 1.38 |
| | (SEQ ID NO: 240) | (SEQ ID NO: 46) | (SEQ ID NO: 143) | |
| RAG3-408 | CAAGCCAGCATGCTGTCAG | CAAGCCAGCAUGCUGUCAGTT | CUGACAGCAUGCUGGCUUGTT | 2.09 |
| | (SEQ ID NO: 241) | (SEQ ID NO: 47) | (SEQ ID NO: 144) | |
| RAG3-405 | GCCAGCATGCTGTCAGAAT | GCCAGCAUGCUGUCAGAAUTT | AUUCUGACAGCAUGCUGGCTT | 1.53 |
| | (SEQ ID NO: 242) | (SEQ ID NO: 48) | (SEQ ID NO: 145) | |
| RAG3-404 | CCAGCATGCTGTCAGAATT | CCAGCAUGCUGUCAGAAUUTT | AAUUCUGACAGCAUGCUGGTT | 1.18 |
| | (SEQ ID NO: 243) | (SEQ ID NO: 49) | (SEQ ID NO: 146) | |
| RAG3-397 | GCTGTCAGAATTCAGCAGC | GCUGUCAGAAUUCAGCAGCTT | GCUGCUGAAUUCUGACAGCTT | 1.34 |
| | (SEQ ID NO: 244) | (SEQ ID NO: 50) | (SEQ ID NO: 147) | |
| RAG3-396 | CTGTCAGAATTCAGCAGCA | CUGUCAGAAUUCAGCAGCATT | UGCUGCUGAAUUCUGACAGTT | 1.17 |
| | (SEQ ID NO: 245) | (SEQ ID NO: 51) | (SEQ ID NO: 148) | |
| RAG3-378 | AGGTATACTGTTCAGGGAA | AGGUAUACUGUUCAGGGAATT | UUCCCUGAACAGUAUACCUTT | 1.36 |
| | (SEQ ID NO: 246) | (SEQ ID NO: 52) | (SEQ ID NO: 149) | |
| RAG3-377 | GGTATACTGTTCAGGGAAA | GGUAUACUGUUCAGGGAAATT | UUUCCCUGAACAGUAUACCTT | 1.32 |
| | (SEQ ID NO: 247) | (SEQ ID NO: 53) | (SEQ ID NO: 150) | |
| RAG3-376 | GTATACTGTTCAGGGAAAA | GUAUACUGUUCAGGGAAAATT | UUUUCCCUGAACAGUAUACTT | 1.67 |
| | (SEQ ID NO: 248) | (SEQ ID NO: 54) | (SEQ ID NO: 151) | |
| RAG3-375 | TATACTGTTCAGGGAAAAG | UAUACUGUUCAGGGAAAAGTT | CUUUUCCCUGAACAGUAUATT | 1.11 |
| | (SEQ ID NO: 249) | (SEQ ID NO: 55) | (SEQ ID NO: 152) | |
| RAG3-360 | AAAGAAATTTGGAGAGCCA | AAAGAAAUUUGGAGAGCCATT | UGGCUCUCCAAAUUUCUUUTT | 1.12 |
| | (SEQ ID NO: 250) | (SEQ ID NO: 56) | (SEQ ID NO: 153) | |
| RAG3-337 | CTGAGACTTCATCCATCTA | CUGAGACUUCAUCCAUCUATT | UAGAUGGAUGAAGUCUCAGTT | 1.15 |
| | (SEQ ID NO: 251) | (SEQ ID NO: 57) | (SEQ ID NO: 154) | |
| RAG3-290 | CCGTATTCTGCCAGATTTC | CCGUAUUCUGCCAGAUUUCTT | GAAAUCUGGCAGAAUACGGTT | 1.14 |
| | (SEQ ID NO: 252) | (SEQ ID NO: 58) | (SEQ ID NO: 155) | |
| RAG3-273 | TCTCCTGGACTAACCCTCT | UCUCCUGGACUAACCCUCUTT | AGAGGGUUAGUCCAGGAGATT | 2.89 |
| | (SEQ ID NO: 253) | (SEQ ID NO: 59) | (SEQ ID NO: 156) | |
| RAG3-272 | CTCCTGGACTAACCCTCTT | CUCCUGGACUAACCCUCUUTT | AAGAGGGUUAGUCCAGGAGTT | 1.89 |
| | (SEQ ID NO: 254) | (SEQ ID NO: 60) | (SEQ ID NO: 157) | |
| RAG3-271 | TCCTGGACTAACCCTCTTC | UCCUGGACUAACCCUCUUCTT | GAAGAGGGUUAGUCCAGGATT | 1.17 |
| | (SEQ ID NO: 255) | (SEQ ID NO: 61) | (SEQ ID NO: 158) | |
| RAG3-244 | ACTTAGGTCCCAGGTTCTC | ACUUAGGUCCCAGGUUCUCTT | GAGAACCUGGGACCUAAGUTT | 1.17 |
| | (SEQ ID NO: 256) | (SEQ ID NO: 62) | (SEQ ID NO: 159) | |
| RAG3-243 | CTTAGGTCCCAGGTTCTCA | CUUAGGUCCCAGGUUCUCATT | UGAGAACCUGGGACCUAAGTT | 1.45 |
| | (SEQ ID NO: 257) | (SEQ ID NO: 63) | (SEQ ID NO: 160) | |
| RAG3-242 | TTAGGTCCCAGGTTCTCAT | UUAGGUCCCAGGUUCUCAUTT | AUGAGAACCUGGGACCUAATT | 1.27 |
| | (SEQ ID NO: 258) | (SEQ ID NO: 64) | (SEQ ID NO: 161) | |
| RAG3-239 | GGTCCCAGGTTCTCATGTC | GGUCCCAGGUUCUCAUGUCTT | GACAUGAGAACCUGGGACCTT | 1.10 |
| | (SEQ ID NO: 259) | (SEQ ID NO: 65) | (SEQ ID NO: 162) | |
| RAG3-229 | TCTCATGTCGCCAGGACAT | UCUCAUGUCGCCAGGACAUTT | AUGUCCUGGCGACAUGAGATT | 1.12 |
| | (SEQ ID NO: 260) | (SEQ ID NO: 66) | (SEQ ID NO: 163) | |
| RAG3-227 | TCATGTCGCCAGGACATGA | UCAUGUCGCCAGGACAUGATT | UCAUGUCCUGGCGACAUGATT | 1.39 |
| | (SEQ ID NO: 261) | (SEQ ID NO: 67) | (SEQ ID NO: 164) | |
| RAG3-226 | CATGTCGCCAGGACATGAG | CAUGUCGCCAGGACAUGAGTT | CUCAUGUCCUGGCGACAUGTT | 1.30 |
| | (SEQ ID NO: 262) | (SEQ ID NO: 68) | (SEQ ID NO: 165) | |
| RAG3-219 | CCAGGACATGAGACAGTTC | CCAGGACAUGAGACAGUUCTT | GAACUGUCUCAUGUCCUGGTT | 1.24 |
| | (SEQ ID NO: 263) | (SEQ ID NO: 69) | (SEQ ID NO: 166) | |
| RAG3-215 | GACATGAGACAGTTCCCGC | GACAUGAGACAGUUCCCGCTT | GCGGGAACUGUCUCAUGUCTT | 1.32 |
| | (SEQ ID NO: 264) | (SEQ ID NO: 70) | (SEQ ID NO: 167) | |
| RAG3-214 | ACATGAGACAGTTCCCGCT | ACAUGAGACAGUUCCCGCUTT | AGCGGGAACUGUCUCAUGUTT | 1.28 |
| | (SEQ ID NO: 265) | (SEQ ID NO: 71) | (SEQ ID NO: 168) | |
| RAG3-206 | CAGTTCCCGCTATACAAAT | CAGUUCCCGCUAUACAAAUTT | AUUUGUAUAGCGGGAACUGTT | 1.31 |
| | (SEQ ID NO: 266) | (SEQ ID NO: 72) | (SEQ ID NO: 169) | |
| RAG3-205 | AGTTCCCGCTATACAAATT | AGUUCCCGCUAUACAAAUUTT | AAUUUGUAUAGCGGGAACUTT | 1.14 |
| | (SEQ ID NO: 267) | (SEQ ID NO: 73) | (SEQ ID NO: 170) | |
| RAG3-204 | GTTCCCGCTATACAAATTC | GUUCCCGCUAUACAAAUUCTT | GAAUUUGUAUAGCGGGAACTT | 1.65 |
| | (SEQ ID NO: 268) | (SEQ ID NO: 74) | (SEQ ID NO: 171) | |
| RAG3-203 | TTCCCGCTATACAAATTCT | UUCCCGCUAUACAAAUUCUTT | AGAAUUUGUAUAGCGGGAATT | 1.42 |
| | (SEQ ID NO: 269) | (SEQ ID NO: 75) | (SEQ ID NO: 172) | |
| RAG3-202 | TCCCGCTATACAAATTCTC | UCCCGCUAUACAAAUUCUCTT | GAGAAUUUGUAUAGCGGGATT | 1.31 |
| | (SEQ ID NO: 270) | (SEQ ID NO: 76) | (SEQ ID NO: 173) | |
| RAG3-164 | GAGGAGCTGGGCTCTATTC | GAGGAGCUGGGCUCUAUUCTT | GAAUAGAGCCCAGCUCCUCTT | 1.13 |
| | (SEQ ID NO: 271) | (SEQ ID NO: 77) | (SEQ ID NO: 174) | |
| RAG3-158 | CTGGGCTCTATTCATGGAC | CUGGGCUCUAUUCAUGGACTT | GUCCAUGAAUAGAGCCCAGTT | 1.17 |
| | (SEQ ID NO: 272) | (SEQ ID NO: 78) | (SEQ ID NO: 175) | |
| RAG3-153 | CTCTATTCATGGACCATGG | CUCUAUUCAUGGACCAUGGTT | CCAUGGUCCAUGAAUAGAGTT | 1.17 |
| | (SEQ ID NO: 273) | (SEQ ID NO: 79) | (SEQ ID NO: 176) | |
| RAG3-152 | TCTATTCATGGACCATGGT | UCUAUUCAUGGACCAUGGUTT | ACCAUGGUCCAUGAAUAGATT | 1.10 |
| | (SEQ ID NO: 274) | (SEQ ID NO: 80) | (SEQ ID NO: 177) | |
| RAG3-151 | CTATTCATGGACCATGGTC | CUAUUCAUGGACCAUGGUCTT | GACCAUGGUCCAUGAAUAGTT | 1.44 |
| | (SEQ ID NO: 275) | (SEQ ID NO: 81) | (SEQ ID NO: 178) | |
| RAG3-150 | TATTCATGGACCATGGTCC | UAUUCAUGGACCAUGGUCCTT | GGACCAUGGUCCAUGAAUATT | 1.24 |
| | (SEQ ID NO: 276) | (SEQ ID NO: 82) | (SEQ ID NO: 179) | |
| RAG3-147 | TCATGGACCATGGTCCAGC | UCAUGGACCAUGGUCCAGCTT | GCUGGACCAUGGUCCAUGATT | 1.31 |
| | (SEQ ID NO: 277) | (SEQ ID NO: 83) | (SEQ ID NO: 180) | |
| RAG3-146 | CATGGACCATGGTCCAGCC | CAUGGACCAUGGUCCAGCCTT | GGCUGGACCAUGGUCCAUGTT | 1.44 |
| | (SEQ ID NO: 278) | (SEQ ID NO: 84) | (SEQ ID NO: 181) | |
| RAG3-142 | GACCATGGTCCAGCCATGG | GACCAUGGUCCAGCCAUGGTT | CCAUGGCUGGACCAUGGUCTT | 1.26 |
| | (SEQ ID NO: 279) | (SEQ ID NO: 85) | (SEQ ID NO: 182) | |
| RAG3-134 | TCCAGCCATGGGCTCTAGT | UCCAGCCAUGGGCUCUAGUTT | ACUAGAGCCCAUGGCUGGATT | 1.41 |
| | (SEQ ID NO: 280) | (SEQ ID NO: 86) | (SEQ ID NO: 183) | |
| RAG3-133 | CCAGCCATGGGCTCTAGTC | CCAGCCAUGGGCUCUAGUCTT | GACUAGAGCCCAUGGCUGGTT | 1.11 |
| | (SEQ ID NO: 281) | (SEQ ID NO: 87) | (SEQ ID NO: 184) | |
| RAG3-129 | CCATGGGCTCTAGTCCCAG | CCAUGGGCUCUAGUCCCAGTT | CUGGGACUAGAGCCCAUGGTT | 1.30 |
| | (SEQ ID NO: 282) | (SEQ ID NO: 88) | (SEQ ID NO: 185) | |
| RAG3-122 | CTCTAGTCCCAGTAGACAG | CUCUAGUCCCAGUAGACAGTT | CUGUCUACUGGGACUAGAGTT | 1.51 |
| | (SEQ ID NO: 283) | (SEQ ID NO: 89) | (SEQ ID NO: 186) | |
| RAG3-121 | TCTAGTCCCAGTAGACAGG | UCUAGUCCCAGUAGACAGGTT | CCUGUCUACUGGGACUAGATT | 1.72 |
| | (SEQ ID NO: 284) | (SEQ ID NO: 90) | (SEQ ID NO: 187) | |
| RAG3-111 | GTAGACAGGAAAAGAAGTC | GUAGACAGGAAAAGAAGUCTT | GACUUCUUUUCCUGUCUACTT | 1.26 |
| | (SEQ ID NO: 285) | (SEQ ID NO: 91) | (SEQ ID NO: 188) | |
| RAG3-110 | TAGACAGGAAAAGAAGTCG | UAGACAGGAAAAGAAGUCGTT | CGACUUCUUUUCCUGUCUATT | 1.25 |
| | (SEQ ID NO: 286) | (SEQ ID NO: 92) | (SEQ ID NO: 189) | |
| RAG3-101 | AAAGAAGTCGTCATTTCCG | AAAGAAGUCGUCAUUUCCGTT | CGGAAAUGACGACUUCUUUTT | 1.14 |
| | (SEQ ID NO: 287) | (SEQ ID NO: 93) | (SEQ ID NO: 190) | |
| RAG3-100 | AAGAAGTCGTCATTTCCGG | AAGAAGUCGUCAUUUCCGGTT | CCGGAAAUGACGACUUCUUTT | 1.15 |
| | (SEQ ID NO: 288) | (SEQ ID NO: 94) | (SEQ ID NO: 191) | |
| RAG3-099 | AGAAGTCGTCATTTCCGGG | AGAAGUCGUCAUUUCCGGGTT | CCCGGAAAUGACGACUUCUTT | 1.12 |
| | (SEQ ID NO: 289) | (SEQ ID NO: 95) | (SEQ ID NO: 192) | |
| RAG3-078 | AGCCTTCACCTGGGCTTGG | AGCCUUCACCUGGGCUUGGTT | CCAAGCCCAGGUGAAGGCUTT | 1.10 |
| | (SEQ ID NO: 290) | (SEQ ID NO: 96) | (SEQ ID NO: 193) | |
| RAG3-071 | ACCTGGGCTTGGTGGCTCT | ACCUGGGCUUGGUGGCUCUTT | AGAGCCACCAAGCCCAGGUTT | 1.10 |
| | (SEQ ID NO: 291) | (SEQ ID NO: 97) | (SEQ ID NO: 194) | |
| RAG3-047 | CTCTGATTGGGCGGAAGTG | CUCUGAUUGGGCGGAAGUGTT | CACUUCCGCCCAAUCAGAGTT | 1.66 |
| | (SEQ ID NO: 292) | (SEQ ID NO: 98) | (SEQ ID NO: 195) | |
| RAG3-046 | TCTGATTGGGCGGAAGTGG | UCUGAUUGGGCGGAAGUGGTT | CCACUUCCGCCCAAUCAGATT | 1.15 |
| | (SEQ ID NO: 293) | (SEQ ID NO: 99) | (SEQ ID NO: 196) | |
| RAG3-044 | TGATTGGGCGGAAGTGGCC | UGAUUGGGCGGAAGUGGCCTT | GGCCACUUCCGCCCAAUCATT | 1.20 |
| | (SEQ ID NO: 294) | (SEQ ID NO: 100) | (SEQ ID NO: 197) | |
| RAG3-023 | GGCAGGCTTGTGACCCTAC | GGCAGGCUUGUGACCCUACTT | GUAGGGUCACAAGCCUGCCTT | 1.27 |
| | (SEQ ID NO: 295) | (SEQ ID NO: 101) | (SEQ ID NO: 198) | |
| RAG3-022 | GCAGGCTTGTGACCCTACT | GCAGGCUUGUGACCCUACUTT | AGUAGGGUCACAAGCCUGCTT | 1.11 |
| | (SEQ ID NO: 296) | (SEQ ID NO: 102) | (SEQ ID NO: 199) | |

It can be clearly seen from the arrangement of activities of the 322 saRNAs according to their positions in the Thpo promoter region that the functional saRNAs are gathered together, that is, in certain promoter regions, the activating saRNAs gathered in specific “hotspot” regions (FIG. 2). As shown in FIG. 2, 3 hotspot regions respectively appeared in region (H1) from −493 to −356, region (H2) from −273 to −183, and region (H3) from −164 to −80 of the promoter, showing a high gathering of the activating saRNAs. The analysis results indicates that the activating saRNAs were not randomly distributed on the promoter but existed in the specific hotspot regions.

The sequence of hotspot H1 (5′ to 3′:−493 to −356) corresponds to position 1 to position 138, from 5′ to 3′, of SEQ ID NO: 2 in the sequence listing:

| | |
|---|---|
| 1 | ggcaatacct actttgccag ttggaaaggg gaaatgccag gaaagacact |
| 51 | cctctggagc aggggaatat agaaggaaaa gcaagcaagc cagcatgctg |
| 101 | tcagaattca gcagcaggta tactgttcag ggaaaaga |

The sequence of the hotspot H2 (5′ to 3′:−273 to −183) corresponds to position 1 to position 91, from 5′ to 3′, of SEQ ID NO: 3 in the sequence listing:

| | |
|---|---|
| 1 | tctcctggac taaccctctt ctctctctca cttaggtccc aggttctcat |
| 51 | gtcgccagga catgagacag ttcccgctat acaaattctc t |

The sequence of the hotspot H3 (5′ to 3′:−164 to −80) corresponds to position 1 to position 85, from 5′ to 3′, of SEQ ID NO: 4 in the sequence listing:

| | |
|---|---|
| 1 | gaggagctgg gctctattca tggaccatgg tccagccatg ggctctagtc |
| 51 | ccagtagaca ggaaaagaag tcgtcatttc cgggg |

### Example 3: SaRNA Promoting Expression of Thpo mRNA in a Mouse Liver Cell

### (1) Cell Culture and Transfection

Cell culture was described in Example 2. Mouse embryonic liver cells (BNL·CL2) were plated into a 6-well plate, at 1×10⁵ cells/well. According to the instructions provided by the manufacturer, RNAiMax (Invitrogen, Carlsbad, Calif.) was used to transfect mouse Thpo saRNA at a concentration of 10 nM, transfection duration was 72 h, and 2 replicate wells were used in each treatment.

### (2) Two-Step RT-qPCR

After transfection, the media were discarded, each well was added with 500 μL of cell lysis buffer, and incubation was performed at room temperature for 5 min. RNA was extracted using Qiagen RNeasy kit, reverse transcribed and subjected to qPCR analysis on ABI 7500 fast real-time PCR system (Applied Biosystems), and each sample was repeatedly amplified in 3 replicate wells. PCR reaction conditions were shown in Tables 5 and 6.

TABLE 5
RT reaction preparation

| Reagent (RT reaction 1) | Volume | Reagent (RT reaction 2) | Volume |
|---|---|---|---|
| 5 × gDNA Eraser buffer | 2 μL | 5 × PrimeScript buffer 2 | 4 μL |
| gDNA Eraser | 1 μl | PrimeScript RT enzyme | 1 μL |
| Total amount of RNA (1 | 7 μL | mixture 1 | |
| μg) + D.W | | RT primer mixture | 1 μL |
| Final volume | 10 μL | No RNase dH₂O | 4 μL |

| | | |
|---|---|---|
| 2 min at 42° C. and stored | RT reaction 1 | 10 μL |
| at 4° C. | Final volume | 20 μL |

15 min at 37° C., 5 s at 85° C. and
stored at 4° C.

TABLE 6
RT-qPCR reaction preparation

| Reagent | Volume |
|---|---|
| SYBR Premix Ex Taq II (2 ×) | 5 μL |
| ROX reference dye II (50 ×) | 0.2 μL |
| Mixture of forward and reverse primers (5 μM) | 0.8 μL |
| cDNA (RT products) | 4 μL |
| Sum | 10 μL |

The reaction conditions were as follows: 30 s at 95° C., 5s at 95° C., 30 s at 60° C., 40 cycles of amplification. The amplification of the Tbp gene with the Thpo F1/R1 primer pair served as an internal control. Amplification primers are shown in a Table 2.

To calculate the expression value (Eᵣₑₗ) of the Thpo (target gene) of an saRNA-transfected sample relative to the control treatment (Mock),Ct values of the target gene and one internal control gene were inserted into Formula 2 for calculation.

Eᵣₑₗ=2^{(CtTm-CtTs)}/2^{(CtRm-CtRs)} Formula 2

FIG. 3 shows the relative mRNA expression values of Thpo after treatment of cells with different mouse saRNAs. The relative mRNA expression of mouse Thpo small interfering RNA (siRNA) in the DS03-432i group was reduced by 53.5% compared to the control group using the control treatment as a blank transfection control. This result indicates that this siRNA was successful as a small interfering RNA control for transfection. The following groups with different saRNAs include DS03-0007 (RAG3-424), DS03-0014 (RAG3-425), DS03-0022 (RAG3-243) and DS03-0024 (RAG3-151), and showed higher relative mRNA expression values when compared to the control group, which increased by 25.5%, 16.5%, 16% and 41%, respectively. This result indicates that randomly selected activating saRNAs are capable of promoting expression of Thpo mRNA in BNL·CL2 cells.

### Example 4: Mouse Thpo saRNA Promoting Expression of Thpo mRNA in LPC-H12 Cells

The cell culture was described in Example 2. Mouse liver cancer cells (LPC-H12) (BNBIO, BNCC101945) were plated into a 6-well plate at 2×10⁵ cells/well. According to the instructions provided by the manufacturer, RNAiMax (Invitrogen, Carlsbad, Calif.) was used to transfect mouse Thpo saRNA at a concentration of 10 nM for a duration of 72h. Two replicate wells were used for each treatment. Two-step RT-qPCR was described in Example 3.

FIG. 4 shows the relative mRNA expression values of Thpo after cells were treated with different saRNAs. The relative mRNA expression of the mouse siRNA (DS03-432i) group was reduced by 47% compared to the control group using the control treatment as a blank transfection control. This result indicates that this siRNA was successful as a small interfering RNA control transfection. The relative mRNA expression values of DS03-0007 (RAG3-424), DS03-0019 (RAG3-453), DS03-0022 (RAG3-243), DS03-0024 (RAG3-151) and DS03-0027 (RAG3-134) groups were higher than those of a control group, and the relative mRNA expression values were increased by 52%, 90%, 30.5%, 11.5% and 34%, respectively, and the activation effect of DS03-0019 was particularly obvious (90%). The results show that randomly selected activating saRNAs are capable of promoting expression of Thpo mRNA to varying degrees in LPC-H12 cells.

### Example 5: Mouse Thpo saRNA Promoting Expression of Thpo mRNA in Primary Mouse Liver Cells

### Culture and Transfection of Primary Mouse Liver Cells

C57 female mice (6 to 8 weeks old, approximately 25 g in weight, purchased from Comparative Medicine Center of Yangzhou University) were acclimated in the mouse animal room for 3 days and primary mouse liver cells were isolated by a two-step perfusion method. The specific operation of the two-step perfusion method is described as follows: Anesthetized mice were maintained using isoflurane and sterilized with alcohol, and the abdominal cavity of the mice was opened with scissors to expose hepatic portal vein and inferior vena cava; a needle was inserted into the hepatic portal vein, and a peristaltic pump was used to infuse pre-perfusion (HBSS+HEPES+EGTA) at 37° C. at a rate of 1 to 2 mL/min, and the mouse liver swelled and turned white, indicating successful perfusion; the inferior vena cava was cut, and the perfusion rate was gradually increased to 7 to 9 mL/min, perfusion was performed for 10 to 15 min; collagenase solution for digestion was added for about 15 min, and the perfusion rate was 5 to 8 mL/min. After digestion, the liver was transferred to a petri dish and liver cells were collected, passed through a 200-mesh filter, washed 3 times with DMEM medium, and centrifuged (100 g) at 4° C. for 5 min. The cells were cultured at 2×10⁵ cells/well (collagen-coated well plate) in DMEM-L medium containing 10% serum, the DMEM-L medium without serum was replaced after 4 to 6 h, and the cells were cultured overnight at 37° C. under 5% CO₂. The next day, RNAiMax (Invitrogen, Carlsbad, Calif.) was used to transfect mouse Thpo saRNA at a concentration of 10 nM for a duration of 72 h. Two replicate wells were used in each treatment. Two-step RT-qPCR was described in Example 3.

FIG. 5 shows the relative mRNA expression values of Thpo after treatment of cells with different mouse saRNAs. The relative mRNA expression values of DS03-0007 (RAG3-424), DS03-0012 (RAG3-204), DS03-0014 (RAG3-425), DS03-0019 (RAG3-453), DS03-0022 (RAG3-243), DS03-0024 (RAG3-151) and DS03-0027 (RAG3-134) groups were higher than control group and all increased by 33.5%, 25.5%, 51.5%, 44.5%, 28.5%, 26% and 34.5%, respectively, compared to the control group using the control treatment as a blank transfection control. The results show that randomly selected activating saRNAs are capable of promoting expression of Thpo mRNA in primary mouse liver cells.

### Example 6: In Vivo Assay of Thpo saRNA Activation of Thpo Gene in Mouse

### Preparation of Oligonucleotide Formulation

Based on the published method of preparing Lipid Nanoparticle (LNP) (see, CN104873464B, CN102625696B, U.S. Pat. No. 9,394,234 B2, WO 2010/144740A1, U.S. Pat. No. 8,158,601 B2, and U.S. Pat. No. 8,802,644 B2), LNP was prepared with an improved method. The resulting LNP was used to entrap oligonucleotide. The specific method used is described as follows.

Stock solution preparation of Thpo siRNA (DS03-432i) and saRNA (RAG3-151/DS03-0024): stock solutions of Thpo siRNA (DS03-432i) and saRNA (RAG3-151/DS03-0024) with 5 mg/mL theoretical final concentration were prepared by dissolving siRNA (DS03-432i) and saRNA (RAG3-151/DS03-0024) with DEPC water. The appropriate amounts of siRNA (DS03-432i) and saRNA (RAG3-151/DS03-0024) were taken and diluted 200-fold with PBS to determine the concentrations of siRNA (DS03-432i) and saRNA (DS03-0024).

TABLE 7
RNA concentrations of siRNA (DS03-432i) and saRNA (DS03-0024)

| | RNA | | |
|---|---|---|---|
| | determination | | Concentration of |
| | concentration | | RNA stock |
| Sample | (ng/μL) | Dilution factor | solution (mg/mL) |
| DS03-432i | 41 | 200 | 8.2 |
| DS03-0024 | 41 | 200 | 8.2 |

TABLE 8
Sequences of dsCon2 and siRNA (DS03-432i)

| RNA | Sense sequence | Antisense sequence |
|---|---|---|
| duplexes | (5′-3′) | (5′-3′) |
| dsCon2 | ACUACUGAGUGACAGU | UCUACUGUCACUCAGU |
| | AGATT | AGUTT |
| | (SEQ ID NO: 303) | (SEQ ID NO: 304) |
| DS03- | GGACUUUAGCCUGGGA | UUCUCCCAGGCUAAAG |
| 432i | GAATT | UCCTT |
| | (SEQ ID NO: 305) | (SEQ ID NO: 306) |

Preparation of D-Lin-MC3 lipid: in a patent publication method (see Lipid Formulation, Publication No.: US 2010/0324120 A1), the D-Lin-MC3 lipid was synthesized chemically as follows.

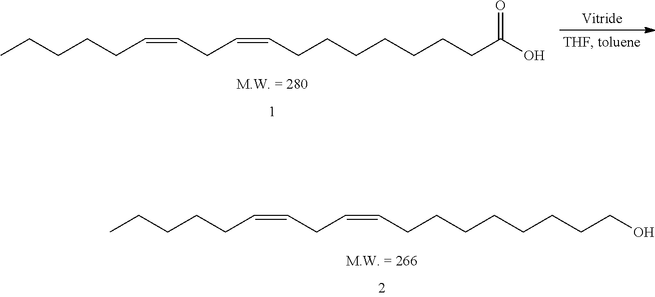 [SMILES: CCCCC/C=C\C/C=C\CCCCCCCC(=O)O ; CCCCC/C=C\C/C=C\CCCCCCCCO]

A dry 1 L glass reactor was purged with dry argon and cooled to 0° C. using an acetone-dry ice bath. 25 g of reaction 1 (linoleic acid) and 300 mL of THF were added. 60 mL of sodium bis(2-methoxyethoxy)aluminum dihydride (70% wt/vol) in toluene was slowly added dropwise to a reactor. After the completion of dropwise addition, the mixture was carried out at room temperature for 2 h. A small sample was taken and starting material reaction was monitored by TLC. After the completion of reaction, saturated sodium sulfate solution was slowly added. After the completion of addition, 130 mL of ethyl acetate was added dropwise over 30 min, and vigorously stirred. The reaction mixture was filtered, the solid was washed with 90 mL of ethyl acetate, and the organic phases were combined and concentrated. The product was dissolved in 80 mL of ethyl acetate, washed twice with water, and dried over anhydrous sodium sulfate. The organic phases were filtered and concentrated, organic solvent was removed using a vacuum pump to give product 2 (17.49 g).

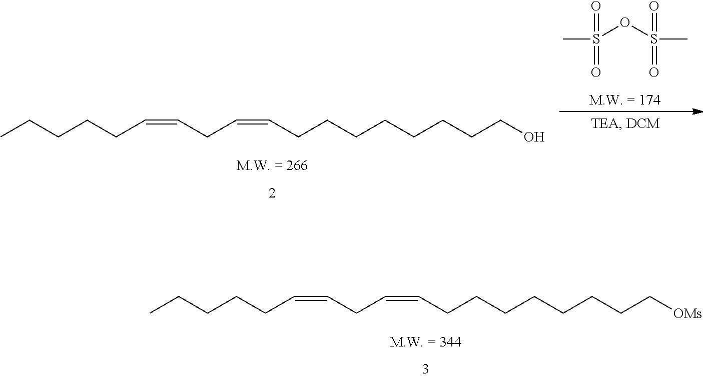

A dry 1 L glass reactor was purged with dry argon, 17 g of product 2 and 150 mL of dichloromethane were then added, 40 mL of triethylamine and 1 g of 4-dimethylaminopyridine were then added, and the mixture was cooled to −10° C. using an acetone-dry ice bath. 30 g of methanesulfonic anhydride was dissolved in 45 mL of dichloromethane and slowly added dropwise to the reactor. After the completion of dropwise addition of the reaction mixture, the reaction was continued at 0° C. for 1 h. Starting material reaction was monitored by TLC. After the completion of reaction, 80 mL of ice water was added to the reaction mixture, the mixture was separated, and the aqueous phase was extracted with 40 mL of dichloromethane. The organic phases were combined, washed with dilute hydrochloric acid, water and saturated NaCl solution, and dried over anhydrous sodium sulfate. The organic phases were filtered and concentrated, organic solvent was removed using a vacuum pump to give product 3 (22.3 g).

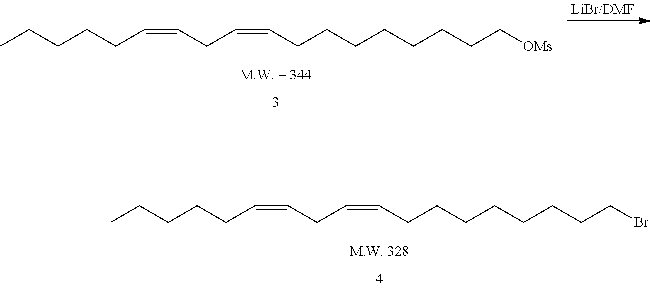 [SMILES: CCCCC/C=C\C/C=C\CCCCCCCCOS(C)(=O)=O ; CCCCC/C=C\C/C=C\CCCCCCCCBr]

A dry 1 L glass reactor was purged with dry argon and then 100 mL of DMF and 22 g of product 3 were added and the solution was cooled to −10° C. using an acetone-dry ice bath. 8.5 g of LiBr was dissolved in 80 mL of DMF, stirred and slowly added dropwise to the reactor. After the completion of dropwise addition, the reaction mixture was heated to 45° C. and stirred for reaction for 18 to 20 h. Starting material reaction was monitored by TLC. After the completion of reaction, 300 mL of water were added and extracted with 240 mL of n-hexane, and the aqueous phase was further extracted with n-hexane. The organic phases were combined, washed with water and saturated NaCl solution and dried over anhydrous sodium sulfate. The organic phases were filtered and concentrated, organic solvent was removed using a vacuum pump to give crude product (16.76 g). The mixture was purified with 60 to 120 mesh silica gel (n-hexane as mobile phase) to give product 4 (13.86 g).

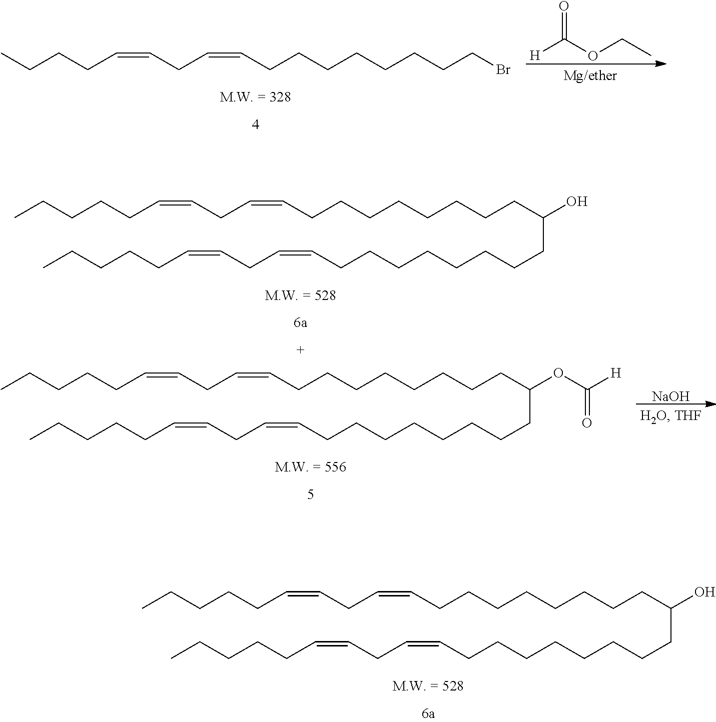

A dry 1 L three-neck flask was purged with dry argon, and a reflux device was additionally arranged. 2 g of magnesium turnings and 12 mL of anhydrous diethyl ether were added into the reactor. 13 g of product 4 were dissolved in 40 mL of anhydrous diethyl ether. Under the protection of argon, 8 mL of reaction mixture was added dropwise to the reactor and 0.2 mL of dibromomethane was added further. The reactor was warmed to 40° C. in a water bath. After the reaction started, the heating was stopped and the remaining 32 mL of the reaction mixture was added dropwise to the reactor, and the mixture was allowed to keep a slight reflux. After the completion of dropwise addition, the reaction mixture was heated to maintain the reflux state (about 45° C.) and reacted for 1 h. A small sample was quenched with water and the starting material reaction was monitored by TLC. After the completion of starting materials reaction, the reaction mixture was cooled to below 10° C. by an ice bath, and then ethyl formate solution was slowly added. After the completion of dropwise addition, the mixture was carried out at room temperature for 1 h. Then 56 mL of ice-water and 10% of sulphuric acid solution were added, the organic phases were separated and the aqueous phase was extracted with diethyl ether. The organic phases were combined, washed with saturated NaCl solution and dried over anhydrous sodium sulfate. The organic phase was filtered and concentrated, organic solvent was removed using a vacuum pump to give crude product (13 g). The crude product was dissolved in 100 mL of 85% ethanol, 7 g of NaOH solid was added, stirred at room temperature for 24 h and the starting material reaction was monitored by TLC. After the completion of reaction, the reaction mixture was extracted with diethyl ether, and the organic phases were combined and washed with saturated NaCl solution. The reaction mixture was dried over anhydrous sodium sulfate. The organic phases were filtered and concentrated. The crude product was purified with 60 to 120 mesh silica gel (4% ether/n-hexane) to give product 6 (10.4 g).

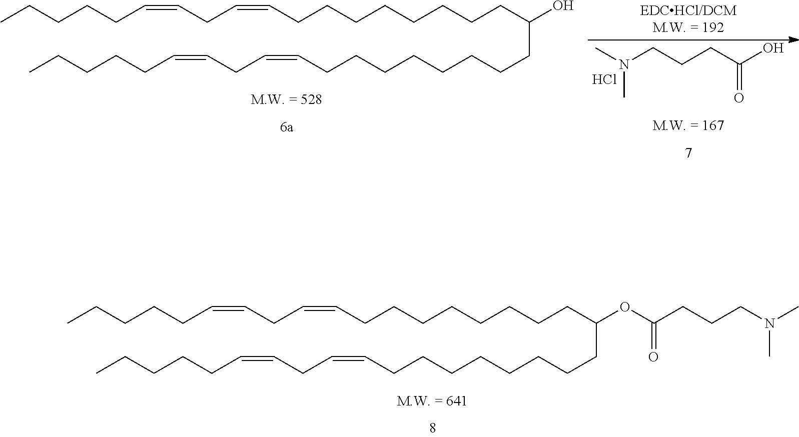

The reaction product 6 (3 g) was added into a 100 mL flask and dissolved in dichloromethane (24 mL), the reaction product 7 (4-(dimethylamino)butanoic acid hydrochloride) (1.1 g) was added, then diisopropylethylamine (1.5 mL) and 4-dimethylaminopyridine (0.09 g) were added. After stirring at room temperature for 5 min, the reaction mixture was added with EDC HCl (1.7 g) and stirred at room temperature overnight. Starting material reaction was monitored by TLC. After the completion of reaction, the reaction mixture was diluted with 20 mL of dichloromethane. Then the reaction mixture was washed with saturated NaHCO₃ solution, water and saturated NaCl solution, dried with anhydrous Na₂SO₄ and filtered, and the organic phases were concentrated to give about crude product (3.3 g). The reaction mixture was isolated using chromatography (serial elution with 1% to 5% methanol/dichloromethane) to give product 8 (Dlin-MC3-DMA, 2.9 g). ¹H-NMR (CDCl₃, 400 MHz), δ=5.44-5.22 (m, 8H), 4.95-4.79 (m, 1H), 2.81-2.63 (m, 4H), 2.36-2.21 (m, 4H), 2.12-1.92 (m, 9H), 1.84-1.67 (m, 2H), 1.48 (d, J=5.4, 4H), 1.36-1.14 (m, 39H), 0.91-0.81 (m, 6H).

Preparation of PEG-c-DMA

PEG-c-DMA was prepared by the chemical method as follows:

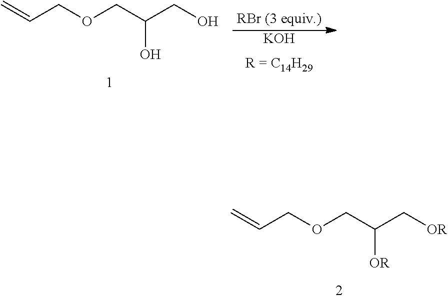

Preparation of Product 2

A dry 1 L glass reactor was purged with dry argon, a mixture of 9 g of the reaction product 1, (3-allyloxy-1,2-propanediol), 60 g of 1-bromotetradecane and 15.5 g of potassium hydroxide was placed in 500 mL of anhydrous benzene, refluxed overnight, and removed with a Dean-Stark splitter. After the mixture was cooled to room temperature, the reaction mixture was diluted with 200 mL of anhydrous benzene. The organic phases were washed with water and saturated NaCl solution, and dried over anhydrous magnesium sulfate. The organic solvent was removed by vacuum pump to give a clear yellow oil (53 g). The reaction mixture was purified with silica gel column (particle size 230 to 400 mesh, 1300 mL) and serial elution with 0 to 5% ethyl acetate/n-hexane was taken as mobile phase. Product 2 was 33 g.

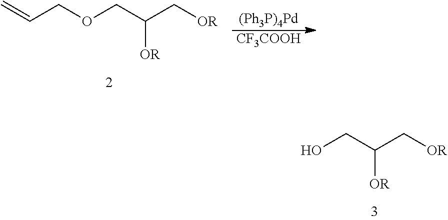

Preparation of Product 3

A dry 1 L glass reactor was purged with dry argon and 45 mL of trifluoroacetic acid was added to give the ethanol solution of product 2 (33 g of product 2 was dissolved in 500 mL of ethanol). After 10 g of tetrakis(triphenylphosphine)palladium was added, the mixture was refluxed overnight in the absence of light. After the completion of reaction, the organic solvent was removed by vacuum pump to give a brown oil. The crude product was purified with silica gel column (particle size 230 to 400 mesh, 1500 mL) and serial elution with 0 to 1% methanol/dichloromethane was taken as mobile phase. The final product 3 (19 g) was a yellow wax.

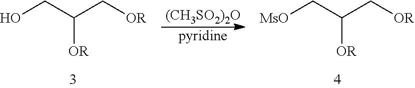

Preparation of Product 4

A dry 1 L glass reactor was purged with dry argon and 13.8 g of methanesulfonic anhydride was dissolved in 150 mL of dichloromethane in the reactor. And 6.4 mL of pyridine was added dropwise to the reactor. 19 g of product 3 was dissolved in 150 mL of methylene chloride and added to reaction system. The reaction mixture was stirred at room temperature under the protection of argon overnight. After the completion of reaction, the product was diluted with 200 mL of dichloromethane, washed with water and saturated NaCl solution, and dried over anhydrous magnesium sulfate. The organic solvent was removed by rotary evaporation to give a crude yellow waxy product 4 (22.1 g).

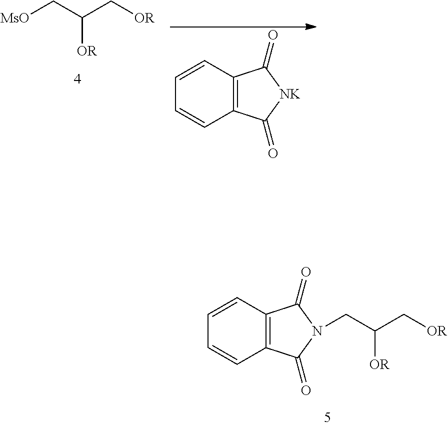

Preparation of Product 5

A dry 1 L glass reactor was purged with dry argon and 22 g of product 4 and 19 g of potassium phthalimide were mixed, dissolved in 400 mL of DMF solution, heated to 70° C. and stirred overnight to give a yellow suspension. After the reaction solution was cooled to room temperature, and poured into 800 mL of ice-water. The aqueous phase was washed with ethyl acetate. The organic phases were combined, washed with water and saturated NaCl solution, and dried over anhydrous magnesium sulfate. The organic solvent was removed by vacuum pump. The mixture was dissolved in 500 mL of n-hexane, filtered and washed with 100 mL of hexane. The organic solvent was removed by rotary evaporation to give 20.7 g of a crude wax. The crude product was purified with silica gel column (particle size 230 to 400 mesh, 600 mL) and serial elution with 0 to 5% ethyl acetate/n-hexane was taken as mobile phase. A pure white waxy product 5 (13.4 g) was given.

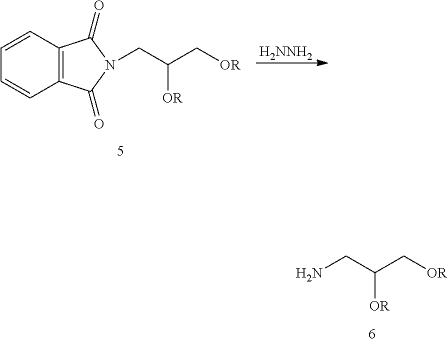

Preparation of Product 6

13.4 g of product 5 and 15.8 mL of hydrazine hydrate was dissolved in 300 mL of ethanol solution at reflux overnight to give a white solid. The reaction mixture was cooled to room temperature and then filtered. The solid was washed twice with ethanol (100 mL). The organic phases were combined, the organic solvent was removed by rotary evaporation and dissolved in 500 mL of chloroform and filtered. The solid was washed once with 100 mL of chloroform. The organic phases were combined, washed with water and saturated NaCl solution, and dried over anhydrous magnesium sulfate. The organic solvent was removed by vacuum pump to give a light-colored oily crude product (10.6 g). The crude product was purified with silica gel column (particle size 230 to 400 mesh, 300 mL) and serial elution with 0 to 10% methanol/chloroform was taken as mobile phase. A light-colored oily crude product (10 g) was given.

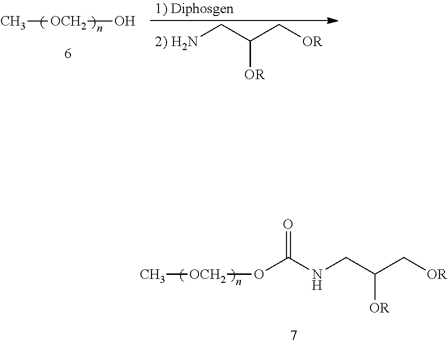

Preparation of Product 7

29.5 g of monomethoxy polyethylene glycol 2000 was dissolved in 150 mL of anhydrous benzene, and the organic solvent was removed by rotary evaporation. The product was dissolved in 250 mL of anhydrous dichloromethane, added into a 1 L dry glass reactor and purged with dry argon. 7.3 g of trichloromethyl chloroformate was added and stirred at room temperature for 3 h. The organic solvent was removed by rotary evaporation, 150 mL of anhydrous benzene was added, and the organic solvent was removed by rotary evaporation. 10 g of product 6 was dissolved in 150 mL of dichloromethane, then added with 3.2 mL of triethylamine and added to 1 L of the reaction under the protection of argon and stirred at room temperature overnight. The reaction product was diluted with 200 mL of dichloromethane. The organic phases were washed with 1% HCl solution, then washed with water, 0.1% sodium carbonate solution, and water and saturated NaCl solution, and finally dried over anhydrous magnesium sulfate. The organic solvent was removed by vacuum pump to give a light-colored slurry crude product (46 g). The crude product was purified with silica gel column (particle size 230 to 400 mesh, 1600 mL). The serial elution with 0 to 10% methanol/chloroform was taken as mobile phase to obtain the product PEG-c-DMA and the remaining reactant which was white or light yellow wax. The waxy mixture was added to 150 mL of diethyl ether to dissolve unreacted reactant. The reaction mixture was filtered, and the solid was washed twice with diethyl ether, dried under vacuum to give product 7 (32.2 g). ¹H NMR (CDCl₃, 400 MHz) δ:5.15 (1H, m, NH), 4.21 (2H, m, CH₂), 3.80-3.1 (8H, m), 3.60 (130H, s, OCH₂CH₂O), 3.34 (3H, s, OCH₃), 2.42 (2H, m, NCH₂), 1.49 (4H, m, 2×CH₂), 1.21 (44H, s, 22×CH₂), 0.84 (6H, t, 2×CH₃) ppm.

Preparation of lipid stock solution: D-Lin-MC3 Lipid, 1,2-distearoyl-sn-glycero-3-phosphocholine, i.e., DSPC (Avanti Polar Lipid, 850365), cholesterol (Sigma, C3045), and PEG-C-DMA were equilibrated at room temperature for about 30 min before weighing. D-Lin-MC3 lipid was formulated into 40 mg/mL of stock solution with 100% ethanol, cholesterol was formulated into 20 mg/mL of stock solution with 100% ethanol, and DSPC was formulated into 20 mg/mL stock solution with 100% ethanol. Long-acting lipid was formulated as 50 mg/mL of stock solution in 100% ethanol.

Preparation of pre-formed vesicles (PFV): D-Lin-MC3, cholesterol, DSPC, PEG-c-DMA and anhydrous ethanol were formulated into lipid ethanol solutions in a molar ratio of 40:40:10:10, and the required amount of each lipid was shown in Table 9. After all lipids were dissolved, the above lipid ethanol solution was added to the formulation buffer (50 mM of citric acid, pH=3.0) with stirring to a final ethanol concentration of 30% (v/v). At room temperature, the Avanti lipid extruder equipped with a layer of Nuclepore Membrane was repeatedly extruded to give PFV with uniform particle size. The PFV extruded by the extruder was measured for particle size using a particle size analyzer.

TABLE 9
Requirements for lipid in F113 formulation
Total volume: 10 mL
Volume of ethanol: 3 mL of ethanol with a concentration of 30% (v/v)

| | Concentration | Required amount |
|---|---|---|
| Lipid | (mg/mL) | (mL) |
| D-Lin-MC3 | 40 | 1.25 |
| DSPC | 20 | 0.462 |
| Cholesterol | 20 | 0.904 |
| PEG-c-DMA | 50 | 0.596 |

| | |
|---|---|
| Addition of ethanol | 0.286 |

To prepare liposome formulations of siRNA (DS03-432i) and saRNA (RAG3-151/DS03-0024), PFV was dispensed into 15 mL Falcon tubes and pre-equilibrated for 5 min in a water bath at 35° C. Two hundred microliters (8.2 mg/mL) of siRNA (DS03-432i) and saRNA (DS03-0024) stock solutions were formulated into 0.4 mL of a solution containing siRNA, formulation buffer, ethanol solution and a solution containing saRNA, formulation buffer, ethanol solution at a ratio of RNA stock solution:formulation buffer:ethanol solution=200 μL:100 μL:100 μL. The siRNA (DS03-432i) or the saRNA (RAG3-151/DS03-0024)/formulation buffer/ethanol solution was slowly added to the above PFV and incubated at 35° C. for 30 min. The siRNA (DS03-432i) and saRNA (RAG3-151/DS03-0024) liposome formulations were then dialyzed overnight using an MD24 dialysis bag, 1× phosphate buffer solution (PBS), and the product recovered after dialysis was the LNP-entrapped oligonucleotide used in the experiment. The LNP-entrapped oligonucleotide was filtered and sterilized using a 0.2 μm needle head-type filter, and 20 μL, of sterilized products was used to determine the encapsulation rate.

(2) LNP-Entrapped Oligonucleotide Injected into Tail Vein of Mice

Fifteen male Balb/c mice (purchased from SPF (Beijing) Biotechnology Co., Ltd.), 6 to 8 weeks old, and weights of 25 g were prepared for the experiments. After acclimation in a mouse animal room for 3 days, the mice were randomly divided into 3 groups with 5 mice in each group based on body weight. A control group (PBS), LNP-entrapped Thpo-siRNA (DS03-432i, 5 mg/kg) and LNP-entrapped Thpo-saRNA (RAG3-151/DS03-0024, 5 mg/kg) was administered respectively by tail vein injection at a dose of 20 mL/kg. Seventy two hours after administration, mice were anesthetized with isoflurane and 200 μL of blood was collected by cardiac puncture for analysis of THPO protein concentration in serum. After blood collection was completed, the mice were sacrificed by carbon dioxide inhalation asphyxiation. The experimental mice to be euthanized were placed in a closable experimental box to release a large amount of carbon dioxide, so that the experimental mice were suffocated and died.

(3) Mouse Serum THPO Protein Determined Using ELISA

Forty microliters of whole blood from mice was let stand at room temperature for 30 min, then centrifuged (4° C., 13000 rpm, 15 min) to collect serum. An ELISA kit with mouse THPO (R&D Systems, MTP00) was used to determine the content of THPO in the collected serum. The procedures were briefly described below, which were according to the instructions provided in the kit. Sufficient strips (provided by the kit) were prepared and 50 μl of RD1-23 was added into two replicate wells for each group. Fifty microliters of standard, control, and sample was added and the mixture was uniformly mixed by lightly beating the strips and then placing them at room temperature for 2h. The liquid was discarded, the mixture was washed 4 times with 400 μL, of wash buffer, and 100 μL, of mouse Thpo conjugate was added to each well and let to stand at room temperature for 2h. Next, the liquid was discarded, the mixture was washed 4 times with 400 μL of wash buffer, and 100 μL of substrate was added to each well and let to stand at room temperature in the absence of light for 30 minutes. After the completion of the reaction, 100 μL of stop solution was added to each well, the mixture was mixed uniformly by lightly beating the strips, and absorbance at 450 nm was determined using an enzyme labeling instrument (Infinite M200 Pro). A guarantee curve was prepared using a known standard concentration (4000 pg/mL), provided by the kit. The absorbance of the sample to be tested was subtracted from that of a blank control (0 pg/mL) and the THPO protein concentration (pg/mL) was calculated from standard curve.

(4) Statistical Analysis

Differences between groups of continuous variables were compared using one-way analysis of variance (ANOVA) and between groups were compared using Tukey's multiple comparisons. The groups with less than 0.05 of P value were significantly different.

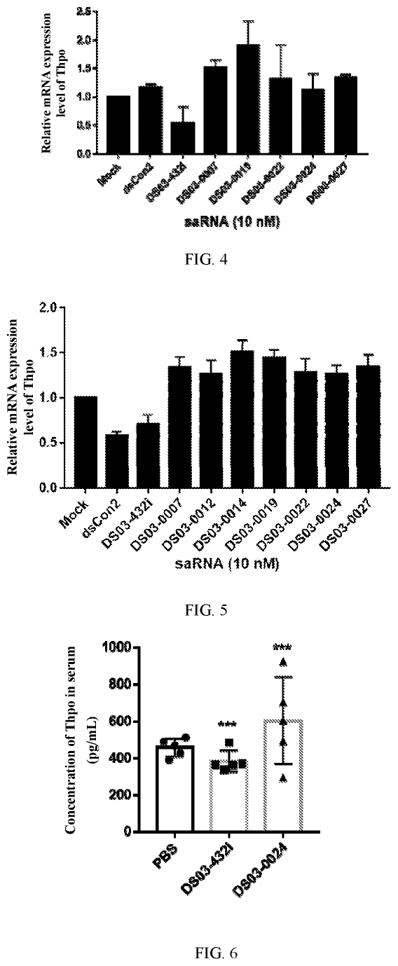

FIG. 6 shows the concentration of Thpo protein in mice serum of different treatment groups. Compared with the control group (PBS), the Thpo protein content of the LNP-entrapped Thpo-siRNA (DS03-432i) group was reduced by 16.1%, and was statistically significant (p<0.001). This result indicates that LNP-entrapped Thpo-siRNA (DS03-432i) can efficiently deliver Thpo-siRNA and significantly reduce the content of Thpo-siRNA protein in mice which is expected and consistent with in vitro cell study results. Compared with the control group, the Thpo protein content of the LNP-entrapped Thpo-saRNA (DS03-0024) group was reduced by 32.9%, and was statistically significant (p<0.001). This result indicates that LNP-entrapped Thpo-saRNA (DS03-0024) can efficiently deliver Thpo-saRNA and significantly increase the content of Thpo protein in mice. N=5; (N, number of samples); *** represents p<0.001.

### Example 7: Tail Vein Delivery of saRNA in Mice Increasing the Level of Serum Thpo and Number of Peripheral Blood Platelets

Thirty two male Balb/c mice (6 to 8 weeks old, 28 to 30 g in weight, purchased from Comparative Medicine Center of Yangzhou University) were prepared. After acclimated in the mouse animal room for 7 days, the above mice were randomly divided into 4 groups with 8 mice in each group based on body weight. The four groups consisted of a control group (PBS) and 3 groups receiving different doses of LNP-entrapped Thpo-saRNA (DS03-0024, 1 mg/kg; DS03-0024, 3 mg/kg, and DS03-0024, 6 mg/kg), respectively. The drug was administered by tail vein injection at a dose of 10 mL/kg. Forty microliters of blood was collected by tail vein 1 day before administration, and at 2 days, 4 days, 6 days and 9 days after dose administration to analyze the concentration of Thpo in the serum. To assay the number of peripheral blood platelets, the mice were anesthetized with isoflurane and 800 μL of blood was collected by cardiac puncture 17 days after the dose administration. After the blood collection was completed, the mice were sacrificed by carbon dioxide inhalation asphyxiation. The experimental mice to be euthanized were placed in a closable experimental box to release a large amount of carbon dioxide, so that the experimental mice were suffocated and died.

(2) Assay of Peripheral Blood Platelet in Mice

To assay peripheral blood platelets in mice, five microliters of whole blood and 5 μL of 0.01% heparin sodium was uniformly mixed and added to 90 μL of physiological saline (1:20 dilution). The mixture was gently mixed and added to 200 μL of the upper layer of blood platelet separation liquid (Solarbio, P1620) and centrifuged at room temperature for 15 min at 250×g. Thereafter, 20 μL of the uppermost blood platelet enrichment layer was collected. Eighty microliters of 1×FASC standing buffer (R&D Systems, FC001) was added to the collected supernatant and 1 μL of CD41a antibody (ThermoFisher, 11-0411-82) was added to bind platelets. The mixture was uniformly mixed and let stand at room temperature in the absence of light for 30 minutes. After completion of the reaction, the mixture was centrifuged at room temperature (800 g, 5 min), the precipitate was resuspended in 200 μL of PBS, and transferred to an enzyme-labeled assay plate determine platelet content. The preparation of the oligonucleotide formulation and methods for injecting LNP-entrapped oligonucleotides into the tail veins of mice are described in Example 6.

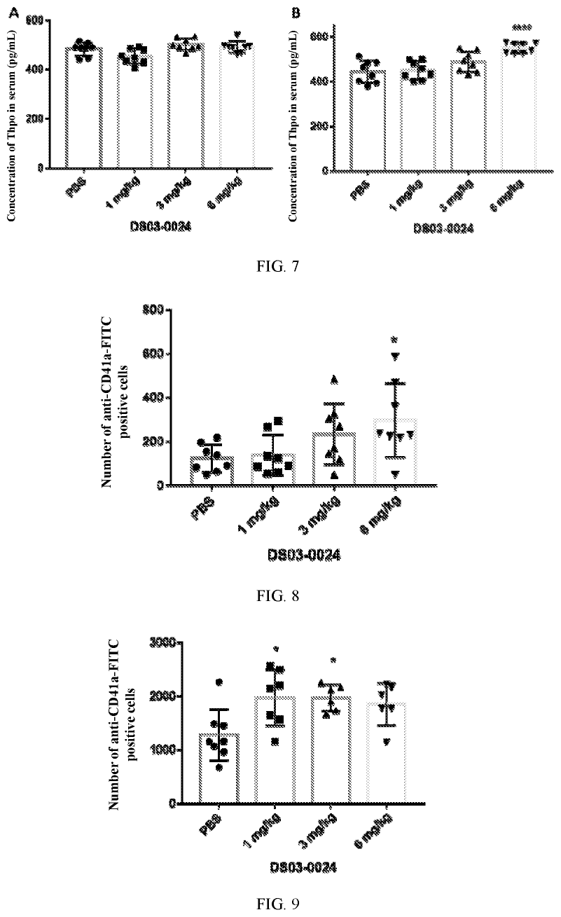

FIG. 7 shows that Thpo saRNA increases the concentration of Thpo protein in mice serum of different treatment groups. As shown in FIG. 7A, the baseline concentration of Thpo protein in mice serum was determined the day before administration and there was no significant difference in the level of serum for Thpo protein in the 4 groups of mice. FIG. 7B shows the concentrations of Thpo protein in mice serum collected 2 days after administration. The level of Thpo protein serum in the DS03-0024-3 mg/kg group is increased compared to the control group (PBS), however not statistically significant. The concentration of Thpo protein in the DS03-0024-6 mg/kg group increased by 23.9%, with a statistical significance of p<0.0001. This result indicates that LNP-entrapped Thpo-saRNA can quickly and efficiently deliver Thpo-saRNA and significantly increase the concentration of Thpo protein in mice serum.

FIG. 8 shows that Thpo saRNA increased the number of platelets in mice 6 days after administration. Compared to the control group (PBS), the two groups of LNP-entrapped Thpo-saRNA, DS03-0024, 1 mg/kg and DS03-0024, 3 mg/kg, 0 increased, though not statistically significant, especially in the number of platelets in the LNP-entrapped Thpo-saRNA group (DS03-0024, 3 mg/kg), which increased by 88.6%. The number of platelets in the LNP-entrapped Thpo-saRNA group (DS03-0024, 6 mg/kg) increased by 138.7%, with a statistical significance of p<0.05. This result indicates that the LNP-entrapped Thpo-saRNA group (DS03-0024, 6 mg/kg) can increase the number of platelets 6 days after administration.

FIG. 9 shows that Thpo saRNA increased the number of platelets in mice 17 days after administration. The number of platelets in mice from the two groups of LNP-entrapped Thpo-saRNA, DS03-0024, 1 mg/kg and DS03-0024, 3 mg/kg, respectively, determined 17 days after administration, increased significantly compared to that of the control group (PBS), increasing by 53.8% and 53.9%, respectively, with a statistical significance (P<0.05). The number of platelets in the LNP-entrapped Thpo-saRNA group (DS03-0024, 6 mg/kg) tended to increase as well. This result indicates that the increases observed in platelet number caused by saRNA can persist for at least 17 days.

### Example 8: Design and Synthesis of saRNA Targeting Human THPO Gene Promoter

To screen functional saRNAs capable of activating human THPO gene expression, a THPO promoter region sequence with a length of 1382 bp was used as a template and target sequences with a length of 19 bp were selected from −1382 bp upstream of TSS. The target sequences were then chosen using the following criteria: (1) having a GC content between 40% and 65%; (2) with less than 5 continuous identical nucleotides; (3) with 3 or less dinucleotide repeat sequences; and (4) with 3 or less trinucleotide repeat sequences. After the selection, the remaining 833 target sequences entered the screening process as candidates. Corresponding double-stranded saRNAs were chemically synthesized based on these candidate sequences.

In the study, each sense strand and antisense strand in the double-stranded saRNA had 21 nucleotides in length. The 19 nucleotides in the 5′ region of the first ribonucleic acid strand (sense strand) of saRNA had 100% identity to the target sequence of the promoter, and the 3′ terminus of the first ribonucleic acid strand contained a TT sequence. The 19 nucleotides in the 5′ region of the second ribonucleic acid strand were complementary to the first ribonucleic acid strand sequence, and the 3′ terminus of the second ribonucleic acid strand contained a TT sequence. The aforementioned two strands of the saRNA were mixed at a molar ratio of 1:1, and after annealing, a double-stranded saRNA was formed.

The sequence of human THPO promoter is shown as follows, which corresponds to position 1 to position −1382 from 5′ to 3′ of SEQ ID No: 600 in the sequence listing:

| | |
|---|---|
| −1382 | gtccatcagc cgatctcccc ctgcctgggc ccacagcgcc ccccaaaccc |
| −1332 | tcaccaccct ctctcactgc ctagcctgcc tccctacctt ctctctgagg |
| −1282 | tcgctcctca ttcttgtgtt acccaggaca gggacctagc cagaaaccgg |
| −1232 | cagcattccc ccttctgtgg agtgacagta tctccctctc attgtaactt |
| −1182 | atcctcaggc gcattcgaca gtcccctctt gctttctcac ccccttcctt |
| −1132 | cacccaaggg accctctgcc tctccagccc actcccagcc tcctttctct |
| −1082 | tggttccctg gtcatgcctg cctccctgtc tcctgtctct ccctcccaca |
| −1032 | cacacccact atcctcccag ctatcccagc accctccttc ctaatcttgg |
| −982 | gagacatctc gtctggctgg acgggaaaat tccaggatct aggccacact |
| −932 | tctcagcaga catgcccatc cttggggagg aggaacagga gagagcctga |
| −882 | ggaagttctg ggggacaggg ggatgatggg atcaaggtca ggccaggaag |
| −832 | cccctgagga cagagactgt ggggagactg ggactgggaa gaaagcaaag |
| −782 | gagctagagc cagggccaaa ggaaaagggg ggccagcagg gaggtatttg |
| −732 | cgggggaggt ccagcagctg tctttcctaa gacagggaca catgggcctg |
| −682 | gttattcctc ttgtcacatg tggaacggta ggagatggaa gacggagaca |
| −632 | gaacaagcaa aggagggccc tgggcacaga ggtctgtgtg tgtagccatc |
| −582 | taagccactg gaccccagca gacgagcacc taagctcagg cttaacccag |
| −532 | tgcacgtgtg cgcacataca tgtgccccgc acctgacagt ccactcaacc |
| −482 | cgtccaaacc ctttccccat aacaccaacc cataacagga gatttctctc |
| −432 | atgtgggcaa tatccgtgtt cccacttcga aagggggaat gacaagatag |
| −382 | gactccctag gggattacag aaagaaaagc aggaaagcaa gcatcctgtt |
| −332 | ggatttcagc agcaggtatg atgtccaggg aaaagaaatt tggatagcca |
| −282 | gggagtgaaa accccaccaa tcttaaacaa gacctctgtg cttcttcccc |
| −232 | agcaacacaa atgtcctgcc agattcctcc tggaaaaaaa cttctgctcc |
| −182 | tgtccccctc caggtcccag gttgcccatg tccaggaaaa gatggatccc |
| −132 | cctatccaaa tcttctccgt ggtgtgtgtg ggtggaggag tggaccctgg |
| −82 | tccaggcagg ggctccaggg aagagaaggc gtcacttccg ggggccttca |
| −32 | ccagtgtctg gtggctccct tctctgattg gg |

### Example 9: High-Throughput Screening of saRNAs Targeting Human THPO Promoter

### (1) Cell Culture and Transfection

A human liver cancer cell HepG2 (ATCC) was cultured in MEM media (Gibco) containing 10% of calf serum (Sigma-Aldrich) and 1% of penicillin/streptomycin (Gibco). The cells were cultured at 37° C. in 5% CO₂. The HepG2 cells were plated at 5000 cells/well in 96-well plates. RNAiMax (Invitrogen, Carlsbad, Calif.) was used to transfect small activating RNAs at a concentration of 25 nM for a duration of 72 h, with 2 replicate wells for each treatment.

### (2) One-Step RT-qPCR

After transfection, the media were discarded, and each well was washed once with 150 μL of PBS once. After washing, 50 μL of cell lysis buffer was added to each well, and incubation was performed at room temperature for 5 min. One microliter of cell lysis buffer was taken from each well and subjected to qPCR analysis on an ABI 7500 fast real-time PCR system (Applied Biosystems) using a one-step TB Green PrimeScrip™ RT-PCR kit II (Takara, RR086A). Each sample was repeatedly amplified in 3 replicate wells. PCR reaction conditions are shown in Table 1.

TABLE 10
PCR reaction preparation

| Reagent | Volume |
|---|---|
| 2 × One-step TB Green RT-PCR buffer 4 | 2.5 μL |
| PrimeScript 1 step enzyme mixture 2 | 0.2 μL |
| Mixture of forward and reverse primers (5 μM) | 0.4 μL |
| No RNase dH₂O | 1.4 μL |
| Crude lysate (RNA) | 0.5 μL |
| Sum | 5 μL |

Reaction conditions were as follows: reverse transcription reaction (stage 1): 5 min at 42° C., 10 s at 95° C.; PCR reaction (stage 2): 5s at 95° C., 20 s at 60° C., 45 cycles of amplification. TBP was used as an internal control gene. PCR primers used for THPO and TBP were shown in Table 10, wherein THPO was amplified using the THPO F1/R1 primer pair.

TABLE 11
Primer sequences for RT-qPCR analysis

| Primer | Sequence No. | Sequence (5′-3′) |
|---|---|---|
| THPO F | SEQ ID NO: 309 | TCGTGACTCCCATGTCCTTC |
| THPO R | SEQ ID NO: 310 | TGCCTTGGTCTCCTCCATCT |
| TBP F | SEQ ID NO: 311 | TGCTCACCCACCAACAATTTAG |
| TBP R | SEQ ID NO: 312 | TCTGCTCTGACTTTAGCACCTG |
| HPRT1 F | SEQ ID NO: 313 | AAAGATGGTCAAGGTCGCAAG |
| HPRT1 R | SEQ ID NO: 314 | TAGTCAAGGGCATATCCTACAAC |

In order to calculate the expression value (Eᵣₑₗ) of the THPO gene (target) of an saRNA-transfected sample relative to control treatment (Mock), the Ct values of the target gene and one internal control gene are substituted into Formula 2 as shown in Example 3 for calculation.

Eᵣₑₗ=2^{(CtTm-CtTs)}/2^{(CtRm-CtRs)} Formula 2

wherein CtTm was the Ct value of the target gene from Mock sample; CtTs was the Ct value of the target gene from the saRNA-treated sample; CtRm was the Ct value of the internal control gene from Mock-treated sample; and CtRs was the Ct value of the internal control gene from the saRNA-treated sample.

(3) Screening of Functional saRNAs

To obtain saRNAs capable of activating THPO transcription, HepG2 cells were transfected by the aforementioned 833 saRNAs with a transfection concentration of 25 nM. Seventy two hours later, using the same method as described above, the cells were lysed and subjected to one-step RT-qPCR analysis to obtain the relative expression value (compared to the control treatment group) of THPO gene in each saRNA-treated sample. As shown in Table 11, 158 (19%) saRNAs showed high activation, 243 (29.2%) saRNAs showed mild activation, and 432 (51.8%) saRNAs did not affect THPO expression. The maximum activation was 5.15-fold; the maximum inhibition was 0.09-fold; and the saRNAs with activation activity were called activating saRNAs.

TABLE 12
High-throughput screening results of human THPO saRNA

| | log₂ value (fold) of changes | Number of | |
|---|---|---|---|
| saRNA activity | in THPO mRNA | saRNAs | Percentage |
| High activation | ≥0.58 (1.50)~≤2.36 (5.15) | 158 | 19% |
| Mild activation | ≥0.13 (1.10)-<0.58 (1.50) | 243 | 29.2% |
| No activating | <0.13 (1.10) | 432 | 51.8% |
| effect | | | |
| Sum | | 833 | 100% |

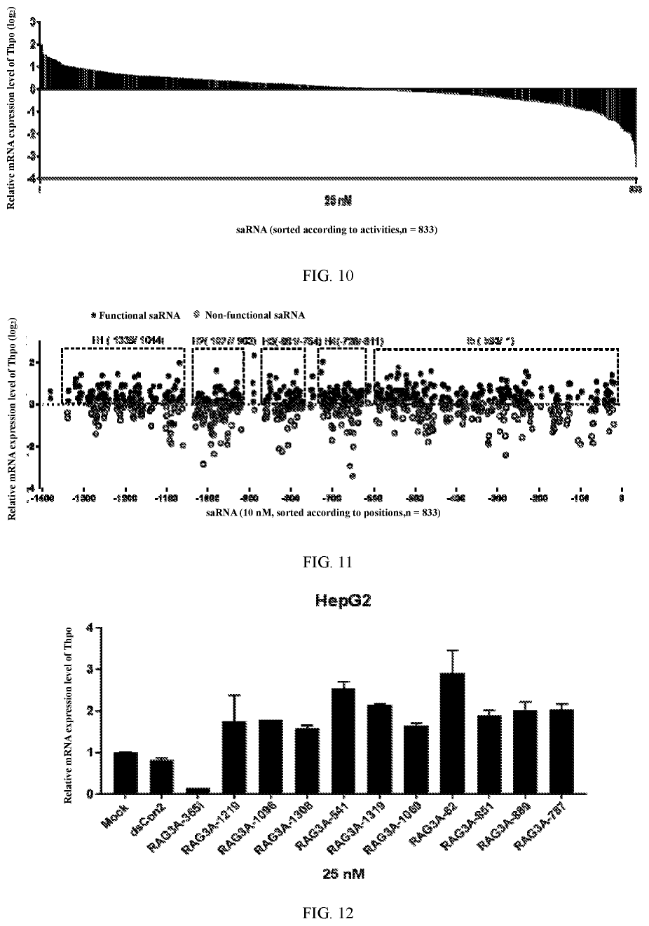

Shown in FIG. 10 is the descending order of changes in THPO expression of mouse THPO saRNA. THPO active saRNA sequence and THPO mRNA expression were changed as shown in Table 13.

TABLE 13
Human active saRNA sequences and target sequences
thereof and changes in expression of THPO mRNA

| | | | | Fold of |
|---|---|---|---|---|
| | | | | changes in |
| | | | | relative |
| | Target sequence | Sense sequence | Antisense sequence | expression |
| saRNA | (5′-3′) | (5′-3′) of saRNA | (5′-3′) of saRNA | of THPO mRNA |
| RAG3A- | AGCCTGAGGAAGTTCTGGG | AGCCUGAGGAAGUUCUGGGTT | CCCAGAACUUCCUCAGGCUT | 5.1 |
| 889 | (SEQ ID NO: 606) | (SEQ ID NO: 1048) | T | |
| | | | (SEQ ID NO: 1490) | |
| RAG3A- | TCCAGCAGCTGTCTTTCCT | UCCAGCAGCUGUCUUUCCUTT | AGGAAAGACAGCUGCUGGAT | 4.1 |
| 723 | (SEQ ID NO: 607) | (SEQ ID NO: 1049) | T | |
| | | | (SEQ ID NO: 1491) | |
| RAG3A- | ATGCCTGCCTCCCTGTCTC | AUGCCUGCCUCCCUGUCUCTT | GAGACAGGGAGGCAGGCAUT | 4 |
| 1069 | (SEQ ID NO: 608) | (SEQ ID NO: 1050) | T | |
| | | | (SEQ ID NO: 1492) | |
| RAG3A- | TTAACCCAGTGCACGTGTG | UUAACCCAGUGCACGUGUGTT | CACACGUGCACUGGGUUAAT | 3.4 |
| 541 | (SEQ ID NO: 609) | (SEQ ID NO: 1051) | T | |
| | | | (SEQ ID NO: 1493) | |
| RAG3A- | TCCAAACCCTTTCCCCATA | UCCAAACCCUUUCCCCAUATT | UAUGGGGAAAGGGUUUGGAT | 3.1 |
| 480 | (SEQ ID NO: 610) | (SEQ ID NO: 1052) | T | |
| | | | (SEQ ID NO: 1494) | |
| RAG3A- | GACATCTCGTCTGGCTGGA | GACAUCUCGUCUGGCUGGATT | UCCAGCCAGACGAGAUGUCT | 3.1 |
| 980 | (SEQ ID NO: 611) | (SEQ ID NO: 1053) | T | |
| | | | (SEQ ID NO: 1495) | |
| RAG3A- | AACACCAACCCATAACAGG | AACACCAACCCAUAACAGGTT | CCUGUUAUGGGUUGGUGUUT | 3 |
| 462 | (SEQ ID NO: 612) | (SEQ ID NO: 1054) | T | |
| | | | (SEQ ID NO: 1496) | |
| RAG3A- | CAAAGGAGCTAGAGCCAG | CAAAGGAGCUAGAGCCAGGTT | CCUGGCUCUAGCUCCUUUGT | 2.9 |
| 787 | G | (SEQ ID NO: 1055) | T | |
| | (SEQ ID NO: 613) | | (SEQ ID NO: 1497) | |
| RAG3A- | AGCAACACAAATGTCCTGC | AGCAACACAAAUGUCCUGCTT | GCAGGACAUUUGUGUUGCUT | 2.9 |
| 232 | (SEQ ID NO: 614) | (SEQ ID NO: 1056) | T | |
| | | | (SEQ ID NO: 1498) | |
| RAG3A- | GAGGTCCAGCAGCTGTCTT | GAGGUCCAGCAGCUGUCUUTT | AAGACAGCUGCUGGACCUCT | 2.9 |
| 727 | (SEQ ID NO: 615) | (SEQ ID NO: 1057) | T | |
| | | | (SEQ ID NO: 1499) | |
| RAG3A- | CTTAACCCAGTGCACGTGT | CUUAACCCAGUGCACGUGUTT | ACACGUGCACUGGGUUAAGT | 2.8 |
| 542 | (SEQ ID NO: 616) | (SEQ ID NO: 1058) | T | |
| | | | (SEQ ID NO: 1500) | |
| RAG3A- | AGCAGGGAGGTATTTGCGG | AGCAGGGAGGUAUUUGCGGTT | CCGCAAAUACCUCCCUGCUTT | 2.8 |
| 748 | (SEQ ID NO: 617) | (SEQ ID NO: 1059) | (SEQ ID NO: 1501) | |
| RAG3A- | TAACCCAGTGCACGTGTGC | UAACCCAGUGCACGUGUGCTT | GCACACGUGCACUGGGUUAT | 2.8 |
| 540 | (SEQ ID NO: 618) | (SEQ ID NO: 1060) | T | |
| | | | (SEQ ID NO: 1502) | |
| RAG3A- | TCTGTGGAGTGACAGTATC | UCUGUGGAGUGACAGUAUCTT | GAUACUGUCACUCCACAGAT | 2.8 |
| 1219 | (SEQ ID NO: 619) | (SEQ ID NO: 1061) | T | |
| | | | (SEQ ID NO: 1503) | |
| RAG3A- | CAACCCGTCCAAACCCTTT | CAACCCGUCCAAACCCUUUTT | AAAGGGUUUGGACGGGUUGT | 2.7 |
| 487 | (SEQ ID NO: 620) | (SEQ ID NO: 1062) | T | |
| | | | (SEQ ID NO: 1504) | |
| RAG3A- | GAGCACCTAAGCTCAGGCT | GAGCACCUAAGCUCAGGCUTT | AGCCUGAGCUUAGGUGCUCT | 2.7 |
| 559 | (SEQ ID NO: 621) | (SEQ ID NO: 1063) | T | |
| | | | (SEQ ID NO: 1505) | |
| RAG3A- | CCTGCCTCCCTACCTTCTC | CCUGCCUCCCUACCUUCUCTT | GAGAAGGUAGGGAGGCAGGT | 2.7 |
| 1308 | (SEQ ID NO: 622) | (SEQ ID NO: 1064) | T | |
| | | | (SEQ ID NO: 1506) | |
| RAG3A- | TGGTGGCTCCCTTCTCTGA | UGGUGGCUCCCUUCUCUGATT | UCAGAGAAGGGAGCCACCAT | 2.6 |
| 24 | (SEQ ID NO: 623) | (SEQ ID NO: 1065) | T | |
| | | | (SEQ ID NO: 1507) | |
| RAG3A- | TTCGACAGTCCCCTCTTGC | UUCGACAGUCCCCUCUUGCTT | GCAAGAGGGGACUGUCGAAT | 2.6 |
| 1169 | (SEQ ID NO: 624) | (SEQ ID NO: 1066) | T | |
| | | | (SEQ ID NO: 1508) | |
| RAG3A- | GACAGGGACCTAGCCAGA | GACAGGGACCUAGCCAGAATT | UUCUGGCUAGGUCCCUGUCT | 2.6 |
| 1256 | A | (SEQ ID NO: 1067) | T | |
| | (SEQ ID NO: 625) | | (SEQ ID NO: 1509) | |
| RAG3A- | CGCACATACATGTGCCCCG | CGCACAUACAUGUGCCCCGTT | CGGGGCACAUGUAUGUGCGT | 2.6 |
| 522 | (SEQ ID NO: 626) | (SEQ ID NO: 1068) | T | |
| | | | (SEQ ID NO: 1510) | |
| RAG3A- | TCAAGGTCAGGCCAGGAA | UCAAGGUCAGGCCAGGAAGTT | CUUCCUGGCCUGACCUUGATT | 2.6 |
| 851 | G | (SEQ ID NO: 1069) | (SEQ ID NO: 1511) | |
| | (SEQ ID NO: 627) | | | |
| RAG3A- | AGCCTCCTTTCTCTTGGTT | AGCCUCCUUUCUCUUGGUUTT | AACCAAGAGAAAGGAGGCUT | 2.5 |
| 1096 | (SEQ ID NO: 628) | (SEQ ID NO: 1070) | T | |
| | | | (SEQ ID NO: 1512) | |
| RAG3A- | CACAAATGTCCTGCCAGAT | CACAAAUGUCCUGCCAGAUTT | AUCUGGCAGGACAUUUGUGT | 2.5 |
| 227 | (SEQ ID NO: 629) | (SEQ ID NO: 1071) | T | |
| | | | (SEQ ID NO: 1513) | |
| RAG3A- | TCAGGCGCATTCGACAGTC | UCAGGCGCAUUCGACAGUCTT | GACUGUCGAAUGCGCCUGAT | 2.5 |
| 1178 | (SEQ ID NO: 630) | (SEQ ID NO: 1072) | T | |
| | | | (SEQ ID NO: 1514) | |
| RAG3A- | GGCTTAACCCAGTGCACGT | GGCUUAACCCAGUGCACGUTT | ACGUGCACUGGGUUAAGCCT | 2.5 |
| 544 | (SEQ ID NO: 631) | (SEQ ID NO: 1073) | T | |
| | | | (SEQ ID NO: 1515) | |
| RAG3A- | GAAGCCCCTGAGGACAGA | GAAGCCCCUGAGGACAGAGTT | CUCUGUCCUCAGGGGCUUCT | 2.4 |
| 836 | G | (SEQ ID NO: 1074) | T | |
| | (SEQ ID NO: 632) | | (SEQ ID NO: 1516) | |
| RAG3A- | CCCAGCAGACGAGCACCTA | CCCAGCAGACGAGCACCUATT | UAGGUGCUCGUCUGCUGGGT | 2.4 |
| 569 | (SEQ ID NO: 633) | (SEQ ID NO: 1075) | T | |
| | | | (SEQ ID NO: 1517) | |
| RAG3A- | CTCACTGCCTAGCCTGCCT | CUCACUGCCUAGCCUGCCUTT | AGGCAGGCUAGGCAGUGAGT | 2.4 |
| 1320 | (SEQ ID NO: 634) | (SEQ ID NO: 1076) | T | |
| | | | (SEQ ID NO: 1518) | |
| RAG3A- | TCACTGCCTAGCCTGCCTC | UCACUGCCUAGCCUGCCUCTT | GAGGCAGGCUAGGCAGUGAT | 2.3 |
| 1319 | (SEQ ID NO: 635) | (SEQ ID NO: 1077) | T | |
| | | | (SEQ ID NO: 1519) | |
| RAG3A- | GATAGCCAGGGAGTGAAA | GAUAGCCAGGGAGUGAAAATT | UUUUCACUCCCUGGCUAUCT | 2.3 |
| 290 | A | (SEQ ID NO: 1078) | T | |
| | (SEQ ID NO: 636) | | (SEQ ID NO: 1520) | |
| RAG3A- | TGGTCATGCCTGCCTCCCT | UGGUCAUGCCUGCCUCCCUTT | AGGGAGGCAGGCAUGACCAT | 2.2 |
| 1074 | (SEQ ID NO: 637) | (SEQ ID NO: 1079) | T | |
| | | | (SEQ ID NO: 1521) | |
| RAG3A- | AGTGAAAACCCCACCAATC | AGUGAAAACCCCACCAAUCTT | GAUUGGUGGGGUUUUCACUT | 2.2 |
| 279 | (SEQ ID NO: 638) | (SEQ ID NO: 1080) | T | |
| | | | (SEQ ID NO: 1522) | |
| RAG3A- | GCACATACATGTGCCCCGC | GCACAUACAUGUGCCCCGCTT | GCGGGGCACAUGUAUGUGCT | 2.2 |
| 521 | (SEQ ID NO: 639) | (SEQ ID NO: 1081) | T | |
| | | | (SEQ ID NO: 1523) | |
| RAG3A- | CTGGTGGCTCCCTTCTCTG | CUGGUGGCUCCCUUCUCUGTT | CAGAGAAGGGAGCCACCAGT | 2.1 |
| 25 | (SEQ ID NO: 640) | (SEQ ID NO: 1082) | T | |
| | | | (SEQ ID NO: 1524) | |
| RAG3A- | AAGAGAAGGCGTCACTTCC | AAGAGAAGGCGUCACUUCCTT | GGAAGUGACGCCUUCUCUUT | 2.1 |
| 62 | (SEQ ID NO: 641) | (SEQ ID NO: 1083) | T | |
| | | | (SEQ ID NO: 1525) | |
| RAG3A- | ATCAAGGTCAGGCCAGGA | AUCAAGGUCAGGCCAGGAATT | UUCCUGGCCUGACCUUGAUT | 2.1 |
| 852 | A | (SEQ ID NO: 1084) | T | |
| | (SEQ ID NO: 642) | | (SEQ ID NO: 1526) | |
| RAG3A- | CTCAGCAGACATGCCCATC | CUCAGCAGACAUGCCCAUCTT | GAUGGGCAUGUCUGCUGAGT | 2.1 |
| 931 | (SEQ ID NO: 643) | (SEQ ID NO: 1085) | T | |
| | | | (SEQ ID NO: 1527) | |
| RAG3A- | GAGAGCCTGAGGAAGTTCT | GAGAGCCUGAGGAAGUUCUTT | AGAACUUCCUCAGGCUCUCT | 2 |
| 892 | (SEQ ID NO: 644) | (SEQ ID NO: 1086) | T | |
| | | | (SEQ ID NO: 1528) | |
| RAG3A- | CCCAGCAACACAAATGTCC | CCCAGCAACACAAAUGUCCTT | GGACAUUUGUGUUGCUGGGT | 2 |
| 235 | (SEQ ID NO: 645) | (SEQ ID NO: 1087) | T | |
| | | | (SEQ ID NO: 1529) | |
| RAG3A- | TAGCCAGAAACCGGCAGC | UAGCCAGAAACCGGCAGCATT | UGCUGCCGGUUUCUGGCUAT | 2 |
| 1246 | A | (SEQ ID NO: 1088) | T | |
| | (SEQ ID NO: 646) | | (SEQ ID NO: 1530) | |
| RAG3A- | GCTCCCTTCTCTGATTGGG | GCUCCCUUCUCUGAUUGGGTT | CCCAAUCAGAGAAGGGAGCT | 2 |
| 19 | (SEQ ID NO: 647) | (SEQ ID NO: 1089) | T | |
| | | | (SEQ ID NO: 1531) | |
| RAG3A- | CTCTCACTGCCTAGCCTGC | CUCUCACUGCCUAGCCUGCTT | GCAGGCUAGGCAGUGAGAGT | 2 |
| 1322 | (SEQ ID NO: 648) | (SEQ ID NO: 1090) | T | |
| | | | (SEQ ID NO: 1532) | |
| RAG3A- | GAAGAAAGCAAAGGAGCT | GAAGAAAGCAAAGGAGCUATT | UAGCUCCUUUGCUUUCUUCT | 2 |
| 795 | A | (SEQ ID NO: 1091) | T | |
| | (SEQ ID NO: 649) | | (SEQ ID NO: 1533) | |
| RAG3A- | ACATGTGCCCCGCACCTGA | ACAUGUGCCCCGCACCUGATT | UCAGGUGCGGGGCACAUGUT | 2 |
| 515 | (SEQ ID NO: 650) | (SEQ ID NO: 1092) | T | |
| | | | (SEQ ID NO: 1534) | |
| RAG3A- | TCCAAATCTTCTCCGTGGT | UCCAAAUCUUCUCCGUGGUTT | ACCACGGAGAAGAUUUGGAT | 2 |
| 128 | (SEQ ID NO: 651) | (SEQ ID NO: 1093) | T | |
| | | | (SEQ ID NO: 1535) | |
| RAG3A- | GGAACGGTAGGAGATGGA | GGAACGGUAGGAGAUGGAATT | UUCCAUCUCCUACCGUUCCTT | 2 |
| 661 | A | (SEQ ID NO: 1094) | (SEQ ID NO: 1536) | |
| | (SEQ ID NO: 652) | | | |
| RAG3A- | AAATCTTCTCCGTGGTGTG | AAAUCUUCUCCGUGGUGUGTT | CACACCACGGAGAAGAUUUT | 2 |
| 125 | (SEQ ID NO: 653) | (SEQ ID NO: 1095) | T | |
| | | | (SEQ ID NO: 1537) | |
| RAG3A- | AGAAACCGGCAGCATTCCC | AGAAACCGGCAGCAUUCCCTT | GGGAAUGCUGCCGGUUUCUT | 2 |
| 1241 | (SEQ ID NO: 654) | (SEQ ID NO: 1096) | T | |
| | | | (SEQ ID NO: 1538) | |
| RAG3A- | TAACACCAACCCATAACAG | UAACACCAACCCAUAACAGTT | CUGUUAUGGGUUGGUGUUAT | 2 |
| 463 | (SEQ ID NO: 655) | (SEQ ID NO: 1097) | T | |
| | | | (SEQ ID NO: 1539) | |
| RAG3A- | TCATGTGGGCAATATCCGT | UCAUGUGGGCAAUAUCCGUTT | ACGGAUAUUGCCCACAUGAT | 1.9 |
| 434 | (SEQ ID NO: 656) | (SEQ ID NO: 1098) | T | |
| | | | (SEQ ID NO: 1540) | |
| RAG3A- | CACATACATGTGCCCCGCA | CACAUACAUGUGCCCCGCATT | UGCGGGGCACAUGUAUGUGT | 1.9 |
| 520 | (SEQ ID NO: 657) | (SEQ ID NO: 1099) | T | |
| | | | (SEQ ID NO: 1541) | |
| RAG3A- | TGACAGTCCACTCAACCCG | UGACAGUCCACUCAACCCGTT | CGGGUUGAGUGGACUGUCAT | 1.9 |
| 499 | (SEQ ID NO: 658) | (SEQ ID NO: 1100) | T | |
| | | | (SEQ ID NO: 1542) | |
| RAG3A- | CACATGTGGAACGGTAGGA | CACAUGUGGAACGGUAGGATT | UCCUACCGUUCCACAUGUGT | 1.9 |
| 668 | (SEQ ID NO: 659) | (SEQ ID NO: 1101) | T | |
| | | | (SEQ ID NO: 1543) | |
| RAG3A- | GTAGCCATCTAAGCCACTG | GUAGCCAUCUAAGCCACUGTT | CAGUGGCUUAGAUGGCUACT | 1.9 |
| 591 | (SEQ ID NO: 660) | (SEQ ID NO: 1102) | T (SEQ ID NO: 1544) | |
| RAG3A- | GACAGTATCTCCCTCTCAT | GACAGUAUCUCCCUCUCAUTT | AUGAGAGGGAGAUACUGUCT | 1.9 |
| 1209 | (SEQ ID NO: 661) | (SEQ ID NO: 1103) | T (SEQ ID NO: 1545) | |
| RAG3A- | GGACAGGGACCTAGCCAG | GGACAGGGACCUAGCCAGATT | UCUGGCUAGGUCCCUGUCCT | 1.9 |
| 1257 | A (SEQ ID NO: 662) | (SEQ ID NO: 1104) | T (SEQ ID NO: 1546) | |
| RAG3A- | ATTCGACAGTCCCCTCTTG | AUUCGACAGUCCCCUCUUGTT | CAAGAGGGGACUGUCGAAUT | 1.9 |
| 1170 | (SEQ ID NO: 663) | (SEQ ID NO: 1105) | T (SEQ ID NO: 1547) | |
| RAG3A- | GCCTTCACCAGTGTCTGGT | GCCUUCACCAGUGUCUGGUTT | ACCAGACACUGGUGAAGGCT | 1.9 |
| 39 | (SEQ ID NO: 664) | (SEQ ID NO: 1106) | T (SEQ ID NO: 1548) | |
| RAG3A- | TGTGGGCAATATCCGTGTT | UGUGGGCAAUAUCCGUGUUT | AACACGGAUAUUGCCCACAT | 1.9 |
| 431 | (SEQ ID NO: 665) | T + B50 | T (SEQ ID NO: 1549) | |
| | | (SEQ ID NO: 1107) | | |
| RAG3A- | GTTCCCTGGTCATGCCTGC | GUUCCCUGGUCAUGCCUGCTT | GCAGGCAUGACCAGGGAACT | 1.9 |
| 1080 | (SEQ ID NO: 666) | (SEQ ID NO: 1108) | T (SEQ ID NO: 1550) | |
| RAG3A- | TGTTACCCAGGACAGGGAC | UGUUACCCAGGACAGGGACTT | GUCCCUGUCCUGGGUAACAT | 1.9 |
| 1266 | (SEQ ID NO: 667) | (SEQ ID NO: 1109) | T (SEQ ID NO: 1551) | |
| RAG3A- | TCTCCCTCTCATTGTAACT | UCUCCCUCUCAUUGUAACUTT | AGUUACAAUGAGAGGGAGAT | 1.9 |
| 1202 | (SEQ ID NO: 668) | (SEQ ID NO: 1110) | T (SEQ ID NO: 1552) | |
| RAG3A- | ACATCTCGTCTGGCTGGAC | ACAUCUCGUCUGGCUGGACTT | GUCCAGCCAGACGAGAUGUT | 1.9 |
| 979 | (SEQ ID NO: 669) | (SEQ ID NO: 1111) | T (SEQ ID NO: 1553) | |
| RAG3A- | AGCATCCTGTTGGATTTCA | AGCAUCCUGUUGGAUUUCATT | UGAAAUCCAACAGGAUGCUT | 1.9 |
| 343 | (SEQ ID NO: 670) | (SEQ ID NO: 1112) | T (SEQ ID NO: 1554) | |
| RAG3A- | GTGGGCAATATCCGTGTTC | GUGGGCAAUAUCCGUGUUCTT | GAACACGGAUAUUGCCCACT | 1.9 |
| 430 | (SEQ ID NO: 671) | (SEQ ID NO: 1113) | T (SEQ ID NO: 1555) | |
| RAG3A- | AGGTATGATGTCCAGGGAA | AGGUAUGAUGUCCAGGGAATT | UUCCCUGGACAUCAUACCUT | 1.8 |
| 319 | (SEQ ID NO: 672) | (SEQ ID NO: 1114) | T (SEQ ID NO: 1556) | |
| RAG3A- | TGGGCACAGAGGTCTGTGT | UGGGCACAGAGGUCUGUGUTT | ACACAGACCUCUGUGCCCATT | 1.8 |
| 612 | (SEQ ID NO: 673) | (SEQ ID NO: 1115) | (SEQ ID NO: 1557) | |
| RAG3A- | CCCATAACAGGAGATTTCT | CCCAUAACAGGAGAUUUCUTT | AGAAAUCUCCUGUUAUGGGT | 1.8 |
| 454 | (SEQ ID NO: 674) | (SEQ ID NO: 1116) | T (SEQ ID NO: 1558) | |
| RAG3A- | CTAGCCTGCCTCCCTACCT | CUAGCCUGCCUCCCUACCUTT | AGGUAGGGAGGCAGGCUAGT | 1.8 |
| 1312 | (SEQ ID NO: 675) | (SEQ ID NO: 1117) | T (SEQ ID NO: 1559) | |
| RAG3A- | GCATCCTGTTGGATTTCAG | GCAUCCUGUUGGAUUUCAGTT | CUGAAAUCCAACAGGAUGCT | 1.8 |
| 342 | (SEQ ID NO: 676) | (SEQ ID NO: 1118) | T (SEQ ID NO: 1560) | |
| RAG3A- | TCTGCCTCTCCAGCCCACT | UCUGCCUCUCCAGCCCACUTT | AGUGGGCUGGAGAGGCAGAT | 1.8 |
| 1118 | (SEQ ID NO: 677) | (SEQ ID NO: 1119) | T (SEQ ID NO: 1561) | |
| RAG3A- | CTGGCTGGACGGGAAAATT | CUGGCUGGACGGGAAAAUUTT | AAUUUUCCCGUCCAGCCAGT | 1.8 |
| 970 | (SEQ ID NO: 678) | (SEQ ID NO: 1120) | T (SEQ ID NO: 1562) | |
| RAG3A- | TCACCCAAGGGACCCTCTG | UCACCCAAGGGACCCUCUGTT | CAGAGGGUCCCUUGGGUGAT | 1.8 |
| 1133 | (SEQ ID NO: 679) | (SEQ ID NO: 1121) | T (SEQ ID NO: 1563) | |
| RAG3A- | GGGCCTGGTTATTCCTCTT | GGGCCUGGUUAUUCCUCUUTT | AAGAGGAAUAACCAGGCCCT | 1.8 |
| 689 | (SEQ ID NO: 680) | (SEQ ID NO: 1122) | T (SEQ ID NO: 1564) | |
| RAG3A- | TCTCACTGCCTAGCCTGCC | UCUCACUGCCUAGCCUGCCTT | GGCAGGCUAGGCAGUGAGAT | 1.8 |
| 1321 | (SEQ ID NO: 681) | (SEQ ID NO: 1123) | T (SEQ ID NO: 1565) | |
| RAG3A- | TGGAACGGTAGGAGATGGA | UGGAACGGUAGGAGAUGGATT | UCCAUCUCCUACCGUUCCATT | 1.8 |
| 662 | (SEQ ID NO: 682) | (SEQ ID NO: 1124) | (SEQ ID NO: 1566) | |
| RAG3A- | ACTGTGGGGAGACTGGGA | ACUGUGGGGAGACUGGGACTT | GUCCCAGUCUCCCCACAGUTT | 1.8 |
| 817 | C (SEQ ID NO: 683) | (SEQ ID NO: 1125) | (SEQ ID NO: 1567) | |
| RAG3A- | CAGAAACCGGCAGCATTCC | CAGAAACCGGCAGCAUUCCTT | GGAAUGCUGCCGGUUUCUGT | 1.8 |
| 1242 | (SEQ ID NO: 684) | (SEQ ID NO: 1126) | T (SEQ ID NO: 1568) | |
| RAG3A- | GTGAAAACCCCACCAATCT | GUGAAAACCCCACCAAUCUTT | AGAUUGGUGGGGUUUUCACT | 1.8 |
| 278 | (SEQ ID NO: 685) | (SEQ ID NO: 1127) | T (SEQ ID NO: 1569) | |
| RAG3A- | TGACAGTATCTCCCTCTCA | UGACAGUAUCUCCCUCUCATT | UGAGAGGGAGAUACUGUCAT | 1.8 |
| 1210 | (SEQ ID NO: 686) | (SEQ ID NO: 1128) | T (SEQ ID NO: 1570) | |
| RAG3A- | CTTGGTTCCCTGGTCATGC | CUUGGUUCCCUGGUCAUGCTT | GCAUGACCAGGGAACCAAGT | 1.8 |
| 1084 | (SEQ ID NO: 687) | (SEQ ID NO: 1129) | T (SEQ ID NO: 1571) | |
| RAG3A- | GACAGTCCCCTCTTGCTTT | GACAGUCCCCUCUUGCUUUTT | AAAGCAAGAGGGGACUGUCT | 1.8 |
| 1166 | (SEQ ID NO: 688) | (SEQ ID NO: 1130) | T (SEQ ID NO: 1572) | |
| RAG3A- | GAGACATCTCGTCTGGCTG | GAGACAUCUCGUCUGGCUGTT | CAGCCAGACGAGAUGUCUCT | 1.8 |
| 982 | (SEQ ID NO: 689) | (SEQ ID NO: 1131) | T (SEQ ID NO: 1573) | |
| RAG3A- | AGGTCCCAGGTTGCCCATG | AGGUCCCAGGUUGCCCAUGTT | CAUGGGCAACCUGGGACCUT | 1.8 |
| 171 | (SEQ ID NO: 690) | (SEQ ID NO: 1132) | T (SEQ ID NO: 1574) | |
| RAG3A- | ACAAGACCTCTGTGCTTCT | ACAAGACCUCUGUGCUUCUTT | AGAAGCACAGAGGUCUUGUT | 1.7 |
| 256 | (SEQ ID NO: 691) | (SEQ ID NO: 1133) | T (SEQ ID NO: 1575) | |
| RAG3A- | AGACGAGCACCTAAGCTCA | AGACGAGCACCUAAGCUCATT | UGAGCUUAGGUGCUCGUCUT | 1.7 |
| 563 | (SEQ ID NO: 692) | (SEQ ID NO: 1134) | T (SEQ ID NO: 1576) | |
| RAG3A- | AGGCGCATTCGACAGTCCC | AGGCGCAUUCGACAGUCCCTT | GGGACUGUCGAAUGCGCCUT | 1.7 |
| 1176 | (SEQ ID NO: 693) | (SEQ ID NO: 1135) | T (SEQ ID NO: 1577) | |
| RAG3A- | CCTAAGACAGGGACACATG | CCUAAGACAGGGACACAUGTT | CAUGUGUCCCUGUCUUAGGT | 1.7 |
| 707 | (SEQ ID NO: 694) | (SEQ ID NO: 1136) | T (SEQ ID NO: 1578) | |
| RAG3A- | ACGAGCACCTAAGCTCAGG | ACGAGCACCUAAGCUCAGGTT | CCUGAGCUUAGGUGCUCGUT | 1.7 |
| 561 | (SEQ ID NO: 695) | (SEQ ID NO: 1137) | T (SEQ ID NO: 1579) | |
| RAG3A- | GCACCTGACAGTCCACTCA | GCACCUGACAGUCCACUCATT | UGAGUGGACUGUCAGGUGCT | 1.7 |
| 504 | (SEQ ID NO: 696) | (SEQ ID NO: 1138) | T (SEQ ID NO: 1580) | |
| RAG3A- | AAACTTCTGCTCCTGTCCC | AAACUUCUGCUCCUGUCCCTT | GGGACAGGAGCAGAAGUUUT | 1.7 |
| 195 | (SEQ ID NO: 697) | (SEQ ID NO: 1139) | T (SEQ ID NO: 1581) | |
| RAG3A- | CAAGGTCAGGCCAGGAAG | CAAGGUCAGGCCAGGAAGCTT | GCUUCCUGGCCUGACCUUGT | 1.7 |
| 850 | C (SEQ ID NO: 698) | (SEQ ID NO: 1140) | T (SEQ ID NO: 1582) | |
| RAG3A- | AGATAGGACTCCCTAGGGG | AGAUAGGACUCCCUAGGGGTT | CCCCUAGGGAGUCCUAUCUT | 1.7 |
| 388 | (SEQ ID NO: 699) | (SEQ ID NO: 1141) | T (SEQ ID NO: 1583) | |
| RAG3A- | TTCCCTGGTCATGCCTGCC | UUCCCUGGUCAUGCCUGCCTT | GGCAGGCAUGACCAGGGAAT | 1.7 |
| 1079 | (SEQ ID NO: 700) | (SEQ ID NO: 1142) | T (SEQ ID NO: 1584) | |
| RAG3A- | TACCCAGGACAGGGACCTA | UACCCAGGACAGGGACCUATT | UAGGUCCCUGUCCUGGGUAT | 1.7 |
| 1263 | (SEQ ID NO: 701) | (SEQ ID NO: 1143) | T (SEQ ID NO: 1585) | |
| RAG3A- | GCACGTGTGCGCACATACA | GCACGUGUGCGCACAUACATT | UGUAUGUGCGCACACGUGCT | 1.7 |
| 531 | (SEQ ID NO: 702) | (SEQ ID NO: 1144) | T (SEQ ID NO: 1586) | |
| RAG3A- | AAGACCTCTGTGCTTCTTC | AAGACCUCUGUGCUUCUUCTT | GAAGAAGCACAGAGGUCUUT | 1.7 |
| 254 | (SEQ ID NO: 703) | (SEQ ID NO: 1145) | T (SEQ ID NO: 1587) | |
| RAG3A- | ATCCGTGTTCCCACTTCGA | AUCCGUGUUCCCACUUCGATT | UCGAAGUGGGAACACGGAUT | 1.7 |
| 421 | (SEQ ID NO: 704) | (SEQ ID NO: 1146) | T (SEQ ID NO: 1588) | |
| RAG3A- | CCTTTCCCCATAACACCAA | CCUUUCCCCAUAACACCAATT | UUGGUGUUAUGGGGAAAGGT | 1.7 |
| 473 | (SEQ ID NO: 705) | (SEQ ID NO: 1147) | T (SEQ ID NO: 1589) | |
| RAG3A- | TGGATAGCCAGGGAGTGAA | UGGAUAGCCAGGGAGUGAATT | UUCACUCCCUGGCUAUCCATT | 1.7 |
| 292 | (SEQ ID NO: 706) | (SEQ ID NO: 1148) | (SEQ ID NO: 1590) | |
| RAG3A- | AGGGAGTGAAAACCCCAC | AGGGAGUGAAAACCCCACCTT | GGUGGGGUUUUCACUCCCUT | 1.7 |
| 283 | C (SEQ ID NO: 707) | (SEQ ID NO: 1149) | T (SEQ ID NO: 1591) | |
| RAG3A- | AAAGGAGCTAGAGCCAGG | AAAGGAGCUAGAGCCAGGGTT | CCCUGGCUCUAGCUCCUUUT | 1.7 |
| 786 | G (SEQ ID NO: 708) | (SEQ ID NO: 1150) | T (SEQ ID NO: 1592) | |
| RAG3A- | TAAGCCACTGGACCCCAGC | UAAGCCACUGGACCCCAGCTT | GCUGGGGUCCAGUGGCUUAT | 1.7 |
| 582 | (SEQ ID NO: 709) | (SEQ ID NO: 1151) | T (SEQ ID NO: 1593) | |
| RAG3A- | TTCTCAGCAGACATGCCCA | UUCUCAGCAGACAUGCCCATT | UGGGCAUGUCUGCUGAGAAT | 1.7 |
| 933 | (SEQ ID NO: 710) | (SEQ ID NO: 1152) | T (SEQ ID NO: 1594) | |
| RAG3A- | CTTTCCCCATAACACCAAC | CUUUCCCCAUAACACCAACTT | GUUGGUGUUAUGGGGAAAGT | 1.7 |
| 472 | (SEQ ID NO: 711) | (SEQ ID NO: 1153) | T (SEQ ID NO: 1595) | |
| RAG3A- | GGAAGACGGAGACAGAAC | GGAAGACGGAGACAGAACATT | UGUUCUGUCUCCGUCUUCCT | 1.6 |
| 646 | A (SEQ ID NO: 712) | (SEQ ID NO: 1154) | T (SEQ ID NO: 1596) | |
| RAG3A- | AAGATAGGACTCCCTAGGG | AAGAUAGGACUCCCUAGGGTT | CCCUAGGGAGUCCUAUCUUT | 1.6 |
| 389 | (SEQ ID NO: 713) | (SEQ ID NO: 1155) | T (SEQ ID NO: 1597) | |
| RAG3A- | TCTGGCTGGACGGGAAAAT | UCUGGCUGGACGGGAAAAUTT | AUUUUCCCGUCCAGCCAGATT | 1.6 |
| 971 | (SEQ ID NO: 714) | (SEQ ID NO: 1156) | (SEQ ID NO: 1598) | |
| RAG3A- | GCCAGCAGGGAGGTATTTG | GCCAGCAGGGAGGUAUUUGTT | CAAAUACCUCCCUGCUGGCT | 1.6 |
| 751 | (SEQ ID NO: 715) | (SEQ ID NO: 1157) | T (SEQ ID NO: 1599) | |
| RAG3A- | AGAAATTTGGATAGCCAGG | AGAAAUUUGGAUAGCCAGGTT | CCUGGCUAUCCAAAUUUCUT | 1.6 |
| 299 | (SEQ ID NO: 716) | (SEQ ID NO: 1158) | T (SEQ ID NO: 1600) | |
| RAG3A- | CCAGCAGGGAGGTATTTGC | CCAGCAGGGAGGUAUUUGCTT | GCAAAUACCUCCCUGCUGGT | 1.6 |
| 750 | (SEQ ID NO: 717) | (SEQ ID NO: 1159) | T (SEQ ID NO: 1601) | |
| RAG3A- | TGTCACATGTGGAACGGTA | UGUCACAUGUGGAACGGUATT | UACCGUUCCACAUGUGACAT | 1.6 |
| 671 | (SEQ ID NO: 718) | (SEQ ID NO: 1160) | T (SEQ ID NO: 1602) | |
| RAG3A- | CAGCAGGGAGGTATTTGCG | CAGCAGGGAGGUAUUUGCGTT | CGCAAAUACCUCCCUGCUGT | 1.6 |
| 749 | (SEQ ID NO: 719) | (SEQ ID NO: 1161) | T (SEQ ID NO: 1603) | |
| RAG3A- | TCAGGCTTAACCCAGTGCA | UCAGGCUUAACCCAGUGCATT | UGCACUGGGUUAAGCCUGAT | 1.6 |
| 547 | (SEQ ID NO: 720) | (SEQ ID NO: 1162) | T (SEQ ID NO: 1604) | |
| RAG3A- | GTAGGAGATGGAAGACGG | GUAGGAGAUGGAAGACGGATT | UCCGUCUUCCAUCUCCUACTT | 1.6 |
| 655 | A (SEQ ID NO: 721) | (SEQ ID NO: 1163) | (SEQ ID NO: 1605) | |
| RAG3A- | GGTCCCAGGTTGCCCATGT | GGUCCCAGGUUGCCCAUGUTT | ACAUGGGCAACCUGGGACCT | 1.6 |
| 170 | (SEQ ID NO: 722) | (SEQ ID NO: 1164) | T (SEQ ID NO: 1606) | |
| RAG3A- | CAAGCAAAGGAGGGCCCT | CAAGCAAAGGAGGGCCCUGTT | CAGGGCCCUCCUUUGCUUGT | 1.6 |
| 629 | G (SEQ ID NO: 723) | (SEQ ID NO: 1165) | T (SEQ ID NO: 1607) | |
| RAG3A- | AGACCTCTGTGCTTCTTCC | AGACCUCUGUGCUUCUUCCTT | GGAAGAAGCACAGAGGUCUT | 1.6 |
| 253 | (SEQ ID NO: 724) | (SEQ ID NO: 1166) | T (SEQ ID NO: 1608) | |
| RAG3A- | ACACATGGGCCTGGTTATT | ACACAUGGGCCUGGUUAUUTT | AAUAACCAGGCCCAUGUGUT | 1.6 |
| 695 | (SEQ ID NO: 725) | (SEQ ID NO: 1167) | T (SEQ ID NO: 1609) | |
| RAG3A- | GCAAAGGAGCTAGAGCCA | GCAAAGGAGCUAGAGCCAGTT | CUGGCUCUAGCUCCUUUGCT | 1.6 |
| 788 | G (SEQ ID NO: 726) | (SEQ ID NO: 1168) | T (SEQ ID NO: 1610) | |
| RAG3A- | GGCCTGGTTATTCCTCTTG | GGCCUGGUUAUUCCUCUUGTT | CAAGAGGAAUAACCAGGCCT | 1.6 |
| 688 | (SEQ ID NO: 727) | (SEQ ID NO: 1169) | T (SEQ ID NO: 1611) | |
| RAG3A- | GGAGGTCCAGCAGCTGTCT | GGAGGUCCAGCAGCUGUCUTT | AGACAGCUGCUGGACCUCCT | 1.6 |
| 728 | (SEQ ID NO: 728) | (SEQ ID NO: 1170) | T (SEQ ID NO: 1612) | |
| RAG3A- | ACATACATGTGCCCCGCAC | ACAUACAUGUGCCCCGCACTT | GUGCGGGGCACAUGUAUGUT | 1.6 |
| 519 | (SEQ ID NO: 729) | (SEQ ID NO: 1171) | T (SEQ ID NO: 1613) | |
| RAG3A- | ACCCCACCAATCTTAAACA | ACCCCACCAAUCUUAAACATT | UGUUUAAGAUUGGUGGGGUT | 1.6 |
| 272 | (SEQ ID NO: 730) | (SEQ ID NO: 1172) | T (SEQ ID NO: 1614) | |
| RAG3A- | GGTATGATGTCCAGGGAAA | GGUAUGAUGUCCAGGGAAATT | UUUCCCUGGACAUCAUACCT | 1.6 |
| 318 | (SEQ ID NO: 731) | (SEQ ID NO: 1173) | T (SEQ ID NO: 1615) | |
| RAG3A- | AAACCCTCACCACCCTCTC | AAACCCUCACCACCCUCUCTT | GAGAGGGUGGUGAGGGUUUT | 1.6 |
| 1338 | (SEQ ID NO: 732) | (SEQ ID NO: 1174) | T (SEQ ID NO: 1616) | |
| RAG3A- | AACCCAGTGCACGTGTGCG | AACCCAGUGCACGUGUGCGTT | CGCACACGUGCACUGGGUUT | 1.6 |
| 539 | (SEQ ID NO: 733) | (SEQ ID NO: 1175) | T (SEQ ID NO: 1617) | |
| RAG3A- | AGGACAGAGACTGTGGGG | AGGACAGAGACUGUGGGGATT | UCCCCACAGUCUCUGUCCUTT | 1.6 |
| 826 | A (SEQ ID NO: 734) | (SEQ ID NO: 1176) | (SEQ ID NO: 1618) | |
| RAG3A- | CTGTTGGATTTCAGCAGCA | CUGUUGGAUUUCAGCAGCATT | UGCUGCUGAAAUCCAACAGT | 1.6 |
| 337 | (SEQ ID NO: 735) | (SEQ ID NO: 1177) | T (SEQ ID NO: 1619) | |
| RAG3A- | AACCCCACCAATCTTAAAC | AACCCCACCAAUCUUAAACTT | GUUUAAGAUUGGUGGGGUUT | 1.6 |
| 273 | (SEQ ID NO: 736) | (SEQ ID NO: 1178) | T (SEQ ID NO: 1620) | |
| RAG3A- | CAAACCCTTTCCCCATAAC | CAAACCCUUUCCCCAUAACTT | GUUAUGGGGAAAGGGUUUGT | 1.6 |
| 478 | (SEQ ID NO: 737) | (SEQ ID NO: 1179) | T (SEQ ID NO: 1621) | |
| RAG3A- | GACGGGAAAATTCCAGGAT | GACGGGAAAAUUCCAGGAUTT | AUCCUGGAAUUUUCCCGUCT | 1.6 |
| 963 | (SEQ ID NO: 738) | (SEQ ID NO: 1180) | T (SEQ ID NO: 1622) | |
| RAG3A- | TCTGTGCTTCTTCCCCAGC | UCUGUGCUUCUUCCCCAGCTT | GCUGGGGAAGAAGCACAGAT | 1.6 |
| 248 | (SEQ ID NO: 739) | (SEQ ID NO: 1181) | T (SEQ ID NO: 1623) | |
| RAG3A- | TCACCAGTGTCTGGTGGCT | UCACCAGUGUCUGGUGGCUTT | AGCCACCAGACACUGGUGAT | 1.5 |
| 35 | (SEQ ID NO: 740) | (SEQ ID NO: 1182) | T (SEQ ID NO: 1624) | |
| RAG3A- | CTGGGCACAGAGGTCTGTG | CUGGGCACAGAGGUCUGUGTT | CACAGACCUCUGUGCCCAGT | 1.5 |
| 613 | (SEQ ID NO: 741) | (SEQ ID NO: 1183) | T (SEQ ID NO: 1625) | |
| RAG3A- | CCCTTCCTTCACCCAAGGG | CCCUUCCUUCACCCAAGGGTT | CCCUUGGGUGAAGGAAGGGT | 1.5 |
| 1141 | (SEQ ID NO: 742) | (SEQ ID NO: 1184) | T (SEQ ID NO: 1626) | |
| RAG3A- | AAACCCTTTCCCCATAACA | AAACCCUUUCCCCAUAACATT | UGUUAUGGGGAAAGGGUUUT | 1.5 |
| 477 | (SEQ ID NO: 743) | (SEQ ID NO: 1185) | T (SEQ ID NO: 1627) | |
| RAG3A- | CAGAACAAGCAAAGGAGG | CAGAACAAGCAAAGGAGGGTT | CCCUCCUUUGCUUGUUCUGT | 1.5 |
| 634 | G (SEQ ID NO: 744) | (SEQ ID NO: 1186) | T (SEQ ID NO: 1628) | |
| RAG3A- | GCTCCTCATTCTTGTGTTA | GCUCCUCAUUCUUGUGUUATT | UAACACAAGAAUGAGGAGCT | 1.5 |
| 1280 | (SEQ ID NO: 745) | (SEQ ID NO: 1187) | T (SEQ ID NO: 1629) | |
| RAG3A- | CCTCCCTGTCTCCTGTCTC | CCUCCCUGUCUCCUGUCUCTT | GAGACAGGAGACAGGGAGGT | 1.5 |
| 1062 | (SEQ ID NO: 746) | (SEQ ID NO: 1188) | T (SEQ ID NO: 1630) | |
| RAG3A- | TAGAGCCAGGGCCAAAGG | UAGAGCCAGGGCCAAAGGATT | UCCUUUGGCCCUGGCUCUATT | 1.5 |
| 778 | A (SEQ ID NO: 747) | (SEQ ID NO: 1189) | (SEQ ID NO: 1631) | |
| RAG3A- | CCACACTTCTCAGCAGACA | CCACACUUCUCAGCAGACATT | UGUCUGCUGAGAAGUGUGGT | 1.5 |
| 939 | (SEQ ID NO: 748) | (SEQ ID NO: 1190) | T (SEQ ID NO: 1632) | |
| RAG3A- | CTAGAGCCAGGGCCAAAG | CUAGAGCCAGGGCCAAAGGTT | CCUUUGGCCCUGGCUCUAGT | 1.5 |
| 779 | G (SEQ ID NO: 749) | (SEQ ID NO: 1191) | T (SEQ ID NO: 1633) | |
| RAG3A- | GGGACTGGGAAGAAAGCA | GGGACUGGGAAGAAAGCAATT | UUGCUUUCUUCCCAGUCCCT | 1.5 |
| 803 | A(SEQ ID NO: 750) | (SEQ ID NO: 1192) | T (SEQ ID NO: 1634) | |
| RAG3A- | GTCCATCAGCCGATCTCCC | GUCCAUCAGCCGAUCUCCCTT | GGGAGAUCGGCUGAUGGACT | 1.5 |
| 1382 | (SEQ ID NO: 751) | (SEQ ID NO: 1193) | T (SEQ ID NO: 1635) | |
| RAG3A- | AGGAGTGGACCCTGGTCCA | AGGAGUGGACCCUGGUCCATT | UGGACCAGGGUCCACUCCUT | 1.5 |
| 97 | (SEQ ID NO: 752) | (SEQ ID NO: 1194) | T (SEQ ID NO: 1636) | |
| RAG3A- | GCTTAACCCAGTGCACGTG | GCUUAACCCAGUGCACGUGTT | CACGUGCACUGGGUUAAGCT | 1.5 |
| 543 | (SEQ ID NO: 753) | (SEQ ID NO: 1195) | T (SEQ ID NO: 1637) | |
| RAG3A- | CACCTAAGCTCAGGCTTAA | CACCUAAGCUCAGGCUUAATT | UUAAGCCUGAGCUUAGGUGT | 1.5 |
| 556 | (SEQ ID NO: 754) | (SEQ ID NO: 1196) | T (SEQ ID NO: 1638) | |
| RAG3A- | GAGAAGGCGTCACTTCCGG | GAGAAGGCGUCACUUCCGGTT | CCGGAAGUGACGCCUUCUCT | 1.5 |
| 60 | (SEQ ID NO: 755) | (SEQ ID NO: 1197) | T (SEQ ID NO: 1639) | |
| RAG3A- | TGGAAGACGGAGACAGAA | UGGAAGACGGAGACAGAACTT | GUUCUGUCUCCGUCUUCCATT | 1.5 |
| 647 | C (SEQ ID NO: 756) | (SEQ ID NO: 1198) | (SEQ ID NO: 1640) | |
| RAG3A- | GCTTCTTCCCCAGCAACAC | GCUUCUUCCCCAGCAACACTT | GUGUUGCUGGGGAAGAAGCT | 1.5 |
| 243 | (SEQ ID NO: 757) | (SEQ ID NO: 1199) | T (SEQ ID NO: 1641) | |
| RAG3A- | GTGCACGTGTGCGCACATA | GUGCACGUGUGCGCACAUATT | UAUGUGCGCACACGUGCACT | 1.5 |
| 533 | (SEQ ID NO: 758) | (SEQ ID NO: 1200) | T (SEQ ID NO: 1642) | |
| RAG3A- | AAGCAAAGGAGGGCCCTG | AAGCAAAGGAGGGCCCUGGTT | CCAGGGCCCUCCUUUGCUUT | 1.5 |
| 628 | G (SEQ ID NO: 759) | (SEQ ID NO: 1201) | T (SEQ ID NO: 1643) | |
| RAG3A- | AAGGTCAGGCCAGGAAGC | AAGGUCAGGCCAGGAAGCCTT | GGCUUCCUGGCCUGACCUUT | 1.5 |
| 849 | C (SEQ ID NO: 760) | (SEQ ID NO: 1202) | T (SEQ ID NO: 1644) | |
| RAG3A- | CTGCCTCCCTACCTTCTCT | CUGCCUCCCUACCUUCUCUTT | AGAGAAGGUAGGGAGGCAGT | 1.5 |
| 1307 | (SEQ ID NO: 761) | (SEQ ID NO: 1203) | T (SEQ ID NO: 1645) | |
| RAG3A- | TTGGATAGCCAGGGAGTGA | UUGGAUAGCCAGGGAGUGATT | UCACUCCCUGGCUAUCCAATT | 1.5 |
| 293 | (SEQ ID NO: 762) | (SEQ ID NO: 1204) | (SEQ ID NO: 1646) | |
| RAG3A- | AGGTCGCTCCTCATTCTTG | AGGUCGCUCCUCAUUCUUGTT | CAAGAAUGAGGAGCGACCUT | 1.5 |
| 1285 | (SEQ ID NO: 763) | (SEQ ID NO: 1205) | T (SEQ ID NO: 1647) | |
| RAG3A- | CCCTTCTGTGGAGTGACAG | CCCUUCUGUGGAGUGACAGTT | CUGUCACUCCACAGAAGGGT | 1.5 |
| 1223 | (SEQ ID NO: 764) | (SEQ ID NO: 1206) | T (SEQ ID NO: 1648) | |
| RAG3A- | CAGGCGCATTCGACAGTCC | CAGGCGCAUUCGACAGUCCTT | GGACUGUCGAAUGCGCCUGT | 1.5 |
| 1177 | (SEQ ID NO: 765) | (SEQ ID NO: 1207) | T (SEQ ID NO: 1649) | |
| RAG3A- | ACAAATGTCCTGCCAGATT | ACAAAUGUCCUGCCAGAUUTT | AAUCUGGCAGGACAUUUGUT | 1.5 |
| 226 | (SEQ ID NO: 766) | (SEQ ID NO: 1208) | T (SEQ ID NO: 1650) | |
| RAG3A- | CAGCCTCCTTTCTCTTGGT | CAGCCUCCUUUCUCUUGGUTT | ACCAAGAGAAAGGAGGCUGT | 1.5 |
| 1097 | (SEQ ID NO: 767) | (SEQ ID NO: 1209) | T (SEQ ID NO: 1651) | |
| RAG3A- | TAACTTATCCTCAGGCGCA | UAACUUAUCCUCAGGCGCATT | UGCGCCUGAGGAUAAGUUAT | 1.5 |
| 1188 | (SEQ ID NO: 768) | (SEQ ID NO: 1210) | T (SEQ ID NO: 1652) | |
| RAG3A- | GTGTAGCCATCTAAGCCAC | GUGUAGCCAUCUAAGCCACTT | GUGGCUUAGAUGGCUACACT | 1.5 |
| 593 | (SEQ ID NO: 769) | (SEQ ID NO: 1211) | T (SEQ ID NO: 1653) | |
| RAG3A- | GTCACATGTGGAACGGTAG | GUCACAUGUGGAACGGUAGTT | CUACCGUUCCACAUGUGACT | 1.5 |
| 670 | (SEQ ID NO: 770) | (SEQ ID NO: 1212) | T (SEQ ID NO: 1654) | |
| RAG3A- | CACCTGACAGTCCACTCAA | CACCUGACAGUCCACUCAATT | UUGAGUGGACUGUCAGGUGT | 1.5 |
| 503 | (SEQ ID NO: 771) | (SEQ ID NO: 1213) | T (SEQ ID NO: 1655) | |
| RAG3A- | CGACAGTCCCCTCTTGCTT | CGACAGUCCCCUCUUGCUUTT | AAGCAAGAGGGGACUGUCGT | 1.5 |
| 1167 | (SEQ ID NO: 772) | (SEQ ID NO: 1214) | T (SEQ ID NO: 1656) | |
| RAG3A- | CTAAGCCACTGGACCCCAG | CUAAGCCACUGGACCCCAGTT | CUGGGGUCCAGUGGCUUAGT | 1.5 |
| 583 | (SEQ ID NO: 773) | (SEQ ID NO: 1215) | T (SEQ ID NO: 1657) | |
| RAG3A- | CTAAGACAGGGACACATGG | CUAAGACAGGGACACAUGGTT | CCAUGUGUCCCUGUCUUAGT | 1.5 |
| 706 | (SEQ ID NO: 774) | (SEQ ID NO: 1216) | T (SEQ ID NO: 1658) | |
| RAG3A- | GGAGACATCTCGTCTGGCT | GGAGACAUCUCGUCUGGCUTT | AGCCAGACGAGAUGUCUCCT | 1.5 |
| 983 | (SEQ ID NO: 775) | (SEQ ID NO: 1217) | T (SEQ ID NO: 1659) | |
| RAG3A- | CAGCTGTCTTTCCTAAGAC | CAGCUGUCUUUCCUAAGACTT | GUCUUAGGAAAGACAGCUGT | 1.5 |
| 718 | (SEQ ID NO: 776) | (SEQ ID NO: 1218) | T (SEQ ID NO: 1660) | |
| RAG3A- | CCCTAGGGGATTACAGAAA | CCCUAGGGGAUUACAGAAATT | UUUCUGUAAUCCCCUAGGGT | 1.5 |
| 378 | (SEQ ID NO: 777) | (SEQ ID NO: 1219) | T (SEQ ID NO: 1661) | |
| RAG3A- | GCCTCCTTTCTCTTGGTTC | GCCUCCUUUCUCUUGGUUCTT | GAACCAAGAGAAAGGAGGCT | 1.4 |
| 1095 | (SEQ ID NO: 778) | (SEQ ID NO: 1220) | T (SEQ ID NO: 1662) | |
| RAG3A- | GGGACACATGGGCCTGGTT | GGGACACAUGGGCCUGGUUTT | AACCAGGCCCAUGUGUCCCT | 1.4 |
| 698 | (SEQ ID NO: 779) | (SEQ ID NO: 1221) | T (SEQ ID NO: 1663) | |
| RAG3A- | TGAAAACCCCACCAATCTT | UGAAAACCCCACCAAUCUUTT | AAGAUUGGUGGGGUUUUCAT | 1.4 |
| 277 | (SEQ ID NO: 780) | (SEQ ID NO: 1222) | T (SEQ ID NO: 1664) | |
| RAG3A- | AAGACGGAGACAGAACAA | AAGACGGAGACAGAACAAGTT | CUUGUUCUGUCUCCGUCUUT | 1.4 |
| 644 | G(SEQ ID NO: 781) | (SEQ ID NO: 1223) | T (SEQ ID NO: 1665) | |
| RAG3A- | GGCCACACTTCTCAGCAGA | GGCCACACUUCUCAGCAGATT | UCUGCUGAGAAGUGUGGCCT | 1.4 |
| 941 | (SEQ ID NO: 782) | (SEQ ID NO: 1224) | T (SEQ ID NO: 1666) | |
| RAG3A- | AAGAAAGCAAAGGAGCTA | AAGAAAGCAAAGGAGCUAGTT | CUAGCUCCUUUGCUUUCUUT | 1.4 |
| 794 | G(SEQ ID NO: 783) | (SEQ ID NO: 1225) | T (SEQ ID NO: 1667) | |
| RAG3A- | GAGCCTGAGGAAGTTCTGG | GAGCCUGAGGAAGUUCUGGTT | CCAGAACUUCCUCAGGCUCT | 1.4 |
| 890 | (SEQ ID NO: 784) | (SEQ ID NO: 1226) | T (SEQ ID NO: 1668) | |
| RAG3A- | AGCCTGCCTCCCTACCTTC | AGCCUGCCUCCCUACCUUCTT | GAAGGUAGGGAGGCAGGCUT | 1.4 |
| 1310 | (SEQ ID NO: 785) | (SEQ ID NO: 1227) | T (SEQ ID NO: 1669) | |
| RAG3A- | GTCTGGTGGCTCCCTTCTC | GUCUGGUGGCUCCCUUCUCTT | GAGAAGGGAGCCACCAGACT | 1.4 |
| 27 | (SEQ ID NO: 786) | (SEQ ID NO: 1228) | T (SEQ ID NO: 1670) | |
| RAG3A- | CCTTCACCAGTGTCTGGTG | CCUUCACCAGUGUCUGGUGTT | CACCAGACACUGGUGAAGGT | 1.4 |
| 38 | (SEQ ID NO: 787) | (SEQ ID NO: 1229) | T (SEQ ID NO: 1671) | |
| RAG3A- | TGCACGTGTGCGCACATAC | UGCACGUGUGCGCACAUACTT | GUAUGUGCGCACACGUGCAT | 1.4 |
| 532 | (SEQ ID NO: 788) | (SEQ ID NO: 1230) | T (SEQ ID NO: 1672) | |
| RAG3A- | TATCCTCCCAGCTATCCCA | UAUCCUCCCAGCUAUCCCATT | UGGGAUAGCUGGGAGGAUAT | 1.4 |
| 1023 | (SEQ ID NO: 789) | (SEQ ID NO: 1231) | T (SEQ ID NO: 1673) | |
| RAG3A- | GGGAAGAAAGCAAAGGAG | GGGAAGAAAGCAAAGGAGCTT | GCUCCUUUGCUUUCUUCCCT | 1.4 |
| 797 | C (SEQ ID NO: 790) | (SEQ ID NO: 1232) | T (SEQ ID NO: 1674) | |
| RAG3A- | GGGGAATGACAAGATAGGA | GGGGAAUGACAAGAUAGGATT | UCCUAUCUUGUCAUUCCCCT | 1.4 |
| 399 | (SEQ ID NO: 791) | (SEQ ID NO: 1233) | T (SEQ ID NO: 1675) | |
| RAG3A- | TCTAGGCCACACTTCTCAG | UCUAGGCCACACUUCUCAGTT | CUGAGAAGUGUGGCCUAGAT | 1.4 |
| 945 | (SEQ ID NO: 792) | (SEQ ID NO: 1234) | T (SEQ ID NO: 1676) | |
| RAG3A- | AAGCATCCTGTTGGATTTC | AAGCAUCCUGUUGGAUUUCTT | GAAAUCCAACAGGAUGCUUT | 1.4 |
| 344 | (SEQ ID NO: 793) | (SEQ ID NO: 1235) | T (SEQ ID NO: 1677) | |
| RAG3A- | ACAGGGACCTAGCCAGAA | ACAGGGACCUAGCCAGAAATT | UUUCUGGCUAGGUCCCUGUT | 1.4 |
| 1255 | A (SEQ ID NO: 794) | (SEQ ID NO: 1236) | T (SEQ ID NO: 1678) | |
| RAG3A- | TGCGCACATACATGTGCCC | UGCGCACAUACAUGUGCCCTT | GGGCACAUGUAUGUGCGCAT | 1.4 |
| 524 | (SEQ ID NO: 795) | (SEQ ID NO: 1237) | T (SEQ ID NO: 1679) | |
| RAG3A- | TGCTTCTTCCCCAGCAACA | UGCUUCUUCCCCAGCAACATT | UGUUGCUGGGGAAGAAGCAT | 1.4 |
| 244 | (SEQ ID NO: 796) | (SEQ ID NO: 1238) | T (SEQ ID NO: 1680) | |
| RAG3A- | ATCCTGTTGGATTTCAGCA | AUCCUGUUGGAUUUCAGCATT | UGCUGAAAUCCAACAGGAUT | 1.4 |
| 340 | (SEQ ID NO: 797) | (SEQ ID NO: 1239) | T (SEQ ID NO: 1681) | |
| RAG3A- | GCCTGGTTATTCCTCTTGT | GCCUGGUUAUUCCUCUUGUTT | ACAAGAGGAAUAACCAGGCT | 1.4 |
| 687 | (SEQ ID NO: 798) | (SEQ ID NO: 1240) | T (SEQ ID NO: 1682) | |
| RAG3A- | CCAGCAGACGAGCACCTAA | CCAGCAGACGAGCACCUAATT | UUAGGUGCUCGUCUGCUGGT | 1.4 |
| 568 | (SEQ ID NO: 799) | (SEQ ID NO: 1241) | T (SEQ ID NO: 1683) | |
| RAG3A- | TGGGACTGGGAAGAAAGC | UGGGACUGGGAAGAAAGCATT | UGCUUUCUUCCCAGUCCCATT | 1.4 |
| 804 | A (SEQ ID NO: 800) | (SEQ ID NO: 1242) | (SEQ ID NO: 1684) | |
| RAG3A- | CAGTCCACTCAACCCGTCC | CAGUCCACUCAACCCGUCCTT | GGACGGGUUGAGUGGACUGT | 1.4 |
| 496 | (SEQ ID NO: 801) | (SEQ ID NO: 1243) | T (SEQ ID NO: 1685) | |
| RAG3A- | GGAGACTGGGACTGGGAA | GGAGACUGGGACUGGGAAGTT | CUUCCCAGUCCCAGUCUCCTT | 1.4 |
| 810 | G (SEQ ID NO: 802) | (SEQ ID NO: 1244) | (SEQ ID NO: 1686) | |
| RAG3A- | AAGCAGGAAAGCAAGCAT | AAGCAGGAAAGCAAGCAUCTT | GAUGCUUGCUUUCCUGCUUT | 1.4 |
| 356 | C (SEQ ID NO: 803) | (SEQ ID NO: 1245) | T (SEQ ID NO: 1687) | |
| RAG3A- | CCTAGCCAGAAACCGGCAG | CCUAGCCAGAAACCGGCAGTT | CUGCCGGUUUCUGGCUAGGT | 1.4 |
| 1248 | (SEQ ID NO: 804) | (SEQ ID NO: 1246) | T (SEQ ID NO: 1688) | |
| RAG3A- | CAGCTATCCCAGCACCCTC | CAGCUAUCCCAGCACCCUCTT | GAGGGUGCUGGGAUAGCUGT | 1.4 |
| 1015 | (SEQ ID NO: 805) | (SEQ ID NO: 1247) | T (SEQ ID NO: 1689) | |
| RAG3A- | GAAGACGGAGACAGAACA | GAAGACGGAGACAGAACAATT | UUGUUCUGUCUCCGUCUUCT | 1.4 |
| 645 | A (SEQ ID NO: 806) | (SEQ ID NO: 1248) | T (SEQ ID NO: 1690) | |
| RAG3A- | ATACATGTGCCCCGCACCT | AUACAUGUGCCCCGCACCUTT | AGGUGCGGGGCACAUGUAUT | 1.4 |
| 517 | (SEQ ID NO: 807) | (SEQ ID NO: 1249) | T (SEQ ID NO: 1691) | |
| RAG3A- | AACTTCTGCTCCTGTCCCC | AACUUCUGCUCCUGUCCCCTT | GGGGACAGGAGCAGAAGUUT | 1.4 |
| 194 | (SEQ ID NO: 808) | (SEQ ID NO: 1250) | T (SEQ ID NO: 1692) | |
| RAG3A- | GCAGACATGCCCATCCTTG | GCAGACAUGCCCAUCCUUGTT | CAAGGAUGGGCAUGUCUGCT | 1.4 |
| 927 | (SEQ ID NO: 809) | (SEQ ID NO: 1251) | T (SEQ ID NO: 1693) | |
| RAG3A- | CCCCACCAATCTTAAACAA | CCCCACCAAUCUUAAACAATT | UUGUUUAAGAUUGGUGGGGT | 1.4 |
| 271 | (SEQ ID NO: 810) | (SEQ ID NO: 1252) | T (SEQ ID NO: 1694) | |
| RAG3A- | GGGAATGACAAGATAGGAC | GGGAAUGACAAGAUAGGACTT | GUCCUAUCUUGUCAUUCCCT | 1.4 |
| 398 | (SEQ ID NO: 811) | (SEQ ID NO: 1253) | T (SEQ ID NO: 1695) | |
| RAG3A- | CAACACAAATGTCCTGCCA | CAACACAAAUGUCCUGCCATT | UGGCAGGACAUUUGUGUUGT | 1.4 |
| 230 | (SEQ ID NO: 812) | (SEQ ID NO: 1254) | T (SEQ ID NO: 1696) | |
| RAG3A- | CCACTCCCAGCCTCCTTTC | CCACUCCCAGCCUCCUUUCTT | GAAAGGAGGCUGGGAGUGGT | 1.4 |
| 1104 | (SEQ ID NO: 813) | (SEQ ID NO: 1255) | T (SEQ ID NO: 1697) | |
| RAG3A- | CAGGGACCTAGCCAGAAA | CAGGGACCUAGCCAGAAACTT | GUUUCUGGCUAGGUCCCUGT | 1.4 |
| 1254 | C (SEQ ID NO: 814) | (SEQ ID NO: 1256) | T (SEQ ID NO: 1698) | |
| RAG3A- | TAGCCAGGGAGTGAAAAC | UAGCCAGGGAGUGAAAACCTT | GGUUUUCACUCCCUGGCUAT | 1.4 |
| 288 | C (SEQ ID NO: 815) | (SEQ ID NO: 1257) | T (SEQ ID NO: 1699) | |
| RAG3A- | GGAAGCCCCTGAGGACAG | GGAAGCCCCUGAGGACAGATT | UCUGUCCUCAGGGGCUUCCT | 1.4 |
| 837 | A (SEQ ID NO: 816) | (SEQ ID NO: 1258) | T (SEQ ID NO: 1700) | |
| RAG3A- | ACTTCTCAGCAGACATGCC | ACUUCUCAGCAGACAUGCCTT | GGCAUGUCUGCUGAGAAGUT | 1.4 |
| 935 | (SEQ ID NO: 817) | (SEQ ID NO: 1259) | T (SEQ ID NO: 1701) | |
| RAG3A- | AACCCTTTCCCCATAACAC | AACCCUUUCCCCAUAACACTT | GUGUUAUGGGGAAAGGGUUT | 1.4 |
| 476 | (SEQ ID NO: 818) | (SEQ ID NO: 1260) | T (SEQ ID NO: 1702) | |
| RAG3A- | AGCCATCTAAGCCACTGGA | AGCCAUCUAAGCCACUGGATT | UCCAGUGGCUUAGAUGGCUT | 1.4 |
| 589 | (SEQ ID NO: 819) | (SEQ ID NO: 1261) | T (SEQ ID NO: 1703) | |
| RAG3A- | CAGTATCTCCCTCTCATTG | CAGUAUCUCCCUCUCAUUGTT | CAAUGAGAGGGAGAUACUGT | 1.4 |
| 1207 | (SEQ ID NO: 820) | (SEQ ID NO: 1262) | T (SEQ ID NO: 1704) | |
| RAG3A- | GGTCGCTCCTCATTCTTGT | GGUCGCUCCUCAUUCUUGUTT | ACAAGAAUGAGGAGCGACCT | 1.4 |
| 1284 | (SEQ ID NO: 821) | (SEQ ID NO: 1263) | T (SEQ ID NO: 1705) | |
| RAG3A- | AAATTCCAGGATCTAGGCC | AAAUUCCAGGAUCUAGGCCTT | GGCCUAGAUCCUGGAAUUUT | 1.4 |
| 956 | (SEQ ID NO: 822) | (SEQ ID NO: 1264) | T (SEQ ID NO: 1706) | |
| RAG3A- | GCACCTAAGCTCAGGCTTA | GCACCUAAGCUCAGGCUUATT | UAAGCCUGAGCUUAGGUGCT | 1.4 |
| 557 | (SEQ ID NO: 823) | (SEQ ID NO: 1265) | T (SEQ ID NO: 1707) | |
| RAG3A- | CTGACAGTCCACTCAACCC | CUGACAGUCCACUCAACCCTT | GGGUUGAGUGGACUGUCAGT | 1.4 |
| 500 | (SEQ ID NO: 824) | (SEQ ID NO: 1266) | T (SEQ ID NO: 1708) | |
| RAG3A- | ACAGTATCTCCCTCTCATT | ACAGUAUCUCCCUCUCAUUTT | AAUGAGAGGGAGAUACUGUT | 1.4 |
| 1208 | (SEQ ID NO: 825) | (SEQ ID NO: 1267) | T (SEQ ID NO: 1709) | |
| RAG3A- | CCTGGTTATTCCTCTTGTC | CCUGGUUAUUCCUCUUGUCTT | GACAAGAGGAAUAACCAGGT | 1.4 |
| 686 | (SEQ ID NO: 826) | (SEQ ID NO: 1268) | T (SEQ ID NO: 1710) | |
| RAG3A- | TCGACAGTCCCCTCTTGCT | UCGACAGUCCCCUCUUGCUTT | AGCAAGAGGGGACUGUCGAT | 1.4 |
| 1168 | (SEQ ID NO: 827) | (SEQ ID NO: 1269) | T (SEQ ID NO: 1711) | |
| RAG3A- | CCTGCCAGATTCCTCCTGG | CCUGCCAGAUUCCUCCUGGTT | CCAGGAGGAAUCUGGCAGGT | 1.4 |
| 218 | (SEQ ID NO: 828) | (SEQ ID NO: 1270) | T (SEQ ID NO: 1712) | |
| RAG3A- | AAGCCCCTGAGGACAGAG | AAGCCCCUGAGGACAGAGATT | UCUCUGUCCUCAGGGGCUUT | 1.4 |
| 835 | A (SEQ ID NO: 829) | (SEQ ID NO: 1271) | T (SEQ ID NO: 1713) | |
| RAG3A- | GGATAGCCAGGGAGTGAAA | GGAUAGCCAGGGAGUGAAATT | UUUCACUCCCUGGCUAUCCT | 1.3 |
| 291 | (SEQ ID NO: 830) | (SEQ ID NO: 1272) | T (SEQ ID NO: 1714) | |
| RAG3A- | CCTTCTGTGGAGTGACAGT | CCUUCUGUGGAGUGACAGUTT | ACUGUCACUCCACAGAAGGT | 1.3 |
| 1222 | (SEQ ID NO: 831) | (SEQ ID NO: 1273) | T (SEQ ID NO: 1715) | |
| RAG3A- | GAAAACCCCACCAATCTTA | GAAAACCCCACCAAUCUUATT | UAAGAUUGGUGGGGUUUUCT | 1.3 |
| 276 | (SEQ ID NO: 832) | (SEQ ID NO: 1274) | T (SEQ ID NO: 1716) | |
| RAG3A- | CATGTCCAGGAAAAGATGG | CAUGUCCAGGAAAAGAUGGTT | CCAUCUUUUCCUGGACAUGT | 1.3 |
| 156 | (SEQ ID NO: 833) | (SEQ ID NO: 1275) | T (SEQ ID NO: 1717) | |
| RAG3A- | ACACTTCTCAGCAGACATG | ACACUUCUCAGCAGACAUGTT | CAUGUCUGCUGAGAAGUGUT | 1.3 |
| 937 | (SEQ ID NO: 834) | (SEQ ID NO: 1276) | T (SEQ ID NO: 1718) | |
| RAG3A- | ACCCAGGACAGGGACCTA | ACCCAGGACAGGGACCUAGTT | CUAGGUCCCUGUCCUGGGUT | 1.3 |
| 1262 | G (SEQ ID NO: 835) | (SEQ ID NO: 1277) | T (SEQ ID NO: 1719) | |
| RAG3A- | CTTCCTTCACCCAAGGGAC | CUUCCUUCACCCAAGGGACTT | GUCCCUUGGGUGAAGGAAGT | 1.3 |
| 1139 | (SEQ ID NO: 836) | (SEQ ID NO: 1278) | T (SEQ ID NO: 1720) | |
| RAG3A- | CAGTGCACGTGTGCGCACA | CAGUGCACGUGUGCGCACATT | UGUGCGCACACGUGCACUGT | 1.3 |
| 535 | (SEQ ID NO: 837) | (SEQ ID NO: 1279) | T (SEQ ID NO: 1721) | |
| RAG3A- | GCAGCTGTCTTTCCTAAGA | GCAGCUGUCUUUCCUAAGATT | UCUUAGGAAAGACAGCUGCT | 1.3 |
| 719 | (SEQ ID NO: 838) | (SEQ ID NO: 1280) | T (SEQ ID NO: 1722) | |
| RAG3A- | AGGTCCAGCAGCTGTCTTT | AGGUCCAGCAGCUGUCUUUTT | AAAGACAGCUGCUGGACCUT | 1.3 |
| 726 | (SEQ ID NO: 839) | (SEQ ID NO: 1281) | T (SEQ ID NO: 1723) | |
| RAG3A- | AGGACTCCCTAGGGGATTA | AGGACUCCCUAGGGGAUUATT | UAAUCCCCUAGGGAGUCCUT | 1.3 |
| 384 | (SEQ ID NO: 840) | (SEQ ID NO: 1282) | T (SEQ ID NO: 1724) | |
| RAG3A- | AGCCCCTGAGGACAGAGA | AGCCCCUGAGGACAGAGACTT | GUCUCUGUCCUCAGGGGCUT | 1.3 |
| 834 | C (SEQ ID NO: 841) | (SEQ ID NO: 1283) | T (SEQ ID NO: 1725) | |
| RAG3A- | GACGAGCACCTAAGCTCAG | GACGAGCACCUAAGCUCAGTT | CUGAGCUUAGGUGCUCGUCT | 1.3 |
| 562 | (SEQ ID NO: 842) | (SEQ ID NO: 1284) | T (SEQ ID NO: 1726) | |
| RAG3A- | GCCACACTTCTCAGCAGAC | GCCACACUUCUCAGCAGACTT | GUCUGCUGAGAAGUGUGGCT | 1.3 |
| 940 | (SEQ ID NO: 843) | (SEQ ID NO: 1285) | T (SEQ ID NO: 1727) | |
| RAG3A- | GAACAAGCAAAGGAGGGC | GAACAAGCAAAGGAGGGCCTT | GGCCCUCCUUUGCUUGUUCT | 1.3 |
| 632 | C (SEQ ID NO: 844) | (SEQ ID NO: 1286) | T (SEQ ID NO: 1728) | |
| RAG3A- | CAGGGAGGTATTTGCGGGG | CAGGGAGGUAUUUGCGGGGTT | CCCCGCAAAUACCUCCCUGTT | 1.3 |
| 746 | (SEQ ID NO: 845) | (SEQ ID NO: 1287) | (SEQ ID NO: 1729) | |
| RAG3A- | GCCTGCCTCCCTACCTTCT | GCCUGCCUCCCUACCUUCUTT | AGAAGGUAGGGAGGCAGGCT | 1.3 |
| 1309 | (SEQ ID NO: 846) | (SEQ ID NO: 1288) | T (SEQ ID NO: 1730) | |
| RAG3A- | GCTAGAGCCAGGGCCAAA | GCUAGAGCCAGGGCCAAAGTT | CUUUGGCCCUGGCUCUAGCT | 1.3 |
| 780 | G (SEQ ID NO: 847) | (SEQ ID NO: 1289) | T (SEQ ID NO: 1731) | |
| RAG3A- | ACATGGGCCTGGTTATTCC | ACAUGGGCCUGGUUAUUCCTT | GGAAUAACCAGGCCCAUGUT | 1.3 |
| 693 | (SEQ ID NO: 848) | (SEQ ID NO: 1290) | T (SEQ ID NO: 1732) | |
| RAG3A- | GACAGAACAAGCAAAGGA | GACAGAACAAGCAAAGGAGTT | CUCCUUUGCUUGUUCUGUCT | 1.3 |
| 636 | G (SEQ ID NO: 849) | (SEQ ID NO: 1291) | T (SEQ ID NO: 1733) | |
| RAG3A- | AAACAAGACCTCTGTGCTT | AAACAAGACCUCUGUGCUUTT | AAGCACAGAGGUCUUGUUUT | 1.3 |
| 258 | (SEQ ID NO: 850) | (SEQ ID NO: 1292) | T (SEQ ID NO: 1734) | |
| RAG3A- | ATCCTCCCAGCTATCCCAG | AUCCUCCCAGCUAUCCCAGTT | CUGGGAUAGCUGGGAGGAUT | 1.3 |
| 1022 | (SEQ ID NO: 851) | (SEQ ID NO: 1293) | T (SEQ ID NO: 1735) | |
| RAG3A- | ACTCCCTAGGGGATTACAG | ACUCCCUAGGGGAUUACAGTT | CUGUAAUCCCCUAGGGAGUT | 1.3 |
| 381 | (SEQ ID NO: 852) | (SEQ ID NO: 1294) | T (SEQ ID NO: 1736) | |
| RAG3A- | TCCCTAGGGGATTACAGAA | UCCCUAGGGGAUUACAGAATT | UUCUGUAAUCCCCUAGGGAT | 1.3 |
| 379 | (SEQ ID NO: 853) | (SEQ ID NO: 1295) | T (SEQ ID NO: 1737) | |
| RAG3A- | GTGGAACGGTAGGAGATGG | GUGGAACGGUAGGAGAUGGTT | CCAUCUCCUACCGUUCCACTT | 1.3 |
| 663 | (SEQ ID NO: 854) | (SEQ ID NO: 1296) | (SEQ ID NO: 1738) | |
| RAG3A- | CCCAGCCTCCTTTCTCTTG | CCCAGCCUCCUUUCUCUUGTT | CAAGAGAAAGGAGGCUGGGT | 1.3 |
| 1099 | (SEQ ID NO: 855) | (SEQ ID NO: 1297) | T (SEQ ID NO: 1739) | |
| RAG3A- | GGGATCAAGGTCAGGCCA | GGGAUCAAGGUCAGGCCAGTT | CUGGCCUGACCUUGAUCCCT | 1.3 |
| 855 | G (SEQ ID NO: 856) | (SEQ ID NO: 1298) | T (SEQ ID NO: 1740) | |
| RAG3A- | CTCCTCATTCTTGTGTTAC | CUCCUCAUUCUUGUGUUACTT | GUAACACAAGAAUGAGGAGT | 1.3 |
| 1279 | (SEQ ID NO: 857) | (SEQ ID NO: 1299) | T (SEQ ID NO: 1741) | |
| RAG3A- | CACCAGTGTCTGGTGGCTC | CACCAGUGUCUGGUGGCUCTT | GAGCCACCAGACACUGGUGT | 1.3 |
| 34 | (SEQ ID NO: 858) | (SEQ ID NO: 1300) | T (SEQ ID NO: 1742) | |
| RAG3A- | AGAACAAGCAAAGGAGGG | AGAACAAGCAAAGGAGGGCTT | GCCCUCCUUUGCUUGUUCUT | 1.3 |
| 633 | C (SEQ ID NO: 859) | (SEQ ID NO: 1301) | T (SEQ ID NO: 1743) | |
| RAG3A- | CCATAACAGGAGATTTCTC | CCAUAACAGGAGAUUUCUCTT | GAGAAAUCUCCUGUUAUGGT | 1.3 |
| 453 | (SEQ ID NO: 860) | (SEQ ID NO: 1302) | T (SEQ ID NO: 1744) | |
| RAG3A- | TGTGCTTCTTCCCCAGCAA | UGUGCUUCUUCCCCAGCAATT | UUGCUGGGGAAGAAGCACAT | 1.3 |
| 246 | (SEQ ID NO: 861) | (SEQ ID NO: 1303) | T (SEQ ID NO: 1745) | |
| RAG3A- | TGTAGCCATCTAAGCCACT | UGUAGCCAUCUAAGCCACUTT | AGUGGCUUAGAUGGCUACAT | 1.3 |
| 592 | (SEQ ID NO: 862) | (SEQ ID NO: 1304) | T (SEQ ID NO: 1746) | |
| RAG3A- | CCCACCAATCTTAAACAAG | CCCACCAAUCUUAAACAAGTT | CUUGUUUAAGAUUGGUGGGT | 1.3 |
| 270 | (SEQ ID NO: 863) | (SEQ ID NO: 1305) | T (SEQ ID NO: 1747) | |
| RAG3A- | ACAAGCAAAGGAGGGCCC | ACAAGCAAAGGAGGGCCCUTT | AGGGCCCUCCUUUGCUUGUT | 1.3 |
| 630 | T (SEQ ID NO: 864) | (SEQ ID NO: 1306) | T (SEQ ID NO: 1748) | |
| RAG3A- | CTGGTTATTCCTCTTGTCA | CUGGUUAUUCCUCUUGUCATT | UGACAAGAGGAAUAACCAGT | 1.3 |
| 685 | (SEQ ID NO: 865) | (SEQ ID NO: 1307) | T (SEQ ID NO: 1749) | |
| RAG3A- | CACTCCCAGCCTCCTTTCT | CACUCCCAGCCUCCUUUCUTT | AGAAAGGAGGCUGGGAGUGT | 1.3 |
| 1103 | (SEQ ID NO: 866) | (SEQ ID NO: 1308) | T (SEQ ID NO: 1750) | |
| RAG3A- | CTGCCTCCCTGTCTCCTGT | CUGCCUCCCUGUCUCCUGUTT | ACAGGAGACAGGGAGGCAGT | 1.3 |
| 1065 | (SEQ ID NO: 867) | (SEQ ID NO: 1309) | T (SEQ ID NO: 1751) | |
| RAG3A- | GCGCACATACATGTGCCCC | GCGCACAUACAUGUGCCCCTT | GGGGCACAUGUAUGUGCGCT | 1.3 |
| 523 | (SEQ ID NO: 868) | (SEQ ID NO: 1310) | T (SEQ ID NO: 1752) | |
| RAG3A- | TAGCCATCTAAGCCACTGG | UAGCCAUCUAAGCCACUGGTT | CCAGUGGCUUAGAUGGCUAT | 1.3 |
| 590 | (SEQ ID NO: 869) | (SEQ ID NO: 1311) | T (SEQ ID NO: 1753) | |
| RAG3A- | CGCACCTGACAGTCCACTC | CGCACCUGACAGUCCACUCTT | GAGUGGACUGUCAGGUGCGT | 1.3 |
| 505 | (SEQ ID NO: 870) | (SEQ ID NO: 1312) | T (SEQ ID NO: 1754) | |
| RAG3A- | CTAGCCAGAAACCGGCAGC | CUAGCCAGAAACCGGCAGCTT | GCUGCCGGUUUCUGGCUAGT | 1.3 |
| 1247 | (SEQ ID NO: 871) | (SEQ ID NO: 1313) | T (SEQ ID NO: 1755) | |
| RAG3A- | CGCTCCTCATTCTTGTGTT | CGCUCCUCAUUCUUGUGUUTT | AACACAAGAAUGAGGAGCGT | 1.3 |
| 1281 | (SEQ ID NO: 872) | (SEQ ID NO: 1314) | T (SEQ ID NO: 1756) | |
| RAG3A- | GCAGGGAGGTATTTGCGGG | GCAGGGAGGUAUUUGCGGGTT | CCCGCAAAUACCUCCCUGCTT | 1.3 |
| 747 | (SEQ ID NO: 873) | (SEQ ID NO: 1315) | (SEQ ID NO: 1757) | |
| RAG3A- | TTCTGTGGAGTGACAGTAT | UUCUGUGGAGUGACAGUAUTT | AUACUGUCACUCCACAGAAT | 1.3 |
| 1220 | (SEQ ID NO: 874) | (SEQ ID NO: 1316) | T (SEQ ID NO: 1758) | |
| RAG3A- | TGTGTTACCCAGGACAGGG | UGUGUUACCCAGGACAGGGTT | CCCUGUCCUGGGUAACACATT | 1.3 |
| 1268 | (SEQ ID NO: 875) | (SEQ ID NO: 1317) | (SEQ ID NO: 1759) | |
| RAG3A- | GAGCTAGAGCCAGGGCCA | GAGCUAGAGCCAGGGCCAATT | UUGGCCCUGGCUCUAGCUCT | 1.3 |
| 782 | A (SEQ ID NO: 876) | (SEQ ID NO: 1318) | T (SEQ ID NO: 1760) | |
| RAG3A- | GTGTGGGTGGAGGAGTGG | GUGUGGGUGGAGGAGUGGATT | UCCACUCCUCCACCCACACTT | 1.3 |
| 107 | A (SEQ ID NO: 877) | (SEQ ID NO: 1319) | (SEQ ID NO: 1761) | |
| RAG3A- | ACATGCCCATCCTTGGGGA | ACAUGCCCAUCCUUGGGGATT | UCCCCAAGGAUGGGCAUGUT | 1.3 |
| 923 | (SEQ ID NO: 878) | (SEQ ID NO: 1320) | T (SEQ ID NO: 1762) | |
| RAG3A- | AAAGCAAGCATCCTGTTGG | AAAGCAAGCAUCCUGUUGGTT | CCAACAGGAUGCUUGCUUUT | 1.3 |
| 349 | (SEQ ID NO: 879) | (SEQ ID NO: 1321) | T (SEQ ID NO: 1763) | |
| RAG3A- | TCTTGGTTCCCTGGTCATG | UCUUGGUUCCCUGGUCAUGTT | CAUGACCAGGGAACCAAGAT | 1.3 |
| 1085 | (SEQ ID NO: 880) | (SEQ ID NO: 1322) | T (SEQ ID NO: 1764) | |
| RAG3A- | ATGTGGGCAATATCCGTGT | AUGUGGGCAAUAUCCGUGUTT | ACACGGAUAUUGCCCACAUT | 1.3 |
| 432 | (SEQ ID NO: 881) | (SEQ ID NO: 1323) | T (SEQ ID NO: 1765) | |
| RAG3A- | ATCCAAATCTTCTCCGTGG | AUCCAAAUCUUCUCCGUGGTT | CCACGGAGAAGAUUUGGAUT | 1.3 |
| 129 | (SEQ ID NO: 882) | (SEQ ID NO: 1324) | T (SEQ ID NO: 1766) | |
| RAG3A- | CCTGAGGACAGAGACTGT | CCUGAGGACAGAGACUGUGTT | CACAGUCUCUGUCCUCAGGT | 1.3 |
| 830 | G (SEQ ID NO: 883) | (SEQ ID NO: 1325) | T (SEQ ID NO: 1767) | |
| RAG3A- | GGGGCTCCAGGGAAGAGA | GGGGCUCCAGGGAAGAGAATT | UUCUCUUCCCUGGAGCCCCTT | 1.3 |
| 74 | A (SEQ ID NO: 884) | (SEQ ID NO: 1326) | (SEQ ID NO: 1768) | |
| RAG3A- | TGGGATCAAGGTCAGGCCA | UGGGAUCAAGGUCAGGCCATT | UGGCCUGACCUUGAUCCCATT | 1.3 |
| 856 | (SEQ ID NO: 885) | (SEQ ID NO: 1327) | (SEQ ID NO: 1769) | |
| RAG3A- | CTCATGTGGGCAATATCCG | CUCAUGUGGGCAAUAUCCGTT | CGGAUAUUGCCCACAUGAGT | 1.3 |
| 435 | (SEQ ID NO: 886) | (SEQ ID NO: 1328) | T (SEQ ID NO: 1770) | |
| RAG3A- | CAGACGAGCACCTAAGCTC | CAGACGAGCACCUAAGCUCTT | GAGCUUAGGUGCUCGUCUGT | 1.3 |
| 564 | (SEQ ID NO: 887) | (SEQ ID NO: 1329) | T | |
| | | | (SEQ ID NO: 1771) | |
| RAG3A- | GGACCTAGCCAGAAACCG | GGACCUAGCCAGAAACCGGTT | CCGGUUUCUGGCUAGGUCCT | 1.3 |
| 1251 | G | (SEQ ID NO: 1330) | T | |
| | (SEQ ID NO: 888) | | (SEQ ID NO: 1772) | |
| RAG3A- | TCTCTGAGGTCGCTCCTCA | UCUCUGAGGUCGCUCCUCATT | UGAGGAGCGACCUCAGAGAT | 1.3 |
| 1291 | (SEQ ID NO: 889) | (SEQ ID NO: 1331) | T | |
| | | | (SEQ ID NO: 1773) | |
| RAG3A- | AGCTAGAGCCAGGGCCAA | AGCUAGAGCCAGGGCCAAATT | UUUGGCCCUGGCUCUAGCUT | 1.3 |
| 781 | A | (SEQ ID NO: 1332) | T | |
| | (SEQ ID NO: 890) | | (SEQ ID NO: 1774) | |
| RAG3A- | CTTAAACAAGACCTCTGTG | CUUAAACAAGACCUCUGUGTT | CACAGAGGUCUUGUUUAAGT | 1.3 |
| 261 | (SEQ ID NO: 891) | (SEQ ID NO: 1333) | T | |
| | | | (SEQ ID NO: 1775) | |
| RAG3A- | CCAGCCTCCTTTCTCTTGG | CCAGCCUCCUUUCUCUUGGTT | CCAAGAGAAAGGAGGCUGGT | 1.3 |
| 1098 | (SEQ ID NO: 892) | (SEQ ID NO: 1334) | T | |
| | | | (SEQ ID NO: 1776) | |
| RAG3A- | GCAGACGAGCACCTAAGCT | GCAGACGAGCACCUAAGCUTT | AGCUUAGGUGCUCGUCUGCT | 1.2 |
| 565 | (SEQ ID NO: 893) | (SEQ ID NO: 1335) | T | |
| | | | (SEQ ID NO: 1777) | |
| RAG3A- | TCAGCAGACATGCCCATCC | UCAGCAGACAUGCCCAUCCTT | GGAUGGGCAUGUCUGCUGAT | 1.2 |
| 930 | (SEQ ID NO: 894) | (SEQ ID NO: 1336) | T | |
| | | | (SEQ ID NO: 1778) | |
| RAG3A- | TATCCGTGTTCCCACTTCG | UAUCCGUGUUCCCACUUCGTT | CGAAGUGGGAACACGGAUAT | 1.2 |
| 422 | (SEQ ID NO: 895) | (SEQ ID NO: 1337) | T | |
| | | | (SEQ ID NO: 1779) | |
| RAG3A- | TACATGTGCCCCGCACCTG | UACAUGUGCCCCGCACCUGTT | CAGGUGCGGGGCACAUGUAT | 1.2 |
| 516 | (SEQ ID NO: 896) | (SEQ ID NO: 1338) | T | |
| | | | (SEQ ID NO: 1780) | |
| RAG3A- | TGTAACTTATCCTCAGGCG | UGUAACUUAUCCUCAGGCGTT | CGCCUGAGGAUAAGUUACAT | 1.2 |
| 1190 | (SEQ ID NO: 897) | (SEQ ID NO: 1339) | T | |
| | | | (SEQ ID NO: 1781) | |
| RAG3A- | AGAAAAGCAGGAAAGCAA | AGAAAAGCAGGAAAGCAAGTT | CUUGCUUUCCUGCUUUUCUT | 1.2 |
| 360 | G | (SEQ ID NO: 1340) | T | |
| | (SEQ ID NO: 898) | | (SEQ ID NO: 1782) | |
| RAG3A- | CAAATCTTCTCCGTGGTGT | CAAAUCUUCUCCGUGGUGUTT | ACACCACGGAGAAGAUUUGT | 1.2 |
| 126 | (SEQ ID NO: 899) | (SEQ ID NO: 1341) | T | |
| | | | (SEQ ID NO: 1783) | |
| RAG3A- | CACACTTCTCAGCAGACAT | CACACUUCUCAGCAGACAUTT | AUGUCUGCUGAGAAGUGUGT | 1.2 |
| 938 | (SEQ ID NO: 900) | (SEQ ID NO: 1342) | T | |
| | | | (SEQ ID NO: 1784) | |
| RAG3A- | GATCAAGGTCAGGCCAGG | GAUCAAGGUCAGGCCAGGATT | UCCUGGCCUGACCUUGAUCT | 1.2 |
| 853 | A | (SEQ ID NO: 1343) | T | |
| | (SEQ ID NO: 901) | | (SEQ ID NO: 1785) | |
| RAG3A- | GCTGTCTTTCCTAAGACAG | GCUGUCUUUCCUAAGACAGTT | CUGUCUUAGGAAAGACAGCT | 1.2 |
| 716 | (SEQ ID NO: 902) | (SEQ ID NO: 1344) | T | |
| | | | (SEQ ID NO: 1786) | |
| RAG3A- | CCACTATCCTCCCAGCTAT | CCACUAUCCUCCCAGCUAUTT | AUAGCUGGGAGGAUAGUGGT | 1.2 |
| 1027 | (SEQ ID NO: 903) | (SEQ ID NO: 1345) | T | |
| | | | (SEQ ID NO: 1787) | |
| RAG3A- | CGGAGACAGAACAAGCAA | CGGAGACAGAACAAGCAAATT | UUUGCUUGUUCUGUCUCCGT | 1.2 |
| 640 | A | (SEQ ID NO: 1346) | T | |
| | (SEQ ID NO: 904) | | (SEQ ID NO: 1788) | |
| RAG3A- | CAGGGAGTGAAAACCCCA | CAGGGAGUGAAAACCCCACTT | GUGGGGUUUUCACUCCCUGT | 1.2 |
| 284 | C | (SEQ ID NO: 1347) | T | |
| | (SEQ ID NO: 905) | | (SEQ ID NO: 1789) | |
| RAG3A- | TAAGACAGGGACACATGGG | UAAGACAGGGACACAUGGGTT | CCCAUGUGUCCCUGUCUUATT | 1.2 |
| 705 | (SEQ ID NO: 906) | (SEQ ID NO: 1348) | (SEQ ID NO: 1790) | |
| RAG3A- | TGCCTAGCCTGCCTCCCTA | UGCCUAGCCUGCCUCCCUATT | UAGGGAGGCAGGCUAGGCAT | 1.2 |
| 1315 | (SEQ ID NO: 907) | (SEQ ID NO: 1349) | T | |
| | | | (SEQ ID NO: 1791) | |
| RAG3A- | TCCATCAGCCGATCTCCCC | UCCAUCAGCCGAUCUCCCCTT | GGGGAGAUCGGCUGAUGGAT | 1.2 |
| 1381 | (SEQ ID NO: 908) | (SEQ ID NO: 1350) | T | |
| | | | (SEQ ID NO: 1792) | |
| RAG3A- | AGCACCTAAGCTCAGGCTT | AGCACCUAAGCUCAGGCUUTT | AAGCCUGAGCUUAGGUGCUT | 1.2 |
| 558 | (SEQ ID NO: 909) | (SEQ ID NO: 1351) | T | |
| | | | (SEQ ID NO: 1793) | |
| RAG3A- | GACAGTCCACTCAACCCGT | GACAGUCCACUCAACCCGUTT | ACGGGUUGAGUGGACUGUCT | 1.2 |
| 498 | (SEQ ID NO: 910) | (SEQ ID NO: 1352) | T | |
| | | | (SEQ ID NO: 1794) | |
| RAG3A- | AAGCCACTGGACCCCAGC | AAGCCACUGGACCCCAGCATT | UGCUGGGGUCCAGUGGCUUT | 1.2 |
| 581 | A | (SEQ ID NO: 1353) | T | |
| | (SEQ ID NO: 911) | | (SEQ ID NO: 1795) | |
| RAG3A- | CTTCACCCAAGGGACCCTC | CUUCACCCAAGGGACCCUCTT | GAGGGUCCCUUGGGUGAAGT | 1.2 |
| 1135 | (SEQ ID NO: 912) | (SEQ ID NO: 1354) | T | |
| | | | (SEQ ID NO: 1796) | |
| RAG3A- | CATGCCTGCCTCCCTGTCT | CAUGCCUGCCUCCCUGUCUTT | AGACAGGGAGGCAGGCAUGT | 1.2 |
| 1070 | (SEQ ID NO: 913) | (SEQ ID NO: 1355) | T | |
| | | | (SEQ ID NO: 1797) | |
| RAG3A- | TCCTCAGGCGCATTCGACA | UCCUCAGGCGCAUUCGACATT | UGUCGAAUGCGCCUGAGGAT | 1.2 |
| 1181 | (SEQ ID NO: 914) | (SEQ ID NO: 1356) | T | |
| | | | (SEQ ID NO: 1798) | |
| RAG3A- | AGTATCTCCCTCTCATTGT | AGUAUCUCCCUCUCAUUGUTT | ACAAUGAGAGGGAGAUACUT | 1.2 |
| 1206 | (SEQ ID NO: 915) | (SEQ ID NO: 1357) | T | |
| | | | (SEQ ID NO: 1799) | |
| RAG3A- | ACACCAACCCATAACAGGA | ACACCAACCCAUAACAGGATT | UCCUGUUAUGGGUUGGUGUT | 1.2 |
| 461 | (SEQ ID NO: 916) | (SEQ ID NO: 1358) | T | |
| | | | (SEQ ID NO: 1800) | |
| RAG3A- | CTTCTTCCCCAGCAACACA | CUUCUUCCCCAGCAACACATT | UGUGUUGCUGGGGAAGAAGT | 1.2 |
| 242 | (SEQ ID NO: 917) | (SEQ ID NO: 1359) | T | |
| | | | (SEQ ID NO: 1801) | |
| RAG3A- | AAGGGACCCTCTGCCTCTC | AAGGGACCCUCUGCCUCUCTT | GAGAGGCAGAGGGUCCCUUT | 1.2 |
| 1127 | (SEQ ID NO: 918) | (SEQ ID NO: 1360) | T | |
| | | | (SEQ ID NO: 1802) | |
| RAG3A- | CCGTGTTCCCACTTCGAAA | CCGUGUUCCCACUUCGAAATT | UUUCGAAGUGGGAACACGGT | 1.2 |
| 419 | (SEQ ID NO: 919) | (SEQ ID NO: 1361) | T | |
| | | | (SEQ ID NO: 1803) | |
| RAG3A- | AAGCAAAGGAGCTAGAGC | AAGCAAAGGAGCUAGAGCCTT | GGCUCUAGCUCCUUUGCUUT | 1.2 |
| 790 | C | (SEQ ID NO: 1362) | T | |
| | (SEQ ID NO: 920) | | (SEQ ID NO: 1804) | |
| RAG3A- | ACAGAACAAGCAAAGGAG | ACAGAACAAGCAAAGGAGGTT | CCUCCUUUGCUUGUUCUGUT | 1.2 |
| 635 | G | (SEQ ID NO: 1363) | T | |
| | (SEQ ID NO: 921) | | (SEQ ID NO: 1805) | |
| RAG3A- | CTTGTCACATGTGGAACGG | CUUGUCACAUGUGGAACGGTT | CCGUUCCACAUGUGACAAGT | 1.2 |
| 673 | (SEQ ID NO: 922) | (SEQ ID NO: 1364) | T | |
| | | | (SEQ ID NO: 1806) | |
| RAG3A- | CCCTCCTTCCTAATCTTGG | CCCUCCUUCCUAAUCUUGGTT | CCAAGAUUAGGAAGGAGGGT | 1.2 |
| 1001 | (SEQ ID NO: 923) | (SEQ ID NO: 1365) | T | |
| | | | (SEQ ID NO: 1807) | |
| RAG3A- | AGCACCCTCCTTCCTAATC | AGCACCCUCCUUCCUAAUCTT | GAUUAGGAAGGAGGGUGCUT | 1.2 |
| 1005 | (SEQ ID NO: 924) | (SEQ ID NO: 1366) | T | |
| | | | (SEQ ID NO: 1808) | |
| RAG3A- | TGCCTCCCTGTCTCCTGTC | UGCCUCCCUGUCUCCUGUCTT | GACAGGAGACAGGGAGGCAT | 1.2 |
| 1064 | (SEQ ID NO: 925) | (SEQ ID NO: 1367) | T | |
| | | | (SEQ ID NO: 1809) | |
| RAG3A- | GACTGGGAAGAAAGCAAA | GACUGGGAAGAAAGCAAAGTT | CUUUGCUUUCUUCCCAGUCT | 1.2 |
| 801 | G | (SEQ ID NO: 1368) | T | |
| | (SEQ ID NO: 926) | | (SEQ ID NO: 1810) | |
| RAG3A- | AACCCGTCCAAACCCTTTC | AACCCGUCCAAACCCUUUCTT | GAAAGGGUUUGGACGGGUUT | 1.2 |
| 486 | (SEQ ID NO: 927) | (SEQ ID NO: 1369) | T | |
| | | | (SEQ ID NO: 1811) | |
| RAG3A- | ACCTAAGCTCAGGCTTAAC | ACCUAAGCUCAGGCUUAACTT | GUUAAGCCUGAGCUUAGGUT | 1.2 |
| 555 | (SEQ ID NO: 928) | (SEQ ID NO: 1370) | T | |
| | | | (SEQ ID NO: 1812) | |
| RAG3A- | GGATCAAGGTCAGGCCAG | GGAUCAAGGUCAGGCCAGGTT | CCUGGCCUGACCUUGAUCCT | 1.2 |
| 854 | G | (SEQ ID NO: 1371) | T | |
| | (SEQ ID NO: 929) | | (SEQ ID NO: 1813) | |
| RAG3A- | GTGTTACCCAGGACAGGGA | GUGUUACCCAGGACAGGGATT | UCCCUGUCCUGGGUAACACT | 1.2 |
| 1267 | (SEQ ID NO: 930) | (SEQ ID NO: 1372) | T | |
| | | | (SEQ ID NO: 1814) | |
| RAG3A- | TTACCCAGGACAGGGACCT | UUACCCAGGACAGGGACCUTT | AGGUCCCUGUCCUGGGUAAT | 1.2 |
| 1264 | (SEQ ID NO: 931) | (SEQ ID NO: 1373) | T | |
| | | | (SEQ ID NO: 1815) | |
| RAG3A- | ATGTGCCCCGCACCTGACA | AUGUGCCCCGCACCUGACATT | UGUCAGGUGCGGGGCACAUT | 1.2 |
| 513 | (SEQ ID NO: 932) | (SEQ ID NO: 1374) | T | |
| | | | (SEQ ID NO: 1816) | |
| RAG3A- | GAGGTCGCTCCTCATTCTT | GAGGUCGCUCCUCAUUCUUTT | AAGAAUGAGGAGCGACCUCT | 1.2 |
| 1286 | (SEQ ID NO: 933) | (SEQ ID NO: 1375) | T | |
| | | | (SEQ ID NO: 1817) | |
| RAG3A- | AGGAGATGGAAGACGGAG | AGGAGAUGGAAGACGGAGATT | UCUCCGUCUUCCAUCUCCUTT | 1.2 |
| 653 | A | (SEQ ID NO: 1376) | (SEQ ID NO: 1818) | |
| | (SEQ ID NO: 934) | | | |
| RAG3A- | AGCTATCCCAGCACCCTCC | AGCUAUCCCAGCACCCUCCTT | GGAGGGUGCUGGGAUAGCUT | 1.2 |
| 1014 | (SEQ ID NO: 935) | (SEQ ID NO: 1377) | T | |
| | | | (SEQ ID NO: 1819) | |
| RAG3A- | AGAGAAGGCGTCACTTCCG | AGAGAAGGCGUCACUUCCGTT | CGGAAGUGACGCCUUCUCUT | 1.2 |
| 61 | (SEQ ID NO: 936) | (SEQ ID NO: 1378) | T | |
| | | | (SEQ ID NO: 1820) | |
| RAG3A- | CATGTGGAACGGTAGGAGA | CAUGUGGAACGGUAGGAGATT | UCUCCUACCGUUCCACAUGT | 1.2 |
| 666 | (SEQ ID NO: 937) | (SEQ ID NO: 1379) | T | |
| | | | (SEQ ID NO: 1821) | |
| RAG3A- | AAGCAAGCATCCTGTTGGA | AAGCAAGCAUCCUGUUGGATT | UCCAACAGGAUGCUUGCUUT | 1.2 |
| 348 | (SEQ ID NO: 938) | (SEQ ID NO: 1380) | T | |
| | | | (SEQ ID NO: 1822) | |
| RAG3A- | AGGCTTAACCCAGTGCACG | AGGCUUAACCCAGUGCACGTT | CGUGCACUGGGUUAAGCCUT | 1.2 |
| 545 | (SEQ ID NO: 939) | (SEQ ID NO: 1381) | T | |
| | | | (SEQ ID NO: 1823) | |
| RAG3A- | TCTAAGCCACTGGACCCCA | UCUAAGCCACUGGACCCCATT | UGGGGUCCAGUGGCUUAGAT | 1.2 |
| 584 | (SEQ ID NO: 940) | (SEQ ID NO: 1382) | T | |
| | | | (SEQ ID NO: 1824) | |
| RAG3A- | GTCCCCTCTTGCTTTCTCA | GUCCCCUCUUGCUUUCUCATT | UGAGAAAGCAAGAGGGGACT | 1.2 |
| 1162 | (SEQ ID NO: 941) | (SEQ ID NO: 1383) | T | |
| | | | (SEQ ID NO: 1825) | |
| RAG3A- | CCAGGGAAAAGAAATTTG | CCAGGGAAAAGAAAUUUGGTT | CCAAAUUUCUUUUCCCUGGT | 1.2 |
| 308 | G | (SEQ ID NO: 1384) | T | |
| | (SEQ ID NO: 942) | | (SEQ ID NO: 1826) | |
| RAG3A- | CCCATGTCCAGGAAAAGAT | CCCAUGUCCAGGAAAAGAUTT | AUCUUUUCCUGGACAUGGGT | 1.2 |
| 158 | (SEQ ID NO: 943) | (SEQ ID NO: 1385) | T | |
| | | | (SEQ ID NO: 1827) | |
| RAG3A- | TTGCCCATGTCCAGGAAAA | UUGCCCAUGUCCAGGAAAATT | UUUUCCUGGACAUGGGCAAT | 1.2 |
| 161 | (SEQ ID NO: 944) | (SEQ ID NO: 1386) | T | |
| | | | (SEQ ID NO: 1828) | |
| RAG3A- | AGGGACCTAGCCAGAAAC | AGGGACCUAGCCAGAAACCTT | GGUUUCUGGCUAGGUCCCUT | 1.2 |
| 1253 | C | (SEQ ID NO: 1387) | T | |
| | (SEQ ID NO: 945) | | (SEQ ID NO: 1829) | |
| RAG3A- | CAGACATGCCCATCCTTGG | CAGACAUGCCCAUCCUUGGTT | CCAAGGAUGGGCAUGUCUGT | 1.2 |
| 926 | (SEQ ID NO: 946) | (SEQ ID NO: 1388) | T | |
| | | | (SEQ ID NO: 1830) | |
| RAG3A- | TTGTAACTTATCCTCAGGC | UUGUAACUUAUCCUCAGGCTT | GCCUGAGGAUAAGUUACAAT | 1.2 |
| 1191 | (SEQ ID NO: 947) | (SEQ ID NO: 1389) | T | |
| | | | (SEQ ID NO: 1831) | |
| RAG3A- | GGACACATGGGCCTGGTTA | GGACACAUGGGCCUGGUUATT | UAACCAGGCCCAUGUGUCCT | 1.2 |
| 697 | (SEQ ID NO: 948) | (SEQ ID NO: 1390) | T | |
| | | | (SEQ ID NO: 1832) | |
| RAG3A- | CCCCTTCTGTGGAGTGACA | CCCCUUCUGUGGAGUGACATT | UGUCACUCCACAGAAGGGGT | 1.2 |
| 1224 | (SEQ ID NO: 949) | (SEQ ID NO: 1391) | T | |
| | | | (SEQ ID NO: 1833) | |
| RAG3A- | GGACGGGAAAATTCCAGG | GGACGGGAAAAUUCCAGGATT | UCCUGGAAUUUUCCCGUCCT | 1.2 |
| 964 | A | (SEQ ID NO: 1392) | T | |
| | (SEQ ID NO: 950) | | (SEQ ID NO: 1834) | |
| RAG3A- | AAAGCAAAGGAGCTAGAG | AAAGCAAAGGAGCUAGAGCTT | GCUCUAGCUCCUUUGCUUUT | 1.2 |
| 791 | C | (SEQ ID NO: 1393) | T | |
| | (SEQ ID NO: 951) | | (SEQ ID NO: 1835) | |
| RAG3A- | TGTGCGCACATACATGTGC | UGUGCGCACAUACAUGUGCTT | GCACAUGUAUGUGCGCACAT | 1.2 |
| 526 | (SEQ ID NO: 952) | (SEQ ID NO: 1394) | T | |
| | | | (SEQ ID NO: 1836) | |
| RAG3A- | GTCTGGCTGGACGGGAAA | GUCUGGCUGGACGGGAAAATT | UUUUCCCGUCCAGCCAGACT | 1.2 |
| 972 | A | (SEQ ID NO: 1395) | T | |
| | (SEQ ID NO: 953) | | (SEQ ID NO: 1837) | |
| RAG3A- | ATAGCCAGGGAGTGAAAAC | AUAGCCAGGGAGUGAAAACTT | GUUUUCACUCCCUGGCUAUT | 1.2 |
| 289 | (SEQ ID NO: 954) | (SEQ ID NO: 1396) | T | |
| | | | (SEQ ID NO: 1838) | |
| RAG3A- | CAAGACCTCTGTGCTTCTT | CAAGACCUCUGUGCUUCUUTT | AAGAAGCACAGAGGUCUUGT | 1.2 |
| 255 | (SEQ ID NO: 955) | (SEQ ID NO: 1397) | T | |
| | | | (SEQ ID NO: 1839) | |
| RAG3A- | CCTCCTTCCTAATCTTGGG | CCUCCUUCCUAAUCUUGGGTT | CCCAAGAUUAGGAAGGAGGT | 1.2 |
| 1000 | (SEQ ID NO: 956) | (SEQ ID NO: 1398) | T | |
| | | | (SEQ ID NO: 1840) | |
| RAG3A- | CACATGGGCCTGGTTATTC | CACAUGGGCCUGGUUAUUCTT | GAAUAACCAGGCCCAUGUGT | 1.2 |
| 694 | (SEQ ID NO: 957) | (SEQ ID NO: 1399) | T | |
| | | | (SEQ ID NO: 1841) | |
| RAG3A- | GCTCAGGCTTAACCCAGTG | GCUCAGGCUUAACCCAGUGTT | CACUGGGUUAAGCCUGAGCT | 1.2 |
| 549 | (SEQ ID NO: 958) | (SEQ ID NO: 1400) | T | |
| | | | (SEQ ID NO: 1842) | |
| RAG3A- | GAAACCGGCAGCATTCCCC | GAAACCGGCAGCAUUCCCCTT | GGGGAAUGCUGCCGGUUUCT | 1.2 |
| 1240 | (SEQ ID NO: 959) | (SEQ ID NO: 1401) | T | |
| | | | (SEQ ID NO: 1843) | |
| RAG3A- | CATCTCGTCTGGCTGGACG | CAUCUCGUCUGGCUGGACGTT | CGUCCAGCCAGACGAGAUGT | 1.2 |
| 978 | (SEQ ID NO: 960) | (SEQ ID NO: 1402) | T | |
| | | | (SEQ ID NO: 1844) | |
| RAG3A- | CTCTGAGGTCGCTCCTCAT | CUCUGAGGUCGCUCCUCAUTT | AUGAGGAGCGACCUCAGAGT | 1.2 |
| 1290 | (SEQ ID NO: 961) | (SEQ ID NO: 1403) | T | |
| | | | (SEQ ID NO: 1845) | |
| RAG3A- | CAGCACCCTCCTTCCTAAT | CAGCACCCUCCUUCCUAAUTT | AUUAGGAAGGAGGGUGCUGT | 1.2 |
| 1006 | (SEQ ID NO: 962) | (SEQ ID NO: 1404) | T | |
| | | | (SEQ ID NO: 1846) | |
| RAG3A- | TAGCCTGCCTCCCTACCTT | UAGCCUGCCUCCCUACCUUTT | AAGGUAGGGAGGCAGGCUAT | 1.2 |
| 1311 | (SEQ ID NO: 963) | (SEQ ID NO: 1405) | T | |
| | | | (SEQ ID NO: 1847) | |
| RAG3A- | GGGGCCTTCACCAGTGTCT | GGGGCCUUCACCAGUGUCUTT | AGACACUGGUGAAGGCCCCT | 1.2 |
| 42 | (SEQ ID NO: 964) | (SEQ ID NO: 1406) | T | |
| | | | (SEQ ID NO: 1848) | |
| RAG3A- | TGGCTCCCTTCTCTGATTG | UGGCUCCCUUCUCUGAUUGTT | CAAUCAGAGAAGGGAGCCAT | 1.2 |
| 21 | (SEQ ID NO: 965) | (SEQ ID NO: 1407) | T | |
| | | | (SEQ ID NO: 1849) | |
| RAG3A- | AGAAGGCGTCACTTCCGGG | AGAAGGCGUCACUUCCGGGTT | CCCGGAAGUGACGCCUUCUT | 1.2 |
| 59 | (SEQ ID NO: 966) | (SEQ ID NO: 1408) | T | |
| | | | (SEQ ID NO: 1850) | |
| RAG3A- | AACTTATCCTCAGGCGCAT | AACUUAUCCUCAGGCGCAUTT | AUGCGCCUGAGGAUAAGUUT | 1.2 |
| 1187 | (SEQ ID NO: 967) | (SEQ ID NO: 1409) | T | |
| | | | (SEQ ID NO: 1851) | |
| RAG3A- | ACTGGACCCCAGCAGACG | ACUGGACCCCAGCAGACGATT | UCGUCUGCUGGGGUCCAGUT | 1.1 |
| 576 | A | (SEQ ID NO: 1410) | T | |
| | (SEQ ID NO: 968) | | (SEQ ID NO: 1852) | |
| RAG3A- | GGAGACAGAACAAGCAAA | GGAGACAGAACAAGCAAAGTT | CUUUGCUUGUUCUGUCUCCT | 1.1 |
| 639 | G | (SEQ ID NO: 1411) | T | |
| | (SEQ ID NO: 969) | | (SEQ ID NO: 1853) | |
| RAG3A- | TGTGGGGAGACTGGGACT | UGUGGGGAGACUGGGACUGTT | CAGUCCCAGUCUCCCCACATT | 1.1 |
| 815 | G | (SEQ ID NO: 1412) | (SEQ ID NO: 1854) | |
| | (SEQ ID NO: 970) | | | |
| RAG3A- | AAAATTCCAGGATCTAGGC | AAAAUUCCAGGAUCUAGGCTT | GCCUAGAUCCUGGAAUUUUT | 1.1 |
| 957 | (SEQ ID NO: 971) | (SEQ ID NO: 1413) | T (SEQ ID NO: 1855) | |
| RAG3A- | TGTGGAGTGACAGTATCTC | UGUGGAGUGACAGUAUCUCTT | GAGAUACUGUCACUCCACAT | 1.1 |
| 1217 | (SEQ ID NO: 972) | (SEQ ID NO: 1414) | T | |
| | | | (SEQ ID NO: 1856) | |
| RAG3A- | ACAGTCCCCTCTTGCTTTC | ACAGUCCCCUCUUGCUUUCTT | GAAAGCAAGAGGGGACUGUT | 1.1 |
| 1165 | (SEQ ID NO: 973) | (SEQ ID NO: 1415) | T | |
| | | | (SEQ ID NO: 1857) | |
| RAG3A- | TTCACCCAAGGGACCCTCT | UUCACCCAAGGGACCCUCUTT | AGAGGGUCCCUUGGGUGAAT | 1.1 |
| 1134 | (SEQ ID NO: 974) | (SEQ ID NO: 1416) | T | |
| | | | (SEQ ID NO: 1858) | |
| RAG3A- | ACATGTGGAACGGTAGGAG | ACAUGUGGAACGGUAGGAGTT | CUCCUACCGUUCCACAUGUT | 1.1 |
| 667 | (SEQ ID NO: 975) | (SEQ ID NO: 1417) | T | |
| | | | (SEQ ID NO: 1859) | |
| RAG3A- | CAAACCCTCACCACCCTCT | CAAACCCUCACCACCCUCUTT | AGAGGGUGGUGAGGGUUUGT | 1.1 |
| 1339 | (SEQ ID NO: 976) | (SEQ ID NO: 1418) | T | |
| | | | (SEQ ID NO: 1860) | |
| RAG3A- | CCTCTTGTCACATGTGGAA | CCUCUUGUCACAUGUGGAATT | UUCCACAUGUGACAAGAGGT | 1.1 |
| 676 | (SEQ ID NO: 977) | (SEQ ID NO: 1419) | T | |
| | | | (SEQ ID NO: 1861) | |
| RAG3A- | GTCCAGGAAAAGATGGATC | GUCCAGGAAAAGAUGGAUCTT | GAUCCAUCUUUUCCUGGACT | 1.1 |
| 153 | (SEQ ID NO: 978) | (SEQ ID NO: 1420) | T | |
| | | | (SEQ ID NO: 1862) | |
| RAG3A- | CAGGTATGATGTCCAGGGA | CAGGUAUGAUGUCCAGGGATT | UCCCUGGACAUCAUACCUGT | 1.1 |
| 320 | (SEQ ID NO: 979) | (SEQ ID NO: 1421) | T | |
| | | | (SEQ ID NO: 1863) | |
| RAG3A- | ATCCCAGCACCCTCCTTCC | AUCCCAGCACCCUCCUUCCTT | GGAAGGAGGGUGCUGGGAUT | 1.1 |
| 1010 | (SEQ ID NO: 980) | (SEQ ID NO: 1422) | T | |
| | | | (SEQ ID NO: 1864) | |
| RAG3A- | CTCCTTCCTAATCTTGGGA | CUCCUUCCUAAUCUUGGGATT | UCCCAAGAUUAGGAAGGAGT | 1.1 |
| 999 | (SEQ ID NO: 981) | (SEQ ID NO: 1423) | T | |
| | | | (SEQ ID NO: 1865) | |
| RAG3A- | CAGCAGACGAGCACCTAA | CAGCAGACGAGCACCUAAGTT | CUUAGGUGCUCGUCUGCUGT | 1.1 |
| 567 | G | (SEQ ID NO: 1424) | T | |
| | (SEQ ID NO: 982) | | (SEQ ID NO: 1866) | |
| RAG3A- | GGATGATGGGATCAAGGTC | GGAUGAUGGGAUCAAGGUCTT | GACCUUGAUCCCAUCAUCCT | 1.1 |
| 862 | (SEQ ID NO: 983) | (SEQ ID NO: 1425) | T | |
| | | | (SEQ ID NO: 1867) | |
| RAG3A- | CACCAACCCATAACAGGAG | CACCAACCCAUAACAGGAGTT | CUCCUGUUAUGGGUUGGUGT | 1.1 |
| 460 | (SEQ ID NO: 984) | (SEQ ID NO: 1426) | T | |
| | | | (SEQ ID NO: 1868) | |
| RAG3A- | ATGCCCATCCTTGGGGAGG | AUGCCCAUCCUUGGGGAGGTT | CCUCCCCAAGGAUGGGCAUT | 1.1 |
| 921 | (SEQ ID NO: 985) | (SEQ ID NO: 1427) | T | |
| | | | (SEQ ID NO: 1869) | |
| RAG3A- | AAGACAGGGACACATGGG | AAGACAGGGACACAUGGGCTT | GCCCAUGUGUCCCUGUCUUT | 1.1 |
| 704 | C | (SEQ ID NO: 1428) | T | |
| | (SEQ ID NO: 986) | | (SEQ ID NO: 1870) | |
| RAG3A- | CAGCAACACAAATGTCCTG | CAGCAACACAAAUGUCCUGTT | CAGGACAUUUGUGUUGCUGT | 1.1 |
| 233 | (SEQ ID NO: 987) | (SEQ ID NO: 1429) | T | |
| | | | (SEQ ID NO: 1871) | |
| RAG3A- | ATCTCCCTCTCATTGTAAC | AUCUCCCUCUCAUUGUAACTT | GUUACAAUGAGAGGGAGAUT | 1.1 |
| 1203 | (SEQ ID NO: 988) | (SEQ ID NO: 1430) | T | |
| | | | (SEQ ID NO: 1872) | |
| RAG3A- | CTGTGCTTCTTCCCCAGCA | CUGUGCUUCUUCCCCAGCATT | UGCUGGGGAAGAAGCACAGT | 1.1 |
| 247 | (SEQ ID NO: 989) | (SEQ ID NO: 1431) | T | |
| | | | (SEQ ID NO: 1873) | |
| RAG3A- | CAGCAGCTGTCTTTCCTAA | CAGCAGCUGUCUUUCCUAATT | UUAGGAAAGACAGCUGCUGT | 1.1 |
| 721 | (SEQ ID NO: 990) | (SEQ ID NO: 1432) | T | |
| | | | (SEQ ID NO: 1874) | |
| RAG3A- | ACAAGATAGGACTCCCTAG | ACAAGAUAGGACUCCCUAGTT | CUAGGGAGUCCUAUCUUGUT | 1.1 |
| 391 | (SEQ ID NO: 991) | (SEQ ID NO: 1433) | T | |
| | | | (SEQ ID NO: 1875) | |
| RAG3A- | GCCCATGTCCAGGAAAAGA | GCCCAUGUCCAGGAAAAGATT | UCUUUUCCUGGACAUGGGCT | 1.1 |
| 159 | (SEQ ID NO: 992) | (SEQ ID NO: 1434) | T | |
| | | | (SEQ ID NO: 1876) | |
| RAG3A- | CTCTCATGTGGGCAATATC | CUCUCAUGUGGGCAAUAUCTT | GAUAUUGCCCACAUGAGAGT | 1.1 |
| 437 | (SEQ ID NO: 993) | (SEQ ID NO: 1435) | T | |
| | | | (SEQ ID NO: 1877) | |
| RAG3A- | GTGTCTGGTGGCTCCCTTC | GUGUCUGGUGGCUCCCUUCTT | GAAGGGAGCCACCAGACACT | 1.1 |
| 29 | (SEQ ID NO: 994) | (SEQ ID NO: 1436) | T | |
| | | | (SEQ ID NO: 1878) | |
| RAG3A- | GAAGAGAAGGCGTCACTT | GAAGAGAAGGCGUCACUUCTT | GAAGUGACGCCUUCUCUUCT | 1.1 |
| 63 | C | (SEQ ID NO: 1437) | T | |
| | (SEQ ID NO: 995) | | (SEQ ID NO: 1879) | |
| RAG3A- | CTTCCTAATCTTGGGAGAC | CUUCCUAAUCUUGGGAGACTT | GUCUCCCAAGAUUAGGAAGT | 1.1 |
| 996 | (SEQ ID NO: 996) | (SEQ ID NO: 1438) | T | |
| | | | (SEQ ID NO: 1880) | |
| RAG3A- | TTGGTTCCCTGGTCATGCC | UUGGUUCCCUGGUCAUGCCTT | GGCAUGACCAGGGAACCAAT | 1.1 |
| 1083 | (SEQ ID NO: 997) | (SEQ ID NO: 1439) | T | |
| | | | (SEQ ID NO: 1881) | |
| RAG3A- | CAAGGGACCCTCTGCCTCT | CAAGGGACCCUCUGCCUCUTT | AGAGGCAGAGGGUCCCUUGT | 1.1 |
| 1128 | (SEQ ID NO: 998) | (SEQ ID NO: 1440) | T | |
| | | | (SEQ ID NO: 1882) | |
| RAG3A- | CCTCCTTTCTCTTGGTTCC | CCUCCUUUCUCUUGGUUCCTT | GGAACCAAGAGAAAGGAGGT | 1.1 |
| 1094 | (SEQ ID NO: 999) | (SEQ ID NO: 1441) | T | |
| | | | (SEQ ID NO: 1883) | |
| RAG3A- | AGGGCCAAAGGAAAAGGG | AGGGCCAAAGGAAAAGGGGTT | CCCCUUUUCCUUUGGCCCUTT | 1.1 |
| 771 | G | (SEQ ID NO: 1442) | (SEQ ID NO: 1884) | |
| | (SEQ ID NO: 1000) | | | |
| RAG3A- | AGCCAGGGAGTGAAAACC | AGCCAGGGAGUGAAAACCCTT | GGGUUUUCACUCCCUGGCUT | 1.1 |
| 287 | C | (SEQ ID NO: 1443) | T | |
| | (SEQ ID NO: 1001) | | (SEQ ID NO: 1885) | |
| RAG3A- | AGACATCTCGTCTGGCTGG | AGACAUCUCGUCUGGCUGGTT | CCAGCCAGACGAGAUGUCUT | 1.1 |
| 981 | (SEQ ID NO: 1002) | (SEQ ID NO: 1444) | T | |
| | | | (SEQ ID NO: 1886) | |
| RAG3A- | GGTGGCTCCCTTCTCTGAT | GGUGGCUCCCUUCUCUGAUTT | AUCAGAGAAGGGAGCCACCT | 1.1 |
| 23 | (SEQ ID NO: 1003) | (SEQ ID NO: 1445) | T | |
| | | | (SEQ ID NO: 1887) | |
| RAG3A- | GTCATGCCTGCCTCCCTGT | GUCAUGCCUGCCUCCCUGUTT | ACAGGGAGGCAGGCAUGACT | 1.1 |
| 1072 | (SEQ ID NO: 1004) | (SEQ ID NO: 1446) | T | |
| | | | (SEQ ID NO: 1888) | |
| RAG3A- | GTTCCCACTTCGAAAGGGG | GUUCCCACUUCGAAAGGGGTT | CCCCUUUCGAAGUGGGAACT | 1.1 |
| 415 | (SEQ ID NO: 1005) | (SEQ ID NO: 1447) | T | |
| | | | (SEQ ID NO: 1889) | |
| RAG3A- | TGAGGTCGCTCCTCATTCT | UGAGGUCGCUCCUCAUUCUTT | AGAAUGAGGAGCGACCUCAT | 1.1 |
| 1287 | (SEQ ID NO: 1006) | (SEQ ID NO: 1448) | T | |
| | | | (SEQ ID NO: 1890) | |
| RAG3A- | CTCCCTCTCATTGTAACTT | CUCCCUCUCAUUGUAACUUTT | AAGUUACAAUGAGAGGGAGT | 1.1 |
| 1201 | (SEQ ID NO: 1007) | (SEQ ID NO: 1449) | T | |
| | | | (SEQ ID NO: 1891) | |
| RAG3A- | CAGGGCCAAAGGAAAAGG | CAGGGCCAAAGGAAAAGGGTT | CCCUUUUCCUUUGGCCCUGT | 1.1 |
| 772 | G | (SEQ ID NO: 1450) | T | |
| | (SEQ ID NO: 1008) | | (SEQ ID NO: 1892) | |
| RAG3A- | ACCTAGCCAGAAACCGGCA | ACCUAGCCAGAAACCGGCATT | UGCCGGUUUCUGGCUAGGUT | 1.1 |
| 1249 | (SEQ ID NO: 1009) | (SEQ ID NO: 1451) | T | |
| | | | (SEQ ID NO: 1893) | |
| RAG3A- | CAGGCTTAACCCAGTGCAC | CAGGCUUAACCCAGUGCACTT | GUGCACUGGGUUAAGCCUGT | 1.1 |
| 546 | (SEQ ID NO: 1010) | (SEQ ID NO: 1452) | T | |
| | | | (SEQ ID NO: 1894) | |
| RAG3A- | TGCCCATGTCCAGGAAAAG | UGCCCAUGUCCAGGAAAAGTT | CUUUUCCUGGACAUGGGCAT | 1.1 |
| 160 | (SEQ ID NO: 1011) | (SEQ ID NO: 1453) | T | |
| | | | (SEQ ID NO: 1895) | |
| RAG3A- | GGAAGAGAAGGCGTCACT | GGAAGAGAAGGCGUCACUUTT | AAGUGACGCCUUCUCUUCCT | 1.1 |
| 64 | T | (SEQ ID NO: 1454) | T | |
| | (SEQ ID NO: 1012) | | (SEQ ID NO: 1896) | |
| RAG3A- | ACCCTCCTTCCTAATCTTG | ACCCUCCUUCCUAAUCUUGTT | CAAGAUUAGGAAGGAGGGUT | 1.1 |
| 1002 | (SEQ ID NO: 1013) | (SEQ ID NO: 1455) | T | |
| | | | (SEQ ID NO: 1897) | |
| RAG3A- | CTGAGGTCGCTCCTCATTC | CUGAGGUCGCUCCUCAUUCTT | GAAUGAGGAGCGACCUCAGT | 1.1 |
| 1288 | (SEQ ID NO: 1014) | (SEQ ID NO: 1456) | T | |
| | | | (SEQ ID NO: 1898) | |
| RAG3A- | AGAGCCTGAGGAAGTTCTG | AGAGCCUGAGGAAGUUCUGTT | CAGAACUUCCUCAGGCUCUT | 1.1 |
| 891 | (SEQ ID NO: 1015) | (SEQ ID NO: 1457) | T | |
| | | | (SEQ ID NO: 1899) | |
| RAG3A- | GCAACACAAATGTCCTGCC | GCAACACAAAUGUCCUGCCTT | GGCAGGACAUUUGUGUUGCT | 1.1 |
| 231 | (SEQ ID NO: 1016) | (SEQ ID NO: 1458) | T | |
| | | | (SEQ ID NO: 1900) | |
| RAG3A- | TCACATGTGGAACGGTAGG | UCACAUGUGGAACGGUAGGTT | CCUACCGUUCCACAUGUGATT | 1.1 |
| 669 | (SEQ ID NO: 1017) | (SEQ ID NO: 1459) | (SEQ ID NO: 1901) | |
| RAG3A- | TCCCAGCACCCTCCTTCCT | UCCCAGCACCCUCCUUCCUTT | AGGAAGGAGGGUGCUGGGAT | 1.1 |
| 1009 | (SEQ ID NO: 1018) | (SEQ ID NO: 1460) | T | |
| | | | (SEQ ID NO: 1902) | |
| RAG3A- | GGTAGGAGATGGAAGACG | GGUAGGAGAUGGAAGACGGTT | CCGUCUUCCAUCUCCUACCTT | 1.1 |
| 656 | G | (SEQ ID NO: 1461) | (SEQ ID NO: 1903) | |
| | (SEQ ID NO: 1019) | | | |
| RAG3A- | AACACAAATGTCCTGCCAG | AACACAAAUGUCCUGCCAGTT | CUGGCAGGACAUUUGUGUUT | 1.1 |
| 229 | (SEQ ID NO: 1020) | (SEQ ID NO: 1462) | T | |
| | | | (SEQ ID NO: 1904) | |
| RAG3A- | CCCGTCCAAACCCTTTCCC | CCCGUCCAAACCCUUUCCCTT | GGGAAAGGGUUUGGACGGGT | 1.1 |
| 484 | (SEQ ID NO: 1021) | (SEQ ID NO: 1463) | T | |
| | | | (SEQ ID NO: 1905) | |
| RAG3A- | CTGGGAAGAAAGCAAAGG | CUGGGAAGAAAGCAAAGGATT | UCCUUUGCUUUCUUCCCAGT | 1.1 |
| 799 | A | (SEQ ID NO: 1464) | T | |
| | (SEQ ID NO: 1022) | | (SEQ ID NO: 1906) | |
| RAG3A- | AATTTGGATAGCCAGGGAG | AAUUUGGAUAGCCAGGGAGTT | CUCCCUGGCUAUCCAAAUUT | 1.1 |
| 296 | (SEQ ID NO: 1023) | (SEQ ID NO: 1465) | T | |
| | | | (SEQ ID NO: 1907) | |
| RAG3A- | TATCCTCAGGCGCATTCGA | UAUCCUCAGGCGCAUUCGATT | UCGAAUGCGCCUGAGGAUAT | 1.1 |
| 1183 | (SEQ ID NO: 1024) | (SEQ ID NO: 1466) | T | |
| | | | (SEQ ID NO: 1908) | |
| RAG3A- | CTCCCTAGGGGATTACAGA | CUCCCUAGGGGAUUACAGATT | UCUGUAAUCCCCUAGGGAGT | 1.1 |
| 380 | (SEQ ID NO: 1025) | (SEQ ID NO: 1467) | T (SEQ ID NO: 1909) | |
| RAG3A- | CCATGTCCAGGAAAAGATG | CCAUGUCCAGGAAAAGAUGTT | CAUCUUUUCCUGGACAUGGT | 1.1 |
| 157 | (SEQ ID NO: 1026) | (SEQ ID NO: 1468) | T | |
| | | | (SEQ ID NO: 1910) | |
| RAG3A- | CTTATCCTCAGGCGCATTC | CUUAUCCUCAGGCGCAUUCTT | GAAUGCGCCUGAGGAUAAGT | 1.1 |
| 1185 | (SEQ ID NO: 1027) | (SEQ ID NO: 1469) | T | |
| | | | (SEQ ID NO: 1911) | |
| RAG3A- | GCCAGAAACCGGCAGCATT | GCCAGAAACCGGCAGCAUUTT | AAUGCUGCCGGUUUCUGGCT | 1.1 |
| 1244 | (SEQ ID NO: 1028) | (SEQ ID NO: 1470) | T | |
| | | | (SEQ ID NO: 1912) | |
| RAG3A- | GCCATCTAAGCCACTGGAC | GCCAUCUAAGCCACUGGACTT | GUCCAGUGGCUUAGAUGGCT | 1.1 |
| 588 | (SEQ ID NO: 1029) | (SEQ ID NO: 1471) | T | |
| | | | (SEQ ID NO: 1913) | |
| RAG3A- | TCTCATGTGGGCAATATCC | UCUCAUGUGGGCAAUAUCCTT | GGAUAUUGCCCACAUGAGAT | 1.1 |
| 436 | (SEQ ID NO: 1030) | (SEQ ID NO: 1472) | T | |
| | | | (SEQ ID NO: 1914) | |
| RAG3A- | ACCCACTATCCTCCCAGCT | ACCCACUAUCCUCCCAGCUTT | AGCUGGGAGGAUAGUGGGUT | 1.1 |
| 1029 | (SEQ ID NO: 1031) | (SEQ ID NO: 1473) | T | |
| | | | (SEQ ID NO: 1915) | |
| RAG3A- | TGTCTGGTGGCTCCCTTCT | UGUCUGGUGGCUCCCUUCUTT | AGAAGGGAGCCACCAGACAT | 1.1 |
| 28 | (SEQ ID NO: 1032) | (SEQ ID NO: 1474) | T | |
| | | | (SEQ ID NO: 1916) | |
| RAG3A- | CCAGCAACACAAATGTCCT | CCAGCAACACAAAUGUCCUTT | AGGACAUUUGUGUUGCUGGT | 1.1 |
| 234 | (SEQ ID NO: 1033) | (SEQ ID NO: 1475) | T | |
| | | | (SEQ ID NO: 1917) | |
| RAG3A- | CTCTGTGCTTCTTCCCCAG | CUCUGUGCUUCUUCCCCAGTT | CUGGGGAAGAAGCACAGAGT | 1.1 |
| 249 | (SEQ ID NO: 1034) | (SEQ ID NO: 1476) | T | |
| | | | (SEQ ID NO: 1918) | |
| RAG3A- | AGCCAGGGCCAAAGGAAA | AGCCAGGGCCAAAGGAAAATT | UUUUCCUUUGGCCCUGGCUT | 1.1 |
| 775 | A | (SEQ ID NO: 1477) | T | |
| | (SEQ ID NO: 1035) | | (SEQ ID NO: 1919) | |
| RAG3A- | CCAGGATCTAGGCCACACT | CCAGGAUCUAGGCCACACUTT | AGUGUGGCCUAGAUCCUGGT | 1.1 |
| 951 | (SEQ ID NO: 1036) | (SEQ ID NO: 1478) | T | |
| | | | (SEQ ID NO: 1920) | |
| RAG3A- | GATGATGGGATCAAGGTCA | GAUGAUGGGAUCAAGGUCATT | UGACCUUGAUCCCAUCAUCT | 1.1 |
| 861 | (SEQ ID NO: 1037) | (SEQ ID NO: 1479) | T | |
| | | | (SEQ ID NO: 1921) | |
| RAG3A- | TAAACAAGACCTCTGTGCT | UAAACAAGACCUCUGUGCUTT | AGCACAGAGGUCUUGUUUAT | 1.1 |
| 259 | (SEQ ID NO: 1038) | (SEQ ID NO: 1480) | T | |
| | | | (SEQ ID NO: 1922) | |
| RAG3A- | ATGACAAGATAGGACTCCC | AUGACAAGAUAGGACUCCCTT | GGGAGUCCUAUCUUGUCAUT | 1.1 |
| 394 | (SEQ ID NO: 1039) | (SEQ ID NO: 1481) | T | |
| | | | (SEQ ID NO: 1923) | |
| RAG3A- | AGTGCACGTGTGCGCACAT | AGUGCACGUGUGCGCACAUTT | AUGUGCGCACACGUGCACUT | 1.1 |
| 534 | (SEQ ID NO: 1040) | (SEQ ID NO: 1482) | T | |
| | | | (SEQ ID NO: 1924) | |
| RAG3A- | TATGATGTCCAGGGAAAAG | UAUGAUGUCCAGGGAAAAGTT | CUUUUCCCUGGACAUCAUAT | 1.1 |
| 316 | (SEQ ID NO: 1041) | (SEQ ID NO: 1483) | T | |
| | | | (SEQ ID NO: 1925) | |
| RAG3A- | CGAGCACCTAAGCTCAGGC | CGAGCACCUAAGCUCAGGCTT | GCCUGAGCUUAGGUGCUCGT | 1.1 |
| 560 | (SEQ ID NO: 1042) | (SEQ ID NO: 1484) | T | |
| | | | (SEQ ID NO: 1926) | |
| RAG3A- | CCCACTCCCAGCCTCCTTT | CCCACUCCCAGCCUCCUUUTT | AAAGGAGGCUGGGAGUGGGT | 1.1 |
| 1105 | (SEQ ID NO: 1043) | (SEQ ID NO: 1485) | T | |
| | | | (SEQ ID NO: 1927) | |
| RAG3A- | TTCCTAAGACAGGGACACA | UUCCUAAGACAGGGACACATT | UGUGUCCCUGUCUUAGGAAT | 1.1 |
| 709 | (SEQ ID NO: 1044) | (SEQ ID NO: 1486) | T | |
| | | | (SEQ ID NO: 1928) | |
| RAG3A- | GGATCTAGGCCACACTTCT | GGAUCUAGGCCACACUUCUTT | AGAAGUGUGGCCUAGAUCCT | 1.1 |
| 948 | (SEQ ID NO: 1045) | (SEQ ID NO: 1487) | T | |
| | | | (SEQ ID NO: 1929) | |
| RAG3A- | TCCTTCCTAATCTTGGGAG | UCCUUCCUAAUCUUGGGAGTT | CUCCCAAGAUUAGGAAGGAT | 1.1 |
| 998 | (SEQ ID NO: 1046) | (SEQ ID NO: 1488) | T | |
| | | | (SEQ ID NO: 1930) | |
| RAG3A- | GATAGGACTCCCTAGGGGA | GAUAGGACUCCCUAGGGGATT | UCCCCUAGGGAGUCCUAUCT | 1.1 |
| 387 | (SEQ ID NO: 1047) | (SEQ ID NO: 1489) | T | |
| | | | (SEQ ID NO: 1931) | |

It can be clearly seen from the arrangement of activities of the 833 saRNAs according to their positions within the THPO promoter region that the functional saRNAs are gathered together, that is, in certain promoter regions, the activating saRNAs gathered in specific “hotspot” regions (FIG. 11). As shown in FIG. 11, 5 hotspot regions appeared in region (H1) from −1339 to −1044, region (H2) from −1027 to −903, region (H3) from −861 to −754, region (H4) from −728 to −611, and region (H5) from −593 to −1 of the promoter, respectively, showing high gathering of the activating saRNAs. These results indicate that the activating saRNAs were not randomly distributed within the promoter, but existed in the specific hotspot regions.

The sequence of the hotspot H1 (5′ to 3′: −1339 to −1044) corresponds to position 1 to position 296, from 5′ to 3′, of SEQ ID NO: 601 in the sequence listing:

| | |
|---|---|
| 1 | caaaccctca ccaccctctc tcactgccta gcctgcctcc ctaccttctc |
| 51 | tctgaggtcg ctcctcattc ttgtgttacc caggacaggg acctagccag |
| 101 | aaaccggcag cattccccct tctgtggagt gacagtatct ccctctcatt |
| 151 | gtaacttatc ctcaggcgca ttcgacagtc ccctcttgct ttctcacccc |
| 201 | cttccttcac ccaagggacc ctctgcctct ccagcccact cccagcctcc |
| 251 | tttctcttgg ttccctggtc atgcctgcct ccctgtctcc tgtctc |

The sequence of the hotspot H2 (5′ to 3′: −1027 to −903) corresponds to position 1 to position 125, from 5′ to 3′, of SEQ ID NO: 602 in the sequence listing:

| | |
|---|---|
| 1 | ccactatcct cccagctatc ccagcaccct ccttcctaat cttgggagac |
| 51 | atctcgtctg gctggacggg aaaattccag gatctaggcc acacttctca |
| 101 | gcagacatgc ccatccttgg ggagg |

The sequence of the hotspot H3 (5′ to 3′: −861 to −754) corresponds to position 1 to position 108, from 5′ to 3′, of SEQ ID NO: 603 in the sequence listing:

| | |
|---|---|
| 1 | gatgatggga tcaaggtcag gccaggaagc ccctgaggac agagactgtg |
| 51 | gggagactgg gactgggaag aaagcaaagg agctagagcc agggccaaag |
| 101 | gaaaaggg |

The sequence of the hotspot H4 (5′ to 3′: −728 to −611) corresponds to position 1 to position 118, from 5′ to 3′, of SEQ ID NO: 604 in the sequence listing:

| | |
|---|---|
| 1 | ggaggtccag cagctgtctt tcctaagaca gggacacatg ggcctggtta |
| 51 | ttcctcttgt cacatgtgga acggtaggag atggaagacg gagacagaac |
| 101 | aagcaaagga gggccctg |

The sequence of the hotspot H5 (5′ to 3′: −593 to −1) corresponds to position 1 to position 593, from 5′ to 3′, of SEQ ID NO: 605 in the sequence listing:

| | |
|---|---|
| 1 | gtgtagccat ctaagccact ggaccccagc agacgagcac ctaagctcag |
| 51 | gcttaaccca gtgcacgtgt gcgcacatac atgtgccccg cacctgacag |
| 101 | tccactcaac ccgtccaaac cctttcccca taacaccaac ccataacagg |
| 151 | agatttctct catgtgggca atatccgtgt tcccacttcg aaagggggaa |
| 201 | tgacaagata ggactcccta ggggattaca gaaagaaaag caggaaagca |
| 251 | agcatcctgt tggatttcag cagcaggtat gatgtccagg gaaaagaaat |
| 301 | ttggatagcc agggagtgaa aaccccacca atcttaaaca agacctctgt |
| 351 | gcttcttccc cagcaacaca aatgtcctgc cagattcctc ctggaaaaaa |
| 401 | acttctgctc ctgtccccct ccaggtccca ggttgcccat gtccaggaaa |
| 451 | agatggatcc ccctatccaa atcttctccg tggtgtgtgt gggtggagga |
| 501 | gtggaccctg gtccaggcag gggctccagg gaagagaagg cgtcacttcc |
| 551 | gggggccttc accagtgtct ggtggctccc ttctctgatt ggg |

### Example 10: Human THPO saRNA Promoting the Expression of THPO mRNA in Human Liver Cancer Cells (HepG2)

(1) Cell Culture and Transfection

Cell culture was described in Example 9. HepG2 cells (purchased from Shanghai Institutes for Biological Sciences, TCHu119) were plated into a 6-well plate at 2×10⁵ cells/well. Following the instructions provided by the manufacturer, RNAiMax (Invitrogen, Carlsbad, Calif.) was used to transfect human THPO saRNA at a concentration of 25 nM (unless otherwise specified), and transfection duration was 72 h, Each treatment was performed in 2 replicate wells.

(2) Two-Step RT-qPCR

Media were discarded after transfection and 500 μL of cell lysis buffer was added to each well and incubated at room temperature for 5 min. RNA was extracted using Qiagen RNeasy kit, reverse transcribed, and subjected to qPCR analysis on a ABI 7500 fast real-time PCR system (Applied Biosystems). Each sample was amplified repeatedly in 3 replicate wells. RT-PCR and qPCR reaction systems are shown in Example 5, Tables 5-6 and the amplification primers are shown in Table 11. TBP and HPRT1 served as internal controls, and THPO was amplified using a THPO F1/R1 primer pair.

The reaction conditions were as follows: 30 s at 95° C., 5 s at 95° C., 30 s at 60° C., with 40 cycles of amplification.

To calculate the expression value (Eᵣₑₗ) of the THPO gene (target) of an saRNA-transfected sample relative to control treatment (Mock), the Ct values of the target gene and two internal control genes were substituted into Formula 2 in Example 3 for calculation.

Eᵣₑₗ=2^{(CtTm-CtTs)}/2^{(CtRm-CtRs)} Formula 2

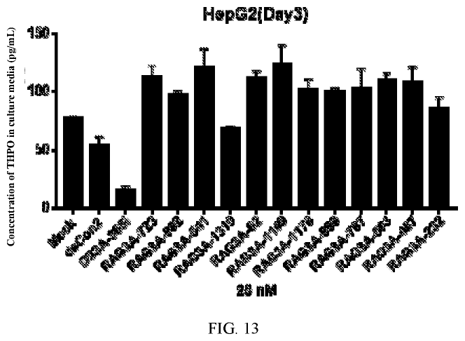

FIG. 12 shows the relative mRNA expression values of THPO after treatment of cells with different saRNAs. The relative mRNA expression of siRNA group (RAG3A-365i) is reduced by 85% compared to the control group which uses a blank transfection control (Mock), indicating that siRNA was successful as a small interfering RNA control transfection. The relative mRNA expression values of RAG3A-1219, RAG3A-1096, RAG3A-1308, RAG3A-1069 and RAG3A-851 groups were all higher than those of the control group, and the relative mRNA expression values increased 1.8, 1.8, 1.6, 1.7 and 1.9, respectively. The relative mRNA expression values of RAG3A-541, RAG3A-1319, RAG3A-62, RAG3A-889 and RAG3A-787 groups were all higher than those of the control group, and increased 2.5, 2.1, 2.9, 2.0 and 2.0, respectively. The activation effect of RAG3A-62 was evident, and the relative mRNA expression value was increased by nearly 3 times. This indicates that randomly selected activating saRNAs could not only be transfected successfully into HepG2 cells, but also can significantly increased expression of THPO mRNA.

### Example 11: Human THPO saRNA Promoting the Expression of THPO Protein Level in Human Liver Cancer Cells (HepG2)

Cell culture methods were described in Example 9 and cell transfection methods were described in Example 10. Assay of THPO protein level in HepG2 cell culture media using ELISA Kit
1) Human THPO protein standard was dissolved using deionized water or distilled water to give a stock solution of 20,000 pg/mL protein standard. One hundred microliters of THPO protein standard stock solution was added to 900 μL of calibration diluent RD6-11 (dilution 1:5) (for cell culture supernatant samples) or calibration diluent RD6-11 (for serum/plasma samples) to give 2000 pg/mL of standard solution, of which 7 serial dilutions were performed with the appropriate calibration diluent as the zero standard (0 pg/mL).

2) Cell supernatants were sampled and centrifuged to remove dead cells and debris.

3) Proper amount of micro-well plate belts from micro-well plate frame were taken out.

4) 50 μL of assay diluent RD1-1 was added to each well.

5) 200 μL of standard, control, or treatment samples were added to each well. The membrane was covered and the samples were incubated at room temperature for 3 h.

6) The liquid was removed from each well and washed 4 times with 400 μL of wash buffer.

7) 200 μL of human THPO antibody was added to each well and incubated for 1 h at room temperature.

8) Step 5 was repeated.

9) 200 μL of substrate solution (Color reagents A and B mixed in equal volumes and used up within 15 min) was added to each well and incubated for 30 min at room temperature in the absence of light.

10) 50 μL of stop solution was added to each well and mixed gently.

11) Absorbance at 450 nM was determined using a multi-functional microplate reader (within 30 min).

FIG. 13 shows the expression value of relative protein level of THPO in cell culture media after treatment of cells with different saRNAs. The relative protein expression of siRNA group (DS3A-365i) group is reduced by 81% compared to the control group which uses a blank transfection control, indicating that siRNA was successful as a small interfering RNA control transfection. The relative protein level expression of THPO of RAG3A-723, RAG3A-892, RAG3A-62, RAG3A-1178, RAG3A-889, RAG3A-563, RAG3A-487 and RAG3A-787 groups were higher than those of the control group, and the relative protein level expression of THPO was increased by 1.46, 1.26, 1.44, 1.31, 1.29, 1.32, 1.42 and 1.40, respectively. This indicates that randomly selected activating saRNAs are capable of promoting the expression of THPO protein to levels of varying degrees in HepG2 cells.

Based on the above results, a plurality of saRNAs capable of remarkably activating the expression of human and mouse THPO/Thpo genes were found through high-throughput screening of saRNAs targeting human and mouse THPO/Thpo gene promoters. It has been found by applicants from multiple studies that both mouse active saRNAs targeting the Thpo gene and human active saRNAs targeting the THPO gene could be efficiently delivered into cells by the liposome system used in the present invention and can significantly up-regulate the expression of the Thpo/THPO gene and protein in cells at the mRNA and protein level. In particular, the active saRNA of the Thpo gene of mouse significantly increases the expression of Thpo mRNA and protein in the mouse and simultaneously up-regulates or increases the number of platelets significantly. Therefore, the mouse Thpo active saRNA and the human THPO active saRNA of the present invention can be used for treating a disease or condition caused by a decrease or insufficient Thpo/THPO protein expression or a disease or condition caused by various factors such as thrombocytopenia, or in the preparation of a drug for treating the aforementioned disease or condition.

### REFERENCES

Bartley, T. D., J. Bogenberger, P. Hunt, Y. S. Li, H. S. Lu, F. Martin, M. S. Chang, B. Samal, J. L. Nichol, S. Swift, and et al. 1994. ‘Identification and cloning of a megakaryocyte growth and development factor that is a ligand for the cytokine receptor Mpl’, Cell, 77: 1117-24.
Basser, R. 2002. ‘The impact of thrombopoietin on clinical practice’, Curr Pharm Des, 8: 369-77.
de Sauvage, F. J., P. E. Hass, S. D. Spencer, B. E. Malloy, A. L. Gurney, S. A. Spencer, W. C. Darbonne, W. J. Henzel, S. C. Wong, W. J. Kuang, and et al. 1994. ‘Stimulation of megakaryocytopoiesis and thrombopoiesis by the c-Mpl ligand’, Nature, 369: 533-8.
Hassan, M. N., and E. K. Waller. 2015. ‘Treating chemotherapy-induced thrombocytopenia: is it time for oncologists to use thrombopoietin agonists?’, Oncology (Williston Park), 29: 295-6.
Kaushansky, K. 2006. ‘Lineage-specific hematopoietic growth factors’, N Engl J Med, 354: 2034-45.

Kuter, D. J. 2009. ‘Thrombopoietin and thrombopoietin mimetics in the treatment of thrombocytopenia’, Annu Rev Med, 60: 193-206.
Laki, K. 1972. ‘Our ancient heritage in blood clotting and some of its consequences’, Ann N Y Acad Sci, 202: 297-307.
Machlus, K. R., J. N. Thon, and J. E. Italiano, Jr. 2014. ‘Interpreting the developmental dance of the megakaryocyte: a review of the cellular and molecular processes mediating platelet formation’, Br J Haematol, 165: 227-36.
Mignotte, V., I. Vigon, E. Boucher de Crevecoeur, P. H. Romeo, V. Lemarchandel, and S. Chretien. 1994. ‘Structure and transcription of the human c-mpl gene (MPL)’, Genomics, 20: 5-12.
Provan, D., R. Stasi, A. C. Newland, V. S. Blanchette, P. Bolton-Maggs, J. B. Bussel, B. H. Chong, D. B. Cines, T. B. Gernsheimer, B. Godeau, J. Grainger, I. Greer, B. J. Hunt, P. A. Imbach, G. Lyons, R. McMillan, F. Rodeghiero, M. A. Sanz, M. Tarantino, S. Watson, J. Young, and D. J. Kuter. 2010. ‘International consensus report on the investigation and management of primary immune thrombocytopenia’, Blood, 115: 168-86.
Seo, A., M. Ben-Harosh, M. Sirin, J. Stein, O. Dgany, J. Kaplelushnik, M. Hoenig, U. Pannicke, M. Lorenz, K. Schwarz, C. Stockklausner, T. Walsh, S. Gulsuner, M. K. Lee, A. Sendamarai, M. Sanchez-Bonilla, M. C. King, H. Cario, A. E. Kulozik, K. M. Debatin, A. Schulz, H. Tamary, and A. Shimamura. 2017. ‘Bone marrow failure unresponsive to bone marrow transplant is caused by mutations in thrombopoietin’, Blood, 130: 875-80.
Solberg, L. A., Jr. 2005. ‘Biologic aspects of thrombopoietins and the development of therapeutic agents’, Curr Hematol Rep, 4: 423-8.

1. A method for treating thrombocytopenia and/or a disease and condition related to insufficient or decreased expression of THPO protein in a subject, comprising administering to the subject a curative dose of a small activating nucleic acid molecule targeting a promoter of THPO, a nucleic acid molecule encoding the small activating nucleic acid molecule, or a composition or formulation comprising the small activating nucleic acid molecule.

2. The method of claim 1, wherein the thrombocytopenia comprises thrombocytopenia caused by various causes, such as congenital (hereditary) or acquired thrombocytopenia, preferably thrombocytopenia caused by myelo-thrombocytopenia, increased peripheral platelet destruction, or splenic sequestration.

3. The method of claim 1, wherein the thrombocytopenia includes, but is not limited to, thrombocytopenia related to insufficient or decreased expression of THPO protein, thrombocytopenia caused by various causes such as immune thrombocytopenia, drug-induced thrombocytopenia, thrombotic thrombocytopenia purpura, or hereditary thrombocytopenia.

4. The method of claim 1, wherein the individual is a mammal.

5. The method of claim 4, wherein the individual is a human.

6. The method of claim 1, wherein the small activating nucleic acid molecule comprises a first nucleic acid strand and a second nucleic acid strand, the first nucleic acid strand having at least 75% (e.g., at least about 79%, about 80%, about 85%, about 90%, about 95%, about 99% or about 100%) homology or complementarity to continuous 16 to 35 nucleotides in length of any one of SEQ ID NO: 601 to SEQ ID NO: 605, and the first nucleic acid strand and the second nucleic acid strand complementarily form a double-stranded nucleic acid structure capable of activating the expression of THPO gene in a cell.

7. The method of claim 6, wherein the first nucleic acid strand of the small activating nucleic acid molecule comprises or is selected from any nucleotide sequence selected from SEQ ID NOs: 1048 to SEQ ID NOs: 1489, and the second nucleic acid strand comprises or is selected from any nucleotide sequence selected from SEQ ID NOs: 1490 to SEQ ID NOs: 1931.

8. The method of claim 6, wherein:
(i) the first nucleic acid strand and the second nucleic acid strand are present on two different nucleic acid strands; or
(ii) the first nucleic acid strand and the second nucleic acid strand are present on the same nucleic acid strand, preferably, the small activating nucleic acid molecule is a hairpin single-stranded nucleic acid molecule, wherein the first nucleic acid strand and the second nucleic acid strand comprise complementary regions forming a double-stranded nucleic acid structure.

9. (canceled)

10. The method of claim 1, wherein at least one strand of the small activating nucleic acid molecule has an overhang of 0 to 6 nucleotides in length at 3′ terminus.

11. The method of claim 10, wherein both strands of the small activating nucleic acid molecule have an overhang of 0 to 6 nucleotides in length at the 3′ terminus, preferably an overhang of 2 or 3 nucleotides in length.

12. The method of claim 6, wherein the first nucleic acid strand and the second nucleic acid strand independently have 16 to 35 nucleotides in length.

13. The method of claim 1, wherein the small activating nucleic acid molecule comprises at least one modification, preferably, the modification is a chemical modification.

14. The method of claim 13, wherein the chemical modification comprises or is selected from one or more modifications selected from the group consisting of:
(1) modification of a phosphodiester bond connecting nucleotides in the nucleotide sequence of the small activating nucleic acid molecule;
(2) modification of 2′-OH of a ribose in the nucleotide sequence of the small activating nucleic acid molecule;
(3) modification of a base in the nucleotide sequence of the small activating nucleic acid molecule; and
(4) at least one nucleotide in the nucleotide sequence of the small activating nucleic acid molecule being a locked nucleic acid.

15. The method of claim 13, wherein the chemical modification comprises one or more modifications selected from the group consisting of: 2′-fluoro modification, 2′-oxymethyl modification, 2′-oxyethylidene methoxy modification, 2,4′-dinitrophenol modification, locked nucleic acid (LNA), 2′-amino modification, 2′-deoxy modification, 5′-bromouracil modification, 5′-iodouracil modification, N-methyluracil modification, 2,6-diaminopurine modification, phosphorothioate modification, and boranophosphate modification.

16. The method of claim 3, wherein the acquired thrombocytopenia includes, but is not limited to, thrombocytopenia caused by aplastic anemia, myelodysplastic syndrome, leukemia, drugs, infection, tumor diseases, radiotherapy, bone marrow transplantation and chronic liver diseases, and immune thrombocytopenia, wherein the factors causing increased peripheral platelet destruction comprise thrombotic thrombocytopenia, heparin-induced thrombocytopenia, drug-induced thrombocytopenia, ITP, and thrombotic thrombocytopenia purpura.

17. A method for activating/up-regulating the expression of THPO gene in a subject or a cell, comprising administering a small activating nucleic acid molecule of the present invention, a nucleic acid coding the small activating nucleic acid molecule of the present invention, or a composition or formulation comprising the small activating nucleic acid molecule of the present invention to the subject or the cell.

18. A nucleic acid coding the small activating nucleic acid molecule of claim 1.

19. A cell comprising the small activating nucleic acid molecule of claim 1 or a nucleic acid encoding the small activating nucleic acid molecule of claim 1.

20. A composition comprising the small activating nucleic acid molecule of claim 1.

21. A formulation comprising the small activating nucleic acid molecule of claim 1, wherein the formulation comprises a liposome, preferably an LNP-entrapped small activating nucleic acid molecule.


SEQUENCE LISTING (1931 sequences)

SEQ ID NO: 1 - DNA, 500 bp, Artificial Sequence
  Mouse THPO Promoter
taatataggcaatacctactttgccagttggaaaggggaaatgccaggaaagacactcctctggagcaggggaatatagaaggaaaagcaagcaagccagcatgctgtcagaattcagcagcaggtatactgttcagggaaaagaaatttggagagccaggaactgagacttcatccatctaaaacaagacctctctttcccagagagaaccgtattctgccagatttctcctggactaaccctcttctctctctcacttaggtcccaggttctcatgtcgccaggacatgagacagttcccgctatacaaattctctctagagtgtgtgtgggtggaggagctgggctctattcatggaccatggtccagccatgggctctagtcccagtagacaggaaaagaagtcgtcatttccgggggagccttcacctgggcttggtggctctctgctctctgattgggcggaagtggcctgggcaggcttgtgaccctactaca

SEQ ID NO: 2 - DNA, 138 bp, Artificial Sequence
  Mouse Hotspot H1
ggcaatacctactttgccagttggaaaggggaaatgccaggaaagacactcctctggagcaggggaatatagaaggaaaagcaagcaagccagcatgctgtcagaattcagcagcaggtatactgttcagggaaaaga

SEQ ID NO: 3 - DNA, 91 bp, Artificial Sequence
  Mouse Hotspot H2
tctcctggactaaccctcttctctctctcacttaggtcccaggttctcatgtcgccaggacatgagacagttcccgctatacaaattctct

SEQ ID NO: 4 - DNA, 85 bp, Artificial Sequence
  Mouse Hotspot H3
gaggagctgggctctattcatggaccatggtccagccatgggctctagtcccagtagacaggaaaagaagtcgtcatttccgggg

SEQ ID NO: 5 - skipped

SEQ ID NO: 6 - DNA, 21 bp, Artificial Sequence
  Synthetic DNA/RNA construct - RAG3-493-saRNA sense sequence
ggcaauaccuacuuugccatt

SEQ ID NO: 7 - DNA, 21 bp, Artificial Sequence
  Synthetic DNA/RNA construct - RAG3-490-saRNA sense sequence
aauaccuacuuugccaguutt

SEQ ID NO: 8 - DNA, 21 bp, Artificial Sequence
  Synthetic DNA/RNA construct - RAG3-486-saRNA sense sequence
ccuacuuugccaguuggaatt

SEQ ID NO: 9 - DNA, 21 bp, Artificial Sequence
  Synthetic DNA/RNA construct - RAG3-484-saRNA sense sequence
uacuuugccaguuggaaagtt

SEQ ID NO: 10 - DNA, 21 bp, Artificial Sequence
  Synthetic DNA/RNA construct - RAG3-483-saRNA sense sequence
acuuugccaguuggaaaggtt

SEQ ID NO: 11 - DNA, 21 bp, Artificial Sequence
  Synthetic DNA/RNA construct - RAG3-479-saRNA sense sequence
ugccaguuggaaaggggaatt

SEQ ID NO: 12 - DNA, 21 bp, Artificial Sequence
  Synthetic DNA/RNA construct - RAG3-474-saRNA sense sequence
guuggaaaggggaaaugcctt

SEQ ID NO: 13 - DNA, 21 bp, Artificial Sequence
  Synthetic DNA/RNA construct - RAG3-472-saRNA sense sequence
uggaaaggggaaaugccagtt

SEQ ID NO: 14 - DNA, 21 bp, Artificial Sequence
  Synthetic DNA/RNA construct - RAG3-471-saRNA sense sequence
ggaaaggggaaaugccaggtt

SEQ ID NO: 15 - DNA, 21 bp, Artificial Sequence
  Synthetic DNA/RNA construct - RAG3-466-saRNA sense sequence
ggggaaaugccaggaaagatt

SEQ ID NO: 16 - DNA, 21 bp, Artificial Sequence
  Synthetic DNA/RNA construct - RAG3-465-saRNA sense sequence
gggaaaugccaggaaagactt

SEQ ID NO: 17 - DNA, 21 bp, Artificial Sequence
  Synthetic DNA/RNA construct - RAG3-461-saRNA sense sequence
aaugccaggaaagacacuctt

SEQ ID NO: 18 - DNA, 21 bp, Artificial Sequence
  Synthetic DNA/RNA construct - RAG3-460-saRNA sense sequence
augccaggaaagacacucctt

SEQ ID NO: 19 - DNA, 21 bp, Artificial Sequence
  Synthetic DNA/RNA construct - RAG3-459-saRNA sense sequence
ugccaggaaagacacuccutt

SEQ ID NO: 20 - DNA, 21 bp, Artificial Sequence
  Synthetic DNA/RNA construct - RAG3-454-saRNA sense sequence
ggaaagacacuccucuggatt

SEQ ID NO: 21 - DNA, 21 bp, Artificial Sequence
  Synthetic DNA/RNA construct - RAG3-453-saRNA sense sequence
gaaagacacuccucuggagtt

SEQ ID NO: 22 - DNA, 21 bp, Artificial Sequence
  Synthetic DNA/RNA construct - RAG3-450-saRNA sense sequence
agacacuccucuggagcagtt

SEQ ID NO: 23 - DNA, 21 bp, Artificial Sequence
  Synthetic DNA/RNA construct - RAG3-449-saRNA sense sequence
gacacuccucuggagcaggtt

SEQ ID NO: 24 - DNA, 21 bp, Artificial Sequence
  Synthetic DNA/RNA construct - RAG3-448-saRNA sense sequence
acacuccucuggagcagggtt

SEQ ID NO: 25 - DNA, 21 bp, Artificial Sequence
  Synthetic DNA/RNA construct - RAG3-446-saRNA sense sequence
acuccucuggagcaggggatt

SEQ ID NO: 26 - DNA, 21 bp, Artificial Sequence
  Synthetic DNA/RNA construct - RAG3-441-saRNA sense sequence
ucuggagcaggggaauauatt

SEQ ID NO: 27 - DNA, 21 bp, Artificial Sequence
  Synthetic DNA/RNA construct - RAG3-440-saRNA sense sequence
cuggagcaggggaauauagtt

SEQ ID NO: 28 - DNA, 21 bp, Artificial Sequence
  Synthetic DNA/RNA construct - RAG3-439-saRNA sense sequence
uggagcaggggaauauagatt

SEQ ID NO: 29 - DNA, 21 bp, Artificial Sequence
  Synthetic DNA/RNA construct - RAG3-437-saRNA sense sequence
gagcaggggaauauagaagtt

SEQ ID NO: 30 - DNA, 21 bp, Artificial Sequence
  Synthetic DNA/RNA construct - RAG3-436-saRNA sense sequence
agcaggggaauauagaaggtt

SEQ ID NO: 31 - DNA, 21 bp, Artificial Sequence
  Synthetic DNA/RNA construct - RAG3-435-saRNA sense sequence
gcaggggaauauagaaggatt

SEQ ID NO: 32 - DNA, 21 bp, Artificial Sequence
  Synthetic DNA/RNA construct - RAG3-434-saRNA sense sequence
caggggaauauagaaggaatt

SEQ ID NO: 33 - DNA, 21 bp, Artificial Sequence
  Synthetic DNA/RNA construct - RAG3-433-saRNA sense sequence
aggggaauauagaaggaaatt

SEQ ID NO: 34 - DNA, 21 bp, Artificial Sequence
  Synthetic DNA/RNA construct - RAG3-432-saRNA sense sequence
ggggaauauagaaggaaaatt

SEQ ID NO: 35 - DNA, 21 bp, Artificial Sequence
  Synthetic DNA/RNA construct - RAG3-430-saRNA sense sequence
ggaauauagaaggaaaagctt

SEQ ID NO: 36 - DNA, 21 bp, Artificial Sequence
  Synthetic DNA/RNA construct - RAG3-426-saRNA sense sequence
uauagaaggaaaagcaagctt

SEQ ID NO: 37 - DNA, 21 bp, Artificial Sequence
  Synthetic DNA/RNA construct - RAG3-425-saRNA sense sequence
auagaaggaaaagcaagcatt

SEQ ID NO: 38 - DNA, 21 bp, Artificial Sequence
  Synthetic DNA/RNA construct - RAG3-424-saRNA sense sequence
uagaaggaaaagcaagcaatt

SEQ ID NO: 39 - DNA, 21 bp, Artificial Sequence
  Synthetic DNA/RNA construct - RAG3-421-saRNA sense sequence
aaggaaaagcaagcaagcctt

SEQ ID NO: 40 - DNA, 21 bp, Artificial Sequence
  Synthetic DNA/RNA construct - RAG3-420-saRNA sense sequence
aggaaaagcaagcaagccatt

SEQ ID NO: 41 - DNA, 21 bp, Artificial Sequence
  Synthetic DNA/RNA construct - RAG3-419-saRNA sense sequence
ggaaaagcaagcaagccagtt

SEQ ID NO: 42 - DNA, 21 bp, Artificial Sequence
  Synthetic DNA/RNA construct - RAG3-418-saRNA sense sequence
gaaaagcaagcaagccagctt

SEQ ID NO: 43 - DNA, 21 bp, Artificial Sequence
  Synthetic DNA/RNA construct - RAG3-417-saRNA sense sequence
aaaagcaagcaagccagcatt

SEQ ID NO: 44 - DNA, 21 bp, Artificial Sequence
  Synthetic DNA/RNA construct - RAG3-414-saRNA sense sequence
agcaagcaagccagcaugctt

SEQ ID NO: 45 - DNA, 21 bp, Artificial Sequence
  Synthetic DNA/RNA construct - RAG3-410-saRNA sense sequence
agcaagccagcaugcuguctt

SEQ ID NO: 46 - DNA, 21 bp, Artificial Sequence
  Synthetic DNA/RNA construct - RAG3-409-saRNA sense sequence
gcaagccagcaugcugucatt

SEQ ID NO: 47 - DNA, 21 bp, Artificial Sequence
  Synthetic DNA/RNA construct - RAG3-408-saRNA sense sequence
caagccagcaugcugucagtt

SEQ ID NO: 48 - DNA, 21 bp, Artificial Sequence
  Synthetic DNA/RNA construct - RAG3-405-saRNA sense sequence
gccagcaugcugucagaautt

SEQ ID NO: 49 - DNA, 21 bp, Artificial Sequence
  Synthetic DNA/RNA construct - RAG3-404-saRNA sense sequence
ccagcaugcugucagaauutt

SEQ ID NO: 50 - DNA, 21 bp, Artificial Sequence
  Synthetic DNA/RNA construct - RAG3-397-saRNA sense sequence
gcugucagaauucagcagctt

SEQ ID NO: 51 - DNA, 21 bp, Artificial Sequence
  Synthetic DNA/RNA construct - RAG3-396-saRNA sense sequence
cugucagaauucagcagcatt

SEQ ID NO: 52 - DNA, 21 bp, Artificial Sequence
  Synthetic DNA/RNA construct - RAG3-378-saRNA sense sequence
agguauacuguucagggaatt

SEQ ID NO: 53 - DNA, 21 bp, Artificial Sequence
  Synthetic DNA/RNA construct - RAG3-377-saRNA sense sequence
gguauacuguucagggaaatt

SEQ ID NO: 54 - DNA, 21 bp, Artificial Sequence
  Synthetic DNA/RNA construct - RAG3-376-saRNA sense sequence
guauacuguucagggaaaatt

SEQ ID NO: 55 - DNA, 21 bp, Artificial Sequence
  Synthetic DNA/RNA construct - RAG3-375-saRNA sense sequence
uauacuguucagggaaaagtt

SEQ ID NO: 56 - DNA, 21 bp, Artificial Sequence
  Synthetic DNA/RNA construct - RAG3-360-saRNA sense sequence
aaagaaauuuggagagccatt

SEQ ID NO: 57 - DNA, 21 bp, Artificial Sequence
  Synthetic DNA/RNA construct - RAG3-337-saRNA sense sequence
cugagacuucauccaucuatt

SEQ ID NO: 58 - DNA, 21 bp, Artificial Sequence
  Synthetic DNA/RNA construct - RAG3-290-saRNA sense sequence
ccguauucugccagauuuctt

SEQ ID NO: 59 - DNA, 21 bp, Artificial Sequence
  Synthetic DNA/RNA construct - RAG3-273-saRNA sense sequence
ucuccuggacuaacccucutt

SEQ ID NO: 60 - DNA, 21 bp, Artificial Sequence
  Synthetic DNA/RNA construct - RAG3-272-saRNA sense sequence
cuccuggacuaacccucuutt

SEQ ID NO: 61 - DNA, 21 bp, Artificial Sequence
  Synthetic DNA/RNA construct - RAG3-271-saRNA sense sequence
uccuggacuaacccucuuctt

SEQ ID NO: 62 - DNA, 21 bp, Artificial Sequence
  Synthetic DNA/RNA construct - RAG3-244-saRNA sense sequence
acuuaggucccagguucuctt

SEQ ID NO: 63 - DNA, 21 bp, Artificial Sequence
  Synthetic DNA/RNA construct - RAG3-243-saRNA sense sequence
cuuaggucccagguucucatt

SEQ ID NO: 64 - DNA, 21 bp, Artificial Sequence
  Synthetic DNA/RNA construct - RAG3-242-saRNA sense sequence
uuaggucccagguucucautt

SEQ ID NO: 65 - DNA, 21 bp, Artificial Sequence
  Synthetic DNA/RNA construct - RAG3-239-saRNA sense sequence
ggucccagguucucauguctt

SEQ ID NO: 66 - DNA, 21 bp, Artificial Sequence
  Synthetic DNA/RNA construct - RAG3-229-saRNA sense sequence
ucucaugucgccaggacautt

SEQ ID NO: 67 - DNA, 21 bp, Artificial Sequence
  Synthetic DNA/RNA construct - RAG3-227-saRNA sense sequence
ucaugucgccaggacaugatt

SEQ ID NO: 68 - DNA, 21 bp, Artificial Sequence
  Synthetic DNA/RNA construct - RAG3-226-saRNA sense sequence
caugucgccaggacaugagtt

SEQ ID NO: 69 - DNA, 21 bp, Artificial Sequence
  Synthetic DNA/RNA construct - RAG3-219-saRNA sense sequence
ccaggacaugagacaguuctt

SEQ ID NO: 70 - DNA, 21 bp, Artificial Sequence
  Synthetic DNA/RNA construct - RAG3-215-saRNA sense sequence
gacaugagacaguucccgctt

SEQ ID NO: 71 - DNA, 21 bp, Artificial Sequence
  Synthetic DNA/RNA construct - RAG3-214-saRNA sense sequence
acaugagacaguucccgcutt

SEQ ID NO: 72 - DNA, 21 bp, Artificial Sequence
  Synthetic DNA/RNA construct - RAG3-206-saRNA sense sequence
caguucccgcuauacaaautt

SEQ ID NO: 73 - DNA, 21 bp, Artificial Sequence
  Synthetic DNA/RNA construct - RAG3-205-saRNA sense sequence
aguucccgcuauacaaauutt

SEQ ID NO: 74 - DNA, 21 bp, Artificial Sequence
  Synthetic DNA/RNA construct - RAG3-204-saRNA sense sequence
guucccgcuauacaaauuctt

SEQ ID NO: 75 - DNA, 21 bp, Artificial Sequence
  Synthetic DNA/RNA construct - RAG3-203-saRNA sense sequence
uucccgcuauacaaauucutt

SEQ ID NO: 76 - DNA, 21 bp, Artificial Sequence
  Synthetic DNA/RNA construct - RAG3-202-saRNA sense sequence
ucccgcuauacaaauucuctt

SEQ ID NO: 77 - DNA, 21 bp, Artificial Sequence
  Synthetic DNA/RNA construct - RAG3-164-saRNA sense sequence
gaggagcugggcucuauuctt

SEQ ID NO: 78 - DNA, 21 bp, Artificial Sequence
  Synthetic DNA/RNA construct - RAG3-158-saRNA sense sequence
cugggcucuauucauggactt

SEQ ID NO: 79 - DNA, 21 bp, Artificial Sequence
  Synthetic DNA/RNA construct - RAG3-153-saRNA sense sequence
cucuauucauggaccauggtt

SEQ ID NO: 80 - DNA, 21 bp, Artificial Sequence
  Synthetic DNA/RNA construct - RAG3-152-saRNA sense sequence
ucuauucauggaccauggutt

SEQ ID NO: 81 - DNA, 21 bp, Artificial Sequence
  Synthetic DNA/RNA construct - RAG3-151-saRNA sense sequence
cuauucauggaccaugguctt

SEQ ID NO: 82 - DNA, 21 bp, Artificial Sequence
  Synthetic DNA/RNA construct - RAG3-150-saRNA sense sequence
uauucauggaccauggucctt

SEQ ID NO: 83 - DNA, 21 bp, Artificial Sequence
  Synthetic DNA/RNA construct - RAG3-147-saRNA sense sequence
ucauggaccaugguccagctt

SEQ ID NO: 84 - DNA, 21 bp, Artificial Sequence
  Synthetic DNA/RNA construct - RAG3-146-saRNA sense sequence
cauggaccaugguccagcctt

SEQ ID NO: 85 - DNA, 21 bp, Artificial Sequence
  Synthetic DNA/RNA construct - RAG3-142-saRNA sense sequence
gaccaugguccagccauggtt

SEQ ID NO: 86 - DNA, 21 bp, Artificial Sequence
  Synthetic DNA/RNA construct - RAG3-134-saRNA sense sequence
uccagccaugggcucuagutt

SEQ ID NO: 87 - DNA, 21 bp, Artificial Sequence
  Synthetic DNA/RNA construct - RAG3-133-saRNA sense sequence
ccagccaugggcucuaguctt

SEQ ID NO: 88 - DNA, 21 bp, Artificial Sequence
  Synthetic DNA/RNA construct - RAG3-129-saRNA sense sequence
ccaugggcucuagucccagtt

SEQ ID NO: 89 - DNA, 21 bp, Artificial Sequence
  Synthetic DNA/RNA construct - RAG3-122-saRNA sense sequence
cucuagucccaguagacagtt

SEQ ID NO: 90 - DNA, 21 bp, Artificial Sequence
  Synthetic DNA/RNA construct - RAG3-121-saRNA sense sequence
ucuagucccaguagacaggtt

SEQ ID NO: 91 - DNA, 21 bp, Artificial Sequence
  Synthetic DNA/RNA construct - RAG3-111-saRNA sense sequence
guagacaggaaaagaaguctt

SEQ ID NO: 92 - DNA, 21 bp, Artificial Sequence
  Synthetic DNA/RNA construct - RAG3-110-saRNA sense sequence
uagacaggaaaagaagucgtt

SEQ ID NO: 93 - DNA, 21 bp, Artificial Sequence
  Synthetic DNA/RNA construct - RAG3-101-saRNA sense sequence
aaagaagucgucauuuccgtt

SEQ ID NO: 94 - DNA, 21 bp, Artificial Sequence
  Synthetic DNA/RNA construct - RAG3-100-saRNA sense sequence
aagaagucgucauuuccggtt

SEQ ID NO: 95 - DNA, 21 bp, Artificial Sequence
  Synthetic DNA/RNA construct - RAG3-099-saRNA sense sequence
agaagucgucauuuccgggtt

SEQ ID NO: 96 - DNA, 21 bp, Artificial Sequence
  Synthetic DNA/RNA construct - RAG3-078-saRNA sense sequence
agccuucaccugggcuuggtt

SEQ ID NO: 97 - DNA, 21 bp, Artificial Sequence
  Synthetic DNA/RNA construct - RAG3-071-saRNA sense sequence
accugggcuugguggcucutt

SEQ ID NO: 98 - DNA, 21 bp, Artificial Sequence
  Synthetic DNA/RNA construct - RAG3-047-saRNA sense sequence
cucugauugggcggaagugtt

SEQ ID NO: 99 - DNA, 21 bp, Artificial Sequence
  Synthetic DNA/RNA construct - RAG3-046-saRNA sense sequence
ucugauugggcggaaguggtt

SEQ ID NO: 100 - DNA, 21 bp, Artificial Sequence
  Synthetic DNA/RNA construct - RAG3-044-saRNA sense sequence
ugauugggcggaaguggcctt

SEQ ID NO: 101 - DNA, 21 bp, Artificial Sequence
  Synthetic DNA/RNA construct - RAG3-023-saRNA sense sequence
ggcaggcuugugacccuactt

SEQ ID NO: 102 - DNA, 21 bp, Artificial Sequence
  Synthetic DNA/RNA construct - RAG3-022-saRNA sense sequence
gcaggcuugugacccuacutt

SEQ ID NO: 103 - DNA, 21 bp, Artificial Sequence
  Synthetic DNA/RNA construct - RAG3-493-saRNA antisense sequence
uggcaaaguagguauugcctt

SEQ ID NO: 104 - DNA, 21 bp, Artificial Sequence
  Synthetic DNA/RNA construct - RAG3-490-saRNA antisense sequence
aacuggcaaaguagguauutt

SEQ ID NO: 105 - DNA, 21 bp, Artificial Sequence
  Synthetic DNA/RNA construct - RAG3-486-saRNA antisense sequence
uuccaacuggcaaaguaggtt

SEQ ID NO: 106 - DNA, 21 bp, Artificial Sequence
  Synthetic DNA/RNA construct - RAG3-484-saRNA antisense sequence
cuuuccaacuggcaaaguatt

SEQ ID NO: 107 - DNA, 21 bp, Artificial Sequence
  Synthetic DNA/RNA construct - RAG3-483-saRNA antisense sequence
ccuuuccaacuggcaaagutt

SEQ ID NO: 108 - DNA, 21 bp, Artificial Sequence
  Synthetic DNA/RNA construct - RAG3-479-saRNA antisense sequence
uuccccuuuccaacuggcatt

SEQ ID NO: 109 - DNA, 21 bp, Artificial Sequence
  Synthetic DNA/RNA construct - RAG3-474-saRNA antisense sequence
ggcauuuccccuuuccaactt

SEQ ID NO: 110 - DNA, 21 bp, Artificial Sequence
  Synthetic DNA/RNA construct - RAG3-472-saRNA antisense sequence
cuggcauuuccccuuuccatt

SEQ ID NO: 111 - DNA, 21 bp, Artificial Sequence
  Synthetic DNA/RNA construct - RAG3-471-saRNA antisense sequence
ccuggcauuuccccuuucctt

SEQ ID NO: 112 - DNA, 21 bp, Artificial Sequence
  Synthetic DNA/RNA construct - RAG3-466-saRNA antisense sequence
ucuuuccuggcauuucccctt

SEQ ID NO: 113 - DNA, 21 bp, Artificial Sequence
  Synthetic DNA/RNA construct - RAG3-465-saRNA antisense sequence
gucuuuccuggcauuuccctt

SEQ ID NO: 114 - DNA, 21 bp, Artificial Sequence
  Synthetic DNA/RNA construct - RAG3-461-saRNA antisense sequence
gagugucuuuccuggcauutt

SEQ ID NO: 115 - DNA, 21 bp, Artificial Sequence
  Synthetic DNA/RNA construct - RAG3-460-saRNA antisense sequence
ggagugucuuuccuggcautt

SEQ ID NO: 116 - DNA, 21 bp, Artificial Sequence
  Synthetic DNA/RNA construct - RAG3-459-saRNA antisense sequence
aggagugucuuuccuggcatt

SEQ ID NO: 117 - DNA, 21 bp, Artificial Sequence
  Synthetic DNA/RNA construct - RAG3-454-saRNA antisense sequence
uccagaggagugucuuucctt

SEQ ID NO: 118 - DNA, 21 bp, Artificial Sequence
  Synthetic DNA/RNA construct - RAG3-453-saRNA antisense sequence
cuccagaggagugucuuuctt

SEQ ID NO: 119 - DNA, 21 bp, Artificial Sequence
  Synthetic DNA/RNA construct - RAG3-450-saRNA antisense sequence
cugcuccagaggagugucutt

SEQ ID NO: 120 - DNA, 21 bp, Artificial Sequence
  Synthetic DNA/RNA construct - RAG3-449-saRNA antisense sequence
ccugcuccagaggaguguctt

SEQ ID NO: 121 - DNA, 21 bp, Artificial Sequence
  Synthetic DNA/RNA construct - RAG3-448-saRNA antisense sequence
cccugcuccagaggagugutt

SEQ ID NO: 122 - DNA, 21 bp, Artificial Sequence
  Synthetic DNA/RNA construct - RAG3-446-saRNA antisense sequence
uccccugcuccagaggagutt

SEQ ID NO: 123 - DNA, 21 bp, Artificial Sequence
  Synthetic DNA/RNA construct - RAG3-441-saRNA antisense sequence
uauauuccccugcuccagatt

SEQ ID NO: 124 - DNA, 21 bp, Artificial Sequence
  Synthetic DNA/RNA construct - RAG3-440-saRNA antisense sequence
cuauauuccccugcuccagtt

SEQ ID NO: 125 - DNA, 21 bp, Artificial Sequence
  Synthetic DNA/RNA construct - RAG3-439-saRNA antisense sequence
ucuauauuccccugcuccatt

SEQ ID NO: 126 - DNA, 21 bp, Artificial Sequence
  Synthetic DNA/RNA construct - RAG3-437-saRNA antisense sequence
cuucuauauuccccugcuctt

SEQ ID NO: 127 - DNA, 21 bp, Artificial Sequence
  Synthetic DNA/RNA construct - RAG3-436-saRNA antisense sequence
ccuucuauauuccccugcutt

SEQ ID NO: 128 - DNA, 21 bp, Artificial Sequence
  Synthetic DNA/RNA construct - RAG3-435-saRNA antisense sequence
uccuucuauauuccccugctt

SEQ ID NO: 129 - DNA, 21 bp, Artificial Sequence
  Synthetic DNA/RNA construct - RAG3-434-saRNA antisense sequence
uuccuucuauauuccccugtt

SEQ ID NO: 130 - DNA, 21 bp, Artificial Sequence
  Synthetic DNA/RNA construct - RAG3-433-saRNA antisense sequence
uuuccuucuauauuccccutt

SEQ ID NO: 131 - DNA, 21 bp, Artificial Sequence
  Synthetic DNA/RNA construct - RAG3-432-saRNA antisense sequence
uuuuccuucuauauucccctt

SEQ ID NO: 132 - DNA, 21 bp, Artificial Sequence
  Synthetic DNA/RNA construct - RAG3-430-saRNA antisense sequence
gcuuuuccuucuauauucctt

SEQ ID NO: 133 - DNA, 21 bp, Artificial Sequence
  Synthetic DNA/RNA construct - RAG3-426-saRNA antisense sequence
gcuugcuuuuccuucuauatt

SEQ ID NO: 134 - DNA, 21 bp, Artificial Sequence
  Synthetic DNA/RNA construct - RAG3-425-saRNA antisense sequence
ugcuugcuuuuccuucuautt

SEQ ID NO: 135 - DNA, 21 bp, Artificial Sequence
  Synthetic DNA/RNA construct - RAG3-424-saRNA antisense sequence
uugcuugcuuuuccuucuatt

SEQ ID NO: 136 - DNA, 21 bp, Artificial Sequence
  Synthetic DNA/RNA construct - RAG3-421-saRNA antisense sequence
ggcuugcuugcuuuuccuutt

SEQ ID NO: 137 - DNA, 21 bp, Artificial Sequence
  Synthetic DNA/RNA construct - RAG3-420-saRNA antisense sequence
uggcuugcuugcuuuuccutt

SEQ ID NO: 138 - DNA, 21 bp, Artificial Sequence
  Synthetic DNA/RNA construct - RAG3-419-saRNA antisense sequence
cuggcuugcuugcuuuucctt

SEQ ID NO: 139 - DNA, 21 bp, Artificial Sequence
  Synthetic DNA/RNA construct - RAG3-418-saRNA antisense sequence
gcuggcuugcuugcuuuuctt

SEQ ID NO: 140 - DNA, 21 bp, Artificial Sequence
  Synthetic DNA/RNA construct - RAG3-417-saRNA antisense sequence
ugcuggcuugcuugcuuuutt

SEQ ID NO: 141 - DNA, 21 bp, Artificial Sequence
  Synthetic DNA/RNA construct - RAG3-414-saRNA antisense sequence
gcaugcuggcuugcuugcutt

SEQ ID NO: 142 - DNA, 21 bp, Artificial Sequence
  Synthetic DNA/RNA construct - RAG3-410-saRNA antisense sequence
gacagcaugcuggcuugcutt

SEQ ID NO: 143 - DNA, 21 bp, Artificial Sequence
  Synthetic DNA/RNA construct - RAG3-409-saRNA antisense sequence
ugacagcaugcuggcuugctt

SEQ ID NO: 144 - DNA, 21 bp, Artificial Sequence
  Synthetic DNA/RNA construct - RAG3-408-saRNA antisense sequence
cugacagcaugcuggcuugtt

SEQ ID NO: 145 - DNA, 21 bp, Artificial Sequence
  Synthetic DNA/RNA construct - RAG3-405-saRNA antisense sequence
auucugacagcaugcuggctt

SEQ ID NO: 146 - DNA, 21 bp, Artificial Sequence
  Synthetic DNA/RNA construct - RAG3-404-saRNA antisense sequence
aauucugacagcaugcuggtt

SEQ ID NO: 147 - DNA, 21 bp, Artificial Sequence
  Synthetic DNA/RNA construct - RAG3-397-saRNA antisense sequence
gcugcugaauucugacagctt

SEQ ID NO: 148 - DNA, 21 bp, Artificial Sequence
  Synthetic DNA/RNA construct - RAG3-396-saRNA antisense sequence
ugcugcugaauucugacagtt

SEQ ID NO: 149 - DNA, 21 bp, Artificial Sequence
  Synthetic DNA/RNA construct - RAG3-378-saRNA antisense sequence
uucccugaacaguauaccutt

SEQ ID NO: 150 - DNA, 21 bp, Artificial Sequence
  Synthetic DNA/RNA construct - RAG3-377-saRNA antisense sequence
uuucccugaacaguauacctt

SEQ ID NO: 151 - DNA, 21 bp, Artificial Sequence
  Synthetic DNA/RNA construct - RAG3-376-saRNA antisense sequence
uuuucccugaacaguauactt

SEQ ID NO: 152 - DNA, 21 bp, Artificial Sequence
  Synthetic DNA/RNA construct - RAG3-375-saRNA antisense sequence
cuuuucccugaacaguauatt

SEQ ID NO: 153 - DNA, 21 bp, Artificial Sequence
  Synthetic DNA/RNA construct - RAG3-360-saRNA antisense sequence
uggcucuccaaauuucuuutt

SEQ ID NO: 154 - DNA, 21 bp, Artificial Sequence
  Synthetic DNA/RNA construct - RAG3-337-saRNA antisense sequence
uagauggaugaagucucagtt

SEQ ID NO: 155 - DNA, 21 bp, Artificial Sequence
  Synthetic DNA/RNA construct - RAG3-290-saRNA antisense sequence
gaaaucuggcagaauacggtt

SEQ ID NO: 156 - DNA, 21 bp, Artificial Sequence
  Synthetic DNA/RNA construct - RAG3-273-saRNA antisense sequence
agaggguuaguccaggagatt

SEQ ID NO: 157 - DNA, 21 bp, Artificial Sequence
  Synthetic DNA/RNA construct - RAG3-272-saRNA antisense sequence
aagaggguuaguccaggagtt

SEQ ID NO: 158 - DNA, 21 bp, Artificial Sequence
  Synthetic DNA/RNA construct - RAG3-271-saRNA antisense sequence
gaagaggguuaguccaggatt

SEQ ID NO: 159 - DNA, 21 bp, Artificial Sequence
  Synthetic DNA/RNA construct - RAG3-244-saRNA antisense sequence
gagaaccugggaccuaagutt

SEQ ID NO: 160 - DNA, 21 bp, Artificial Sequence
  Synthetic DNA/RNA construct - RAG3-243-saRNA antisense sequence
ugagaaccugggaccuaagtt

SEQ ID NO: 161 - DNA, 21 bp, Artificial Sequence
  Synthetic DNA/RNA construct - RAG3-242-saRNA antisense sequence
augagaaccugggaccuaatt

SEQ ID NO: 162 - DNA, 21 bp, Artificial Sequence
  Synthetic DNA/RNA construct - RAG3-239-saRNA antisense sequence
gacaugagaaccugggacctt

SEQ ID NO: 163 - DNA, 21 bp, Artificial Sequence
  Synthetic DNA/RNA construct - RAG3-229-saRNA antisense sequence
auguccuggcgacaugagatt

SEQ ID NO: 164 - DNA, 21 bp, Artificial Sequence
  Synthetic DNA/RNA construct - RAG3-227-saRNA antisense sequence
ucauguccuggcgacaugatt

SEQ ID NO: 165 - DNA, 21 bp, Artificial Sequence
  Synthetic DNA/RNA construct - RAG3-226-saRNA antisense sequence
cucauguccuggcgacaugtt

SEQ ID NO: 166 - DNA, 21 bp, Artificial Sequence
  Synthetic DNA/RNA construct - RAG3-219-saRNA antisense sequence
gaacugucucauguccuggtt

SEQ ID NO: 167 - DNA, 21 bp, Artificial Sequence
  Synthetic DNA/RNA construct - RAG3-215-saRNA antisense sequence
gcgggaacugucucauguctt

SEQ ID NO: 168 - DNA, 21 bp, Artificial Sequence
  Synthetic DNA/RNA construct - RAG3-214-saRNA antisense sequence
agcgggaacugucucaugutt

SEQ ID NO: 169 - DNA, 21 bp, Artificial Sequence
  Synthetic DNA/RNA construct - RAG3-206-saRNA antisense sequence
auuuguauagcgggaacugtt

SEQ ID NO: 170 - DNA, 21 bp, Artificial Sequence
  Synthetic DNA/RNA construct - RAG3-205-saRNA antisense sequence
aauuuguauagcgggaacutt

SEQ ID NO: 171 - DNA, 21 bp, Artificial Sequence
  Synthetic DNA/RNA construct - RAG3-204-saRNA antisense sequence
gaauuuguauagcgggaactt

SEQ ID NO: 172 - DNA, 21 bp, Artificial Sequence
  Synthetic DNA/RNA construct - RAG3-203-saRNA antisense sequence
agaauuuguauagcgggaatt

SEQ ID NO: 173 - DNA, 21 bp, Artificial Sequence
  Synthetic DNA/RNA construct - RAG3-202-saRNA antisense sequence
gagaauuuguauagcgggatt

SEQ ID NO: 174 - DNA, 21 bp, Artificial Sequence
  Synthetic DNA/RNA construct - RAG3-164-saRNA antisense sequence
gaauagagcccagcuccuctt

SEQ ID NO: 175 - DNA, 21 bp, Artificial Sequence
  Synthetic DNA/RNA construct - RAG3-158-saRNA antisense sequence
guccaugaauagagcccagtt

SEQ ID NO: 176 - DNA, 21 bp, Artificial Sequence
  Synthetic DNA/RNA construct - RAG3-153-saRNA antisense sequence
ccaugguccaugaauagagtt

SEQ ID NO: 177 - DNA, 21 bp, Artificial Sequence
  Synthetic DNA/RNA construct - RAG3-152-saRNA antisense sequence
accaugguccaugaauagatt

SEQ ID NO: 178 - DNA, 21 bp, Artificial Sequence
  Synthetic DNA/RNA construct - RAG3-151-saRNA antisense sequence
gaccaugguccaugaauagtt

SEQ ID NO: 179 - DNA, 21 bp, Artificial Sequence
  Synthetic DNA/RNA construct - RAG3-150-saRNA antisense sequence
ggaccaugguccaugaauatt

SEQ ID NO: 180 - DNA, 21 bp, Artificial Sequence
  Synthetic DNA/RNA construct - RAG3-147-saRNA antisense sequence
gcuggaccaugguccaugatt

SEQ ID NO: 181 - DNA, 21 bp, Artificial Sequence
  Synthetic DNA/RNA construct - RAG3-146-saRNA antisense sequence
ggcuggaccaugguccaugtt

SEQ ID NO: 182 - DNA, 21 bp, Artificial Sequence
  Synthetic DNA/RNA construct - RAG3-142-saRNA antisense sequence
ccauggcuggaccaugguctt

SEQ ID NO: 183 - DNA, 21 bp, Artificial Sequence
  Synthetic DNA/RNA construct - RAG3-134-saRNA antisense sequence
acuagagcccauggcuggatt

SEQ ID NO: 184 - DNA, 21 bp, Artificial Sequence
  Synthetic DNA/RNA construct - RAG3-133-saRNA antisense sequence
gacuagagcccauggcuggtt

SEQ ID NO: 185 - DNA, 21 bp, Artificial Sequence
  Synthetic DNA/RNA construct - RAG3-129-saRNA antisense sequence
cugggacuagagcccauggtt

SEQ ID NO: 186 - DNA, 21 bp, Artificial Sequence
  Synthetic DNA/RNA construct - RAG3-122-saRNA antisense sequence
cugucuacugggacuagagtt

SEQ ID NO: 187 - DNA, 21 bp, Artificial Sequence
  Synthetic DNA/RNA construct - RAG3-121-saRNA antisense sequence
ccugucuacugggacuagatt

SEQ ID NO: 188 - DNA, 21 bp, Artificial Sequence
  Synthetic DNA/RNA construct - RAG3-111-saRNA antisense sequence
gacuucuuuuccugucuactt

SEQ ID NO: 189 - DNA, 21 bp, Artificial Sequence
  Synthetic DNA/RNA construct - RAG3-110-saRNA antisense sequence
cgacuucuuuuccugucuatt

SEQ ID NO: 190 - DNA, 21 bp, Artificial Sequence
  Synthetic DNA/RNA construct - RAG3-101-saRNA antisense sequence
cggaaaugacgacuucuuutt

SEQ ID NO: 191 - DNA, 21 bp, Artificial Sequence
  Synthetic DNA/RNA construct - RAG3-100-saRNA antisense sequence
ccggaaaugacgacuucuutt

SEQ ID NO: 192 - DNA, 21 bp, Artificial Sequence
  Synthetic DNA/RNA construct - RAG3-099-saRNA antisense sequence
cccggaaaugacgacuucutt

SEQ ID NO: 193 - DNA, 21 bp, Artificial Sequence
  Synthetic DNA/RNA construct - RAG3-078-saRNA antisense sequence
ccaagcccaggugaaggcutt

SEQ ID NO: 194 - DNA, 21 bp, Artificial Sequence
  Synthetic DNA/RNA construct - RAG3-071-saRNA antisense sequence
agagccaccaagcccaggutt

SEQ ID NO: 195 - DNA, 21 bp, Artificial Sequence
  Synthetic DNA/RNA construct - RAG3-047-saRNA antisense sequence
cacuuccgcccaaucagagtt

SEQ ID NO: 196 - DNA, 21 bp, Artificial Sequence
  Synthetic DNA/RNA construct - RAG3-046-saRNA antisense sequence
ccacuuccgcccaaucagatt

SEQ ID NO: 197 - DNA, 21 bp, Artificial Sequence
  Synthetic DNA/RNA construct - RAG3-044-saRNA antisense sequence
ggccacuuccgcccaaucatt

SEQ ID NO: 198 - DNA, 21 bp, Artificial Sequence
  Synthetic DNA/RNA construct - RAG3-023-saRNA antisense sequence
guagggucacaagccugcctt

SEQ ID NO: 199 - DNA, 21 bp, Artificial Sequence
  Synthetic DNA/RNA construct - RAG3-022-saRNA antisense sequence
aguagggucacaagccugctt

SEQ ID NO: 200 - DNA, 19 bp, Artificial Sequence
  RAG3-493 target sequence
ggcaatacctactttgcca

SEQ ID NO: 201 - DNA, 19 bp, Artificial Sequence
  RAG3-490 target sequence
aatacctactttgccagtt

SEQ ID NO: 202 - DNA, 19 bp, Artificial Sequence
  RAG3-486 target sequence
cctactttgccagttggaa

SEQ ID NO: 203 - DNA, 19 bp, Artificial Sequence
  RAG3-484 target sequence
tactttgccagttggaaag

SEQ ID NO: 204 - DNA, 19 bp, Artificial Sequence
  RAG3-483 target sequence
actttgccagttggaaagg

SEQ ID NO: 205 - DNA, 19 bp, Artificial Sequence
  RAG3-479 target sequence
tgccagttggaaaggggaa

SEQ ID NO: 206 - DNA, 19 bp, Artificial Sequence
  RAG3-474 target sequence
gttggaaaggggaaatgcc

SEQ ID NO: 207 - DNA, 19 bp, Artificial Sequence
  RAG3-472 target sequence
tggaaaggggaaatgccag

SEQ ID NO: 208 - DNA, 19 bp, Artificial Sequence
  RAG3-471 target sequence
ggaaaggggaaatgccagg

SEQ ID NO: 209 - DNA, 19 bp, Artificial Sequence
  RAG3-466 target sequence
ggggaaatgccaggaaaga

SEQ ID NO: 210 - DNA, 19 bp, Artificial Sequence
  RAG3-465 target sequence
gggaaatgccaggaaagac

SEQ ID NO: 211 - DNA, 19 bp, Artificial Sequence
  RAG3-461 target sequence
aatgccaggaaagacactc

SEQ ID NO: 212 - DNA, 19 bp, Artificial Sequence
  RAG3-460 target sequence
atgccaggaaagacactcc

SEQ ID NO: 213 - DNA, 19 bp, Artificial Sequence
  RAG3-459 target sequence
tgccaggaaagacactcct

SEQ ID NO: 214 - DNA, 19 bp, Artificial Sequence
  RAG3-454 target sequence
ggaaagacactcctctgga

SEQ ID NO: 215 - DNA, 19 bp, Artificial Sequence
  RAG3-453 target sequence
gaaagacactcctctggag

SEQ ID NO: 216 - DNA, 19 bp, Artificial Sequence
  RAG3-450 target sequence
agacactcctctggagcag

SEQ ID NO: 217 - DNA, 19 bp, Artificial Sequence
  RAG3-449 target sequence
gacactcctctggagcagg

SEQ ID NO: 218 - DNA, 19 bp, Artificial Sequence
  RAG3-448 target sequence
acactcctctggagcaggg

SEQ ID NO: 219 - DNA, 19 bp, Artificial Sequence
  RAG3-446 target sequence
actcctctggagcagggga

SEQ ID NO: 220 - DNA, 19 bp, Artificial Sequence
  RAG3-441 target sequence
tctggagcaggggaatata

SEQ ID NO: 221 - DNA, 19 bp, Artificial Sequence
  RAG3-440 target sequence
ctggagcaggggaatatag

SEQ ID NO: 222 - DNA, 19 bp, Artificial Sequence
  RAG3-439 target sequence
tggagcaggggaatataga

SEQ ID NO: 223 - DNA, 19 bp, Artificial Sequence
  RAG3-437 target sequence
gagcaggggaatatagaag

SEQ ID NO: 224 - DNA, 19 bp, Artificial Sequence
  RAG3-436 target sequence
agcaggggaatatagaagg

SEQ ID NO: 225 - DNA, 19 bp, Artificial Sequence
  RAG3-435 target sequence
gcaggggaatatagaagga

SEQ ID NO: 226 - DNA, 19 bp, Artificial Sequence
  RAG3-434 target sequence
caggggaatatagaaggaa

SEQ ID NO: 227 - DNA, 19 bp, Artificial Sequence
  RAG3-433 target sequence
aggggaatatagaaggaaa

SEQ ID NO: 228 - DNA, 19 bp, Artificial Sequence
  RAG3-432 target sequence
ggggaatatagaaggaaaa

SEQ ID NO: 229 - DNA, 19 bp, Artificial Sequence
  RAG3-430 target sequence
ggaatatagaaggaaaagc

SEQ ID NO: 230 - DNA, 19 bp, Artificial Sequence
  RAG3-426 target sequence
tatagaaggaaaagcaagc

SEQ ID NO: 231 - DNA, 19 bp, Artificial Sequence
  RAG3-425 target sequence
atagaaggaaaagcaagca

SEQ ID NO: 232 - DNA, 19 bp, Artificial Sequence
  RAG3-424 target sequence
tagaaggaaaagcaagcaa

SEQ ID NO: 233 - DNA, 19 bp, Artificial Sequence
  RAG3-421 target sequence
aaggaaaagcaagcaagcc

SEQ ID NO: 234 - DNA, 19 bp, Artificial Sequence
  RAG3-420 target sequence
aggaaaagcaagcaagcca

SEQ ID NO: 235 - DNA, 19 bp, Artificial Sequence
  RAG3-419 target sequence
ggaaaagcaagcaagccag

SEQ ID NO: 236 - DNA, 19 bp, Artificial Sequence
  RAG3-418 target sequence
gaaaagcaagcaagccagc

SEQ ID NO: 237 - DNA, 19 bp, Artificial Sequence
  RAG3-417 target sequence
aaaagcaagcaagccagca

SEQ ID NO: 238 - DNA, 19 bp, Artificial Sequence
  RAG3-414 target sequence
agcaagcaagccagcatgc

SEQ ID NO: 239 - DNA, 19 bp, Artificial Sequence
  RAG3-410 target sequence
agcaagccagcatgctgtc

SEQ ID NO: 240 - DNA, 19 bp, Artificial Sequence
  RAG3-409 target sequence
gcaagccagcatgctgtca

SEQ ID NO: 241 - DNA, 19 bp, Artificial Sequence
  RAG3-408 target sequence
caagccagcatgctgtcag

SEQ ID NO: 242 - DNA, 19 bp, Artificial Sequence
  RAG3-405 target sequence
gccagcatgctgtcagaat

SEQ ID NO: 243 - DNA, 19 bp, Artificial Sequence
  RAG3-404 target sequence
ccagcatgctgtcagaatt

SEQ ID NO: 244 - DNA, 19 bp, Artificial Sequence
  RAG3-397 target sequence
gctgtcagaattcagcagc

SEQ ID NO: 245 - DNA, 19 bp, Artificial Sequence
  RAG3-396 target sequence
ctgtcagaattcagcagca

SEQ ID NO: 246 - DNA, 19 bp, Artificial Sequence
  RAG3-378 target sequence
aggtatactgttcagggaa

SEQ ID NO: 247 - DNA, 19 bp, Artificial Sequence
  RAG3-377 target sequence
ggtatactgttcagggaaa

SEQ ID NO: 248 - DNA, 19 bp, Artificial Sequence
  RAG3-376 target sequence
gtatactgttcagggaaaa

SEQ ID NO: 249 - DNA, 19 bp, Artificial Sequence
  RAG3-375 target sequence
tatactgttcagggaaaag

SEQ ID NO: 250 - DNA, 19 bp, Artificial Sequence
  RAG3-360 target sequence
aaagaaatttggagagcca

SEQ ID NO: 251 - DNA, 19 bp, Artificial Sequence
  RAG3-337 target sequence
ctgagacttcatccatcta

SEQ ID NO: 252 - DNA, 19 bp, Artificial Sequence
  RAG3-290 target sequence
ccgtattctgccagatttc

SEQ ID NO: 253 - DNA, 19 bp, Artificial Sequence
  RAG3-273 target sequence
tctcctggactaaccctct

SEQ ID NO: 254 - DNA, 19 bp, Artificial Sequence
  RAG3-272 target sequence
ctcctggactaaccctctt

SEQ ID NO: 255 - DNA, 19 bp, Artificial Sequence
  RAG3-271 target sequence
tcctggactaaccctcttc

SEQ ID NO: 256 - DNA, 19 bp, Artificial Sequence
  RAG3-244 target sequence
acttaggtcccaggttctc

SEQ ID NO: 257 - DNA, 19 bp, Artificial Sequence
  RAG3-243 target sequence
cttaggtcccaggttctca

SEQ ID NO: 258 - DNA, 19 bp, Artificial Sequence
  RAG3-242 target sequence
ttaggtcccaggttctcat

SEQ ID NO: 259 - DNA, 19 bp, Artificial Sequence
  RAG3-239 target sequence
ggtcccaggttctcatgtc

SEQ ID NO: 260 - DNA, 19 bp, Artificial Sequence
  RAG3-229 target sequence
tctcatgtcgccaggacat

SEQ ID NO: 261 - DNA, 19 bp, Artificial Sequence
  RAG3-227 target sequence
tcatgtcgccaggacatga

SEQ ID NO: 262 - DNA, 19 bp, Artificial Sequence
  RAG3-226 target sequence
catgtcgccaggacatgag

SEQ ID NO: 263 - DNA, 19 bp, Artificial Sequence
  RAG3-219 target sequence
ccaggacatgagacagttc

SEQ ID NO: 264 - DNA, 19 bp, Artificial Sequence
  RAG3-215 target sequence
gacatgagacagttcccgc

SEQ ID NO: 265 - DNA, 19 bp, Artificial Sequence
  RAG3-214 target sequence
acatgagacagttcccgct

SEQ ID NO: 266 - DNA, 19 bp, Artificial Sequence
  RAG3-206 target sequence
cagttcccgctatacaaat

SEQ ID NO: 267 - DNA, 19 bp, Artificial Sequence
  RAG3-205 target sequence
agttcccgctatacaaatt

SEQ ID NO: 268 - DNA, 19 bp, Artificial Sequence
  RAG3-204 target sequence
gttcccgctatacaaattc

SEQ ID NO: 269 - DNA, 19 bp, Artificial Sequence
  RAG3-203 target sequence
ttcccgctatacaaattct

SEQ ID NO: 270 - DNA, 19 bp, Artificial Sequence
  RAG3-202 target sequence
tcccgctatacaaattctc

SEQ ID NO: 271 - DNA, 19 bp, Artificial Sequence
  RAG3-164 target sequence
gaggagctgggctctattc

SEQ ID NO: 272 - DNA, 19 bp, Artificial Sequence
  RAG3-158 target sequence
ctgggctctattcatggac

SEQ ID NO: 273 - DNA, 19 bp, Artificial Sequence
  RAG3-153 target sequence
ctctattcatggaccatgg

SEQ ID NO: 274 - DNA, 19 bp, Artificial Sequence
  RAG3-152 target sequence
tctattcatggaccatggt

SEQ ID NO: 275 - DNA, 19 bp, Artificial Sequence
  RAG3-151 target sequence
ctattcatggaccatggtc

SEQ ID NO: 276 - DNA, 19 bp, Artificial Sequence
  RAG3-150 target sequence
tattcatggaccatggtcc

SEQ ID NO: 277 - DNA, 19 bp, Artificial Sequence
  RAG3-147 target sequence
tcatggaccatggtccagc

SEQ ID NO: 278 - DNA, 19 bp, Artificial Sequence
  RAG3-146 target sequence
catggaccatggtccagcc

SEQ ID NO: 279 - DNA, 19 bp, Artificial Sequence
  RAG3-142 target sequence
gaccatggtccagccatgg

SEQ ID NO: 280 - DNA, 19 bp, Artificial Sequence
  RAG3-134 target sequence
tccagccatgggctctagt

SEQ ID NO: 281 - DNA, 19 bp, Artificial Sequence
  RAG3-133 target sequence
ccagccatgggctctagtc

SEQ ID NO: 282 - DNA, 19 bp, Artificial Sequence
  RAG3-129 target sequence
ccatgggctctagtcccag

SEQ ID NO: 283 - DNA, 19 bp, Artificial Sequence
  RAG3-122 target sequence
ctctagtcccagtagacag

SEQ ID NO: 284 - DNA, 19 bp, Artificial Sequence
  RAG3-121 target sequence
tctagtcccagtagacagg

SEQ ID NO: 285 - DNA, 19 bp, Artificial Sequence
  RAG3-111 target sequence
gtagacaggaaaagaagtc

SEQ ID NO: 286 - DNA, 19 bp, Artificial Sequence
  RAG3-110 target sequence
tagacaggaaaagaagtcg

SEQ ID NO: 287 - DNA, 19 bp, Artificial Sequence
  RAG3-101 target sequence
aaagaagtcgtcatttccg

SEQ ID NO: 288 - DNA, 19 bp, Artificial Sequence
  RAG3-100 target sequence
aagaagtcgtcatttccgg

SEQ ID NO: 289 - DNA, 19 bp, Artificial Sequence
  RAG3-099 target sequence
agaagtcgtcatttccggg

SEQ ID NO: 290 - DNA, 19 bp, Artificial Sequence
  RAG3-078 target sequence
agccttcacctgggcttgg

SEQ ID NO: 291 - DNA, 19 bp, Artificial Sequence
  RAG3-071 target sequence
acctgggcttggtggctct

SEQ ID NO: 292 - DNA, 19 bp, Artificial Sequence
  RAG3-047 target sequence
ctctgattgggcggaagtg

SEQ ID NO: 293 - DNA, 19 bp, Artificial Sequence
  RAG3-046 target sequence
tctgattgggcggaagtgg

SEQ ID NO: 294 - DNA, 19 bp, Artificial Sequence
  RAG3-044 target sequence
tgattgggcggaagtggcc

SEQ ID NO: 295 - DNA, 19 bp, Artificial Sequence
  RAG3-023 target sequence
ggcaggcttgtgaccctac

SEQ ID NO: 296 - DNA, 19 bp, Artificial Sequence
  RAG3-022 target sequence
gcaggcttgtgaccctact

SEQ ID NO: 297 - DNA, 23 bp, Artificial Sequence
  Thpo F1
tggagctgactgatttgctcctg

SEQ ID NO: 298 - DNA, 22 bp, Artificial Sequence
  Thpo R1
aacagggatagacaaagggtcg

SEQ ID NO: 299 - DNA, 21 bp, Artificial Sequence
  Sdha F
atggtcactagggctggtttg

SEQ ID NO: 300 - DNA, 21 bp, Artificial Sequence
  Sdha R
acgacacccttctgtgatgag

SEQ ID NO: 301 - DNA, 22 bp, Artificial Sequence
  Tbp F
ccgtgaatcttggctgtaaact

SEQ ID NO: 302 - DNA, 20 bp, Artificial Sequence
  Tbp R
tgtccgtggctctcttattc

SEQ ID NO: 303 - DNA, 21 bp, Artificial Sequence
  dsCon2 sense
acuacugagugacaguagatt

SEQ ID NO: 304 - DNA, 21 bp, Artificial Sequence
  dsCon2 antisense
ucuacugucacucaguagutt

SEQ ID NO: 305 - DNA, 21 bp, Artificial Sequence
  DS03-432i sense
ggacuuuagccugggagaatt

SEQ ID NO: 306 - DNA, 21 bp, Artificial Sequence
  DS03-432i antisense
uucucccaggcuaaagucctt

SEQ ID NO: 307-308 - skipped (2 entries)

SEQ ID NO: 309 - DNA, 20 bp, Artificial Sequence
  THPO F
tcgtgactcccatgtccttc

SEQ ID NO: 310 - DNA, 20 bp, Artificial Sequence
  THPO R
tgccttggtctcctccatct

SEQ ID NO: 311 - DNA, 22 bp, Artificial Sequence
  TBP  F
tgctcacccaccaacaatttag

SEQ ID NO: 312 - DNA, 22 bp, Artificial Sequence
  TBP  R
tctgctctgactttagcacctg

SEQ ID NO: 313 - DNA, 21 bp, Artificial Sequence
  HPRT1 F
aaagatggtcaaggtcgcaag

SEQ ID NO: 314 - DNA, 23 bp, Artificial Sequence
  HPRT1 R
tagtcaagggcatatcctacaac

SEQ ID NO: 315-599 - skipped (285 entries)

SEQ ID NO: 600 - DNA, 1382 bp, Artificial Sequence
  Human THPO Promoter
gtccatcagccgatctccccctgcctgggcccacagcgccccccaaaccctcaccaccctctctcactgcctagcctgcctccctaccttctctctgaggtcgctcctcattcttgtgttacccaggacagggacctagccagaaaccggcagcattcccccttctgtggagtgacagtatctccctctcattgtaacttatcctcaggcgcattcgacagtcccctcttgctttctcacccccttccttcacccaagggaccctctgcctctccagcccactcccagcctcctttctcttggttccctggtcatgcctgcctccctgtctcctgtctctccctcccacacacacccactatcctcccagctatcccagcaccctccttcctaatcttgggagacatctcgtctggctggacgggaaaattccaggatctaggccacacttctcagcagacatgcccatccttggggaggaggaacaggagagagcctgaggaagttctgggggacagggggatgatgggatcaaggtcaggccaggaagcccctgaggacagagactgtggggagactgggactgggaagaaagcaaaggagctagagccagggccaaaggaaaaggggggccagcagggaggtatttgcgggggaggtccagcagctgtctttcctaagacagggacacatgggcctggttattcctcttgtcacatgtggaacggtaggagatggaagacggagacagaacaagcaaaggagggccctgggcacagaggtctgtgtgtgtagccatctaagccactggaccccagcagacgagcacctaagctcaggcttaacccagtgcacgtgtgcgcacatacatgtgccccgcacctgacagtccactcaacccgtccaaaccctttccccataacaccaacccataacaggagatttctctcatgtgggcaatatccgtgttcccacttcgaaagggggaatgacaagataggactccctaggggattacagaaagaaaagcaggaaagcaagcatcctgttggatttcagcagcaggtatgatgtccagggaaaagaaatttggatagccagggagtgaaaaccccaccaatcttaaacaagacctctgtgcttcttccccagcaacacaaatgtcctgccagattcctcctggaaaaaaacttctgctcctgtccccctccaggtcccaggttgcccatgtccaggaaaagatggatccccctatccaaatcttctccgtggtgtgtgtgggtggaggagtggaccctggtccaggcaggggctccagggaagagaaggcgtcacttccgggggccttcaccagtgtctggtggctcccttctctgattggg

SEQ ID NO: 601 - DNA, 296 bp, Artificial Sequence
  Human Hotspot H1
caaaccctcaccaccctctctcactgcctagcctgcctccctaccttctctctgaggtcgctcctcattcttgtgttacccaggacagggacctagccagaaaccggcagcattcccccttctgtggagtgacagtatctccctctcattgtaacttatcctcaggcgcattcgacagtcccctcttgctttctcacccccttccttcacccaagggaccctctgcctctccagcccactcccagcctcctttctcttggttccctggtcatgcctgcctccctgtctcctgtctc

SEQ ID NO: 602 - DNA, 125 bp, Artificial Sequence
  Human Hotspot H2
ccactatcctcccagctatcccagcaccctccttcctaatcttgggagacatctcgtctggctggacgggaaaattccaggatctaggccacacttctcagcagacatgcccatccttggggagg

SEQ ID NO: 603 - DNA, 108 bp, Artificial Sequence
  Human Hotspot H3
gatgatgggatcaaggtcaggccaggaagcccctgaggacagagactgtggggagactgggactgggaagaaagcaaaggagctagagccagggccaaaggaaaaggg

SEQ ID NO: 604 - DNA, 118 bp, Artificial Sequence
  Human Hotspot H4
ggaggtccagcagctgtctttcctaagacagggacacatgggcctggttattcctcttgtcacatgtggaacggtaggagatggaagacggagacagaacaagcaaaggagggccctg

SEQ ID NO: 605 - DNA, 593 bp, Artificial Sequence
  Human Hotspot H5
gtgtagccatctaagccactggaccccagcagacgagcacctaagctcaggcttaacccagtgcacgtgtgcgcacatacatgtgccccgcacctgacagtccactcaacccgtccaaaccctttccccataacaccaacccataacaggagatttctctcatgtgggcaatatccgtgttcccacttcgaaagggggaatgacaagataggactccctaggggattacagaaagaaaagcaggaaagcaagcatcctgttggatttcagcagcaggtatgatgtccagggaaaagaaatttggatagccagggagtgaaaaccccaccaatcttaaacaagacctctgtgcttcttccccagcaacacaaatgtcctgccagattcctcctggaaaaaaacttctgctcctgtccccctccaggtcccaggttgcccatgtccaggaaaagatggatccccctatccaaatcttctccgtggtgtgtgtgggtggaggagtggaccctggtccaggcaggggctccagggaagagaaggcgtcacttccgggggccttcaccagtgtctggtggctcccttctctgattggg

SEQ ID NO: 606 - DNA, 19 bp, Artificial Sequence
  RAG3A-889 target sequence
agcctgaggaagttctggg

SEQ ID NO: 607 - DNA, 19 bp, Artificial Sequence
  RAG3A-723 target sequence
tccagcagctgtctttcct

SEQ ID NO: 608 - DNA, 19 bp, Artificial Sequence
  RAG3A-1069 target sequence
atgcctgcctccctgtctc

SEQ ID NO: 609 - DNA, 19 bp, Artificial Sequence
  RAG3A-541 target sequence
ttaacccagtgcacgtgtg

SEQ ID NO: 610 - DNA, 19 bp, Artificial Sequence
  RAG3A-480 target sequence
tccaaaccctttccccata

SEQ ID NO: 611 - DNA, 19 bp, Artificial Sequence
  RAG3A-980 target sequence
gacatctcgtctggctgga

SEQ ID NO: 612 - DNA, 19 bp, Artificial Sequence
  RAG3A-462 target sequence
aacaccaacccataacagg

SEQ ID NO: 613 - DNA, 19 bp, Artificial Sequence
  RAG3A-787 target sequence
caaaggagctagagccagg

SEQ ID NO: 614 - DNA, 19 bp, Artificial Sequence
  RAG3A-232 target sequence
agcaacacaaatgtcctgc

SEQ ID NO: 615 - DNA, 19 bp, Artificial Sequence
  RAG3A-727 target sequence
gaggtccagcagctgtctt

SEQ ID NO: 616 - DNA, 19 bp, Artificial Sequence
  RAG3A-542 target sequence
cttaacccagtgcacgtgt

SEQ ID NO: 617 - DNA, 19 bp, Artificial Sequence
  RAG3A-748 target sequence
agcagggaggtatttgcgg

SEQ ID NO: 618 - DNA, 19 bp, Artificial Sequence
  RAG3A-540 target sequence
taacccagtgcacgtgtgc

SEQ ID NO: 619 - DNA, 19 bp, Artificial Sequence
  RAG3A-1219
tctgtggagtgacagtatc

SEQ ID NO: 620 - DNA, 19 bp, Artificial Sequence
  RAG3A-487 target sequence
caacccgtccaaacccttt

SEQ ID NO: 621 - DNA, 19 bp, Artificial Sequence
  RAG3A-559 target sequence
gagcacctaagctcaggct

SEQ ID NO: 622 - DNA, 19 bp, Artificial Sequence
  RAG3A-1308 target sequence
cctgcctccctaccttctc

SEQ ID NO: 623 - DNA, 19 bp, Artificial Sequence
  RAG3A-24 target sequence
tggtggctcccttctctga

SEQ ID NO: 624 - DNA, 19 bp, Artificial Sequence
  RAG3A-1169 target sequence
ttcgacagtcccctcttgc

SEQ ID NO: 625 - DNA, 19 bp, Artificial Sequence
  RAG3A-1256 target sequence
gacagggacctagccagaa

SEQ ID NO: 626 - DNA, 19 bp, Artificial Sequence
  RAG3A-522 target sequence
cgcacatacatgtgccccg

SEQ ID NO: 627 - DNA, 19 bp, Artificial Sequence
  RAG3A-851
tcaaggtcaggccaggaag

SEQ ID NO: 628 - DNA, 19 bp, Artificial Sequence
  RAG3A-1096 target sequence
agcctcctttctcttggtt

SEQ ID NO: 629 - DNA, 19 bp, Artificial Sequence
  RAG3A-227
cacaaatgtcctgccagat

SEQ ID NO: 630 - DNA, 19 bp, Artificial Sequence
  RAG3A-1178 target sequence
tcaggcgcattcgacagtc

SEQ ID NO: 631 - DNA, 19 bp, Artificial Sequence
  RAG3A-544 target sequence
ggcttaacccagtgcacgt

SEQ ID NO: 632 - DNA, 19 bp, Artificial Sequence
  RAG3A-836 target sequence
gaagcccctgaggacagag

SEQ ID NO: 633 - DNA, 19 bp, Artificial Sequence
  RAG3A-569 target sequence
cccagcagacgagcaccta

SEQ ID NO: 634 - DNA, 19 bp, Artificial Sequence
  RAG3A-1320 target sequence
ctcactgcctagcctgcct

SEQ ID NO: 635 - DNA, 19 bp, Artificial Sequence
  RAG3A-1319 target sequence
tcactgcctagcctgcctc

SEQ ID NO: 636 - DNA, 19 bp, Artificial Sequence
  RAG3A-290 target sequence
gatagccagggagtgaaaa

SEQ ID NO: 637 - DNA, 19 bp, Artificial Sequence
  RAG3A-1074
tggtcatgcctgcctccct

SEQ ID NO: 638 - DNA, 19 bp, Artificial Sequence
  RAG3A-279 target sequence
agtgaaaaccccaccaatc

SEQ ID NO: 639 - DNA, 19 bp, Artificial Sequence
  RAG3A-521 target sequence
gcacatacatgtgccccgc

SEQ ID NO: 640 - DNA, 19 bp, Artificial Sequence
  RAG3A-25 target sequence
ctggtggctcccttctctg

SEQ ID NO: 641 - DNA, 19 bp, Artificial Sequence
  RAG3A-62 target sequence
aagagaaggcgtcacttcc

SEQ ID NO: 642 - DNA, 19 bp, Artificial Sequence
  RAG3A-852 target sequence
atcaaggtcaggccaggaa

SEQ ID NO: 643 - DNA, 19 bp, Artificial Sequence
  RAG3A-931 target sequence
ctcagcagacatgcccatc

SEQ ID NO: 644 - DNA, 19 bp, Artificial Sequence
  RAG3A-892 target sequence
gagagcctgaggaagttct

SEQ ID NO: 645 - DNA, 19 bp, Artificial Sequence
  RAG3A-235 target sequence
cccagcaacacaaatgtcc

SEQ ID NO: 646 - DNA, 19 bp, Artificial Sequence
  RAG3A-1246 target sequence
tagccagaaaccggcagca

SEQ ID NO: 647 - DNA, 19 bp, Artificial Sequence
  RAG3A-19 target sequence
gctcccttctctgattggg

SEQ ID NO: 648 - DNA, 19 bp, Artificial Sequence
  RAG3A-1322 target sequence
ctctcactgcctagcctgc

SEQ ID NO: 649 - DNA, 19 bp, Artificial Sequence
  RAG3A-795 target sequence
gaagaaagcaaaggagcta

SEQ ID NO: 650 - DNA, 19 bp, Artificial Sequence
  RAG3A-515 target sequence
acatgtgccccgcacctga

SEQ ID NO: 651 - DNA, 19 bp, Artificial Sequence
  RAG3A-128 target sequence
tccaaatcttctccgtggt

SEQ ID NO: 652 - DNA, 19 bp, Artificial Sequence
  RAG3A-661 target sequence
ggaacggtaggagatggaa

SEQ ID NO: 653 - DNA, 19 bp, Artificial Sequence
  RAG3A-125 target sequence
aaatcttctccgtggtgtg

SEQ ID NO: 654 - DNA, 19 bp, Artificial Sequence
  RAG3A-1241 target sequence
agaaaccggcagcattccc

SEQ ID NO: 655 - DNA, 19 bp, Artificial Sequence
  RAG3A-463 target sequence
taacaccaacccataacag

SEQ ID NO: 656 - DNA, 19 bp, Artificial Sequence
  RAG3A-434 target sequence
tcatgtgggcaatatccgt

SEQ ID NO: 657 - DNA, 19 bp, Artificial Sequence
  RAG3A-520 target sequence
cacatacatgtgccccgca

SEQ ID NO: 658 - DNA, 19 bp, Artificial Sequence
  RAG3A-499
tgacagtccactcaacccg

SEQ ID NO: 659 - DNA, 19 bp, Artificial Sequence
  RAG3A-668 target sequence
cacatgtggaacggtagga

SEQ ID NO: 660 - DNA, 19 bp, Artificial Sequence
  RAG3A-591 target sequence
gtagccatctaagccactg

SEQ ID NO: 661 - DNA, 19 bp, Artificial Sequence
  RAG3A-1209 target sequence
gacagtatctccctctcat

SEQ ID NO: 662 - DNA, 19 bp, Artificial Sequence
  RAG3A-1257
ggacagggacctagccaga

SEQ ID NO: 663 - DNA, 19 bp, Artificial Sequence
  RAG3A-1170 target sequence
attcgacagtcccctcttg

SEQ ID NO: 664 - DNA, 19 bp, Artificial Sequence
  RAG3A-39 target sequence
gccttcaccagtgtctggt

SEQ ID NO: 665 - DNA, 19 bp, Artificial Sequence
  RAG3A-431 target sequence
tgtgggcaatatccgtgtt

SEQ ID NO: 666 - DNA, 19 bp, Artificial Sequence
  RAG3A-1080 target sequence
gttccctggtcatgcctgc

SEQ ID NO: 667 - DNA, 19 bp, Artificial Sequence
  RAG3A-1266
tgttacccaggacagggac

SEQ ID NO: 668 - DNA, 19 bp, Artificial Sequence
  RAG3A-1202
tctccctctcattgtaact

SEQ ID NO: 669 - DNA, 19 bp, Artificial Sequence
  RAG3A-979
acatctcgtctggctggac

SEQ ID NO: 670 - DNA, 19 bp, Artificial Sequence
  RAG3A-343 target sequence
agcatcctgttggatttca

SEQ ID NO: 671 - DNA, 19 bp, Artificial Sequence
  RAG3A-430 target sequence
gtgggcaatatccgtgttc

SEQ ID NO: 672 - DNA, 19 bp, Artificial Sequence
  RAG3A-319 target sequence
aggtatgatgtccagggaa

SEQ ID NO: 673 - DNA, 19 bp, Artificial Sequence
  RAG3A-612 target sequence
tgggcacagaggtctgtgt

SEQ ID NO: 674 - DNA, 19 bp, Artificial Sequence
  RAG3A-454 target sequence
cccataacaggagatttct

SEQ ID NO: 675 - DNA, 19 bp, Artificial Sequence
  RAG3A-1312 target sequence
ctagcctgcctccctacct

SEQ ID NO: 676 - DNA, 19 bp, Artificial Sequence
  RAG3A-342 target sequence
gcatcctgttggatttcag

SEQ ID NO: 677 - DNA, 19 bp, Artificial Sequence
  RAG3A-1118 target sequence
tctgcctctccagcccact

SEQ ID NO: 678 - DNA, 19 bp, Artificial Sequence
  RAG3A-970 target sequence
ctggctggacgggaaaatt

SEQ ID NO: 679 - DNA, 19 bp, Artificial Sequence
  RAG3A-1133 target sequence
tcacccaagggaccctctg

SEQ ID NO: 680 - DNA, 19 bp, Artificial Sequence
  RAG3A-689 target sequence
gggcctggttattcctctt

SEQ ID NO: 681 - DNA, 19 bp, Artificial Sequence
  RAG3A-1321 target sequence
tctcactgcctagcctgcc

SEQ ID NO: 682 - DNA, 19 bp, Artificial Sequence
  RAG3A-662 target sequence
tggaacggtaggagatgga

SEQ ID NO: 683 - DNA, 19 bp, Artificial Sequence
  RAG3A-817 target sequence
actgtggggagactgggac

SEQ ID NO: 684 - DNA, 19 bp, Artificial Sequence
  RAG3A-1242 target sequence
cagaaaccggcagcattcc

SEQ ID NO: 685 - DNA, 19 bp, Artificial Sequence
  RAG3A-278 target sequence
gtgaaaaccccaccaatct

SEQ ID NO: 686 - DNA, 19 bp, Artificial Sequence
  RAG3A-1210 target sequence
tgacagtatctccctctca

SEQ ID NO: 687 - DNA, 19 bp, Artificial Sequence
  RAG3A-1084 target sequence
cttggttccctggtcatgc

SEQ ID NO: 688 - DNA, 19 bp, Artificial Sequence
  RAG3A-1166 target sequence
gacagtcccctcttgcttt

SEQ ID NO: 689 - DNA, 19 bp, Artificial Sequence
  RAG3A-982 target sequence
gagacatctcgtctggctg

SEQ ID NO: 690 - DNA, 19 bp, Artificial Sequence
  RAG3A-171 target sequence
aggtcccaggttgcccatg

SEQ ID NO: 691 - DNA, 19 bp, Artificial Sequence
  RAG3A-256 target sequence
acaagacctctgtgcttct

SEQ ID NO: 692 - DNA, 19 bp, Artificial Sequence
  RAG3A-563 target sequence
agacgagcacctaagctca

SEQ ID NO: 693 - DNA, 19 bp, Artificial Sequence
  RAG3A-1176 target sequence
aggcgcattcgacagtccc

SEQ ID NO: 694 - DNA, 19 bp, Artificial Sequence
  RAG3A-707
cctaagacagggacacatg

SEQ ID NO: 695 - DNA, 19 bp, Artificial Sequence
  RAG3A-561 target sequence
acgagcacctaagctcagg

SEQ ID NO: 696 - DNA, 19 bp, Artificial Sequence
  RAG3A-504 target sequence
gcacctgacagtccactca

SEQ ID NO: 697 - DNA, 19 bp, Artificial Sequence
  RAG3A-195 target sequence
aaacttctgctcctgtccc

SEQ ID NO: 698 - DNA, 19 bp, Artificial Sequence
  RAG3A-850 target sequence
caaggtcaggccaggaagc

SEQ ID NO: 699 - DNA, 19 bp, Artificial Sequence
  RAG3A-388 target sequence
agataggactccctagggg

SEQ ID NO: 700 - DNA, 19 bp, Artificial Sequence
  RAG3A-1079 target sequence
ttccctggtcatgcctgcc

SEQ ID NO: 701 - DNA, 19 bp, Artificial Sequence
  RAG3A-1263 target sequence
tacccaggacagggaccta

SEQ ID NO: 702 - DNA, 19 bp, Artificial Sequence
  RAG3A-531 target sequence
gcacgtgtgcgcacataca

SEQ ID NO: 703 - DNA, 19 bp, Artificial Sequence
  RAG3A-254 target sequence
aagacctctgtgcttcttc

SEQ ID NO: 704 - DNA, 19 bp, Artificial Sequence
  RAG3A-421 target sequence
atccgtgttcccacttcga

SEQ ID NO: 705 - DNA, 19 bp, Artificial Sequence
  RAG3A-473 target sequence
cctttccccataacaccaa

SEQ ID NO: 706 - DNA, 19 bp, Artificial Sequence
  RAG3A-292 target sequence
tggatagccagggagtgaa

SEQ ID NO: 707 - DNA, 19 bp, Artificial Sequence
  RAG3A-283 target sequence
agggagtgaaaaccccacc

SEQ ID NO: 708 - DNA, 19 bp, Artificial Sequence
  RAG3A-786 target sequence
aaaggagctagagccaggg

SEQ ID NO: 709 - DNA, 19 bp, Artificial Sequence
  RAG3A-582 target sequence
taagccactggaccccagc

SEQ ID NO: 710 - DNA, 19 bp, Artificial Sequence
  RAG3A-933 target sequence
ttctcagcagacatgccca

SEQ ID NO: 711 - DNA, 19 bp, Artificial Sequence
  RAG3A-472 target sequence
ctttccccataacaccaac

SEQ ID NO: 712 - DNA, 19 bp, Artificial Sequence
  RAG3A-646 target sequence
ggaagacggagacagaaca

SEQ ID NO: 713 - DNA, 19 bp, Artificial Sequence
  RAG3A-389 target sequence
aagataggactccctaggg

SEQ ID NO: 714 - DNA, 19 bp, Artificial Sequence
  RAG3A-971 target sequence
tctggctggacgggaaaat

SEQ ID NO: 715 - DNA, 19 bp, Artificial Sequence
  RAG3A-751 target sequence
gccagcagggaggtatttg

SEQ ID NO: 716 - DNA, 19 bp, Artificial Sequence
  RAG3A-299
agaaatttggatagccagg

SEQ ID NO: 717 - DNA, 19 bp, Artificial Sequence
  RAG3A-750 target sequence
ccagcagggaggtatttgc

SEQ ID NO: 718 - DNA, 19 bp, Artificial Sequence
  RAG3A-671 target sequence
tgtcacatgtggaacggta

SEQ ID NO: 719 - DNA, 19 bp, Artificial Sequence
  RAG3A-749 target sequence
cagcagggaggtatttgcg

SEQ ID NO: 720 - DNA, 19 bp, Artificial Sequence
  RAG3A-547
tcaggcttaacccagtgca

SEQ ID NO: 721 - DNA, 19 bp, Artificial Sequence
  RAG3A-655 target sequence
gtaggagatggaagacgga

SEQ ID NO: 722 - DNA, 19 bp, Artificial Sequence
  RAG3A-170 target sequence
ggtcccaggttgcccatgt

SEQ ID NO: 723 - DNA, 19 bp, Artificial Sequence
  RAG3A-629 target sequence
caagcaaaggagggccctg

SEQ ID NO: 724 - DNA, 19 bp, Artificial Sequence
  RAG3A-253 target sequence
agacctctgtgcttcttcc

SEQ ID NO: 725 - DNA, 19 bp, Artificial Sequence
  RAG3A-695 target sequence
acacatgggcctggttatt

SEQ ID NO: 726 - DNA, 19 bp, Artificial Sequence
  RAG3A-788 target sequence
gcaaaggagctagagccag

SEQ ID NO: 727 - DNA, 19 bp, Artificial Sequence
  RAG3A-688 target sequence
ggcctggttattcctcttg

SEQ ID NO: 728 - DNA, 19 bp, Artificial Sequence
  RAG3A-728 target sequence
ggaggtccagcagctgtct

SEQ ID NO: 729 - DNA, 19 bp, Artificial Sequence
  RAG3A-519 target sequence
acatacatgtgccccgcac

SEQ ID NO: 730 - DNA, 19 bp, Artificial Sequence
  RAG3A-272 target sequence
accccaccaatcttaaaca

SEQ ID NO: 731 - DNA, 19 bp, Artificial Sequence
  RAG3A-318 target sequence
ggtatgatgtccagggaaa

SEQ ID NO: 732 - DNA, 19 bp, Artificial Sequence
  RAG3A-1338 target sequence
aaaccctcaccaccctctc

SEQ ID NO: 733 - DNA, 19 bp, Artificial Sequence
  RAG3A-539 target sequence
aacccagtgcacgtgtgcg

SEQ ID NO: 734 - DNA, 19 bp, Artificial Sequence
  RAG3A-826 target sequence
aggacagagactgtgggga

SEQ ID NO: 735 - DNA, 19 bp, Artificial Sequence
  RAG3A-337 target sequence
ctgttggatttcagcagca

SEQ ID NO: 736 - DNA, 19 bp, Artificial Sequence
  RAG3A-273 target sequence
aaccccaccaatcttaaac

SEQ ID NO: 737 - DNA, 19 bp, Artificial Sequence
  RAG3A-478 target sequence
caaaccctttccccataac

SEQ ID NO: 738 - DNA, 19 bp, Artificial Sequence
  RAG3A-963 target sequence
gacgggaaaattccaggat

SEQ ID NO: 739 - DNA, 19 bp, Artificial Sequence
  RAG3A-248 target sequence
tctgtgcttcttccccagc

SEQ ID NO: 740 - DNA, 19 bp, Artificial Sequence
  RAG3A-35 target sequence
tcaccagtgtctggtggct

SEQ ID NO: 741 - DNA, 19 bp, Artificial Sequence
  RAG3A-613 target sequence
ctgggcacagaggtctgtg

SEQ ID NO: 742 - DNA, 19 bp, Artificial Sequence
  RAG3A-1141 target sequence
cccttccttcacccaaggg

SEQ ID NO: 743 - DNA, 19 bp, Artificial Sequence
  RAG3A-477 target sequence
aaaccctttccccataaca

SEQ ID NO: 744 - DNA, 19 bp, Artificial Sequence
  RAG3A-634 target sequence
cagaacaagcaaaggaggg

SEQ ID NO: 745 - DNA, 19 bp, Artificial Sequence
  RAG3A-1280 target sequence
gctcctcattcttgtgtta

SEQ ID NO: 746 - DNA, 19 bp, Artificial Sequence
  RAG3A-1062 target sequence
cctccctgtctcctgtctc

SEQ ID NO: 747 - DNA, 19 bp, Artificial Sequence
  RAG3A-778 target sequence
tagagccagggccaaagga

SEQ ID NO: 748 - DNA, 19 bp, Artificial Sequence
  RAG3A-939 target sequence
ccacacttctcagcagaca

SEQ ID NO: 749 - DNA, 19 bp, Artificial Sequence
  RAG3A-779 target sequence
ctagagccagggccaaagg

SEQ ID NO: 750 - DNA, 19 bp, Artificial Sequence
  RAG3A-803 target sequence
gggactgggaagaaagcaa

SEQ ID NO: 751 - DNA, 19 bp, Artificial Sequence
  RAG3A-1382 target sequence
gtccatcagccgatctccc

SEQ ID NO: 752 - DNA, 19 bp, Artificial Sequence
  RAG3A-97 target sequence
aggagtggaccctggtcca

SEQ ID NO: 753 - DNA, 19 bp, Artificial Sequence
  RAG3A-543 target sequence
gcttaacccagtgcacgtg

SEQ ID NO: 754 - DNA, 19 bp, Artificial Sequence
  RAG3A-556 target sequence
cacctaagctcaggcttaa

SEQ ID NO: 755 - DNA, 19 bp, Artificial Sequence
  RAG3A-60 target sequence
gagaaggcgtcacttccgg

SEQ ID NO: 756 - DNA, 19 bp, Artificial Sequence
  RAG3A-647 target sequence
tggaagacggagacagaac

SEQ ID NO: 757 - DNA, 19 bp, Artificial Sequence
  RAG3A-243 target sequence
gcttcttccccagcaacac

SEQ ID NO: 758 - DNA, 19 bp, Artificial Sequence
  RAG3A-533 target sequence
gtgcacgtgtgcgcacata

SEQ ID NO: 759 - DNA, 19 bp, Artificial Sequence
  RAG3A-628 target sequence
aagcaaaggagggccctgg

SEQ ID NO: 760 - DNA, 19 bp, Artificial Sequence
  RAG3A-849 target sequence
aaggtcaggccaggaagcc

SEQ ID NO: 761 - DNA, 19 bp, Artificial Sequence
  RAG3A-1307 target sequence
ctgcctccctaccttctct

SEQ ID NO: 762 - DNA, 19 bp, Artificial Sequence
  RAG3A-293 target sequence
ttggatagccagggagtga

SEQ ID NO: 763 - DNA, 19 bp, Artificial Sequence
  RAG3A-1285 target sequence
aggtcgctcctcattcttg

SEQ ID NO: 764 - DNA, 19 bp, Artificial Sequence
  RAG3A-1223 target sequence
cccttctgtggagtgacag

SEQ ID NO: 765 - DNA, 19 bp, Artificial Sequence
  RAG3A-1177 target sequence
caggcgcattcgacagtcc

SEQ ID NO: 766 - DNA, 19 bp, Artificial Sequence
  RAG3A-226 target sequence
acaaatgtcctgccagatt

SEQ ID NO: 767 - DNA, 19 bp, Artificial Sequence
  RAG3A-1097 target sequence
cagcctcctttctcttggt

SEQ ID NO: 768 - DNA, 19 bp, Artificial Sequence
  RAG3A-1188 target sequence
taacttatcctcaggcgca

SEQ ID NO: 769 - DNA, 19 bp, Artificial Sequence
  RAG3A-593 target sequence
gtgtagccatctaagccac

SEQ ID NO: 770 - DNA, 19 bp, Artificial Sequence
  RAG3A-670 target sequence
gtcacatgtggaacggtag

SEQ ID NO: 771 - DNA, 19 bp, Artificial Sequence
  RAG3A-503 target sequence
cacctgacagtccactcaa

SEQ ID NO: 772 - DNA, 19 bp, Artificial Sequence
  RAG3A-1167 target sequence
cgacagtcccctcttgctt

SEQ ID NO: 773 - DNA, 19 bp, Artificial Sequence
  RAG3A-583 target sequence
ctaagccactggaccccag

SEQ ID NO: 774 - DNA, 19 bp, Artificial Sequence
  RAG3A-706 target sequence
ctaagacagggacacatgg

SEQ ID NO: 775 - DNA, 19 bp, Artificial Sequence
  RAG3A-983 target sequence
ggagacatctcgtctggct

SEQ ID NO: 776 - DNA, 19 bp, Artificial Sequence
  RAG3A-718 target sequence
cagctgtctttcctaagac

SEQ ID NO: 777 - DNA, 19 bp, Artificial Sequence
  RAG3A-378 target sequence
ccctaggggattacagaaa

SEQ ID NO: 778 - DNA, 19 bp, Artificial Sequence
  RAG3A-1095 target sequence
gcctcctttctcttggttc

SEQ ID NO: 779 - DNA, 19 bp, Artificial Sequence
  RAG3A-698 target sequence
gggacacatgggcctggtt

SEQ ID NO: 780 - DNA, 19 bp, Artificial Sequence
  RAG3A-277 target sequence
tgaaaaccccaccaatctt

SEQ ID NO: 781 - DNA, 19 bp, Artificial Sequence
  RAG3A-644 target sequence
aagacggagacagaacaag

SEQ ID NO: 782 - DNA, 19 bp, Artificial Sequence
  RAG3A-941 target sequence
ggccacacttctcagcaga

SEQ ID NO: 783 - DNA, 19 bp, Artificial Sequence
  RAG3A-794 target sequence
aagaaagcaaaggagctag

SEQ ID NO: 784 - DNA, 19 bp, Artificial Sequence
  RAG3A-890 target sequence
gagcctgaggaagttctgg

SEQ ID NO: 785 - DNA, 19 bp, Artificial Sequence
  RAG3A-1310 target sequence
agcctgcctccctaccttc

SEQ ID NO: 786 - DNA, 19 bp, Artificial Sequence
  RAG3A-27 target sequence
gtctggtggctcccttctc

SEQ ID NO: 787 - DNA, 19 bp, Artificial Sequence
  RAG3A-38 target sequence
ccttcaccagtgtctggtg

SEQ ID NO: 788 - DNA, 19 bp, Artificial Sequence
  RAG3A-532 target sequence
tgcacgtgtgcgcacatac

SEQ ID NO: 789 - DNA, 19 bp, Artificial Sequence
  RAG3A-1023 target sequence
tatcctcccagctatccca

SEQ ID NO: 790 - DNA, 19 bp, Artificial Sequence
  RAG3A-797 target sequence
gggaagaaagcaaaggagc

SEQ ID NO: 791 - DNA, 19 bp, Artificial Sequence
  RAG3A-399 target sequence
ggggaatgacaagatagga

SEQ ID NO: 792 - DNA, 19 bp, Artificial Sequence
  RAG3A-945 target sequence
tctaggccacacttctcag

SEQ ID NO: 793 - DNA, 19 bp, Artificial Sequence
  RAG3A-344 target sequence
aagcatcctgttggatttc

SEQ ID NO: 794 - DNA, 19 bp, Artificial Sequence
  RAG3A-1255 target sequence
acagggacctagccagaaa

SEQ ID NO: 795 - DNA, 19 bp, Artificial Sequence
  RAG3A-524 target sequence
tgcgcacatacatgtgccc

SEQ ID NO: 796 - DNA, 19 bp, Artificial Sequence
  RAG3A-244 target sequence
tgcttcttccccagcaaca

SEQ ID NO: 797 - DNA, 19 bp, Artificial Sequence
  RAG3A-340 target sequence
atcctgttggatttcagca

SEQ ID NO: 798 - DNA, 19 bp, Artificial Sequence
  RAG3A-687 target sequence
gcctggttattcctcttgt

SEQ ID NO: 799 - DNA, 19 bp, Artificial Sequence
  RAG3A-568 target sequence
ccagcagacgagcacctaa

SEQ ID NO: 800 - DNA, 19 bp, Artificial Sequence
  RAG3A-804 target sequence
tgggactgggaagaaagca

SEQ ID NO: 801 - DNA, 19 bp, Artificial Sequence
  RAG3A-496 target sequence
cagtccactcaacccgtcc

SEQ ID NO: 802 - DNA, 19 bp, Artificial Sequence
  RAG3A-810 target sequence
ggagactgggactgggaag

SEQ ID NO: 803 - DNA, 19 bp, Artificial Sequence
  RAG3A-356 target sequence
aagcaggaaagcaagcatc

SEQ ID NO: 804 - DNA, 19 bp, Artificial Sequence
  RAG3A-1248 target sequence
cctagccagaaaccggcag

SEQ ID NO: 805 - DNA, 19 bp, Artificial Sequence
  RAG3A-1015 target sequence
cagctatcccagcaccctc

SEQ ID NO: 806 - DNA, 19 bp, Artificial Sequence
  RAG3A-645 target sequence
gaagacggagacagaacaa

SEQ ID NO: 807 - DNA, 19 bp, Artificial Sequence
  RAG3A-517 target sequence
atacatgtgccccgcacct

SEQ ID NO: 808 - DNA, 19 bp, Artificial Sequence
  RAG3A-194 target sequence
aacttctgctcctgtcccc

SEQ ID NO: 809 - DNA, 19 bp, Artificial Sequence
  RAG3A-927 target sequence
gcagacatgcccatccttg

SEQ ID NO: 810 - DNA, 19 bp, Artificial Sequence
  RAG3A-271 target sequence
ccccaccaatcttaaacaa

SEQ ID NO: 811 - DNA, 19 bp, Artificial Sequence
  RAG3A-398 target sequence
gggaatgacaagataggac

SEQ ID NO: 812 - DNA, 19 bp, Artificial Sequence
  RAG3A-230 target sequence
caacacaaatgtcctgcca

SEQ ID NO: 813 - DNA, 19 bp, Artificial Sequence
  RAG3A-1104 target sequence
ccactcccagcctcctttc

SEQ ID NO: 814 - DNA, 19 bp, Artificial Sequence
  RAG3A-1254 target sequence
cagggacctagccagaaac

SEQ ID NO: 815 - DNA, 19 bp, Artificial Sequence
  RAG3A-288 target sequence
tagccagggagtgaaaacc

SEQ ID NO: 816 - DNA, 19 bp, Artificial Sequence
  RAG3A-837 target sequence
ggaagcccctgaggacaga

SEQ ID NO: 817 - DNA, 19 bp, Artificial Sequence
  RAG3A-935 target sequence
acttctcagcagacatgcc

SEQ ID NO: 818 - DNA, 19 bp, Artificial Sequence
  RAG3A-476 target sequence
aaccctttccccataacac

SEQ ID NO: 819 - DNA, 19 bp, Artificial Sequence
  RAG3A-589 target sequence
agccatctaagccactgga

SEQ ID NO: 820 - DNA, 19 bp, Artificial Sequence
  RAG3A-1207 target sequence
cagtatctccctctcattg

SEQ ID NO: 821 - DNA, 19 bp, Artificial Sequence
  RAG3A-1284 target sequence
ggtcgctcctcattcttgt

SEQ ID NO: 822 - DNA, 19 bp, Artificial Sequence
  RAG3A-956 target sequence
aaattccaggatctaggcc

SEQ ID NO: 823 - DNA, 19 bp, Artificial Sequence
  RAG3A-557 target sequence
gcacctaagctcaggctta

SEQ ID NO: 824 - DNA, 19 bp, Artificial Sequence
  RAG3A-500 target sequence
ctgacagtccactcaaccc

SEQ ID NO: 825 - DNA, 19 bp, Artificial Sequence
  RAG3A-1208 target sequence
acagtatctccctctcatt

SEQ ID NO: 826 - DNA, 19 bp, Artificial Sequence
  RAG3A-686 target sequence
cctggttattcctcttgtc

SEQ ID NO: 827 - DNA, 19 bp, Artificial Sequence
  RAG3A-1168 target sequence
tcgacagtcccctcttgct

SEQ ID NO: 828 - DNA, 19 bp, Artificial Sequence
  RAG3A-218 target sequence
cctgccagattcctcctgg

SEQ ID NO: 829 - DNA, 19 bp, Artificial Sequence
  RAG3A-835 target sequence
aagcccctgaggacagaga

SEQ ID NO: 830 - DNA, 19 bp, Artificial Sequence
  RAG3A-291 target sequence
ggatagccagggagtgaaa

SEQ ID NO: 831 - DNA, 19 bp, Artificial Sequence
  RAG3A-1222 target sequence
ccttctgtggagtgacagt

SEQ ID NO: 832 - DNA, 19 bp, Artificial Sequence
  RAG3A-276 target sequence
gaaaaccccaccaatctta

SEQ ID NO: 833 - DNA, 19 bp, Artificial Sequence
  RAG3A-156 target sequence
catgtccaggaaaagatgg

SEQ ID NO: 834 - DNA, 19 bp, Artificial Sequence
  RAG3A-937 target sequence
acacttctcagcagacatg

SEQ ID NO: 835 - DNA, 19 bp, Artificial Sequence
  RAG3A-1262 target sequence
acccaggacagggacctag

SEQ ID NO: 836 - DNA, 19 bp, Artificial Sequence
  RAG3A-1139 target sequence
cttccttcacccaagggac

SEQ ID NO: 837 - DNA, 19 bp, Artificial Sequence
  RAG3A-535 target sequence
cagtgcacgtgtgcgcaca

SEQ ID NO: 838 - DNA, 19 bp, Artificial Sequence
  RAG3A-719 target sequence
gcagctgtctttcctaaga

SEQ ID NO: 839 - DNA, 19 bp, Artificial Sequence
  RAG3A-726 target sequence
aggtccagcagctgtcttt

SEQ ID NO: 840 - DNA, 19 bp, Artificial Sequence
  RAG3A-384 target sequence
aggactccctaggggatta

SEQ ID NO: 841 - DNA, 19 bp, Artificial Sequence
  RAG3A-834 target sequence
agcccctgaggacagagac

SEQ ID NO: 842 - DNA, 19 bp, Artificial Sequence
  RAG3A-562 target sequence
gacgagcacctaagctcag

SEQ ID NO: 843 - DNA, 19 bp, Artificial Sequence
  RAG3A-940 target sequence
gccacacttctcagcagac

SEQ ID NO: 844 - DNA, 19 bp, Artificial Sequence
  RAG3A-632 target sequence
gaacaagcaaaggagggcc

SEQ ID NO: 845 - DNA, 19 bp, Artificial Sequence
  RAG3A-746 target sequence
cagggaggtatttgcgggg

SEQ ID NO: 846 - DNA, 19 bp, Artificial Sequence
  RAG3A-1309 target sequence
gcctgcctccctaccttct

SEQ ID NO: 847 - DNA, 19 bp, Artificial Sequence
  RAG3A-780 target sequence
gctagagccagggccaaag

SEQ ID NO: 848 - DNA, 19 bp, Artificial Sequence
  RAG3A-693 target sequence
acatgggcctggttattcc

SEQ ID NO: 849 - DNA, 19 bp, Artificial Sequence
  RAG3A-636 target sequence
gacagaacaagcaaaggag

SEQ ID NO: 850 - DNA, 19 bp, Artificial Sequence
  RAG3A-258 target sequence
aaacaagacctctgtgctt

SEQ ID NO: 851 - DNA, 19 bp, Artificial Sequence
  RAG3A-1022 target sequence
atcctcccagctatcccag

SEQ ID NO: 852 - DNA, 19 bp, Artificial Sequence
  RAG3A-381 target sequence
actccctaggggattacag

SEQ ID NO: 853 - DNA, 19 bp, Artificial Sequence
  RAG3A-379 target sequence
tccctaggggattacagaa

SEQ ID NO: 854 - DNA, 19 bp, Artificial Sequence
  RAG3A-663 target sequence
gtggaacggtaggagatgg

SEQ ID NO: 855 - DNA, 19 bp, Artificial Sequence
  RAG3A-1099 target sequence
cccagcctcctttctcttg

SEQ ID NO: 856 - DNA, 19 bp, Artificial Sequence
  RAG3A-855 target sequence
gggatcaaggtcaggccag

SEQ ID NO: 857 - DNA, 19 bp, Artificial Sequence
  RAG3A-1279 target sequence
ctcctcattcttgtgttac

SEQ ID NO: 858 - DNA, 19 bp, Artificial Sequence
  RAG3A-34 target sequence
caccagtgtctggtggctc

SEQ ID NO: 859 - DNA, 19 bp, Artificial Sequence
  RAG3A-633 target sequence
agaacaagcaaaggagggc

SEQ ID NO: 860 - DNA, 19 bp, Artificial Sequence
  RAG3A-453 target sequence
ccataacaggagatttctc

SEQ ID NO: 861 - DNA, 19 bp, Artificial Sequence
  RAG3A-246 target sequence
tgtgcttcttccccagcaa

SEQ ID NO: 862 - DNA, 19 bp, Artificial Sequence
  RAG3A-592 target sequence
tgtagccatctaagccact

SEQ ID NO: 863 - DNA, 19 bp, Artificial Sequence
  RAG3A-270 target sequence
cccaccaatcttaaacaag

SEQ ID NO: 864 - DNA, 19 bp, Artificial Sequence
  RAG3A-630 target sequence
acaagcaaaggagggccct

SEQ ID NO: 865 - DNA, 19 bp, Artificial Sequence
  RAG3A-685 target sequence
ctggttattcctcttgtca

SEQ ID NO: 866 - DNA, 19 bp, Artificial Sequence
  RAG3A-1103 target sequence
cactcccagcctcctttct

SEQ ID NO: 867 - DNA, 19 bp, Artificial Sequence
  RAG3A-1065 target sequence
ctgcctccctgtctcctgt

SEQ ID NO: 868 - DNA, 19 bp, Artificial Sequence
  RAG3A-523 target sequence
gcgcacatacatgtgcccc

SEQ ID NO: 869 - DNA, 19 bp, Artificial Sequence
  RAG3A-590 target sequence
tagccatctaagccactgg

SEQ ID NO: 870 - DNA, 19 bp, Artificial Sequence
  RAG3A-505 target sequence
cgcacctgacagtccactc

SEQ ID NO: 871 - DNA, 19 bp, Artificial Sequence
  RAG3A-1247 target sequence
ctagccagaaaccggcagc

SEQ ID NO: 872 - DNA, 19 bp, Artificial Sequence
  RAG3A-1281 target sequence
cgctcctcattcttgtgtt

SEQ ID NO: 873 - DNA, 19 bp, Artificial Sequence
  RAG3A-747 target sequence
gcagggaggtatttgcggg

SEQ ID NO: 874 - DNA, 19 bp, Artificial Sequence
  RAG3A-1220 target sequence
ttctgtggagtgacagtat

SEQ ID NO: 875 - DNA, 19 bp, Artificial Sequence
  RAG3A-1268 target sequence
tgtgttacccaggacaggg

SEQ ID NO: 876 - DNA, 19 bp, Artificial Sequence
  RAG3A-782 target sequence
gagctagagccagggccaa

SEQ ID NO: 877 - DNA, 19 bp, Artificial Sequence
  RAG3A-107 target sequence
gtgtgggtggaggagtgga

SEQ ID NO: 878 - DNA, 19 bp, Artificial Sequence
  RAG3A-923 target sequence
acatgcccatccttgggga

SEQ ID NO: 879 - DNA, 19 bp, Artificial Sequence
  RAG3A-349 target sequence
aaagcaagcatcctgttgg

SEQ ID NO: 880 - DNA, 19 bp, Artificial Sequence
  RAG3A-1085 target sequence
tcttggttccctggtcatg

SEQ ID NO: 881 - DNA, 19 bp, Artificial Sequence
  RAG3A-432 target sequence
atgtgggcaatatccgtgt

SEQ ID NO: 882 - DNA, 19 bp, Artificial Sequence
  RAG3A-129 target sequence
atccaaatcttctccgtgg

SEQ ID NO: 883 - DNA, 19 bp, Artificial Sequence
  RAG3A-830 target sequence
cctgaggacagagactgtg

SEQ ID NO: 884 - DNA, 19 bp, Artificial Sequence
  RAG3A-74 target sequence
ggggctccagggaagagaa

SEQ ID NO: 885 - DNA, 19 bp, Artificial Sequence
  RAG3A-856 target sequence
tgggatcaaggtcaggcca

SEQ ID NO: 886 - DNA, 19 bp, Artificial Sequence
  RAG3A-435 target sequence
ctcatgtgggcaatatccg

SEQ ID NO: 887 - DNA, 19 bp, Artificial Sequence
  RAG3A-564 target sequence
cagacgagcacctaagctc

SEQ ID NO: 888 - DNA, 19 bp, Artificial Sequence
  RAG3A-1251 target sequence
ggacctagccagaaaccgg

SEQ ID NO: 889 - DNA, 19 bp, Artificial Sequence
  RAG3A-1291 target sequence
tctctgaggtcgctcctca

SEQ ID NO: 890 - DNA, 19 bp, Artificial Sequence
  RAG3A-781 target sequence
agctagagccagggccaaa

SEQ ID NO: 891 - DNA, 19 bp, Artificial Sequence
  RAG3A-261 target sequence
cttaaacaagacctctgtg

SEQ ID NO: 892 - DNA, 19 bp, Artificial Sequence
  RAG3A-1098 target sequence
ccagcctcctttctcttgg

SEQ ID NO: 893 - DNA, 19 bp, Artificial Sequence
  RAG3A-565 target sequence
gcagacgagcacctaagct

SEQ ID NO: 894 - DNA, 19 bp, Artificial Sequence
  RAG3A-930 target sequence
tcagcagacatgcccatcc

SEQ ID NO: 895 - DNA, 19 bp, Artificial Sequence
  RAG3A-422 target sequence
tatccgtgttcccacttcg

SEQ ID NO: 896 - DNA, 19 bp, Artificial Sequence
  RAG3A-516 target sequence
tacatgtgccccgcacctg

SEQ ID NO: 897 - DNA, 19 bp, Artificial Sequence
  RAG3A-1190 target sequence
tgtaacttatcctcaggcg

SEQ ID NO: 898 - DNA, 19 bp, Artificial Sequence
  RAG3A-360 target sequence
agaaaagcaggaaagcaag

SEQ ID NO: 899 - DNA, 19 bp, Artificial Sequence
  RAG3A-126 target sequence
caaatcttctccgtggtgt

SEQ ID NO: 900 - DNA, 19 bp, Artificial Sequence
  RAG3A-938 target sequence
cacacttctcagcagacat

SEQ ID NO: 901 - DNA, 19 bp, Artificial Sequence
  RAG3A-853 target sequence
gatcaaggtcaggccagga

SEQ ID NO: 902 - DNA, 19 bp, Artificial Sequence
  RAG3A-716 target sequence
gctgtctttcctaagacag

SEQ ID NO: 903 - DNA, 19 bp, Artificial Sequence
  RAG3A-1027 target sequence
ccactatcctcccagctat

SEQ ID NO: 904 - DNA, 19 bp, Artificial Sequence
  RAG3A-640 target sequence
cggagacagaacaagcaaa

SEQ ID NO: 905 - DNA, 19 bp, Artificial Sequence
  RAG3A-284 target sequence
cagggagtgaaaaccccac

SEQ ID NO: 906 - DNA, 19 bp, Artificial Sequence
  RAG3A-705 target sequence
taagacagggacacatggg

SEQ ID NO: 907 - DNA, 19 bp, Artificial Sequence
  RAG3A-1315 target sequence
tgcctagcctgcctcccta

SEQ ID NO: 908 - DNA, 19 bp, Artificial Sequence
  RAG3A-1381 target sequence
tccatcagccgatctcccc

SEQ ID NO: 909 - DNA, 19 bp, Artificial Sequence
  RAG3A-558 target sequence
agcacctaagctcaggctt

SEQ ID NO: 910 - DNA, 19 bp, Artificial Sequence
  RAG3A-498 target sequence
gacagtccactcaacccgt

SEQ ID NO: 911 - DNA, 19 bp, Artificial Sequence
  RAG3A-581 target sequence
aagccactggaccccagca

SEQ ID NO: 912 - DNA, 19 bp, Artificial Sequence
  RAG3A-1135 target sequence
cttcacccaagggaccctc

SEQ ID NO: 913 - DNA, 19 bp, Artificial Sequence
  RAG3A-1070 target sequence
catgcctgcctccctgtct

SEQ ID NO: 914 - DNA, 19 bp, Artificial Sequence
  RAG3A-1181 target sequence
tcctcaggcgcattcgaca

SEQ ID NO: 915 - DNA, 19 bp, Artificial Sequence
  RAG3A-1206 target sequence
agtatctccctctcattgt

SEQ ID NO: 916 - DNA, 19 bp, Artificial Sequence
  RAG3A-461 target sequence
acaccaacccataacagga

SEQ ID NO: 917 - DNA, 19 bp, Artificial Sequence
  RAG3A-242 target sequence
cttcttccccagcaacaca

SEQ ID NO: 918 - DNA, 19 bp, Artificial Sequence
  RAG3A-1127 target sequence
aagggaccctctgcctctc

SEQ ID NO: 919 - DNA, 19 bp, Artificial Sequence
  RAG3A-419 target sequence
ccgtgttcccacttcgaaa

SEQ ID NO: 920 - DNA, 19 bp, Artificial Sequence
  RAG3A-790 target sequence
aagcaaaggagctagagcc

SEQ ID NO: 921 - DNA, 19 bp, Artificial Sequence
  RAG3A-635 target sequence
acagaacaagcaaaggagg

SEQ ID NO: 922 - DNA, 19 bp, Artificial Sequence
  RAG3A-673 target sequence
cttgtcacatgtggaacgg

SEQ ID NO: 923 - DNA, 19 bp, Artificial Sequence
  RAG3A-1001 target sequence
ccctccttcctaatcttgg

SEQ ID NO: 924 - DNA, 19 bp, Artificial Sequence
  RAG3A-1005 target sequence
agcaccctccttcctaatc

SEQ ID NO: 925 - DNA, 19 bp, Artificial Sequence
  RAG3A-1064 target sequence
tgcctccctgtctcctgtc

SEQ ID NO: 926 - DNA, 19 bp, Artificial Sequence
  RAG3A-801 target sequence
gactgggaagaaagcaaag

SEQ ID NO: 927 - DNA, 19 bp, Artificial Sequence
  RAG3A-486 target sequence
aacccgtccaaaccctttc

SEQ ID NO: 928 - DNA, 19 bp, Artificial Sequence
  RAG3A-555 target sequence
acctaagctcaggcttaac

SEQ ID NO: 929 - DNA, 19 bp, Artificial Sequence
  RAG3A-854 target sequence
ggatcaaggtcaggccagg

SEQ ID NO: 930 - DNA, 19 bp, Artificial Sequence
  RAG3A-1267 target sequence
gtgttacccaggacaggga

SEQ ID NO: 931 - DNA, 19 bp, Artificial Sequence
  RAG3A-1264 target sequence
ttacccaggacagggacct

SEQ ID NO: 932 - DNA, 19 bp, Artificial Sequence
  RAG3A-513 target sequence
atgtgccccgcacctgaca

SEQ ID NO: 933 - DNA, 19 bp, Artificial Sequence
  RAG3A-1286 target sequence
gaggtcgctcctcattctt

SEQ ID NO: 934 - DNA, 19 bp, Artificial Sequence
  RAG3A-653 target sequence
aggagatggaagacggaga

SEQ ID NO: 935 - DNA, 19 bp, Artificial Sequence
  RAG3A-1014 target sequence
agctatcccagcaccctcc

SEQ ID NO: 936 - DNA, 19 bp, Artificial Sequence
  RAG3A-61 target sequence
agagaaggcgtcacttccg

SEQ ID NO: 937 - DNA, 19 bp, Artificial Sequence
  RAG3A-666 target sequence
catgtggaacggtaggaga

SEQ ID NO: 938 - DNA, 19 bp, Artificial Sequence
  RAG3A-348 target sequence
aagcaagcatcctgttgga

SEQ ID NO: 939 - DNA, 19 bp, Artificial Sequence
  RAG3A-545 target sequence
aggcttaacccagtgcacg

SEQ ID NO: 940 - DNA, 19 bp, Artificial Sequence
  RAG3A-584 target sequence
tctaagccactggacccca

SEQ ID NO: 941 - DNA, 19 bp, Artificial Sequence
  RAG3A-1162 target sequence
gtcccctcttgctttctca

SEQ ID NO: 942 - DNA, 19 bp, Artificial Sequence
  RAG3A-308 target sequence
ccagggaaaagaaatttgg

SEQ ID NO: 943 - DNA, 19 bp, Artificial Sequence
  RAG3A-158 target sequence
cccatgtccaggaaaagat

SEQ ID NO: 944 - DNA, 19 bp, Artificial Sequence
  RAG3A-161 target sequence
ttgcccatgtccaggaaaa

SEQ ID NO: 945 - DNA, 19 bp, Artificial Sequence
  RAG3A-1253 target sequence
agggacctagccagaaacc

SEQ ID NO: 946 - DNA, 19 bp, Artificial Sequence
  RAG3A-926 target sequence
cagacatgcccatccttgg

SEQ ID NO: 947 - DNA, 19 bp, Artificial Sequence
  RAG3A-1191 target sequence
ttgtaacttatcctcaggc

SEQ ID NO: 948 - DNA, 19 bp, Artificial Sequence
  RAG3A-697 target sequence
ggacacatgggcctggtta

SEQ ID NO: 949 - DNA, 19 bp, Artificial Sequence
  RAG3A-1224 target sequence
ccccttctgtggagtgaca

SEQ ID NO: 950 - DNA, 19 bp, Artificial Sequence
  RAG3A-964 target sequence
ggacgggaaaattccagga

SEQ ID NO: 951 - DNA, 19 bp, Artificial Sequence
  RAG3A-791 target sequence
aaagcaaaggagctagagc

SEQ ID NO: 952 - DNA, 19 bp, Artificial Sequence
  RAG3A-526 target sequence
tgtgcgcacatacatgtgc

SEQ ID NO: 953 - DNA, 19 bp, Artificial Sequence
  RAG3A-972 target sequence
gtctggctggacgggaaaa

SEQ ID NO: 954 - DNA, 19 bp, Artificial Sequence
  RAG3A-289 target sequence
atagccagggagtgaaaac

SEQ ID NO: 955 - DNA, 19 bp, Artificial Sequence
  RAG3A-255 target sequence
caagacctctgtgcttctt

SEQ ID NO: 956 - DNA, 19 bp, Artificial Sequence
  RAG3A-1000 target sequence
cctccttcctaatcttggg

SEQ ID NO: 957 - DNA, 19 bp, Artificial Sequence
  RAG3A-694 target sequence
cacatgggcctggttattc

SEQ ID NO: 958 - DNA, 19 bp, Artificial Sequence
  RAG3A-549 target sequence
gctcaggcttaacccagtg

SEQ ID NO: 959 - DNA, 19 bp, Artificial Sequence
  RAG3A-1240 target sequence
gaaaccggcagcattcccc

SEQ ID NO: 960 - DNA, 19 bp, Artificial Sequence
  RAG3A-978 target sequence
catctcgtctggctggacg

SEQ ID NO: 961 - DNA, 19 bp, Artificial Sequence
  RAG3A-1290 target sequence
ctctgaggtcgctcctcat

SEQ ID NO: 962 - DNA, 19 bp, Artificial Sequence
  RAG3A-1006 target sequence
cagcaccctccttcctaat

SEQ ID NO: 963 - DNA, 19 bp, Artificial Sequence
  RAG3A-1311 target sequence
tagcctgcctccctacctt

SEQ ID NO: 964 - DNA, 19 bp, Artificial Sequence
  RAG3A-42 target sequence
ggggccttcaccagtgtct

SEQ ID NO: 965 - DNA, 19 bp, Artificial Sequence
  RAG3A-21 target sequence
tggctcccttctctgattg

SEQ ID NO: 966 - DNA, 19 bp, Artificial Sequence
  RAG3A-59 target sequence
agaaggcgtcacttccggg

SEQ ID NO: 967 - DNA, 19 bp, Artificial Sequence
  RAG3A-1187 target sequence
aacttatcctcaggcgcat

SEQ ID NO: 968 - DNA, 19 bp, Artificial Sequence
  RAG3A-576 target sequence
actggaccccagcagacga

SEQ ID NO: 969 - DNA, 19 bp, Artificial Sequence
  RAG3A-639 target sequence
ggagacagaacaagcaaag

SEQ ID NO: 970 - DNA, 19 bp, Artificial Sequence
  RAG3A-815 target sequence
tgtggggagactgggactg

SEQ ID NO: 971 - DNA, 19 bp, Artificial Sequence
  RAG3A-957 target sequence
aaaattccaggatctaggc

SEQ ID NO: 972 - DNA, 19 bp, Artificial Sequence
  RAG3A-1217 target sequence
tgtggagtgacagtatctc

SEQ ID NO: 973 - DNA, 19 bp, Artificial Sequence
  RAG3A-1165 target sequence
acagtcccctcttgctttc

SEQ ID NO: 974 - DNA, 19 bp, Artificial Sequence
  RAG3A-1134 target sequence
ttcacccaagggaccctct

SEQ ID NO: 975 - DNA, 19 bp, Artificial Sequence
  RAG3A-667 target sequence
acatgtggaacggtaggag

SEQ ID NO: 976 - DNA, 19 bp, Artificial Sequence
  RAG3A-1339 target sequence
caaaccctcaccaccctct

SEQ ID NO: 977 - DNA, 19 bp, Artificial Sequence
  RAG3A-676 target sequence
cctcttgtcacatgtggaa

SEQ ID NO: 978 - DNA, 19 bp, Artificial Sequence
  RAG3A-153 target sequence
gtccaggaaaagatggatc

SEQ ID NO: 979 - DNA, 19 bp, Artificial Sequence
  RAG3A-320 target sequence
caggtatgatgtccaggga

SEQ ID NO: 980 - DNA, 19 bp, Artificial Sequence
  RAG3A-1010 target sequence
atcccagcaccctccttcc

SEQ ID NO: 981 - DNA, 19 bp, Artificial Sequence
  RAG3A-999 target sequence
ctccttcctaatcttggga

SEQ ID NO: 982 - DNA, 19 bp, Artificial Sequence
  RAG3A-567 target sequence
cagcagacgagcacctaag

SEQ ID NO: 983 - DNA, 19 bp, Artificial Sequence
  RAG3A-862 target sequence
ggatgatgggatcaaggtc

SEQ ID NO: 984 - DNA, 19 bp, Artificial Sequence
  RAG3A-460 target sequence
caccaacccataacaggag

SEQ ID NO: 985 - DNA, 19 bp, Artificial Sequence
  RAG3A-921 target sequence
atgcccatccttggggagg

SEQ ID NO: 986 - DNA, 19 bp, Artificial Sequence
  RAG3A-704 target sequence
aagacagggacacatgggc

SEQ ID NO: 987 - DNA, 19 bp, Artificial Sequence
  RAG3A-233 target sequence
cagcaacacaaatgtcctg

SEQ ID NO: 988 - DNA, 19 bp, Artificial Sequence
  RAG3A-1203 target sequence
atctccctctcattgtaac

SEQ ID NO: 989 - DNA, 19 bp, Artificial Sequence
  RAG3A-247 target sequence
ctgtgcttcttccccagca

SEQ ID NO: 990 - DNA, 19 bp, Artificial Sequence
  RAG3A-721 target sequence
cagcagctgtctttcctaa

SEQ ID NO: 991 - DNA, 19 bp, Artificial Sequence
  RAG3A-391 target sequence
acaagataggactccctag

SEQ ID NO: 992 - DNA, 19 bp, Artificial Sequence
  RAG3A-159 target sequence
gcccatgtccaggaaaaga

SEQ ID NO: 993 - DNA, 19 bp, Artificial Sequence
  RAG3A-437 target sequence
ctctcatgtgggcaatatc

SEQ ID NO: 994 - DNA, 19 bp, Artificial Sequence
  RAG3A-29 target sequence
gtgtctggtggctcccttc

SEQ ID NO: 995 - DNA, 19 bp, Artificial Sequence
  RAG3A-63 target sequence
gaagagaaggcgtcacttc

SEQ ID NO: 996 - DNA, 19 bp, Artificial Sequence
  RAG3A-996 target sequence
cttcctaatcttgggagac

SEQ ID NO: 997 - DNA, 19 bp, Artificial Sequence
  RAG3A-1083 target sequence
ttggttccctggtcatgcc

SEQ ID NO: 998 - DNA, 19 bp, Artificial Sequence
  RAG3A-1128 target sequence
caagggaccctctgcctct

SEQ ID NO: 999 - DNA, 19 bp, Artificial Sequence
  RAG3A-1094 target sequence
cctcctttctcttggttcc

SEQ ID NO: 1000 - DNA, 19 bp, Artificial Sequence
  RAG3A-771 target sequence
agggccaaaggaaaagggg

SEQ ID NO: 1001 - DNA, 19 bp, Artificial Sequence
  RAG3A-287 target sequence
agccagggagtgaaaaccc

SEQ ID NO: 1002 - DNA, 19 bp, Artificial Sequence
  RAG3A-981 target sequence
agacatctcgtctggctgg

SEQ ID NO: 1003 - DNA, 19 bp, Artificial Sequence
  RAG3A-23 target sequence
ggtggctcccttctctgat

SEQ ID NO: 1004 - DNA, 19 bp, Artificial Sequence
  RAG3A-1072 target sequence
gtcatgcctgcctccctgt

SEQ ID NO: 1005 - DNA, 19 bp, Artificial Sequence
  RAG3A-415 target sequence
gttcccacttcgaaagggg

SEQ ID NO: 1006 - DNA, 19 bp, Artificial Sequence
  RAG3A-1287 target sequence
tgaggtcgctcctcattct

SEQ ID NO: 1007 - DNA, 19 bp, Artificial Sequence
  RAG3A-1201 target sequence
ctccctctcattgtaactt

SEQ ID NO: 1008 - DNA, 19 bp, Artificial Sequence
  RAG3A-772 target sequence
cagggccaaaggaaaaggg

SEQ ID NO: 1009 - DNA, 19 bp, Artificial Sequence
  RAG3A-1249 target sequence
acctagccagaaaccggca

SEQ ID NO: 1010 - DNA, 19 bp, Artificial Sequence
  RAG3A-546 target sequence
caggcttaacccagtgcac

SEQ ID NO: 1011 - DNA, 19 bp, Artificial Sequence
  RAG3A-160 target sequence
tgcccatgtccaggaaaag

SEQ ID NO: 1012 - DNA, 19 bp, Artificial Sequence
  RAG3A-64 target sequence
ggaagagaaggcgtcactt

SEQ ID NO: 1013 - DNA, 19 bp, Artificial Sequence
  RAG3A-1002 target sequence
accctccttcctaatcttg

SEQ ID NO: 1014 - DNA, 19 bp, Artificial Sequence
  RAG3A-1288 target sequence
ctgaggtcgctcctcattc

SEQ ID NO: 1015 - DNA, 19 bp, Artificial Sequence
  RAG3A-891 target sequence
agagcctgaggaagttctg

SEQ ID NO: 1016 - DNA, 19 bp, Artificial Sequence
  RAG3A-231 target sequence
gcaacacaaatgtcctgcc

SEQ ID NO: 1017 - DNA, 19 bp, Artificial Sequence
  RAG3A-669 target sequence
tcacatgtggaacggtagg

SEQ ID NO: 1018 - DNA, 19 bp, Artificial Sequence
  RAG3A-1009 target sequence
tcccagcaccctccttcct

SEQ ID NO: 1019 - DNA, 19 bp, Artificial Sequence
  RAG3A-656 target sequence
ggtaggagatggaagacgg

SEQ ID NO: 1020 - DNA, 19 bp, Artificial Sequence
  RAG3A-229 target sequence
aacacaaatgtcctgccag

SEQ ID NO: 1021 - DNA, 19 bp, Artificial Sequence
  RAG3A-484 target sequence
cccgtccaaaccctttccc

SEQ ID NO: 1022 - DNA, 19 bp, Artificial Sequence
  RAG3A-799 target sequence
ctgggaagaaagcaaagga

SEQ ID NO: 1023 - DNA, 19 bp, Artificial Sequence
  RAG3A-296 target sequence
aatttggatagccagggag

SEQ ID NO: 1024 - DNA, 19 bp, Artificial Sequence
  RAG3A-1183 target sequence
tatcctcaggcgcattcga

SEQ ID NO: 1025 - DNA, 19 bp, Artificial Sequence
  RAG3A-380 target sequence
ctccctaggggattacaga

SEQ ID NO: 1026 - DNA, 19 bp, Artificial Sequence
  RAG3A-157 target sequence
ccatgtccaggaaaagatg

SEQ ID NO: 1027 - DNA, 19 bp, Artificial Sequence
  RAG3A-1185 target sequence
cttatcctcaggcgcattc

SEQ ID NO: 1028 - DNA, 19 bp, Artificial Sequence
  RAG3A-1244 target sequence
gccagaaaccggcagcatt

SEQ ID NO: 1029 - DNA, 19 bp, Artificial Sequence
  RAG3A-588 target sequence
gccatctaagccactggac

SEQ ID NO: 1030 - DNA, 19 bp, Artificial Sequence
  RAG3A-436 target sequence
tctcatgtgggcaatatcc

SEQ ID NO: 1031 - DNA, 19 bp, Artificial Sequence
  RAG3A-1029 target sequence
acccactatcctcccagct

SEQ ID NO: 1032 - DNA, 19 bp, Artificial Sequence
  RAG3A-28 target sequence
tgtctggtggctcccttct

SEQ ID NO: 1033 - DNA, 19 bp, Artificial Sequence
  RAG3A-234 target sequence
ccagcaacacaaatgtcct

SEQ ID NO: 1034 - DNA, 19 bp, Artificial Sequence
  RAG3A-249 target sequence
ctctgtgcttcttccccag

SEQ ID NO: 1035 - DNA, 19 bp, Artificial Sequence
  RAG3A-775 target sequence
agccagggccaaaggaaaa

SEQ ID NO: 1036 - DNA, 19 bp, Artificial Sequence
  RAG3A-951 target sequence
ccaggatctaggccacact

SEQ ID NO: 1037 - DNA, 19 bp, Artificial Sequence
  RAG3A-861 target sequence
gatgatgggatcaaggtca

SEQ ID NO: 1038 - DNA, 19 bp, Artificial Sequence
  RAG3A-259 target sequence
taaacaagacctctgtgct

SEQ ID NO: 1039 - DNA, 19 bp, Artificial Sequence
  RAG3A-394 target sequence
atgacaagataggactccc

SEQ ID NO: 1040 - DNA, 19 bp, Artificial Sequence
  RAG3A-534 target sequence
agtgcacgtgtgcgcacat

SEQ ID NO: 1041 - DNA, 19 bp, Artificial Sequence
  RAG3A-316 target sequence
tatgatgtccagggaaaag

SEQ ID NO: 1042 - DNA, 19 bp, Artificial Sequence
  RAG3A-560 target sequence
cgagcacctaagctcaggc

SEQ ID NO: 1043 - DNA, 19 bp, Artificial Sequence
  RAG3A-1105 target sequence
cccactcccagcctccttt

SEQ ID NO: 1044 - DNA, 19 bp, Artificial Sequence
  RAG3A-709 target sequence
ttcctaagacagggacaca

SEQ ID NO: 1045 - DNA, 19 bp, Artificial Sequence
  RAG3A-948 target sequence
ggatctaggccacacttct

SEQ ID NO: 1046 - DNA, 19 bp, Artificial Sequence
  RAG3A-998 target sequence
tccttcctaatcttgggag

SEQ ID NO: 1047 - DNA, 19 bp, Artificial Sequence
  RAG3A-387 target sequence
gataggactccctagggga

SEQ ID NO: 1048 - DNA, 21 bp, Artificial Sequence
  Synthetic DNA/RNA construct - RAG3A-889-saRNA sense sequence
agccugaggaaguucugggtt

SEQ ID NO: 1049 - DNA, 21 bp, Artificial Sequence
  Synthetic DNA/RNA construct - RAG3A-723-saRNA sense sequence
uccagcagcugucuuuccutt

SEQ ID NO: 1050 - DNA, 21 bp, Artificial Sequence
  Synthetic DNA/RNA construct - RAG3A-1069-saRNA sense sequence
augccugccucccugucuctt

SEQ ID NO: 1051 - DNA, 21 bp, Artificial Sequence
  Synthetic DNA/RNA construct - RAG3A-541-saRNA sense sequence
uuaacccagugcacgugugtt

SEQ ID NO: 1052 - DNA, 21 bp, Artificial Sequence
  Synthetic DNA/RNA construct - RAG3A-480-saRNA sense sequence
uccaaacccuuuccccauatt

SEQ ID NO: 1053 - DNA, 21 bp, Artificial Sequence
  Synthetic DNA/RNA construct - RAG3A-980-saRNA sense sequence
gacaucucgucuggcuggatt

SEQ ID NO: 1054 - DNA, 21 bp, Artificial Sequence
  Synthetic DNA/RNA construct - RAG3A-462-saRNA sense sequence
aacaccaacccauaacaggtt

SEQ ID NO: 1055 - DNA, 21 bp, Artificial Sequence
  Synthetic DNA/RNA construct - RAG3A-787-saRNA sense sequence
caaaggagcuagagccaggtt

SEQ ID NO: 1056 - DNA, 21 bp, Artificial Sequence
  Synthetic DNA/RNA construct - RAG3A-232-saRNA sense sequence
agcaacacaaauguccugctt

SEQ ID NO: 1057 - DNA, 21 bp, Artificial Sequence
  Synthetic DNA/RNA construct - RAG3A-727-saRNA sense sequence
gagguccagcagcugucuutt

SEQ ID NO: 1058 - DNA, 21 bp, Artificial Sequence
  Synthetic DNA/RNA construct - RAG3A-542-saRNA sense sequence
cuuaacccagugcacgugutt

SEQ ID NO: 1059 - DNA, 21 bp, Artificial Sequence
  Synthetic DNA/RNA construct - RAG3A-748-saRNA sense sequence
agcagggagguauuugcggtt

SEQ ID NO: 1060 - DNA, 21 bp, Artificial Sequence
  Synthetic DNA/RNA construct - RAG3A-540-saRNA sense sequence
uaacccagugcacgugugctt

SEQ ID NO: 1061 - DNA, 21 bp, Artificial Sequence
  Synthetic DNA/RNA construct - RAG3A-1219-saRNA sense sequence
ucuguggagugacaguauctt

SEQ ID NO: 1062 - DNA, 21 bp, Artificial Sequence
  Synthetic DNA/RNA construct - RAG3A-487-saRNA sense sequence
caacccguccaaacccuuutt

SEQ ID NO: 1063 - DNA, 21 bp, Artificial Sequence
  Synthetic DNA/RNA construct - RAG3A-559-saRNA sense sequence
gagcaccuaagcucaggcutt

SEQ ID NO: 1064 - DNA, 21 bp, Artificial Sequence
  Synthetic DNA/RNA construct - RAG3A-1308-saRNA sense sequence
ccugccucccuaccuucuctt

SEQ ID NO: 1065 - DNA, 21 bp, Artificial Sequence
  Synthetic DNA/RNA construct - RAG3A-24-saRNA sense sequence
ugguggcucccuucucugatt

SEQ ID NO: 1066 - DNA, 21 bp, Artificial Sequence
  Synthetic DNA/RNA construct - RAG3A-1169-saRNA sense sequence
uucgacaguccccucuugctt

SEQ ID NO: 1067 - DNA, 21 bp, Artificial Sequence
  Synthetic DNA/RNA construct - RAG3A-1256-saRNA sense sequence
gacagggaccuagccagaatt

SEQ ID NO: 1068 - DNA, 21 bp, Artificial Sequence
  Synthetic DNA/RNA construct - RAG3A-522-saRNA sense sequence
cgcacauacaugugccccgtt

SEQ ID NO: 1069 - DNA, 21 bp, Artificial Sequence
  Synthetic DNA/RNA construct - RAG3A-851-saRNA sense sequence
ucaaggucaggccaggaagtt

SEQ ID NO: 1070 - DNA, 21 bp, Artificial Sequence
  Synthetic DNA/RNA construct - RAG3A-1096-saRNA sense sequence
agccuccuuucucuugguutt

SEQ ID NO: 1071 - DNA, 21 bp, Artificial Sequence
  Synthetic DNA/RNA construct - RAG3A-227-saRNA sense sequence
cacaaauguccugccagautt

SEQ ID NO: 1072 - DNA, 21 bp, Artificial Sequence
  Synthetic DNA/RNA construct - RAG3A-1178-saRNA sense sequence
ucaggcgcauucgacaguctt

SEQ ID NO: 1073 - DNA, 21 bp, Artificial Sequence
  Synthetic DNA/RNA construct - RAG3A-544-saRNA sense sequence
ggcuuaacccagugcacgutt

SEQ ID NO: 1074 - DNA, 21 bp, Artificial Sequence
  Synthetic DNA/RNA construct - RAG3A-836-saRNA sense sequence
gaagccccugaggacagagtt

SEQ ID NO: 1075 - DNA, 21 bp, Artificial Sequence
  Synthetic DNA/RNA construct - RAG3A-569-saRNA sense sequence
cccagcagacgagcaccuatt

SEQ ID NO: 1076 - DNA, 21 bp, Artificial Sequence
  Synthetic DNA/RNA construct - RAG3A-1320-saRNA sense sequence
cucacugccuagccugccutt

SEQ ID NO: 1077 - DNA, 21 bp, Artificial Sequence
  Synthetic DNA/RNA construct - RAG3A-1319-saRNA sense sequence
ucacugccuagccugccuctt

SEQ ID NO: 1078 - DNA, 21 bp, Artificial Sequence
  Synthetic DNA/RNA construct - RAG3A-290-saRNA sense sequence
gauagccagggagugaaaatt

SEQ ID NO: 1079 - DNA, 21 bp, Artificial Sequence
  Synthetic DNA/RNA construct - RAG3A-1074-saRNA sense sequence
uggucaugccugccucccutt

SEQ ID NO: 1080 - DNA, 21 bp, Artificial Sequence
  Synthetic DNA/RNA construct - RAG3A-279-saRNA sense sequence
agugaaaaccccaccaauctt

SEQ ID NO: 1081 - DNA, 21 bp, Artificial Sequence
  Synthetic DNA/RNA construct - RAG3A-521-saRNA sense sequence
gcacauacaugugccccgctt

SEQ ID NO: 1082 - DNA, 21 bp, Artificial Sequence
  Synthetic DNA/RNA construct - RAG3A-25-saRNA sense sequence
cugguggcucccuucucugtt

SEQ ID NO: 1083 - DNA, 21 bp, Artificial Sequence
  Synthetic DNA/RNA construct - RAG3A-62-saRNA sense sequence
aagagaaggcgucacuucctt

SEQ ID NO: 1084 - DNA, 21 bp, Artificial Sequence
  Synthetic DNA/RNA construct - RAG3A-852-saRNA sense sequence
aucaaggucaggccaggaatt

SEQ ID NO: 1085 - DNA, 21 bp, Artificial Sequence
  Synthetic DNA/RNA construct - RAG3A-931-saRNA sense sequence
cucagcagacaugcccauctt

SEQ ID NO: 1086 - DNA, 21 bp, Artificial Sequence
  Synthetic DNA/RNA construct - RAG3A-892-saRNA sense sequence
gagagccugaggaaguucutt

SEQ ID NO: 1087 - DNA, 21 bp, Artificial Sequence
  Synthetic DNA/RNA construct - RAG3A-235-saRNA sense sequence
cccagcaacacaaaugucctt

SEQ ID NO: 1088 - DNA, 21 bp, Artificial Sequence
  Synthetic DNA/RNA construct - RAG3A-1246-saRNA sense sequence
uagccagaaaccggcagcatt

SEQ ID NO: 1089 - DNA, 21 bp, Artificial Sequence
  Synthetic DNA/RNA construct - RAG3A-19-saRNA sense sequence
gcucccuucucugauugggtt

SEQ ID NO: 1090 - DNA, 21 bp, Artificial Sequence
  Synthetic DNA/RNA construct - RAG3A-1322-saRNA sense sequence
cucucacugccuagccugctt

SEQ ID NO: 1091 - DNA, 21 bp, Artificial Sequence
  Synthetic DNA/RNA construct - RAG3A-795-saRNA sense sequence
gaagaaagcaaaggagcuatt

SEQ ID NO: 1092 - DNA, 21 bp, Artificial Sequence
  Synthetic DNA/RNA construct - RAG3A-515-saRNA sense sequence
acaugugccccgcaccugatt

SEQ ID NO: 1093 - DNA, 21 bp, Artificial Sequence
  Synthetic DNA/RNA construct - RAG3A-128-saRNA sense sequence
uccaaaucuucuccguggutt

SEQ ID NO: 1094 - DNA, 21 bp, Artificial Sequence
  Synthetic DNA/RNA construct - RAG3A-661-saRNA sense sequence
ggaacgguaggagauggaatt

SEQ ID NO: 1095 - DNA, 21 bp, Artificial Sequence
  Synthetic DNA/RNA construct - RAG3A-125-saRNA sense sequence
aaaucuucuccguggugugtt

SEQ ID NO: 1096 - DNA, 21 bp, Artificial Sequence
  Synthetic DNA/RNA construct - RAG3A-1241-saRNA sense sequence
agaaaccggcagcauuccctt

SEQ ID NO: 1097 - DNA, 21 bp, Artificial Sequence
  Synthetic DNA/RNA construct - RAG3A-463-saRNA sense sequence
uaacaccaacccauaacagtt

SEQ ID NO: 1098 - DNA, 21 bp, Artificial Sequence
  Synthetic DNA/RNA construct - RAG3A-434-saRNA sense sequence
ucaugugggcaauauccgutt

SEQ ID NO: 1099 - DNA, 21 bp, Artificial Sequence
  Synthetic DNA/RNA construct - RAG3A-520-saRNA sense sequence
cacauacaugugccccgcatt

SEQ ID NO: 1100 - DNA, 21 bp, Artificial Sequence
  Synthetic DNA/RNA construct - RAG3A-499-saRNA sense sequence
ugacaguccacucaacccgtt

SEQ ID NO: 1101 - DNA, 21 bp, Artificial Sequence
  Synthetic DNA/RNA construct - RAG3A-668-saRNA sense sequence
cacauguggaacgguaggatt

SEQ ID NO: 1102 - DNA, 21 bp, Artificial Sequence
  Synthetic DNA/RNA construct - RAG3A-591-saRNA sense sequence
guagccaucuaagccacugtt

SEQ ID NO: 1103 - DNA, 21 bp, Artificial Sequence
  Synthetic DNA/RNA construct - RAG3A-1209-saRNA sense sequence
gacaguaucucccucucautt

SEQ ID NO: 1104 - DNA, 21 bp, Artificial Sequence
  Synthetic DNA/RNA construct - RAG3A-1257-saRNA sense sequence
ggacagggaccuagccagatt

SEQ ID NO: 1105 - DNA, 21 bp, Artificial Sequence
  Synthetic DNA/RNA construct - RAG3A-1170-saRNA sense sequence
auucgacaguccccucuugtt

SEQ ID NO: 1106 - DNA, 21 bp, Artificial Sequence
  Synthetic DNA/RNA construct - RAG3A-39-saRNA sense sequence
gccuucaccagugucuggutt

SEQ ID NO: 1107 - DNA, 21 bp, Artificial Sequence
  Synthetic DNA/RNA construct - RAG3A-431-saRNA sense sequence
ugugggcaauauccguguutt

SEQ ID NO: 1108 - DNA, 21 bp, Artificial Sequence
  Synthetic DNA/RNA construct - RAG3A-1080-saRNA sense sequence
guucccuggucaugccugctt

SEQ ID NO: 1109 - DNA, 21 bp, Artificial Sequence
  Synthetic DNA/RNA construct - RAG3A-1266-saRNA sense sequence
uguuacccaggacagggactt

SEQ ID NO: 1110 - DNA, 21 bp, Artificial Sequence
  Synthetic DNA/RNA construct - RAG3A-1202-saRNA sense sequence
ucucccucucauuguaacutt

SEQ ID NO: 1111 - DNA, 21 bp, Artificial Sequence
  Synthetic DNA/RNA construct - RAG3A-979-saRNA sense sequence
acaucucgucuggcuggactt

SEQ ID NO: 1112 - DNA, 21 bp, Artificial Sequence
  Synthetic DNA/RNA construct - RAG3A-343-saRNA sense sequence
agcauccuguuggauuucatt

SEQ ID NO: 1113 - DNA, 21 bp, Artificial Sequence
  Synthetic DNA/RNA construct - RAG3A-430-saRNA sense sequence
gugggcaauauccguguuctt

SEQ ID NO: 1114 - DNA, 21 bp, Artificial Sequence
  Synthetic DNA/RNA construct - RAG3A-319-saRNA sense sequence
agguaugauguccagggaatt

SEQ ID NO: 1115 - DNA, 21 bp, Artificial Sequence
  Synthetic DNA/RNA construct - RAG3A-612-saRNA sense sequence
ugggcacagaggucugugutt

SEQ ID NO: 1116 - DNA, 21 bp, Artificial Sequence
  Synthetic DNA/RNA construct - RAG3A-454-saRNA sense sequence
cccauaacaggagauuucutt

SEQ ID NO: 1117 - DNA, 21 bp, Artificial Sequence
  Synthetic DNA/RNA construct - RAG3A-1312-saRNA sense sequence
cuagccugccucccuaccutt

SEQ ID NO: 1118 - DNA, 21 bp, Artificial Sequence
  Synthetic DNA/RNA construct - RAG3A-342-saRNA sense sequence
gcauccuguuggauuucagtt

SEQ ID NO: 1119 - DNA, 21 bp, Artificial Sequence
  Synthetic DNA/RNA construct - RAG3A-1118-saRNA sense sequence
ucugccucuccagcccacutt

SEQ ID NO: 1120 - DNA, 21 bp, Artificial Sequence
  Synthetic DNA/RNA construct - RAG3A-970-saRNA sense sequence
cuggcuggacgggaaaauutt

SEQ ID NO: 1121 - DNA, 21 bp, Artificial Sequence
  Synthetic DNA/RNA construct - RAG3A-1133-saRNA sense sequence
ucacccaagggacccucugtt

SEQ ID NO: 1122 - DNA, 21 bp, Artificial Sequence
  Synthetic DNA/RNA construct - RAG3A-689-saRNA sense sequence
gggccugguuauuccucuutt

SEQ ID NO: 1123 - DNA, 21 bp, Artificial Sequence
  Synthetic DNA/RNA construct - RAG3A-1321-saRNA sense sequence
ucucacugccuagccugcctt

SEQ ID NO: 1124 - DNA, 21 bp, Artificial Sequence
  Synthetic DNA/RNA construct - RAG3A-662-saRNA sense sequence
uggaacgguaggagauggatt

SEQ ID NO: 1125 - DNA, 21 bp, Artificial Sequence
  Synthetic DNA/RNA construct - RAG3A-817-saRNA sense sequence
acuguggggagacugggactt

SEQ ID NO: 1126 - DNA, 21 bp, Artificial Sequence
  Synthetic DNA/RNA construct - RAG3A-1242-saRNA sense sequence
cagaaaccggcagcauucctt

SEQ ID NO: 1127 - DNA, 21 bp, Artificial Sequence
  Synthetic DNA/RNA construct - RAG3A-278-saRNA sense sequence
gugaaaaccccaccaaucutt

SEQ ID NO: 1128 - DNA, 21 bp, Artificial Sequence
  Synthetic DNA/RNA construct - RAG3A-1210-saRNA sense sequence
ugacaguaucucccucucatt

SEQ ID NO: 1129 - DNA, 21 bp, Artificial Sequence
  Synthetic DNA/RNA construct - RAG3A-1084-saRNA sense sequence
cuugguucccuggucaugctt

SEQ ID NO: 1130 - DNA, 21 bp, Artificial Sequence
  Synthetic DNA/RNA construct - RAG3A-1166-saRNA sense sequence
gacaguccccucuugcuuutt

SEQ ID NO: 1131 - DNA, 21 bp, Artificial Sequence
  Synthetic DNA/RNA construct - RAG3A-982-saRNA sense sequence
gagacaucucgucuggcugtt

SEQ ID NO: 1132 - DNA, 21 bp, Artificial Sequence
  Synthetic DNA/RNA construct - RAG3A-171-saRNA sense sequence
aggucccagguugcccaugtt

SEQ ID NO: 1133 - DNA, 21 bp, Artificial Sequence
  Synthetic DNA/RNA construct - RAG3A-256-saRNA sense sequence
acaagaccucugugcuucutt

SEQ ID NO: 1134 - DNA, 21 bp, Artificial Sequence
  Synthetic DNA/RNA construct - RAG3A-563-saRNA sense sequence
agacgagcaccuaagcucatt

SEQ ID NO: 1135 - DNA, 21 bp, Artificial Sequence
  Synthetic DNA/RNA construct - RAG3A-1176-saRNA sense sequence
aggcgcauucgacaguccctt

SEQ ID NO: 1136 - DNA, 21 bp, Artificial Sequence
  Synthetic DNA/RNA construct - RAG3A-707-saRNA sense sequence
ccuaagacagggacacaugtt

SEQ ID NO: 1137 - DNA, 21 bp, Artificial Sequence
  Synthetic DNA/RNA construct - RAG3A-561-saRNA sense sequence
acgagcaccuaagcucaggtt

SEQ ID NO: 1138 - DNA, 21 bp, Artificial Sequence
  Synthetic DNA/RNA construct - RAG3A-504-saRNA sense sequence
gcaccugacaguccacucatt

SEQ ID NO: 1139 - DNA, 21 bp, Artificial Sequence
  Synthetic DNA/RNA construct - RAG3A-195-saRNA sense sequence
aaacuucugcuccuguccctt

SEQ ID NO: 1140 - DNA, 21 bp, Artificial Sequence
  Synthetic DNA/RNA construct - RAG3A-850-saRNA sense sequence
caaggucaggccaggaagctt

SEQ ID NO: 1141 - DNA, 21 bp, Artificial Sequence
  Synthetic DNA/RNA construct - RAG3A-388-saRNA sense sequence
agauaggacucccuaggggtt

SEQ ID NO: 1142 - DNA, 21 bp, Artificial Sequence
  Synthetic DNA/RNA construct - RAG3A-1079-saRNA sense sequence
uucccuggucaugccugcctt

SEQ ID NO: 1143 - DNA, 21 bp, Artificial Sequence
  Synthetic DNA/RNA construct - RAG3A-1263-saRNA sense sequence
uacccaggacagggaccuatt

SEQ ID NO: 1144 - DNA, 21 bp, Artificial Sequence
  Synthetic DNA/RNA construct - RAG3A-531-saRNA sense sequence
gcacgugugcgcacauacatt

SEQ ID NO: 1145 - DNA, 21 bp, Artificial Sequence
  Synthetic DNA/RNA construct - RAG3A-254-saRNA sense sequence
aagaccucugugcuucuuctt

SEQ ID NO: 1146 - DNA, 21 bp, Artificial Sequence
  Synthetic DNA/RNA construct - RAG3A-421-saRNA sense sequence
auccguguucccacuucgatt

SEQ ID NO: 1147 - DNA, 21 bp, Artificial Sequence
  Synthetic DNA/RNA construct - RAG3A-473-saRNA sense sequence
ccuuuccccauaacaccaatt

SEQ ID NO: 1148 - DNA, 21 bp, Artificial Sequence
  Synthetic DNA/RNA construct - RAG3A-292-saRNA sense sequence
uggauagccagggagugaatt

SEQ ID NO: 1149 - DNA, 21 bp, Artificial Sequence
  Synthetic DNA/RNA construct - RAG3A-283-saRNA sense sequence
agggagugaaaaccccacctt

SEQ ID NO: 1150 - DNA, 21 bp, Artificial Sequence
  Synthetic DNA/RNA construct - RAG3A-786-saRNA sense sequence
aaaggagcuagagccagggtt

SEQ ID NO: 1151 - DNA, 21 bp, Artificial Sequence
  Synthetic DNA/RNA construct - RAG3A-582-saRNA sense sequence
uaagccacuggaccccagctt

SEQ ID NO: 1152 - DNA, 21 bp, Artificial Sequence
  Synthetic DNA/RNA construct - RAG3A-933-saRNA sense sequence
uucucagcagacaugcccatt

SEQ ID NO: 1153 - DNA, 21 bp, Artificial Sequence
  Synthetic DNA/RNA construct - RAG3A-472-saRNA sense sequence
cuuuccccauaacaccaactt

SEQ ID NO: 1154 - DNA, 21 bp, Artificial Sequence
  Synthetic DNA/RNA construct - RAG3A-646-saRNA sense sequence
ggaagacggagacagaacatt

SEQ ID NO: 1155 - DNA, 21 bp, Artificial Sequence
  Synthetic DNA/RNA construct - RAG3A-389-saRNA sense sequence
aagauaggacucccuagggtt

SEQ ID NO: 1156 - DNA, 21 bp, Artificial Sequence
  Synthetic DNA/RNA construct - RAG3A-971-saRNA sense sequence
ucuggcuggacgggaaaautt

SEQ ID NO: 1157 - DNA, 21 bp, Artificial Sequence
  Synthetic DNA/RNA construct - RAG3A-751-saRNA sense sequence
gccagcagggagguauuugtt

SEQ ID NO: 1158 - DNA, 21 bp, Artificial Sequence
  Synthetic DNA/RNA construct - RAG3A-299-saRNA sense sequence
agaaauuuggauagccaggtt

SEQ ID NO: 1159 - DNA, 21 bp, Artificial Sequence
  Synthetic DNA/RNA construct - RAG3A-750-saRNA sense sequence
ccagcagggagguauuugctt

SEQ ID NO: 1160 - DNA, 21 bp, Artificial Sequence
  Synthetic DNA/RNA construct - RAG3A-671-saRNA sense sequence
ugucacauguggaacgguatt

SEQ ID NO: 1161 - DNA, 21 bp, Artificial Sequence
  Synthetic DNA/RNA construct - RAG3A-749-saRNA sense sequence
cagcagggagguauuugcgtt

SEQ ID NO: 1162 - DNA, 21 bp, Artificial Sequence
  Synthetic DNA/RNA construct - RAG3A-547-saRNA sense sequence
ucaggcuuaacccagugcatt

SEQ ID NO: 1163 - DNA, 21 bp, Artificial Sequence
  Synthetic DNA/RNA construct - RAG3A-655-saRNA sense sequence
guaggagauggaagacggatt

SEQ ID NO: 1164 - DNA, 21 bp, Artificial Sequence
  Synthetic DNA/RNA construct - RAG3A-170-saRNA sense sequence
ggucccagguugcccaugutt

SEQ ID NO: 1165 - DNA, 21 bp, Artificial Sequence
  Synthetic DNA/RNA construct - RAG3A-629-saRNA sense sequence
caagcaaaggagggcccugtt

SEQ ID NO: 1166 - DNA, 21 bp, Artificial Sequence
  Synthetic DNA/RNA construct - RAG3A-253-saRNA sense sequence
agaccucugugcuucuucctt

SEQ ID NO: 1167 - DNA, 21 bp, Artificial Sequence
  Synthetic DNA/RNA construct - RAG3A-695-saRNA sense sequence
acacaugggccugguuauutt

SEQ ID NO: 1168 - DNA, 21 bp, Artificial Sequence
  Synthetic DNA/RNA construct - RAG3A-788-saRNA sense sequence
gcaaaggagcuagagccagtt

SEQ ID NO: 1169 - DNA, 21 bp, Artificial Sequence
  Synthetic DNA/RNA construct - RAG3A-688-saRNA sense sequence
ggccugguuauuccucuugtt

SEQ ID NO: 1170 - DNA, 21 bp, Artificial Sequence
  Synthetic DNA/RNA construct - RAG3A-728-saRNA sense sequence
ggagguccagcagcugucutt

SEQ ID NO: 1171 - DNA, 21 bp, Artificial Sequence
  Synthetic DNA/RNA construct - RAG3A-519-saRNA sense sequence
acauacaugugccccgcactt

SEQ ID NO: 1172 - DNA, 21 bp, Artificial Sequence
  Synthetic DNA/RNA construct - RAG3A-272-saRNA sense sequence
accccaccaaucuuaaacatt

SEQ ID NO: 1173 - DNA, 21 bp, Artificial Sequence
  Synthetic DNA/RNA construct - RAG3A-318-saRNA sense sequence
gguaugauguccagggaaatt

SEQ ID NO: 1174 - DNA, 21 bp, Artificial Sequence
  Synthetic DNA/RNA construct - RAG3A-1338-saRNA sense sequence
aaacccucaccacccucuctt

SEQ ID NO: 1175 - DNA, 21 bp, Artificial Sequence
  Synthetic DNA/RNA construct - RAG3A-539-saRNA sense sequence
aacccagugcacgugugcgtt

SEQ ID NO: 1176 - DNA, 21 bp, Artificial Sequence
  Synthetic DNA/RNA construct - RAG3A-826-saRNA sense sequence
aggacagagacuguggggatt

SEQ ID NO: 1177 - DNA, 21 bp, Artificial Sequence
  Synthetic DNA/RNA construct - RAG3A-337-saRNA sense sequence
cuguuggauuucagcagcatt

SEQ ID NO: 1178 - DNA, 21 bp, Artificial Sequence
  Synthetic DNA/RNA construct - RAG3A-273-saRNA sense sequence
aaccccaccaaucuuaaactt

SEQ ID NO: 1179 - DNA, 21 bp, Artificial Sequence
  Synthetic DNA/RNA construct - RAG3A-478-saRNA sense sequence
caaacccuuuccccauaactt

SEQ ID NO: 1180 - DNA, 21 bp, Artificial Sequence
  Synthetic DNA/RNA construct - RAG3A-963-saRNA sense sequence
gacgggaaaauuccaggautt

SEQ ID NO: 1181 - DNA, 21 bp, Artificial Sequence
  Synthetic DNA/RNA construct - RAG3A-248-saRNA sense sequence
ucugugcuucuuccccagctt

SEQ ID NO: 1182 - DNA, 21 bp, Artificial Sequence
  Synthetic DNA/RNA construct - RAG3A-35-saRNA sense sequence
ucaccagugucugguggcutt

SEQ ID NO: 1183 - DNA, 21 bp, Artificial Sequence
  Synthetic DNA/RNA construct - RAG3A-613-saRNA sense sequence
cugggcacagaggucugugtt

SEQ ID NO: 1184 - DNA, 21 bp, Artificial Sequence
  Synthetic DNA/RNA construct - RAG3A-1141-saRNA sense sequence
cccuuccuucacccaagggtt

SEQ ID NO: 1185 - DNA, 21 bp, Artificial Sequence
  Synthetic DNA/RNA construct - RAG3A-477-saRNA sense sequence
aaacccuuuccccauaacatt

SEQ ID NO: 1186 - DNA, 21 bp, Artificial Sequence
  Synthetic DNA/RNA construct - RAG3A-634-saRNA sense sequence
cagaacaagcaaaggagggtt

SEQ ID NO: 1187 - DNA, 21 bp, Artificial Sequence
  Synthetic DNA/RNA construct - RAG3A-1280-saRNA sense sequence
gcuccucauucuuguguuatt

SEQ ID NO: 1188 - DNA, 21 bp, Artificial Sequence
  Synthetic DNA/RNA construct - RAG3A-1062-saRNA sense sequence
ccucccugucuccugucuctt

SEQ ID NO: 1189 - DNA, 21 bp, Artificial Sequence
  Synthetic DNA/RNA construct - RAG3A-778-saRNA sense sequence
uagagccagggccaaaggatt

SEQ ID NO: 1190 - DNA, 21 bp, Artificial Sequence
  Synthetic DNA/RNA construct - RAG3A-939-saRNA sense sequence
ccacacuucucagcagacatt

SEQ ID NO: 1191 - DNA, 21 bp, Artificial Sequence
  Synthetic DNA/RNA construct - RAG3A-779-saRNA sense sequence
cuagagccagggccaaaggtt

SEQ ID NO: 1192 - DNA, 21 bp, Artificial Sequence
  Synthetic DNA/RNA construct - RAG3A-803-saRNA sense sequence
gggacugggaagaaagcaatt

SEQ ID NO: 1193 - DNA, 21 bp, Artificial Sequence
  Synthetic DNA/RNA construct - RAG3A-1382-saRNA sense sequence
guccaucagccgaucuccctt

SEQ ID NO: 1194 - DNA, 21 bp, Artificial Sequence
  Synthetic DNA/RNA construct - RAG3A-97-saRNA sense sequence
aggaguggacccugguccatt

SEQ ID NO: 1195 - DNA, 21 bp, Artificial Sequence
  Synthetic DNA/RNA construct - RAG3A-543-saRNA sense sequence
gcuuaacccagugcacgugtt

SEQ ID NO: 1196 - DNA, 21 bp, Artificial Sequence
  Synthetic DNA/RNA construct - RAG3A-556-saRNA sense sequence
caccuaagcucaggcuuaatt

SEQ ID NO: 1197 - DNA, 21 bp, Artificial Sequence
  Synthetic DNA/RNA construct - RAG3A-60-saRNA sense sequence
gagaaggcgucacuuccggtt

SEQ ID NO: 1198 - DNA, 21 bp, Artificial Sequence
  Synthetic DNA/RNA construct - RAG3A-647-saRNA sense sequence
uggaagacggagacagaactt

SEQ ID NO: 1199 - DNA, 21 bp, Artificial Sequence
  Synthetic DNA/RNA construct - RAG3A-243-saRNA sense sequence
gcuucuuccccagcaacactt

SEQ ID NO: 1200 - DNA, 21 bp, Artificial Sequence
  Synthetic DNA/RNA construct - RAG3A-533-saRNA sense sequence
gugcacgugugcgcacauatt

SEQ ID NO: 1201 - DNA, 21 bp, Artificial Sequence
  Synthetic DNA/RNA construct - RAG3A-628-saRNA sense sequence
aagcaaaggagggcccuggtt

SEQ ID NO: 1202 - DNA, 21 bp, Artificial Sequence
  Synthetic DNA/RNA construct - RAG3A-849-saRNA sense sequence
aaggucaggccaggaagcctt

SEQ ID NO: 1203 - DNA, 21 bp, Artificial Sequence
  Synthetic DNA/RNA construct - RAG3A-1307-saRNA sense sequence
cugccucccuaccuucucutt

SEQ ID NO: 1204 - DNA, 21 bp, Artificial Sequence
  Synthetic DNA/RNA construct - RAG3A-293-saRNA sense sequence
uuggauagccagggagugatt

SEQ ID NO: 1205 - DNA, 21 bp, Artificial Sequence
  Synthetic DNA/RNA construct - RAG3A-1285-saRNA sense sequence
aggucgcuccucauucuugtt

SEQ ID NO: 1206 - DNA, 21 bp, Artificial Sequence
  Synthetic DNA/RNA construct - RAG3A-1223-saRNA sense sequence
cccuucuguggagugacagtt

SEQ ID NO: 1207 - DNA, 21 bp, Artificial Sequence
  Synthetic DNA/RNA construct - RAG3A-1177-saRNA sense sequence
caggcgcauucgacagucctt

SEQ ID NO: 1208 - DNA, 21 bp, Artificial Sequence
  Synthetic DNA/RNA construct - RAG3A-226-saRNA sense sequence
acaaauguccugccagauutt

SEQ ID NO: 1209 - DNA, 21 bp, Artificial Sequence
  Synthetic DNA/RNA construct - RAG3A-1097-saRNA sense sequence
cagccuccuuucucuuggutt

SEQ ID NO: 1210 - DNA, 21 bp, Artificial Sequence
  Synthetic DNA/RNA construct - RAG3A-1188-saRNA sense sequence
uaacuuauccucaggcgcatt

SEQ ID NO: 1211 - DNA, 21 bp, Artificial Sequence
  Synthetic DNA/RNA construct - RAG3A-593-saRNA sense sequence
guguagccaucuaagccactt

SEQ ID NO: 1212 - DNA, 21 bp, Artificial Sequence
  Synthetic DNA/RNA construct - RAG3A-670-saRNA sense sequence
gucacauguggaacgguagtt

SEQ ID NO: 1213 - DNA, 21 bp, Artificial Sequence
  Synthetic DNA/RNA construct - RAG3A-503-saRNA sense sequence
caccugacaguccacucaatt

SEQ ID NO: 1214 - DNA, 21 bp, Artificial Sequence
  Synthetic DNA/RNA construct - RAG3A-1167-saRNA sense sequence
cgacaguccccucuugcuutt

SEQ ID NO: 1215 - DNA, 21 bp, Artificial Sequence
  Synthetic DNA/RNA construct - RAG3A-583-saRNA sense sequence
cuaagccacuggaccccagtt

SEQ ID NO: 1216 - DNA, 21 bp, Artificial Sequence
  Synthetic DNA/RNA construct - RAG3A-706-saRNA sense sequence
cuaagacagggacacauggtt

SEQ ID NO: 1217 - DNA, 21 bp, Artificial Sequence
  Synthetic DNA/RNA construct - RAG3A-983-saRNA sense sequence
ggagacaucucgucuggcutt

SEQ ID NO: 1218 - DNA, 21 bp, Artificial Sequence
  Synthetic DNA/RNA construct - RAG3A-718-saRNA sense sequence
cagcugucuuuccuaagactt

SEQ ID NO: 1219 - DNA, 21 bp, Artificial Sequence
  Synthetic DNA/RNA construct - RAG3A-378-saRNA sense sequence
cccuaggggauuacagaaatt

SEQ ID NO: 1220 - DNA, 21 bp, Artificial Sequence
  Synthetic DNA/RNA construct - RAG3A-1095-saRNA sense sequence
gccuccuuucucuugguuctt

SEQ ID NO: 1221 - DNA, 21 bp, Artificial Sequence
  Synthetic DNA/RNA construct - RAG3A-698-saRNA sense sequence
gggacacaugggccugguutt

SEQ ID NO: 1222 - DNA, 21 bp, Artificial Sequence
  Synthetic DNA/RNA construct - RAG3A-277-saRNA sense sequence
ugaaaaccccaccaaucuutt

SEQ ID NO: 1223 - DNA, 21 bp, Artificial Sequence
  Synthetic DNA/RNA construct - RAG3A-644-saRNA sense sequence
aagacggagacagaacaagtt

SEQ ID NO: 1224 - DNA, 21 bp, Artificial Sequence
  Synthetic DNA/RNA construct - RAG3A-941-saRNA sense sequence
ggccacacuucucagcagatt

SEQ ID NO: 1225 - DNA, 21 bp, Artificial Sequence
  Synthetic DNA/RNA construct - RAG3A-794-saRNA sense sequence
aagaaagcaaaggagcuagtt

SEQ ID NO: 1226 - DNA, 21 bp, Artificial Sequence
  Synthetic DNA/RNA construct - RAG3A-890-saRNA sense sequence
gagccugaggaaguucuggtt

SEQ ID NO: 1227 - DNA, 21 bp, Artificial Sequence
  Synthetic DNA/RNA construct - RAG3A-1310-saRNA sense sequence
agccugccucccuaccuuctt

SEQ ID NO: 1228 - DNA, 21 bp, Artificial Sequence
  Synthetic DNA/RNA construct - RAG3A-27-saRNA sense sequence
gucugguggcucccuucuctt

SEQ ID NO: 1229 - DNA, 21 bp, Artificial Sequence
  Synthetic DNA/RNA construct - RAG3A-38-saRNA sense sequence
ccuucaccagugucuggugtt

SEQ ID NO: 1230 - DNA, 21 bp, Artificial Sequence
  Synthetic DNA/RNA construct - RAG3A-532-saRNA sense sequence
ugcacgugugcgcacauactt

SEQ ID NO: 1231 - DNA, 21 bp, Artificial Sequence
  Synthetic DNA/RNA construct - RAG3A-1023-saRNA sense sequence
uauccucccagcuaucccatt

SEQ ID NO: 1232 - DNA, 21 bp, Artificial Sequence
  Synthetic DNA/RNA construct - RAG3A-797-saRNA sense sequence
gggaagaaagcaaaggagctt

SEQ ID NO: 1233 - DNA, 21 bp, Artificial Sequence
  Synthetic DNA/RNA construct - RAG3A-399-saRNA sense sequence
ggggaaugacaagauaggatt

SEQ ID NO: 1234 - DNA, 21 bp, Artificial Sequence
  Synthetic DNA/RNA construct - RAG3A-945-saRNA sense sequence
ucuaggccacacuucucagtt

SEQ ID NO: 1235 - DNA, 21 bp, Artificial Sequence
  Synthetic DNA/RNA construct - RAG3A-344-saRNA sense sequence
aagcauccuguuggauuuctt

SEQ ID NO: 1236 - DNA, 21 bp, Artificial Sequence
  Synthetic DNA/RNA construct - RAG3A-1255-saRNA sense sequence
acagggaccuagccagaaatt

SEQ ID NO: 1237 - DNA, 21 bp, Artificial Sequence
  Synthetic DNA/RNA construct - RAG3A-524-saRNA sense sequence
ugcgcacauacaugugccctt

SEQ ID NO: 1238 - DNA, 21 bp, Artificial Sequence
  Synthetic DNA/RNA construct - RAG3A-244-saRNA sense sequence
ugcuucuuccccagcaacatt

SEQ ID NO: 1239 - DNA, 21 bp, Artificial Sequence
  Synthetic DNA/RNA construct - RAG3A-340-saRNA sense sequence
auccuguuggauuucagcatt

SEQ ID NO: 1240 - DNA, 21 bp, Artificial Sequence
  Synthetic DNA/RNA construct - RAG3A-687-saRNA sense sequence
gccugguuauuccucuugutt

SEQ ID NO: 1241 - DNA, 21 bp, Artificial Sequence
  Synthetic DNA/RNA construct - RAG3A-568-saRNA sense sequence
ccagcagacgagcaccuaatt

SEQ ID NO: 1242 - DNA, 21 bp, Artificial Sequence
  Synthetic DNA/RNA construct - RAG3A-804-saRNA sense sequence
ugggacugggaagaaagcatt

SEQ ID NO: 1243 - DNA, 21 bp, Artificial Sequence
  Synthetic DNA/RNA construct - RAG3A-496-saRNA sense sequence
caguccacucaacccgucctt

SEQ ID NO: 1244 - DNA, 21 bp, Artificial Sequence
  Synthetic DNA/RNA construct - RAG3A-810-saRNA sense sequence
ggagacugggacugggaagtt

SEQ ID NO: 1245 - DNA, 21 bp, Artificial Sequence
  Synthetic DNA/RNA construct - RAG3A-356-saRNA sense sequence
aagcaggaaagcaagcauctt

SEQ ID NO: 1246 - DNA, 21 bp, Artificial Sequence
  Synthetic DNA/RNA construct - RAG3A-1248-saRNA sense sequence
ccuagccagaaaccggcagtt

SEQ ID NO: 1247 - DNA, 21 bp, Artificial Sequence
  Synthetic DNA/RNA construct - RAG3A-1015-saRNA sense sequence
cagcuaucccagcacccuctt

SEQ ID NO: 1248 - DNA, 21 bp, Artificial Sequence
  Synthetic DNA/RNA construct - RAG3A-645-saRNA sense sequence
gaagacggagacagaacaatt

SEQ ID NO: 1249 - DNA, 21 bp, Artificial Sequence
  Synthetic DNA/RNA construct - RAG3A-517-saRNA sense sequence
auacaugugccccgcaccutt

SEQ ID NO: 1250 - DNA, 21 bp, Artificial Sequence
  Synthetic DNA/RNA construct - RAG3A-194-saRNA sense sequence
aacuucugcuccugucccctt

SEQ ID NO: 1251 - DNA, 21 bp, Artificial Sequence
  Synthetic DNA/RNA construct - RAG3A-927-saRNA sense sequence
gcagacaugcccauccuugtt

SEQ ID NO: 1252 - DNA, 21 bp, Artificial Sequence
  Synthetic DNA/RNA construct - RAG3A-271-saRNA sense sequence
ccccaccaaucuuaaacaatt

SEQ ID NO: 1253 - DNA, 21 bp, Artificial Sequence
  Synthetic DNA/RNA construct - RAG3A-398-saRNA sense sequence
gggaaugacaagauaggactt

SEQ ID NO: 1254 - DNA, 21 bp, Artificial Sequence
  Synthetic DNA/RNA construct - RAG3A-230-saRNA sense sequence
caacacaaauguccugccatt

SEQ ID NO: 1255 - DNA, 21 bp, Artificial Sequence
  Synthetic DNA/RNA construct - RAG3A-1104-saRNA sense sequence
ccacucccagccuccuuuctt

SEQ ID NO: 1256 - DNA, 21 bp, Artificial Sequence
  Synthetic DNA/RNA construct - RAG3A-1254-saRNA sense sequence
cagggaccuagccagaaactt

SEQ ID NO: 1257 - DNA, 21 bp, Artificial Sequence
  Synthetic DNA/RNA construct - RAG3A-288-saRNA sense sequence
uagccagggagugaaaacctt

SEQ ID NO: 1258 - DNA, 21 bp, Artificial Sequence
  Synthetic DNA/RNA construct - RAG3A-837-saRNA sense sequence
ggaagccccugaggacagatt

SEQ ID NO: 1259 - DNA, 21 bp, Artificial Sequence
  Synthetic DNA/RNA construct - RAG3A-935-saRNA sense sequence
acuucucagcagacaugcctt

SEQ ID NO: 1260 - DNA, 21 bp, Artificial Sequence
  Synthetic DNA/RNA construct - RAG3A-476-saRNA sense sequence
aacccuuuccccauaacactt

SEQ ID NO: 1261 - DNA, 21 bp, Artificial Sequence
  Synthetic DNA/RNA construct - RAG3A-589-saRNA sense sequence
agccaucuaagccacuggatt

SEQ ID NO: 1262 - DNA, 21 bp, Artificial Sequence
  Synthetic DNA/RNA construct - RAG3A-1207-saRNA sense sequence
caguaucucccucucauugtt

SEQ ID NO: 1263 - DNA, 21 bp, Artificial Sequence
  Synthetic DNA/RNA construct - RAG3A-1284-saRNA sense sequence
ggucgcuccucauucuugutt

SEQ ID NO: 1264 - DNA, 21 bp, Artificial Sequence
  Synthetic DNA/RNA construct - RAG3A-956-saRNA sense sequence
aaauuccaggaucuaggcctt

SEQ ID NO: 1265 - DNA, 21 bp, Artificial Sequence
  Synthetic DNA/RNA construct - RAG3A-557-saRNA sense sequence
gcaccuaagcucaggcuuatt

SEQ ID NO: 1266 - DNA, 21 bp, Artificial Sequence
  Synthetic DNA/RNA construct - RAG3A-500-saRNA sense sequence
cugacaguccacucaaccctt

SEQ ID NO: 1267 - DNA, 21 bp, Artificial Sequence
  Synthetic DNA/RNA construct - RAG3A-1208-saRNA sense sequence
acaguaucucccucucauutt

SEQ ID NO: 1268 - DNA, 21 bp, Artificial Sequence
  Synthetic DNA/RNA construct - RAG3A-686-saRNA sense sequence
ccugguuauuccucuuguctt

SEQ ID NO: 1269 - DNA, 21 bp, Artificial Sequence
  Synthetic DNA/RNA construct - RAG3A-1168-saRNA sense sequence
ucgacaguccccucuugcutt

SEQ ID NO: 1270 - DNA, 21 bp, Artificial Sequence
  Synthetic DNA/RNA construct - RAG3A-218-saRNA sense sequence
ccugccagauuccuccuggtt

SEQ ID NO: 1271 - DNA, 21 bp, Artificial Sequence
  Synthetic DNA/RNA construct - RAG3A-835-saRNA sense sequence
aagccccugaggacagagatt

SEQ ID NO: 1272 - DNA, 21 bp, Artificial Sequence
  Synthetic DNA/RNA construct - RAG3A-291-saRNA sense sequence
ggauagccagggagugaaatt

SEQ ID NO: 1273 - DNA, 21 bp, Artificial Sequence
  Synthetic DNA/RNA construct - RAG3A-1222-saRNA sense sequence
ccuucuguggagugacagutt

SEQ ID NO: 1274 - DNA, 21 bp, Artificial Sequence
  Synthetic DNA/RNA construct - RAG3A-276-saRNA sense sequence
gaaaaccccaccaaucuuatt

SEQ ID NO: 1275 - DNA, 21 bp, Artificial Sequence
  Synthetic DNA/RNA construct - RAG3A-156-saRNA sense sequence
cauguccaggaaaagauggtt

SEQ ID NO: 1276 - DNA, 21 bp, Artificial Sequence
  Synthetic DNA/RNA construct - RAG3A-937-saRNA sense sequence
acacuucucagcagacaugtt

SEQ ID NO: 1277 - DNA, 21 bp, Artificial Sequence
  Synthetic DNA/RNA construct - RAG3A-1262-saRNA sense sequence
acccaggacagggaccuagtt

SEQ ID NO: 1278 - DNA, 21 bp, Artificial Sequence
  Synthetic DNA/RNA construct - RAG3A-1139-saRNA sense sequence
cuuccuucacccaagggactt

SEQ ID NO: 1279 - DNA, 21 bp, Artificial Sequence
  Synthetic DNA/RNA construct - RAG3A-535-saRNA sense sequence
cagugcacgugugcgcacatt

SEQ ID NO: 1280 - DNA, 21 bp, Artificial Sequence
  Synthetic DNA/RNA construct - RAG3A-719-saRNA sense sequence
gcagcugucuuuccuaagatt

SEQ ID NO: 1281 - DNA, 21 bp, Artificial Sequence
  Synthetic DNA/RNA construct - RAG3A-726-saRNA sense sequence
agguccagcagcugucuuutt

SEQ ID NO: 1282 - DNA, 21 bp, Artificial Sequence
  Synthetic DNA/RNA construct - RAG3A-384-saRNA sense sequence
aggacucccuaggggauuatt

SEQ ID NO: 1283 - DNA, 21 bp, Artificial Sequence
  Synthetic DNA/RNA construct - RAG3A-834-saRNA sense sequence
agccccugaggacagagactt

SEQ ID NO: 1284 - DNA, 21 bp, Artificial Sequence
  Synthetic DNA/RNA construct - RAG3A-562-saRNA sense sequence
gacgagcaccuaagcucagtt

SEQ ID NO: 1285 - DNA, 21 bp, Artificial Sequence
  Synthetic DNA/RNA construct - RAG3A-940-saRNA sense sequence
gccacacuucucagcagactt

SEQ ID NO: 1286 - DNA, 21 bp, Artificial Sequence
  Synthetic DNA/RNA construct - RAG3A-632-saRNA sense sequence
gaacaagcaaaggagggcctt

SEQ ID NO: 1287 - DNA, 21 bp, Artificial Sequence
  Synthetic DNA/RNA construct - RAG3A-746-saRNA sense sequence
cagggagguauuugcggggtt

SEQ ID NO: 1288 - DNA, 21 bp, Artificial Sequence
  Synthetic DNA/RNA construct - RAG3A-1309-saRNA sense sequence
gccugccucccuaccuucutt

SEQ ID NO: 1289 - DNA, 21 bp, Artificial Sequence
  Synthetic DNA/RNA construct - RAG3A-780-saRNA sense sequence
gcuagagccagggccaaagtt

SEQ ID NO: 1290 - DNA, 21 bp, Artificial Sequence
  Synthetic DNA/RNA construct - RAG3A-693-saRNA sense sequence
acaugggccugguuauucctt

SEQ ID NO: 1291 - DNA, 21 bp, Artificial Sequence
  Synthetic DNA/RNA construct - RAG3A-636-saRNA sense sequence
gacagaacaagcaaaggagtt

SEQ ID NO: 1292 - DNA, 21 bp, Artificial Sequence
  Synthetic DNA/RNA construct - RAG3A-258-saRNA sense sequence
aaacaagaccucugugcuutt

SEQ ID NO: 1293 - DNA, 21 bp, Artificial Sequence
  Synthetic DNA/RNA construct - RAG3A-1022-saRNA sense sequence
auccucccagcuaucccagtt

SEQ ID NO: 1294 - DNA, 21 bp, Artificial Sequence
  Synthetic DNA/RNA construct - RAG3A-381-saRNA sense sequence
acucccuaggggauuacagtt

SEQ ID NO: 1295 - DNA, 21 bp, Artificial Sequence
  Synthetic DNA/RNA construct - RAG3A-379-saRNA sense sequence
ucccuaggggauuacagaatt

SEQ ID NO: 1296 - DNA, 21 bp, Artificial Sequence
  Synthetic DNA/RNA construct - RAG3A-663-saRNA sense sequence
guggaacgguaggagauggtt

SEQ ID NO: 1297 - DNA, 21 bp, Artificial Sequence
  Synthetic DNA/RNA construct - RAG3A-1099-saRNA sense sequence
cccagccuccuuucucuugtt

SEQ ID NO: 1298 - DNA, 21 bp, Artificial Sequence
  Synthetic DNA/RNA construct - RAG3A-855-saRNA sense sequence
gggaucaaggucaggccagtt

SEQ ID NO: 1299 - DNA, 21 bp, Artificial Sequence
  Synthetic DNA/RNA construct - RAG3A-1279-saRNA sense sequence
cuccucauucuuguguuactt

SEQ ID NO: 1300 - DNA, 21 bp, Artificial Sequence
  Synthetic DNA/RNA construct - RAG3A-34-saRNA sense sequence
caccagugucugguggcuctt

SEQ ID NO: 1301 - DNA, 21 bp, Artificial Sequence
  Synthetic DNA/RNA construct - RAG3A-633-saRNA sense sequence
agaacaagcaaaggagggctt

SEQ ID NO: 1302 - DNA, 21 bp, Artificial Sequence
  Synthetic DNA/RNA construct - RAG3A-453-saRNA sense sequence
ccauaacaggagauuucuctt

SEQ ID NO: 1303 - DNA, 21 bp, Artificial Sequence
  Synthetic DNA/RNA construct - RAG3A-246-saRNA sense sequence
ugugcuucuuccccagcaatt

SEQ ID NO: 1304 - DNA, 21 bp, Artificial Sequence
  Synthetic DNA/RNA construct - RAG3A-592-saRNA sense sequence
uguagccaucuaagccacutt

SEQ ID NO: 1305 - DNA, 21 bp, Artificial Sequence
  Synthetic DNA/RNA construct - RAG3A-270-saRNA sense sequence
cccaccaaucuuaaacaagtt

SEQ ID NO: 1306 - DNA, 21 bp, Artificial Sequence
  Synthetic DNA/RNA construct - RAG3A-630-saRNA sense sequence
acaagcaaaggagggcccutt

SEQ ID NO: 1307 - DNA, 21 bp, Artificial Sequence
  Synthetic DNA/RNA construct - RAG3A-685-saRNA sense sequence
cugguuauuccucuugucatt

SEQ ID NO: 1308 - DNA, 21 bp, Artificial Sequence
  Synthetic DNA/RNA construct - RAG3A-1103-saRNA sense sequence
cacucccagccuccuuucutt

SEQ ID NO: 1309 - DNA, 21 bp, Artificial Sequence
  Synthetic DNA/RNA construct - RAG3A-1065-saRNA sense sequence
cugccucccugucuccugutt

SEQ ID NO: 1310 - DNA, 21 bp, Artificial Sequence
  Synthetic DNA/RNA construct - RAG3A-523-saRNA sense sequence
gcgcacauacaugugcccctt

SEQ ID NO: 1311 - DNA, 21 bp, Artificial Sequence
  Synthetic DNA/RNA construct - RAG3A-590-saRNA sense sequence
uagccaucuaagccacuggtt

SEQ ID NO: 1312 - DNA, 21 bp, Artificial Sequence
  Synthetic DNA/RNA construct - RAG3A-505-saRNA sense sequence
cgcaccugacaguccacuctt

SEQ ID NO: 1313 - DNA, 21 bp, Artificial Sequence
  Synthetic DNA/RNA construct - RAG3A-1247-saRNA sense sequence
cuagccagaaaccggcagctt

SEQ ID NO: 1314 - DNA, 21 bp, Artificial Sequence
  Synthetic DNA/RNA construct - RAG3A-1281-saRNA sense sequence
cgcuccucauucuuguguutt

SEQ ID NO: 1315 - DNA, 21 bp, Artificial Sequence
  Synthetic DNA/RNA construct - RAG3A-747-saRNA sense sequence
gcagggagguauuugcgggtt

SEQ ID NO: 1316 - DNA, 21 bp, Artificial Sequence
  Synthetic DNA/RNA construct - RAG3A-1220-saRNA sense sequence
uucuguggagugacaguautt

SEQ ID NO: 1317 - DNA, 21 bp, Artificial Sequence
  Synthetic DNA/RNA construct - RAG3A-1268-saRNA sense sequence
uguguuacccaggacagggtt

SEQ ID NO: 1318 - DNA, 21 bp, Artificial Sequence
  Synthetic DNA/RNA construct - RAG3A-782-saRNA sense sequence
gagcuagagccagggccaatt

SEQ ID NO: 1319 - DNA, 21 bp, Artificial Sequence
  Synthetic DNA/RNA construct - RAG3A-107-saRNA sense sequence
guguggguggaggaguggatt

SEQ ID NO: 1320 - DNA, 21 bp, Artificial Sequence
  Synthetic DNA/RNA construct - RAG3A-923-saRNA sense sequence
acaugcccauccuuggggatt

SEQ ID NO: 1321 - DNA, 21 bp, Artificial Sequence
  Synthetic DNA/RNA construct - RAG3A-349-saRNA sense sequence
aaagcaagcauccuguuggtt

SEQ ID NO: 1322 - DNA, 21 bp, Artificial Sequence
  Synthetic DNA/RNA construct - RAG3A-1085-saRNA sense sequence
ucuugguucccuggucaugtt

SEQ ID NO: 1323 - DNA, 21 bp, Artificial Sequence
  Synthetic DNA/RNA construct - RAG3A-432-saRNA sense sequence
augugggcaauauccgugutt

SEQ ID NO: 1324 - DNA, 21 bp, Artificial Sequence
  Synthetic DNA/RNA construct - RAG3A-129-saRNA sense sequence
auccaaaucuucuccguggtt

SEQ ID NO: 1325 - DNA, 21 bp, Artificial Sequence
  Synthetic DNA/RNA construct - RAG3A-830-saRNA sense sequence
ccugaggacagagacugugtt

SEQ ID NO: 1326 - DNA, 21 bp, Artificial Sequence
  Synthetic DNA/RNA construct - RAG3A-74-saRNA sense sequence
ggggcuccagggaagagaatt

SEQ ID NO: 1327 - DNA, 21 bp, Artificial Sequence
  Synthetic DNA/RNA construct - RAG3A-856-saRNA sense sequence
ugggaucaaggucaggccatt

SEQ ID NO: 1328 - DNA, 21 bp, Artificial Sequence
  Synthetic DNA/RNA construct - RAG3A-435-saRNA sense sequence
cucaugugggcaauauccgtt

SEQ ID NO: 1329 - DNA, 21 bp, Artificial Sequence
  Synthetic DNA/RNA construct - RAG3A-564-saRNA sense sequence
cagacgagcaccuaagcuctt

SEQ ID NO: 1330 - DNA, 21 bp, Artificial Sequence
  Synthetic DNA/RNA construct - RAG3A-1251-saRNA sense sequence
ggaccuagccagaaaccggtt

SEQ ID NO: 1331 - DNA, 21 bp, Artificial Sequence
  Synthetic DNA/RNA construct - RAG3A-1291-saRNA sense sequence
ucucugaggucgcuccucatt

SEQ ID NO: 1332 - DNA, 21 bp, Artificial Sequence
  Synthetic DNA/RNA construct - RAG3A-781-saRNA sense sequence
agcuagagccagggccaaatt

SEQ ID NO: 1333 - DNA, 21 bp, Artificial Sequence
  Synthetic DNA/RNA construct - RAG3A-261-saRNA sense sequence
cuuaaacaagaccucugugtt

SEQ ID NO: 1334 - DNA, 21 bp, Artificial Sequence
  Synthetic DNA/RNA construct - RAG3A-1098-saRNA sense sequence
ccagccuccuuucucuuggtt

SEQ ID NO: 1335 - DNA, 21 bp, Artificial Sequence
  Synthetic DNA/RNA construct - RAG3A-565-saRNA sense sequence
gcagacgagcaccuaagcutt

SEQ ID NO: 1336 - DNA, 21 bp, Artificial Sequence
  Synthetic DNA/RNA construct - RAG3A-930-saRNA sense sequence
ucagcagacaugcccaucctt

SEQ ID NO: 1337 - DNA, 21 bp, Artificial Sequence
  Synthetic DNA/RNA construct - RAG3A-422-saRNA sense sequence
uauccguguucccacuucgtt

SEQ ID NO: 1338 - DNA, 21 bp, Artificial Sequence
  Synthetic DNA/RNA construct - RAG3A-516-saRNA sense sequence
uacaugugccccgcaccugtt

SEQ ID NO: 1339 - DNA, 21 bp, Artificial Sequence
  Synthetic DNA/RNA construct - RAG3A-1190-saRNA sense sequence
uguaacuuauccucaggcgtt

SEQ ID NO: 1340 - DNA, 21 bp, Artificial Sequence
  Synthetic DNA/RNA construct - RAG3A-360-saRNA sense sequence
agaaaagcaggaaagcaagtt

SEQ ID NO: 1341 - DNA, 21 bp, Artificial Sequence
  Synthetic DNA/RNA construct - RAG3A-126-saRNA sense sequence
caaaucuucuccguggugutt

SEQ ID NO: 1342 - DNA, 21 bp, Artificial Sequence
  Synthetic DNA/RNA construct - RAG3A-938-saRNA sense sequence
cacacuucucagcagacautt

SEQ ID NO: 1343 - DNA, 21 bp, Artificial Sequence
  Synthetic DNA/RNA construct - RAG3A-853-saRNA sense sequence
gaucaaggucaggccaggatt

SEQ ID NO: 1344 - DNA, 21 bp, Artificial Sequence
  Synthetic DNA/RNA construct - RAG3A-716-saRNA sense sequence
gcugucuuuccuaagacagtt

SEQ ID NO: 1345 - DNA, 21 bp, Artificial Sequence
  Synthetic DNA/RNA construct - RAG3A-1027-saRNA sense sequence
ccacuauccucccagcuautt

SEQ ID NO: 1346 - DNA, 21 bp, Artificial Sequence
  Synthetic DNA/RNA construct - RAG3A-640-saRNA sense sequence
cggagacagaacaagcaaatt

SEQ ID NO: 1347 - DNA, 21 bp, Artificial Sequence
  Synthetic DNA/RNA construct - RAG3A-284-saRNA sense sequence
cagggagugaaaaccccactt

SEQ ID NO: 1348 - DNA, 21 bp, Artificial Sequence
  Synthetic DNA/RNA construct - RAG3A-705-saRNA sense sequence
uaagacagggacacaugggtt

SEQ ID NO: 1349 - DNA, 21 bp, Artificial Sequence
  Synthetic DNA/RNA construct - RAG3A-1315-saRNA sense sequence
ugccuagccugccucccuatt

SEQ ID NO: 1350 - DNA, 21 bp, Artificial Sequence
  Synthetic DNA/RNA construct - RAG3A-1381-saRNA sense sequence
uccaucagccgaucucccctt

SEQ ID NO: 1351 - DNA, 21 bp, Artificial Sequence
  Synthetic DNA/RNA construct - RAG3A-558-saRNA sense sequence
agcaccuaagcucaggcuutt

SEQ ID NO: 1352 - DNA, 21 bp, Artificial Sequence
  Synthetic DNA/RNA construct - RAG3A-498-saRNA sense sequence
gacaguccacucaacccgutt

SEQ ID NO: 1353 - DNA, 21 bp, Artificial Sequence
  Synthetic DNA/RNA construct - RAG3A-581-saRNA sense sequence
aagccacuggaccccagcatt

SEQ ID NO: 1354 - DNA, 21 bp, Artificial Sequence
  Synthetic DNA/RNA construct - RAG3A-1135-saRNA sense sequence
cuucacccaagggacccuctt

SEQ ID NO: 1355 - DNA, 21 bp, Artificial Sequence
  Synthetic DNA/RNA construct - RAG3A-1070-saRNA sense sequence
caugccugccucccugucutt

SEQ ID NO: 1356 - DNA, 21 bp, Artificial Sequence
  Synthetic DNA/RNA construct - RAG3A-1181-saRNA sense sequence
uccucaggcgcauucgacatt

SEQ ID NO: 1357 - DNA, 21 bp, Artificial Sequence
  Synthetic DNA/RNA construct - RAG3A-1206-saRNA sense sequence
aguaucucccucucauugutt

SEQ ID NO: 1358 - DNA, 21 bp, Artificial Sequence
  Synthetic DNA/RNA construct - RAG3A-461-saRNA sense sequence
acaccaacccauaacaggatt

SEQ ID NO: 1359 - DNA, 21 bp, Artificial Sequence
  Synthetic DNA/RNA construct - RAG3A-242-saRNA sense sequence
cuucuuccccagcaacacatt

SEQ ID NO: 1360 - DNA, 21 bp, Artificial Sequence
  Synthetic DNA/RNA construct - RAG3A-1127-saRNA sense sequence
aagggacccucugccucuctt

SEQ ID NO: 1361 - DNA, 21 bp, Artificial Sequence
  Synthetic DNA/RNA construct - RAG3A-419-saRNA sense sequence
ccguguucccacuucgaaatt

SEQ ID NO: 1362 - DNA, 21 bp, Artificial Sequence
  Synthetic DNA/RNA construct - RAG3A-790-saRNA sense sequence
aagcaaaggagcuagagcctt

SEQ ID NO: 1363 - DNA, 21 bp, Artificial Sequence
  Synthetic DNA/RNA construct - RAG3A-635-saRNA sense sequence
acagaacaagcaaaggaggtt

SEQ ID NO: 1364 - DNA, 21 bp, Artificial Sequence
  Synthetic DNA/RNA construct - RAG3A-673-saRNA sense sequence
cuugucacauguggaacggtt

SEQ ID NO: 1365 - DNA, 21 bp, Artificial Sequence
  Synthetic DNA/RNA construct - RAG3A-1001-saRNA sense sequence
cccuccuuccuaaucuuggtt

SEQ ID NO: 1366 - DNA, 21 bp, Artificial Sequence
  Synthetic DNA/RNA construct - RAG3A-1005-saRNA sense sequence
agcacccuccuuccuaauctt

SEQ ID NO: 1367 - DNA, 21 bp, Artificial Sequence
  Synthetic DNA/RNA construct - RAG3A-1064-saRNA sense sequence
ugccucccugucuccuguctt

SEQ ID NO: 1368 - DNA, 21 bp, Artificial Sequence
  Synthetic DNA/RNA construct - RAG3A-801-saRNA sense sequence
gacugggaagaaagcaaagtt

SEQ ID NO: 1369 - DNA, 21 bp, Artificial Sequence
  Synthetic DNA/RNA construct - RAG3A-486-saRNA sense sequence
aacccguccaaacccuuuctt

SEQ ID NO: 1370 - DNA, 21 bp, Artificial Sequence
  Synthetic DNA/RNA construct - RAG3A-555-saRNA sense sequence
accuaagcucaggcuuaactt

SEQ ID NO: 1371 - DNA, 21 bp, Artificial Sequence
  Synthetic DNA/RNA construct - RAG3A-854-saRNA sense sequence
ggaucaaggucaggccaggtt

SEQ ID NO: 1372 - DNA, 21 bp, Artificial Sequence
  Synthetic DNA/RNA construct - RAG3A-1267-saRNA sense sequence
guguuacccaggacagggatt

SEQ ID NO: 1373 - DNA, 21 bp, Artificial Sequence
  Synthetic DNA/RNA construct - RAG3A-1264-saRNA sense sequence
uuacccaggacagggaccutt

SEQ ID NO: 1374 - DNA, 21 bp, Artificial Sequence
  Synthetic DNA/RNA construct - RAG3A-513-saRNA sense sequence
augugccccgcaccugacatt

SEQ ID NO: 1375 - DNA, 21 bp, Artificial Sequence
  Synthetic DNA/RNA construct - RAG3A-1286-saRNA sense sequence
gaggucgcuccucauucuutt

SEQ ID NO: 1376 - DNA, 21 bp, Artificial Sequence
  Synthetic DNA/RNA construct - RAG3A-653-saRNA sense sequence
aggagauggaagacggagatt

SEQ ID NO: 1377 - DNA, 21 bp, Artificial Sequence
  Synthetic DNA/RNA construct - RAG3A-1014-saRNA sense sequence
agcuaucccagcacccucctt

SEQ ID NO: 1378 - DNA, 21 bp, Artificial Sequence
  Synthetic DNA/RNA construct - RAG3A-61-saRNA sense sequence
agagaaggcgucacuuccgtt

SEQ ID NO: 1379 - DNA, 21 bp, Artificial Sequence
  Synthetic DNA/RNA construct - RAG3A-666-saRNA sense sequence
cauguggaacgguaggagatt

SEQ ID NO: 1380 - DNA, 21 bp, Artificial Sequence
  Synthetic DNA/RNA construct - RAG3A-348-saRNA sense sequence
aagcaagcauccuguuggatt

SEQ ID NO: 1381 - DNA, 21 bp, Artificial Sequence
  Synthetic DNA/RNA construct - RAG3A-545-saRNA sense sequence
aggcuuaacccagugcacgtt

SEQ ID NO: 1382 - DNA, 21 bp, Artificial Sequence
  Synthetic DNA/RNA construct - RAG3A-584-saRNA sense sequence
ucuaagccacuggaccccatt

SEQ ID NO: 1383 - DNA, 21 bp, Artificial Sequence
  Synthetic DNA/RNA construct - RAG3A-1162-saRNA sense sequence
guccccucuugcuuucucatt

SEQ ID NO: 1384 - DNA, 21 bp, Artificial Sequence
  Synthetic DNA/RNA construct - RAG3A-308-saRNA sense sequence
ccagggaaaagaaauuuggtt

SEQ ID NO: 1385 - DNA, 21 bp, Artificial Sequence
  Synthetic DNA/RNA construct - RAG3A-158-saRNA sense sequence
cccauguccaggaaaagautt

SEQ ID NO: 1386 - DNA, 21 bp, Artificial Sequence
  Synthetic DNA/RNA construct - RAG3A-161-saRNA sense sequence
uugcccauguccaggaaaatt

SEQ ID NO: 1387 - DNA, 21 bp, Artificial Sequence
  Synthetic DNA/RNA construct - RAG3A-1253-saRNA sense sequence
agggaccuagccagaaacctt

SEQ ID NO: 1388 - DNA, 21 bp, Artificial Sequence
  Synthetic DNA/RNA construct - RAG3A-926-saRNA sense sequence
cagacaugcccauccuuggtt

SEQ ID NO: 1389 - DNA, 21 bp, Artificial Sequence
  Synthetic DNA/RNA construct - RAG3A-1191-saRNA sense sequence
uuguaacuuauccucaggctt

SEQ ID NO: 1390 - DNA, 21 bp, Artificial Sequence
  Synthetic DNA/RNA construct - RAG3A-697-saRNA sense sequence
ggacacaugggccugguuatt

SEQ ID NO: 1391 - DNA, 21 bp, Artificial Sequence
  Synthetic DNA/RNA construct - RAG3A-1224-saRNA sense sequence
ccccuucuguggagugacatt

SEQ ID NO: 1392 - DNA, 21 bp, Artificial Sequence
  Synthetic DNA/RNA construct - RAG3A-964-saRNA sense sequence
ggacgggaaaauuccaggatt

SEQ ID NO: 1393 - DNA, 21 bp, Artificial Sequence
  Synthetic DNA/RNA construct - RAG3A-791-saRNA sense sequence
aaagcaaaggagcuagagctt

SEQ ID NO: 1394 - DNA, 21 bp, Artificial Sequence
  Synthetic DNA/RNA construct - RAG3A-526-saRNA sense sequence
ugugcgcacauacaugugctt

SEQ ID NO: 1395 - DNA, 21 bp, Artificial Sequence
  Synthetic DNA/RNA construct - RAG3A-972-saRNA sense sequence
gucuggcuggacgggaaaatt

SEQ ID NO: 1396 - DNA, 21 bp, Artificial Sequence
  Synthetic DNA/RNA construct - RAG3A-289-saRNA sense sequence
auagccagggagugaaaactt

SEQ ID NO: 1397 - DNA, 21 bp, Artificial Sequence
  Synthetic DNA/RNA construct - RAG3A-255-saRNA sense sequence
caagaccucugugcuucuutt

SEQ ID NO: 1398 - DNA, 21 bp, Artificial Sequence
  Synthetic DNA/RNA construct - RAG3A-1000-saRNA sense sequence
ccuccuuccuaaucuugggtt

SEQ ID NO: 1399 - DNA, 21 bp, Artificial Sequence
  Synthetic DNA/RNA construct - RAG3A-694-saRNA sense sequence
cacaugggccugguuauuctt

SEQ ID NO: 1400 - DNA, 21 bp, Artificial Sequence
  Synthetic DNA/RNA construct - RAG3A-549-saRNA sense sequence
gcucaggcuuaacccagugtt

SEQ ID NO: 1401 - DNA, 21 bp, Artificial Sequence
  Synthetic DNA/RNA construct - RAG3A-1240-saRNA sense sequence
gaaaccggcagcauucccctt

SEQ ID NO: 1402 - DNA, 21 bp, Artificial Sequence
  Synthetic DNA/RNA construct - RAG3A-978-saRNA sense sequence
caucucgucuggcuggacgtt

SEQ ID NO: 1403 - DNA, 21 bp, Artificial Sequence
  Synthetic DNA/RNA construct - RAG3A-1290-saRNA sense sequence
cucugaggucgcuccucautt

SEQ ID NO: 1404 - DNA, 21 bp, Artificial Sequence
  Synthetic DNA/RNA construct - RAG3A-1006-saRNA sense sequence
cagcacccuccuuccuaautt

SEQ ID NO: 1405 - DNA, 21 bp, Artificial Sequence
  Synthetic DNA/RNA construct - RAG3A-1311-saRNA sense sequence
uagccugccucccuaccuutt

SEQ ID NO: 1406 - DNA, 21 bp, Artificial Sequence
  Synthetic DNA/RNA construct - RAG3A-42-saRNA sense sequence
ggggccuucaccagugucutt

SEQ ID NO: 1407 - DNA, 21 bp, Artificial Sequence
  Synthetic DNA/RNA construct - RAG3A-21-saRNA sense sequence
uggcucccuucucugauugtt

SEQ ID NO: 1408 - DNA, 21 bp, Artificial Sequence
  Synthetic DNA/RNA construct - RAG3A-59-saRNA sense sequence
agaaggcgucacuuccgggtt

SEQ ID NO: 1409 - DNA, 21 bp, Artificial Sequence
  Synthetic DNA/RNA construct - RAG3A-1187-saRNA sense sequence
aacuuauccucaggcgcautt

SEQ ID NO: 1410 - DNA, 21 bp, Artificial Sequence
  Synthetic DNA/RNA construct - RAG3A-576-saRNA sense sequence
acuggaccccagcagacgatt

SEQ ID NO: 1411 - DNA, 21 bp, Artificial Sequence
  Synthetic DNA/RNA construct - RAG3A-639-saRNA sense sequence
ggagacagaacaagcaaagtt

SEQ ID NO: 1412 - DNA, 21 bp, Artificial Sequence
  Synthetic DNA/RNA construct - RAG3A-815-saRNA sense sequence
uguggggagacugggacugtt

SEQ ID NO: 1413 - DNA, 21 bp, Artificial Sequence
  Synthetic DNA/RNA construct - RAG3A-957-saRNA sense sequence
aaaauuccaggaucuaggctt

SEQ ID NO: 1414 - DNA, 21 bp, Artificial Sequence
  Synthetic DNA/RNA construct - RAG3A-1217-saRNA sense sequence
uguggagugacaguaucuctt

SEQ ID NO: 1415 - DNA, 21 bp, Artificial Sequence
  Synthetic DNA/RNA construct - RAG3A-1165-saRNA sense sequence
acaguccccucuugcuuuctt

SEQ ID NO: 1416 - DNA, 21 bp, Artificial Sequence
  Synthetic DNA/RNA construct - RAG3A-1134-saRNA sense sequence
uucacccaagggacccucutt

SEQ ID NO: 1417 - DNA, 21 bp, Artificial Sequence
  Synthetic DNA/RNA construct - RAG3A-667-saRNA sense sequence
acauguggaacgguaggagtt

SEQ ID NO: 1418 - DNA, 21 bp, Artificial Sequence
  Synthetic DNA/RNA construct - RAG3A-1339-saRNA sense sequence
caaacccucaccacccucutt

SEQ ID NO: 1419 - DNA, 21 bp, Artificial Sequence
  Synthetic DNA/RNA construct - RAG3A-676-saRNA sense sequence
ccucuugucacauguggaatt

SEQ ID NO: 1420 - DNA, 21 bp, Artificial Sequence
  Synthetic DNA/RNA construct - RAG3A-153-saRNA sense sequence
guccaggaaaagauggauctt

SEQ ID NO: 1421 - DNA, 21 bp, Artificial Sequence
  Synthetic DNA/RNA construct - RAG3A-320-saRNA sense sequence
cagguaugauguccagggatt

SEQ ID NO: 1422 - DNA, 21 bp, Artificial Sequence
  Synthetic DNA/RNA construct - RAG3A-1010-saRNA sense sequence
aucccagcacccuccuucctt

SEQ ID NO: 1423 - DNA, 21 bp, Artificial Sequence
  Synthetic DNA/RNA construct - RAG3A-999-saRNA sense sequence
cuccuuccuaaucuugggatt

SEQ ID NO: 1424 - DNA, 21 bp, Artificial Sequence
  Synthetic DNA/RNA construct - RAG3A-567-saRNA sense sequence
cagcagacgagcaccuaagtt

SEQ ID NO: 1425 - DNA, 21 bp, Artificial Sequence
  Synthetic DNA/RNA construct - RAG3A-862-saRNA sense sequence
ggaugaugggaucaagguctt

SEQ ID NO: 1426 - DNA, 21 bp, Artificial Sequence
  Synthetic DNA/RNA construct - RAG3A-460-saRNA sense sequence
caccaacccauaacaggagtt

SEQ ID NO: 1427 - DNA, 21 bp, Artificial Sequence
  Synthetic DNA/RNA construct - RAG3A-921-saRNA sense sequence
augcccauccuuggggaggtt

SEQ ID NO: 1428 - DNA, 21 bp, Artificial Sequence
  Synthetic DNA/RNA construct - RAG3A-704-saRNA sense sequence
aagacagggacacaugggctt

SEQ ID NO: 1429 - DNA, 21 bp, Artificial Sequence
  Synthetic DNA/RNA construct - RAG3A-233-saRNA sense sequence
cagcaacacaaauguccugtt

SEQ ID NO: 1430 - DNA, 21 bp, Artificial Sequence
  Synthetic DNA/RNA construct - RAG3A-1203-saRNA sense sequence
aucucccucucauuguaactt

SEQ ID NO: 1431 - DNA, 21 bp, Artificial Sequence
  Synthetic DNA/RNA construct - RAG3A-247-saRNA sense sequence
cugugcuucuuccccagcatt

SEQ ID NO: 1432 - DNA, 21 bp, Artificial Sequence
  Synthetic DNA/RNA construct - RAG3A-721-saRNA sense sequence
cagcagcugucuuuccuaatt

SEQ ID NO: 1433 - DNA, 21 bp, Artificial Sequence
  Synthetic DNA/RNA construct - RAG3A-391-saRNA sense sequence
acaagauaggacucccuagtt

SEQ ID NO: 1434 - DNA, 21 bp, Artificial Sequence
  Synthetic DNA/RNA construct - RAG3A-159-saRNA sense sequence
gcccauguccaggaaaagatt

SEQ ID NO: 1435 - DNA, 21 bp, Artificial Sequence
  Synthetic DNA/RNA construct - RAG3A-437-saRNA sense sequence
cucucaugugggcaauauctt

SEQ ID NO: 1436 - DNA, 21 bp, Artificial Sequence
  Synthetic DNA/RNA construct - RAG3A-29-saRNA sense sequence
gugucugguggcucccuuctt

SEQ ID NO: 1437 - DNA, 21 bp, Artificial Sequence
  Synthetic DNA/RNA construct - RAG3A-63-saRNA sense sequence
gaagagaaggcgucacuuctt

SEQ ID NO: 1438 - DNA, 21 bp, Artificial Sequence
  Synthetic DNA/RNA construct - RAG3A-996-saRNA sense sequence
cuuccuaaucuugggagactt

SEQ ID NO: 1439 - DNA, 21 bp, Artificial Sequence
  Synthetic DNA/RNA construct - RAG3A-1083-saRNA sense sequence
uugguucccuggucaugcctt

SEQ ID NO: 1440 - DNA, 21 bp, Artificial Sequence
  Synthetic DNA/RNA construct - RAG3A-1128-saRNA sense sequence
caagggacccucugccucutt

SEQ ID NO: 1441 - DNA, 21 bp, Artificial Sequence
  Synthetic DNA/RNA construct - RAG3A-1094-saRNA sense sequence
ccuccuuucucuugguucctt

SEQ ID NO: 1442 - DNA, 21 bp, Artificial Sequence
  Synthetic DNA/RNA construct - RAG3A-771-saRNA sense sequence
agggccaaaggaaaaggggtt

SEQ ID NO: 1443 - DNA, 21 bp, Artificial Sequence
  Synthetic DNA/RNA construct - RAG3A-287-saRNA sense sequence
agccagggagugaaaaccctt

SEQ ID NO: 1444 - DNA, 21 bp, Artificial Sequence
  Synthetic DNA/RNA construct - RAG3A-981-saRNA sense sequence
agacaucucgucuggcuggtt

SEQ ID NO: 1445 - DNA, 21 bp, Artificial Sequence
  Synthetic DNA/RNA construct - RAG3A-23-saRNA sense sequence
gguggcucccuucucugautt

SEQ ID NO: 1446 - DNA, 21 bp, Artificial Sequence
  Synthetic DNA/RNA construct - RAG3A-1072-saRNA sense sequence
gucaugccugccucccugutt

SEQ ID NO: 1447 - DNA, 21 bp, Artificial Sequence
  Synthetic DNA/RNA construct - RAG3A-415-saRNA sense sequence
guucccacuucgaaaggggtt

SEQ ID NO: 1448 - DNA, 21 bp, Artificial Sequence
  Synthetic DNA/RNA construct - RAG3A-1287-saRNA sense sequence
ugaggucgcuccucauucutt

SEQ ID NO: 1449 - DNA, 21 bp, Artificial Sequence
  Synthetic DNA/RNA construct - RAG3A-1201-saRNA sense sequence
cucccucucauuguaacuutt

SEQ ID NO: 1450 - DNA, 21 bp, Artificial Sequence
  Synthetic DNA/RNA construct - RAG3A-772-saRNA sense sequence
cagggccaaaggaaaagggtt

SEQ ID NO: 1451 - DNA, 21 bp, Artificial Sequence
  Synthetic DNA/RNA construct - RAG3A-1249-saRNA sense sequence
accuagccagaaaccggcatt

SEQ ID NO: 1452 - DNA, 21 bp, Artificial Sequence
  Synthetic DNA/RNA construct - RAG3A-546-saRNA sense sequence
caggcuuaacccagugcactt

SEQ ID NO: 1453 - DNA, 21 bp, Artificial Sequence
  Synthetic DNA/RNA construct - RAG3A-160-saRNA sense sequence
ugcccauguccaggaaaagtt

SEQ ID NO: 1454 - DNA, 21 bp, Artificial Sequence
  Synthetic DNA/RNA construct - RAG3A-64-saRNA sense sequence
ggaagagaaggcgucacuutt

SEQ ID NO: 1455 - DNA, 21 bp, Artificial Sequence
  Synthetic DNA/RNA construct - RAG3A-1002-saRNA sense sequence
acccuccuuccuaaucuugtt

SEQ ID NO: 1456 - DNA, 21 bp, Artificial Sequence
  Synthetic DNA/RNA construct - RAG3A-1288-saRNA sense sequence
cugaggucgcuccucauuctt

SEQ ID NO: 1457 - DNA, 21 bp, Artificial Sequence
  Synthetic DNA/RNA construct - RAG3A-891-saRNA sense sequence
agagccugaggaaguucugtt

SEQ ID NO: 1458 - DNA, 21 bp, Artificial Sequence
  Synthetic DNA/RNA construct - RAG3A-231-saRNA sense sequence
gcaacacaaauguccugcctt

SEQ ID NO: 1459 - DNA, 21 bp, Artificial Sequence
  Synthetic DNA/RNA construct - RAG3A-669-saRNA sense sequence
ucacauguggaacgguaggtt

SEQ ID NO: 1460 - DNA, 21 bp, Artificial Sequence
  Synthetic DNA/RNA construct - RAG3A-1009-saRNA
ucccagcacccuccuuccutt

SEQ ID NO: 1461 - DNA, 21 bp, Artificial Sequence
  Synthetic DNA/RNA construct - RAG3A-656-saRNA sense sequence
gguaggagauggaagacggtt

SEQ ID NO: 1462 - DNA, 21 bp, Artificial Sequence
  Synthetic DNA/RNA construct - RAG3A-229-saRNA sense sequence
aacacaaauguccugccagtt

SEQ ID NO: 1463 - DNA, 21 bp, Artificial Sequence
  Synthetic DNA/RNA construct - RAG3A-484-saRNA sense sequence
cccguccaaacccuuuccctt

SEQ ID NO: 1464 - DNA, 21 bp, Artificial Sequence
  Synthetic DNA/RNA construct - RAG3A-799-saRNA sense sequence
cugggaagaaagcaaaggatt

SEQ ID NO: 1465 - DNA, 21 bp, Artificial Sequence
  Synthetic DNA/RNA construct - RAG3A-296-saRNA sense sequence
aauuuggauagccagggagtt

SEQ ID NO: 1466 - DNA, 21 bp, Artificial Sequence
  Synthetic DNA/RNA construct - RAG3A-1183-saRNA sense sequence
uauccucaggcgcauucgatt

SEQ ID NO: 1467 - DNA, 21 bp, Artificial Sequence
  Synthetic DNA/RNA construct - RAG3A-380-saRNA sense sequence
cucccuaggggauuacagatt

SEQ ID NO: 1468 - DNA, 21 bp, Artificial Sequence
  Synthetic DNA/RNA construct - RAG3A-157-saRNA sense sequence
ccauguccaggaaaagaugtt

SEQ ID NO: 1469 - DNA, 21 bp, Artificial Sequence
  Synthetic DNA/RNA construct - RAG3A-1185-saRNA sense sequence
cuuauccucaggcgcauuctt

SEQ ID NO: 1470 - DNA, 21 bp, Artificial Sequence
  Synthetic DNA/RNA construct - RAG3A-1244-saRNA sense sequence
gccagaaaccggcagcauutt

SEQ ID NO: 1471 - DNA, 21 bp, Artificial Sequence
  Synthetic DNA/RNA construct - RAG3A-588-saRNA sense sequence
gccaucuaagccacuggactt

SEQ ID NO: 1472 - DNA, 21 bp, Artificial Sequence
  Synthetic DNA/RNA construct - RAG3A-436-saRNA sense sequence
ucucaugugggcaauaucctt

SEQ ID NO: 1473 - DNA, 21 bp, Artificial Sequence
  Synthetic DNA/RNA construct - RAG3A-1029-saRNA sense sequence
acccacuauccucccagcutt

SEQ ID NO: 1474 - DNA, 21 bp, Artificial Sequence
  Synthetic DNA/RNA construct - RAG3A-28-saRNA sense sequence
ugucugguggcucccuucutt

SEQ ID NO: 1475 - DNA, 21 bp, Artificial Sequence
  Synthetic DNA/RNA construct - RAG3A-234-saRNA sense sequence
ccagcaacacaaauguccutt

SEQ ID NO: 1476 - DNA, 21 bp, Artificial Sequence
  Synthetic DNA/RNA construct - RAG3A-249-saRNA sense sequence
cucugugcuucuuccccagtt

SEQ ID NO: 1477 - DNA, 21 bp, Artificial Sequence
  Synthetic DNA/RNA construct - RAG3A-775-saRNA sense sequence
agccagggccaaaggaaaatt

SEQ ID NO: 1478 - DNA, 21 bp, Artificial Sequence
  Synthetic DNA/RNA construct - RAG3A-951-saRNA sense sequence
ccaggaucuaggccacacutt

SEQ ID NO: 1479 - DNA, 21 bp, Artificial Sequence
  Synthetic DNA/RNA construct - RAG3A-861-saRNA sense sequence
gaugaugggaucaaggucatt

SEQ ID NO: 1480 - DNA, 21 bp, Artificial Sequence
  Synthetic DNA/RNA construct - RAG3A-259-saRNA sense sequence
uaaacaagaccucugugcutt

SEQ ID NO: 1481 - DNA, 21 bp, Artificial Sequence
  Synthetic DNA/RNA construct - RAG3A-394-saRNA sense sequence
augacaagauaggacuccctt

SEQ ID NO: 1482 - DNA, 21 bp, Artificial Sequence
  Synthetic DNA/RNA construct - RAG3A-534-saRNA sense sequence
agugcacgugugcgcacautt

SEQ ID NO: 1483 - DNA, 21 bp, Artificial Sequence
  Synthetic DNA/RNA construct - RAG3A-316-saRNA sense sequence
uaugauguccagggaaaagtt

SEQ ID NO: 1484 - DNA, 21 bp, Artificial Sequence
  Synthetic DNA/RNA construct - RAG3A-560-saRNA sense sequence
cgagcaccuaagcucaggctt

SEQ ID NO: 1485 - DNA, 21 bp, Artificial Sequence
  Synthetic DNA/RNA construct - RAG3A-1105-saRNA sense sequence
cccacucccagccuccuuutt

SEQ ID NO: 1486 - DNA, 21 bp, Artificial Sequence
  Synthetic DNA/RNA construct - RAG3A-709-saRNA sense sequence
uuccuaagacagggacacatt

SEQ ID NO: 1487 - DNA, 21 bp, Artificial Sequence
  Synthetic DNA/RNA construct - RAG3A-948-saRNA sense sequence
ggaucuaggccacacuucutt

SEQ ID NO: 1488 - DNA, 21 bp, Artificial Sequence
  Synthetic DNA/RNA construct - RAG3A-998-saRNA sense sequence
uccuuccuaaucuugggagtt

SEQ ID NO: 1489 - DNA, 21 bp, Artificial Sequence
  Synthetic DNA/RNA construct - RAG3A-387-saRNA sense sequence
gauaggacucccuaggggatt

SEQ ID NO: 1490 - DNA, 21 bp, Artificial Sequence
  Synthetic DNA/RNA construct - RAG3A-889-saRNA antisense sequence
cccagaacuuccucaggcutt

SEQ ID NO: 1491 - DNA, 21 bp, Artificial Sequence
  Synthetic DNA/RNA construct - RAG3A-723-saRNA antisense sequence
aggaaagacagcugcuggatt

SEQ ID NO: 1492 - DNA, 21 bp, Artificial Sequence
  Synthetic DNA/RNA construct - RAG3A-1069-saRNA antisense sequence
gagacagggaggcaggcautt

SEQ ID NO: 1493 - DNA, 21 bp, Artificial Sequence
  Synthetic DNA/RNA construct - RAG3A-541-saRNA antisense sequence
cacacgugcacuggguuaatt

SEQ ID NO: 1494 - DNA, 21 bp, Artificial Sequence
  Synthetic DNA/RNA construct - RAG3A-480-saRNA antisense sequence
uauggggaaaggguuuggatt

SEQ ID NO: 1495 - DNA, 21 bp, Artificial Sequence
  Synthetic DNA/RNA construct - RAG3A-980-saRNA antisense sequence
uccagccagacgagauguctt

SEQ ID NO: 1496 - DNA, 21 bp, Artificial Sequence
  Synthetic DNA/RNA construct - RAG3A-462-saRNA antisense sequence
ccuguuauggguugguguutt

SEQ ID NO: 1497 - DNA, 21 bp, Artificial Sequence
  Synthetic DNA/RNA construct - RAG3A-787-saRNA antisense sequence
ccuggcucuagcuccuuugtt

SEQ ID NO: 1498 - DNA, 21 bp, Artificial Sequence
  Synthetic DNA/RNA construct - RAG3A-232-saRNA antisense sequence
gcaggacauuuguguugcutt

SEQ ID NO: 1499 - DNA, 21 bp, Artificial Sequence
  Synthetic DNA/RNA construct - RAG3A-727-saRNA antisense sequence
aagacagcugcuggaccuctt

SEQ ID NO: 1500 - DNA, 21 bp, Artificial Sequence
  Synthetic DNA/RNA construct - RAG3A-542-saRNA antisense sequence
acacgugcacuggguuaagtt

SEQ ID NO: 1501 - DNA, 21 bp, Artificial Sequence
  Synthetic DNA/RNA construct - RAG3A-748-saRNA antisense sequence
ccgcaaauaccucccugcutt

SEQ ID NO: 1502 - DNA, 21 bp, Artificial Sequence
  Synthetic DNA/RNA construct - RAG3A-540-saRNA antisense sequence
gcacacgugcacuggguuatt

SEQ ID NO: 1503 - DNA, 21 bp, Artificial Sequence
  Synthetic DNA/RNA construct - RAG3A-1219-saRNA antisense sequence
gauacugucacuccacagatt

SEQ ID NO: 1504 - DNA, 21 bp, Artificial Sequence
  Synthetic DNA/RNA construct - RAG3A-487-saRNA antisense sequence
aaaggguuuggacggguugtt

SEQ ID NO: 1505 - DNA, 21 bp, Artificial Sequence
  Synthetic DNA/RNA construct - RAG3A-559-saRNA antisense sequence
agccugagcuuaggugcuctt

SEQ ID NO: 1506 - DNA, 21 bp, Artificial Sequence
  Synthetic DNA/RNA construct - RAG3A-1308-saRNA antisense sequence
gagaagguagggaggcaggtt

SEQ ID NO: 1507 - DNA, 21 bp, Artificial Sequence
  Synthetic DNA/RNA construct - RAG3A-24-saRNA antisense sequence
ucagagaagggagccaccatt

SEQ ID NO: 1508 - DNA, 21 bp, Artificial Sequence
  Synthetic DNA/RNA construct - RAG3A-1169-saRNA antisense sequence
gcaagaggggacugucgaatt

SEQ ID NO: 1509 - DNA, 21 bp, Artificial Sequence
  Synthetic DNA/RNA construct - RAG3A-1256-saRNA antisense sequence
uucuggcuaggucccuguctt

SEQ ID NO: 1510 - DNA, 21 bp, Artificial Sequence
  Synthetic DNA/RNA construct - RAG3A-522-saRNA antisense sequence
cggggcacauguaugugcgtt

SEQ ID NO: 1511 - DNA, 21 bp, Artificial Sequence
  Synthetic DNA/RNA construct - RAG3A-851-saRNA antisense sequence
cuuccuggccugaccuugatt

SEQ ID NO: 1512 - DNA, 21 bp, Artificial Sequence
  Synthetic DNA/RNA construct - RAG3A-1096-saRNA antisense sequence
aaccaagagaaaggaggcutt

SEQ ID NO: 1513 - DNA, 21 bp, Artificial Sequence
  Synthetic DNA/RNA construct - RAG3A-227-saRNA antisense sequence
aucuggcaggacauuugugtt

SEQ ID NO: 1514 - DNA, 21 bp, Artificial Sequence
  Synthetic DNA/RNA construct - RAG3A-1178-saRNA antisense sequence
gacugucgaaugcgccugatt

SEQ ID NO: 1515 - DNA, 21 bp, Artificial Sequence
  Synthetic DNA/RNA construct - RAG3A-544-saRNA antisense sequence
acgugcacuggguuaagcctt

SEQ ID NO: 1516 - DNA, 21 bp, Artificial Sequence
  Synthetic DNA/RNA construct - RAG3A-836-saRNA antisense sequence
cucuguccucaggggcuuctt

SEQ ID NO: 1517 - DNA, 21 bp, Artificial Sequence
  Synthetic DNA/RNA construct - RAG3A-569-saRNA antisense sequence
uaggugcucgucugcugggtt

SEQ ID NO: 1518 - DNA, 21 bp, Artificial Sequence
  Synthetic DNA/RNA construct - RAG3A-1320-saRNA antisense sequence
aggcaggcuaggcagugagtt

SEQ ID NO: 1519 - DNA, 21 bp, Artificial Sequence
  Synthetic DNA/RNA construct - RAG3A-1319-saRNA antisense sequence
gaggcaggcuaggcagugatt

SEQ ID NO: 1520 - DNA, 21 bp, Artificial Sequence
  Synthetic DNA/RNA construct - RAG3A-290-saRNA antisense sequence
uuuucacucccuggcuauctt

SEQ ID NO: 1521 - DNA, 21 bp, Artificial Sequence
  RAG3A-1074-saRNA antisense sequence
agggaggcaggcaugaccatt

SEQ ID NO: 1522 - DNA, 21 bp, Artificial Sequence
  Synthetic DNA/RNA construct - RAG3A-279-saRNA antisense sequence
gauuggugggguuuucacutt

SEQ ID NO: 1523 - DNA, 21 bp, Artificial Sequence
  Synthetic DNA/RNA construct - RAG3A-521-saRNA antisense sequence
gcggggcacauguaugugctt

SEQ ID NO: 1524 - DNA, 21 bp, Artificial Sequence
  Synthetic DNA/RNA construct - RAG3A-25-saRNA antisense sequence
cagagaagggagccaccagtt

SEQ ID NO: 1525 - DNA, 21 bp, Artificial Sequence
  Synthetic DNA/RNA construct - RAG3A-62-saRNA antisense sequence
ggaagugacgccuucucuutt

SEQ ID NO: 1526 - DNA, 21 bp, Artificial Sequence
  Synthetic DNA/RNA construct - RAG3A-852-saRNA antisense sequence
uuccuggccugaccuugautt

SEQ ID NO: 1527 - DNA, 21 bp, Artificial Sequence
  Synthetic DNA/RNA construct - RAG3A-931-saRNA antisense sequence
gaugggcaugucugcugagtt

SEQ ID NO: 1528 - DNA, 21 bp, Artificial Sequence
  Synthetic DNA/RNA construct - RAG3A-892-saRNA antisense sequence
agaacuuccucaggcucuctt

SEQ ID NO: 1529 - DNA, 21 bp, Artificial Sequence
  Synthetic DNA/RNA construct - RAG3A-235-saRNA antisense sequence
ggacauuuguguugcugggtt

SEQ ID NO: 1530 - DNA, 21 bp, Artificial Sequence
  Synthetic DNA/RNA construct - RAG3A-1246-saRNA antisense sequence
ugcugccgguuucuggcuatt

SEQ ID NO: 1531 - DNA, 21 bp, Artificial Sequence
  Synthetic DNA/RNA construct - RAG3A-19-saRNA antisense sequence
cccaaucagagaagggagctt

SEQ ID NO: 1532 - DNA, 21 bp, Artificial Sequence
  Synthetic DNA/RNA construct - RAG3A-1322-saRNA antisense sequence
gcaggcuaggcagugagagtt

SEQ ID NO: 1533 - DNA, 21 bp, Artificial Sequence
  Synthetic DNA/RNA construct - RAG3A-795-saRNA antisense sequence
uagcuccuuugcuuucuuctt

SEQ ID NO: 1534 - DNA, 21 bp, Artificial Sequence
  Synthetic DNA/RNA construct - RAG3A-515-saRNA antisense sequence
ucaggugcggggcacaugutt

SEQ ID NO: 1535 - DNA, 21 bp, Artificial Sequence
  Synthetic DNA/RNA construct - RAG3A-128-saRNA antisense sequence
accacggagaagauuuggatt

SEQ ID NO: 1536 - DNA, 21 bp, Artificial Sequence
  Synthetic DNA/RNA construct - RAG3A-661-saRNA antisense sequence
uuccaucuccuaccguucctt

SEQ ID NO: 1537 - DNA, 21 bp, Artificial Sequence
  Synthetic DNA/RNA construct - RAG3A-125-saRNA antisense sequence
cacaccacggagaagauuutt

SEQ ID NO: 1538 - DNA, 21 bp, Artificial Sequence
  Synthetic DNA/RNA construct - RAG3A-1241-saRNA antisense sequence
gggaaugcugccgguuucutt

SEQ ID NO: 1539 - DNA, 21 bp, Artificial Sequence
  Synthetic DNA/RNA construct - RAG3A-463-saRNA antisense sequence
cuguuauggguugguguuatt

SEQ ID NO: 1540 - DNA, 21 bp, Artificial Sequence
  Synthetic DNA/RNA construct - RAG3A-434-saRNA antisense sequence
acggauauugcccacaugatt

SEQ ID NO: 1541 - DNA, 21 bp, Artificial Sequence
  Synthetic DNA/RNA construct - RAG3A-520-saRNA antisense sequence
ugcggggcacauguaugugtt

SEQ ID NO: 1542 - DNA, 21 bp, Artificial Sequence
  Synthetic DNA/RNA construct - RAG3A-499-saRNA antisense sequence
cggguugaguggacugucatt

SEQ ID NO: 1543 - DNA, 21 bp, Artificial Sequence
  Synthetic DNA/RNA construct - RAG3A-668-saRNA antisense sequence
uccuaccguuccacaugugtt

SEQ ID NO: 1544 - DNA, 21 bp, Artificial Sequence
  Synthetic DNA/RNA construct - RAG3A-591-saRNA antisense sequence
caguggcuuagauggcuactt

SEQ ID NO: 1545 - DNA, 21 bp, Artificial Sequence
  Synthetic DNA/RNA construct - RAG3A-1209-saRNA antisense sequence
augagagggagauacuguctt

SEQ ID NO: 1546 - DNA, 21 bp, Artificial Sequence
  Synthetic DNA/RNA construct - RAG3A-1257-saRNA antisense sequence
ucuggcuaggucccugucctt

SEQ ID NO: 1547 - DNA, 21 bp, Artificial Sequence
  Synthetic DNA/RNA construct - RAG3A-1170-saRNA antisense sequence
caagaggggacugucgaautt

SEQ ID NO: 1548 - DNA, 21 bp, Artificial Sequence
  Synthetic DNA/RNA construct - RAG3A-39-saRNA antisense sequence
accagacacuggugaaggctt

SEQ ID NO: 1549 - DNA, 21 bp, Artificial Sequence
  Synthetic DNA/RNA construct - RAG3A-431-saRNA antisense sequence
aacacggauauugcccacatt

SEQ ID NO: 1550 - DNA, 21 bp, Artificial Sequence
  Synthetic DNA/RNA construct - RAG3A-1080-saRNA antisense sequence
gcaggcaugaccagggaactt

SEQ ID NO: 1551 - DNA, 21 bp, Artificial Sequence
  Synthetic DNA/RNA construct - RAG3A-1266-saRNA antisense sequence
gucccuguccuggguaacatt

SEQ ID NO: 1552 - DNA, 21 bp, Artificial Sequence
  Synthetic DNA/RNA construct - RAG3A-1202-saRNA antisense sequence
aguuacaaugagagggagatt

SEQ ID NO: 1553 - DNA, 21 bp, Artificial Sequence
  Synthetic DNA/RNA construct - RAG3A-979-saRNA antisense sequence
guccagccagacgagaugutt

SEQ ID NO: 1554 - DNA, 21 bp, Artificial Sequence
  Synthetic DNA/RNA construct - RAG3A-343-saRNA antisense sequence
ugaaauccaacaggaugcutt

SEQ ID NO: 1555 - DNA, 21 bp, Artificial Sequence
  Synthetic DNA/RNA construct - RAG3A-430-saRNA antisense sequence
gaacacggauauugcccactt

SEQ ID NO: 1556 - DNA, 21 bp, Artificial Sequence
  Synthetic DNA/RNA construct - RAG3A-319-saRNA antisense sequence
uucccuggacaucauaccutt

SEQ ID NO: 1557 - DNA, 21 bp, Artificial Sequence
  Synthetic DNA/RNA construct - RAG3A-612-saRNA antisense sequence
acacagaccucugugcccatt

SEQ ID NO: 1558 - DNA, 21 bp, Artificial Sequence
  Synthetic DNA/RNA construct - RAG3A-454-saRNA antisense sequence
agaaaucuccuguuaugggtt

SEQ ID NO: 1559 - DNA, 21 bp, Artificial Sequence
  Synthetic DNA/RNA construct - RAG3A-1312-saRNA antisense sequence
agguagggaggcaggcuagtt

SEQ ID NO: 1560 - DNA, 21 bp, Artificial Sequence
  Synthetic DNA/RNA construct - RAG3A-342-saRNA antisense sequence
cugaaauccaacaggaugctt

SEQ ID NO: 1561 - DNA, 21 bp, Artificial Sequence
  Synthetic DNA/RNA construct - RAG3A-1118-saRNA antisense sequence
agugggcuggagaggcagatt

SEQ ID NO: 1562 - DNA, 21 bp, Artificial Sequence
  Synthetic DNA/RNA construct - RAG3A-970-saRNA antisense sequence
aauuuucccguccagccagtt

SEQ ID NO: 1563 - DNA, 21 bp, Artificial Sequence
  Synthetic DNA/RNA construct - RAG3A-1133-saRNA antisense sequence
cagagggucccuugggugatt

SEQ ID NO: 1564 - DNA, 21 bp, Artificial Sequence
  Synthetic DNA/RNA construct - RAG3A-689-saRNA antisense sequence
aagaggaauaaccaggccctt

SEQ ID NO: 1565 - DNA, 21 bp, Artificial Sequence
  Synthetic DNA/RNA construct - RAG3A-1321-saRNA antisense sequence
ggcaggcuaggcagugagatt

SEQ ID NO: 1566 - DNA, 21 bp, Artificial Sequence
  Synthetic DNA/RNA construct - RAG3A-662-saRNA antisense sequence
uccaucuccuaccguuccatt

SEQ ID NO: 1567 - DNA, 21 bp, Artificial Sequence
  Synthetic DNA/RNA construct - RAG3A-817-saRNA antisense sequence
gucccagucuccccacagutt

SEQ ID NO: 1568 - DNA, 21 bp, Artificial Sequence
  Synthetic DNA/RNA construct - RAG3A-1242-saRNA antisense sequence
ggaaugcugccgguuucugtt

SEQ ID NO: 1569 - DNA, 21 bp, Artificial Sequence
  Synthetic DNA/RNA construct - RAG3A-278-saRNA antisense sequence
agauuggugggguuuucactt

SEQ ID NO: 1570 - DNA, 21 bp, Artificial Sequence
  Synthetic DNA/RNA construct - RAG3A-1210-saRNA antisense sequence
ugagagggagauacugucatt

SEQ ID NO: 1571 - DNA, 21 bp, Artificial Sequence
  Synthetic DNA/RNA construct - RAG3A-1084-saRNA antisense sequence
gcaugaccagggaaccaagtt

SEQ ID NO: 1572 - DNA, 21 bp, Artificial Sequence
  Synthetic DNA/RNA construct - RAG3A-1166-saRNA antisense sequence
aaagcaagaggggacuguctt

SEQ ID NO: 1573 - DNA, 21 bp, Artificial Sequence
  Synthetic DNA/RNA construct - RAG3A-982-saRNA antisense sequence
cagccagacgagaugucuctt

SEQ ID NO: 1574 - DNA, 21 bp, Artificial Sequence
  Synthetic DNA/RNA construct - RAG3A-171-saRNA antisense sequence
caugggcaaccugggaccutt

SEQ ID NO: 1575 - DNA, 21 bp, Artificial Sequence
  Synthetic DNA/RNA construct - RAG3A-256-saRNA antisense sequence
agaagcacagaggucuugutt

SEQ ID NO: 1576 - DNA, 21 bp, Artificial Sequence
  Synthetic DNA/RNA construct - RAG3A-563-saRNA antisense sequence
ugagcuuaggugcucgucutt

SEQ ID NO: 1577 - DNA, 21 bp, Artificial Sequence
  Synthetic DNA/RNA construct - RAG3A-1176-saRNA antisense sequence
gggacugucgaaugcgccutt

SEQ ID NO: 1578 - DNA, 21 bp, Artificial Sequence
  Synthetic DNA/RNA construct - RAG3A-707-saRNA antisense sequence
caugugucccugucuuaggtt

SEQ ID NO: 1579 - DNA, 21 bp, Artificial Sequence
  Synthetic DNA/RNA construct - RAG3A-561-saRNA antisense sequence
ccugagcuuaggugcucgutt

SEQ ID NO: 1580 - DNA, 21 bp, Artificial Sequence
  Synthetic DNA/RNA construct - RAG3A-504-saRNA antisense sequence
ugaguggacugucaggugctt

SEQ ID NO: 1581 - DNA, 21 bp, Artificial Sequence
  Synthetic DNA/RNA construct - RAG3A-195-saRNA antisense sequence
gggacaggagcagaaguuutt

SEQ ID NO: 1582 - DNA, 21 bp, Artificial Sequence
  Synthetic DNA/RNA construct - RAG3A-850-saRNA antisense sequence
gcuuccuggccugaccuugtt

SEQ ID NO: 1583 - DNA, 21 bp, Artificial Sequence
  Synthetic DNA/RNA construct - RAG3A-388-saRNA antisense sequence
ccccuagggaguccuaucutt

SEQ ID NO: 1584 - DNA, 21 bp, Artificial Sequence
  Synthetic DNA/RNA construct - RAG3A-1079-saRNA antisense sequence
ggcaggcaugaccagggaatt

SEQ ID NO: 1585 - DNA, 21 bp, Artificial Sequence
  Synthetic DNA/RNA construct - RAG3A-1263-saRNA antisense sequence
uaggucccuguccuggguatt

SEQ ID NO: 1586 - DNA, 21 bp, Artificial Sequence
  Synthetic DNA/RNA construct - RAG3A-531-saRNA antisense sequence
uguaugugcgcacacgugctt

SEQ ID NO: 1587 - DNA, 21 bp, Artificial Sequence
  Synthetic DNA/RNA construct - RAG3A-254-saRNA antisense sequence
gaagaagcacagaggucuutt

SEQ ID NO: 1588 - DNA, 21 bp, Artificial Sequence
  Synthetic DNA/RNA construct - RAG3A-421-saRNA antisense sequence
ucgaagugggaacacggautt

SEQ ID NO: 1589 - DNA, 21 bp, Artificial Sequence
  Synthetic DNA/RNA construct - RAG3A-473-saRNA antisense sequence
uugguguuauggggaaaggtt

SEQ ID NO: 1590 - DNA, 21 bp, Artificial Sequence
  Synthetic DNA/RNA construct - RAG3A-292-saRNA antisense sequence
uucacucccuggcuauccatt

SEQ ID NO: 1591 - DNA, 21 bp, Artificial Sequence
  Synthetic DNA/RNA construct - RAG3A-283-saRNA antisense sequence
ggugggguuuucacucccutt

SEQ ID NO: 1592 - DNA, 21 bp, Artificial Sequence
  Synthetic DNA/RNA construct - RAG3A-786-saRNA antisense sequence
cccuggcucuagcuccuuutt

SEQ ID NO: 1593 - DNA, 21 bp, Artificial Sequence
  Synthetic DNA/RNA construct - RAG3A-582-saRNA antisense sequence
gcugggguccaguggcuuatt

SEQ ID NO: 1594 - DNA, 21 bp, Artificial Sequence
  Synthetic DNA/RNA construct - RAG3A-933-saRNA antisense sequence
ugggcaugucugcugagaatt

SEQ ID NO: 1595 - DNA, 21 bp, Artificial Sequence
  Synthetic DNA/RNA construct - RAG3A-472-saRNA antisense sequence
guugguguuauggggaaagtt

SEQ ID NO: 1596 - DNA, 21 bp, Artificial Sequence
  Synthetic DNA/RNA construct - RAG3A-646-saRNA antisense sequence
uguucugucuccgucuucctt

SEQ ID NO: 1597 - DNA, 21 bp, Artificial Sequence
  Synthetic DNA/RNA construct - RAG3A-389-saRNA antisense sequence
cccuagggaguccuaucuutt

SEQ ID NO: 1598 - DNA, 21 bp, Artificial Sequence
  Synthetic DNA/RNA construct - RAG3A-971-saRNA antisense sequence
auuuucccguccagccagatt

SEQ ID NO: 1599 - DNA, 21 bp, Artificial Sequence
  Synthetic DNA/RNA construct - RAG3A-751-saRNA antisense sequence
caaauaccucccugcuggctt

SEQ ID NO: 1600 - DNA, 21 bp, Artificial Sequence
  Synthetic DNA/RNA construct - RAG3A-299-saRNA antisense sequence
ccuggcuauccaaauuucutt

SEQ ID NO: 1601 - DNA, 21 bp, Artificial Sequence
  Synthetic DNA/RNA construct - RAG3A-750-saRNA antisense sequence
gcaaauaccucccugcuggtt

SEQ ID NO: 1602 - DNA, 21 bp, Artificial Sequence
  Synthetic DNA/RNA construct - RAG3A-671-saRNA antisense sequence
uaccguuccacaugugacatt

SEQ ID NO: 1603 - DNA, 21 bp, Artificial Sequence
  Synthetic DNA/RNA construct - RAG3A-749-saRNA antisense sequence
cgcaaauaccucccugcugtt

SEQ ID NO: 1604 - DNA, 21 bp, Artificial Sequence
  Synthetic DNA/RNA construct - RAG3A-547-saRNA antisense sequence
ugcacuggguuaagccugatt

SEQ ID NO: 1605 - DNA, 21 bp, Artificial Sequence
  Synthetic DNA/RNA construct - RAG3A-655-saRNA antisense sequence
uccgucuuccaucuccuactt

SEQ ID NO: 1606 - DNA, 21 bp, Artificial Sequence
  Synthetic DNA/RNA construct - RAG3A-170-saRNA antisense sequence
acaugggcaaccugggacctt

SEQ ID NO: 1607 - DNA, 21 bp, Artificial Sequence
  Synthetic DNA/RNA construct - RAG3A-629-saRNA antisense sequence
cagggcccuccuuugcuugtt

SEQ ID NO: 1608 - DNA, 21 bp, Artificial Sequence
  Synthetic DNA/RNA construct - RAG3A-253-saRNA antisense sequence
ggaagaagcacagaggucutt

SEQ ID NO: 1609 - DNA, 21 bp, Artificial Sequence
  Synthetic DNA/RNA construct - RAG3A-695-saRNA antisense sequence
aauaaccaggcccaugugutt

SEQ ID NO: 1610 - DNA, 21 bp, Artificial Sequence
  Synthetic DNA/RNA construct - RAG3A-788-saRNA antisense sequence
cuggcucuagcuccuuugctt

SEQ ID NO: 1611 - DNA, 21 bp, Artificial Sequence
  Synthetic DNA/RNA construct - RAG3A-688-saRNA antisense sequence
caagaggaauaaccaggcctt

SEQ ID NO: 1612 - DNA, 21 bp, Artificial Sequence
  Synthetic DNA/RNA construct - RAG3A-728-saRNA antisense sequence
agacagcugcuggaccucctt

SEQ ID NO: 1613 - DNA, 21 bp, Artificial Sequence
  Synthetic DNA/RNA construct - RAG3A-519-saRNA antisense sequence
gugcggggcacauguaugutt

SEQ ID NO: 1614 - DNA, 21 bp, Artificial Sequence
  Synthetic DNA/RNA construct - RAG3A-272-saRNA antisense sequence
uguuuaagauugguggggutt

SEQ ID NO: 1615 - DNA, 21 bp, Artificial Sequence
  Synthetic DNA/RNA construct - RAG3A-318-saRNA antisense sequence
uuucccuggacaucauacctt

SEQ ID NO: 1616 - DNA, 21 bp, Artificial Sequence
  Synthetic DNA/RNA construct - RAG3A-1338-saRNA antisense sequence
gagaggguggugaggguuutt

SEQ ID NO: 1617 - DNA, 21 bp, Artificial Sequence
  Synthetic DNA/RNA construct - RAG3A-539-saRNA antisense sequence
cgcacacgugcacuggguutt

SEQ ID NO: 1618 - DNA, 21 bp, Artificial Sequence
  Synthetic DNA/RNA construct - RAG3A-826-saRNA antisense sequence
uccccacagucucuguccutt

SEQ ID NO: 1619 - DNA, 21 bp, Artificial Sequence
  Synthetic DNA/RNA construct - RAG3A-337-saRNA antisense sequence
ugcugcugaaauccaacagtt

SEQ ID NO: 1620 - DNA, 21 bp, Artificial Sequence
  Synthetic DNA/RNA construct - RAG3A-273-saRNA antisense sequence
guuuaagauuggugggguutt

SEQ ID NO: 1621 - DNA, 21 bp, Artificial Sequence
  Synthetic DNA/RNA construct - RAG3A-478-saRNA antisense sequence
guuauggggaaaggguuugtt

SEQ ID NO: 1622 - DNA, 21 bp, Artificial Sequence
  Synthetic DNA/RNA construct - RAG3A-963-saRNA antisense sequence
auccuggaauuuucccguctt

SEQ ID NO: 1623 - DNA, 21 bp, Artificial Sequence
  Synthetic DNA/RNA construct - RAG3A-248-saRNA antisense sequence
gcuggggaagaagcacagatt

SEQ ID NO: 1624 - DNA, 21 bp, Artificial Sequence
  Synthetic DNA/RNA construct - RAG3A-35-saRNA antisense sequence
agccaccagacacuggugatt

SEQ ID NO: 1625 - DNA, 21 bp, Artificial Sequence
  Synthetic DNA/RNA construct - RAG3A-613-saRNA antisense sequence
cacagaccucugugcccagtt

SEQ ID NO: 1626 - DNA, 21 bp, Artificial Sequence
  Synthetic DNA/RNA construct - RAG3A-1141-saRNA antisense sequence
cccuugggugaaggaagggtt

SEQ ID NO: 1627 - DNA, 21 bp, Artificial Sequence
  Synthetic DNA/RNA construct - RAG3A-477-saRNA antisense sequence
uguuauggggaaaggguuutt

SEQ ID NO: 1628 - DNA, 21 bp, Artificial Sequence
  Synthetic DNA/RNA construct - RAG3A-634-saRNA antisense sequence
cccuccuuugcuuguucugtt

SEQ ID NO: 1629 - DNA, 21 bp, Artificial Sequence
  Synthetic DNA/RNA construct - RAG3A-1280-saRNA antisense sequence
uaacacaagaaugaggagctt

SEQ ID NO: 1630 - DNA, 21 bp, Artificial Sequence
  Synthetic DNA/RNA construct - RAG3A-1062-saRNA antisense sequence
gagacaggagacagggaggtt

SEQ ID NO: 1631 - DNA, 21 bp, Artificial Sequence
  Synthetic DNA/RNA construct - RAG3A-778-saRNA antisense sequence
uccuuuggcccuggcucuatt

SEQ ID NO: 1632 - DNA, 21 bp, Artificial Sequence
  Synthetic DNA/RNA construct - RAG3A-939-saRNA antisense sequence
ugucugcugagaaguguggtt

SEQ ID NO: 1633 - DNA, 21 bp, Artificial Sequence
  Synthetic DNA/RNA construct - RAG3A-779-saRNA antisense sequence
ccuuuggcccuggcucuagtt

SEQ ID NO: 1634 - DNA, 21 bp, Artificial Sequence
  Synthetic DNA/RNA construct - RAG3A-803-saRNA antisense sequence
uugcuuucuucccaguccctt

SEQ ID NO: 1635 - DNA, 21 bp, Artificial Sequence
  Synthetic DNA/RNA construct - RAG3A-1382-saRNA antisense sequence
gggagaucggcugauggactt

SEQ ID NO: 1636 - DNA, 21 bp, Artificial Sequence
  Synthetic DNA/RNA construct - RAG3A-97-saRNA antisense sequence
uggaccaggguccacuccutt

SEQ ID NO: 1637 - DNA, 21 bp, Artificial Sequence
  Synthetic DNA/RNA construct - RAG3A-543-saRNA antisense sequence
cacgugcacuggguuaagctt

SEQ ID NO: 1638 - DNA, 21 bp, Artificial Sequence
  Synthetic DNA/RNA construct - RAG3A-556-saRNA antisense sequence
uuaagccugagcuuaggugtt

SEQ ID NO: 1639 - DNA, 21 bp, Artificial Sequence
  Synthetic DNA/RNA construct - RAG3A-60-saRNA antisense sequence
ccggaagugacgccuucuctt

SEQ ID NO: 1640 - DNA, 21 bp, Artificial Sequence
  Synthetic DNA/RNA construct - RAG3A-647-saRNA antisense sequence
guucugucuccgucuuccatt

SEQ ID NO: 1641 - DNA, 21 bp, Artificial Sequence
  Synthetic DNA/RNA construct - RAG3A-243-saRNA antisense sequence
guguugcuggggaagaagctt

SEQ ID NO: 1642 - DNA, 21 bp, Artificial Sequence
  Synthetic DNA/RNA construct - RAG3A-533-saRNA antisense sequence
uaugugcgcacacgugcactt

SEQ ID NO: 1643 - DNA, 21 bp, Artificial Sequence
  Synthetic DNA/RNA construct - RAG3A-628-saRNA antisense sequence
ccagggcccuccuuugcuutt

SEQ ID NO: 1644 - DNA, 21 bp, Artificial Sequence
  Synthetic DNA/RNA construct - RAG3A-849-saRNA antisense sequence
ggcuuccuggccugaccuutt

SEQ ID NO: 1645 - DNA, 21 bp, Artificial Sequence
  Synthetic DNA/RNA construct - RAG3A-1307-saRNA antisense sequence
agagaagguagggaggcagtt

SEQ ID NO: 1646 - DNA, 21 bp, Artificial Sequence
  Synthetic DNA/RNA construct - RAG3A-293-saRNA antisense sequence
ucacucccuggcuauccaatt

SEQ ID NO: 1647 - DNA, 21 bp, Artificial Sequence
  Synthetic DNA/RNA construct - RAG3A-1285-saRNA antisense sequence
caagaaugaggagcgaccutt

SEQ ID NO: 1648 - DNA, 21 bp, Artificial Sequence
  Synthetic DNA/RNA construct - RAG3A-1223-saRNA antisense sequence
cugucacuccacagaagggtt

SEQ ID NO: 1649 - DNA, 21 bp, Artificial Sequence
  Synthetic DNA/RNA construct - RAG3A-1177-saRNA antisense sequence
ggacugucgaaugcgccugtt

SEQ ID NO: 1650 - DNA, 21 bp, Artificial Sequence
  Synthetic DNA/RNA construct - RAG3A-226-saRNA antisense sequence
aaucuggcaggacauuugutt

SEQ ID NO: 1651 - DNA, 21 bp, Artificial Sequence
  Synthetic DNA/RNA construct - RAG3A-1097-saRNA antisense sequence
accaagagaaaggaggcugtt

SEQ ID NO: 1652 - DNA, 21 bp, Artificial Sequence
  Synthetic DNA/RNA construct - RAG3A-1188-saRNA antisense sequence
ugcgccugaggauaaguuatt

SEQ ID NO: 1653 - DNA, 21 bp, Artificial Sequence
  Synthetic DNA/RNA construct - RAG3A-593-saRNA antisense sequence
guggcuuagauggcuacactt

SEQ ID NO: 1654 - DNA, 21 bp, Artificial Sequence
  Synthetic DNA/RNA construct - RAG3A-670-saRNA antisense sequence
cuaccguuccacaugugactt

SEQ ID NO: 1655 - DNA, 21 bp, Artificial Sequence
  Synthetic DNA/RNA construct - RAG3A-503-saRNA antisense sequence
uugaguggacugucaggugtt

SEQ ID NO: 1656 - DNA, 21 bp, Artificial Sequence
  Synthetic DNA/RNA construct - RAG3A-1167-saRNA antisense sequence
aagcaagaggggacugucgtt

SEQ ID NO: 1657 - DNA, 21 bp, Artificial Sequence
  Synthetic DNA/RNA construct - RAG3A-583-saRNA antisense sequence
cugggguccaguggcuuagtt

SEQ ID NO: 1658 - DNA, 21 bp, Artificial Sequence
  Synthetic DNA/RNA construct - RAG3A-706-saRNA antisense sequence
ccaugugucccugucuuagtt

SEQ ID NO: 1659 - DNA, 21 bp, Artificial Sequence
  Synthetic DNA/RNA construct - RAG3A-983-saRNA antisense sequence
agccagacgagaugucucctt

SEQ ID NO: 1660 - DNA, 21 bp, Artificial Sequence
  Synthetic DNA/RNA construct - RAG3A-718-saRNA antisense sequence
gucuuaggaaagacagcugtt

SEQ ID NO: 1661 - DNA, 21 bp, Artificial Sequence
  Synthetic DNA/RNA construct - RAG3A-378-saRNA antisense sequence
uuucuguaauccccuagggtt

SEQ ID NO: 1662 - DNA, 21 bp, Artificial Sequence
  Synthetic DNA/RNA construct - RAG3A-1095-saRNA antisense sequence
gaaccaagagaaaggaggctt

SEQ ID NO: 1663 - DNA, 21 bp, Artificial Sequence
  Synthetic DNA/RNA construct - RAG3A-698-saRNA antisense sequence
aaccaggcccauguguccctt

SEQ ID NO: 1664 - DNA, 21 bp, Artificial Sequence
  Synthetic DNA/RNA construct - RAG3A-277-saRNA antisense sequence
aagauuggugggguuuucatt

SEQ ID NO: 1665 - DNA, 21 bp, Artificial Sequence
  Synthetic DNA/RNA construct - RAG3A-644-saRNA antisense sequence
cuuguucugucuccgucuutt

SEQ ID NO: 1666 - DNA, 21 bp, Artificial Sequence
  Synthetic DNA/RNA construct - RAG3A-941-saRNA antisense sequence
ucugcugagaaguguggcctt

SEQ ID NO: 1667 - DNA, 21 bp, Artificial Sequence
  Synthetic DNA/RNA construct - RAG3A-794-saRNA antisense sequence
cuagcuccuuugcuuucuutt

SEQ ID NO: 1668 - DNA, 21 bp, Artificial Sequence
  Synthetic DNA/RNA construct - RAG3A-890-saRNA antisense sequence
ccagaacuuccucaggcuctt

SEQ ID NO: 1669 - DNA, 21 bp, Artificial Sequence
  Synthetic DNA/RNA construct - RAG3A-1310-saRNA antisense sequence
gaagguagggaggcaggcutt

SEQ ID NO: 1670 - DNA, 21 bp, Artificial Sequence
  Synthetic DNA/RNA construct - RAG3A-27-saRNA antisense sequence
gagaagggagccaccagactt

SEQ ID NO: 1671 - DNA, 21 bp, Artificial Sequence
  Synthetic DNA/RNA construct - RAG3A-38-saRNA antisense sequence
caccagacacuggugaaggtt

SEQ ID NO: 1672 - DNA, 21 bp, Artificial Sequence
  Synthetic DNA/RNA construct - RAG3A-532-saRNA antisense sequence
guaugugcgcacacgugcatt

SEQ ID NO: 1673 - DNA, 21 bp, Artificial Sequence
  Synthetic DNA/RNA construct - RAG3A-1023-saRNA antisense sequence
ugggauagcugggaggauatt

SEQ ID NO: 1674 - DNA, 21 bp, Artificial Sequence
  Synthetic DNA/RNA construct - RAG3A-797-saRNA antisense sequence
gcuccuuugcuuucuuccctt

SEQ ID NO: 1675 - DNA, 21 bp, Artificial Sequence
  Synthetic DNA/RNA construct - RAG3A-399-saRNA antisense sequence
uccuaucuugucauucccctt

SEQ ID NO: 1676 - DNA, 21 bp, Artificial Sequence
  Synthetic DNA/RNA construct - RAG3A-945-saRNA antisense sequence
cugagaaguguggccuagatt

SEQ ID NO: 1677 - DNA, 21 bp, Artificial Sequence
  Synthetic DNA/RNA construct - RAG3A-344-saRNA antisense sequence
gaaauccaacaggaugcuutt

SEQ ID NO: 1678 - DNA, 21 bp, Artificial Sequence
  Synthetic DNA/RNA construct - RAG3A-1255-saRNA antisense sequence
uuucuggcuaggucccugutt

SEQ ID NO: 1679 - DNA, 21 bp, Artificial Sequence
  Synthetic DNA/RNA construct - RAG3A-524-saRNA antisense sequence
gggcacauguaugugcgcatt

SEQ ID NO: 1680 - DNA, 21 bp, Artificial Sequence
  Synthetic DNA/RNA construct - RAG3A-244-saRNA antisense sequence
uguugcuggggaagaagcatt

SEQ ID NO: 1681 - DNA, 21 bp, Artificial Sequence
  Synthetic DNA/RNA construct - RAG3A-340-saRNA antisense sequence
ugcugaaauccaacaggautt

SEQ ID NO: 1682 - DNA, 21 bp, Artificial Sequence
  Synthetic DNA/RNA construct - RAG3A-687-saRNA antisense sequence
acaagaggaauaaccaggctt

SEQ ID NO: 1683 - DNA, 21 bp, Artificial Sequence
  Synthetic DNA/RNA construct - RAG3A-568-saRNA antisense sequence
uuaggugcucgucugcuggtt

SEQ ID NO: 1684 - DNA, 21 bp, Artificial Sequence
  Synthetic DNA/RNA construct - RAG3A-804-saRNA antisense sequence
ugcuuucuucccagucccatt

SEQ ID NO: 1685 - DNA, 21 bp, Artificial Sequence
  Synthetic DNA/RNA construct - RAG3A-496-saRNA antisense sequence
ggacggguugaguggacugtt

SEQ ID NO: 1686 - DNA, 21 bp, Artificial Sequence
  Synthetic DNA/RNA construct - RAG3A-810-saRNA antisense sequence
cuucccagucccagucucctt

SEQ ID NO: 1687 - DNA, 21 bp, Artificial Sequence
  Synthetic DNA/RNA construct - RAG3A-356-saRNA antisense sequence
gaugcuugcuuuccugcuutt

SEQ ID NO: 1688 - DNA, 21 bp, Artificial Sequence
  Synthetic DNA/RNA construct - RAG3A-1248-saRNA antisense sequence
cugccgguuucuggcuaggtt

SEQ ID NO: 1689 - DNA, 21 bp, Artificial Sequence
  Synthetic DNA/RNA construct - RAG3A-1015-saRNA antisense sequence
gagggugcugggauagcugtt

SEQ ID NO: 1690 - DNA, 21 bp, Artificial Sequence
  Synthetic DNA/RNA construct - RAG3A-645-saRNA antisense sequence
uuguucugucuccgucuuctt

SEQ ID NO: 1691 - DNA, 21 bp, Artificial Sequence
  Synthetic DNA/RNA construct - RAG3A-517-saRNA antisense sequence
aggugcggggcacauguautt

SEQ ID NO: 1692 - DNA, 21 bp, Artificial Sequence
  Synthetic DNA/RNA construct - RAG3A-194-saRNA antisense sequence
ggggacaggagcagaaguutt

SEQ ID NO: 1693 - DNA, 21 bp, Artificial Sequence
  Synthetic DNA/RNA construct - RAG3A-927-saRNA antisense sequence
caaggaugggcaugucugctt

SEQ ID NO: 1694 - DNA, 21 bp, Artificial Sequence
  Synthetic DNA/RNA construct - RAG3A-271-saRNA antisense sequence
uuguuuaagauugguggggtt

SEQ ID NO: 1695 - DNA, 21 bp, Artificial Sequence
  Synthetic DNA/RNA construct - RAG3A-398-saRNA antisense sequence
guccuaucuugucauuccctt

SEQ ID NO: 1696 - DNA, 21 bp, Artificial Sequence
  Synthetic DNA/RNA construct - RAG3A-230-saRNA antisense sequence
uggcaggacauuuguguugtt

SEQ ID NO: 1697 - DNA, 21 bp, Artificial Sequence
  Synthetic DNA/RNA construct - RAG3A-1104-saRNA antisense sequence
gaaaggaggcugggaguggtt

SEQ ID NO: 1698 - DNA, 21 bp, Artificial Sequence
  Synthetic DNA/RNA construct - RAG3A-1254-saRNA antisense sequence
guuucuggcuaggucccugtt

SEQ ID NO: 1699 - DNA, 21 bp, Artificial Sequence
  Synthetic DNA/RNA construct - RAG3A-288-saRNA antisense sequence
gguuuucacucccuggcuatt

SEQ ID NO: 1700 - DNA, 21 bp, Artificial Sequence
  Synthetic DNA/RNA construct - RAG3A-837-saRNA antisense sequence
ucuguccucaggggcuucctt

SEQ ID NO: 1701 - DNA, 21 bp, Artificial Sequence
  Synthetic DNA/RNA construct - RAG3A-935-saRNA antisense sequence
ggcaugucugcugagaagutt

SEQ ID NO: 1702 - DNA, 21 bp, Artificial Sequence
  Synthetic DNA/RNA construct - RAG3A-476-saRNA antisense sequence
guguuauggggaaaggguutt

SEQ ID NO: 1703 - DNA, 21 bp, Artificial Sequence
  Synthetic DNA/RNA construct - RAG3A-589-saRNA antisense sequence
uccaguggcuuagauggcutt

SEQ ID NO: 1704 - DNA, 21 bp, Artificial Sequence
  Synthetic DNA/RNA construct - RAG3A-1207-saRNA antisense sequence
caaugagagggagauacugtt

SEQ ID NO: 1705 - DNA, 21 bp, Artificial Sequence
  Synthetic DNA/RNA construct - RAG3A-1284-saRNA antisense sequence
acaagaaugaggagcgacctt

SEQ ID NO: 1706 - DNA, 21 bp, Artificial Sequence
  Synthetic DNA/RNA construct - RAG3A-956-saRNA antisense sequence
ggccuagauccuggaauuutt

SEQ ID NO: 1707 - DNA, 21 bp, Artificial Sequence
  Synthetic DNA/RNA construct - RAG3A-557-saRNA antisense sequence
uaagccugagcuuaggugctt

SEQ ID NO: 1708 - DNA, 21 bp, Artificial Sequence
  Synthetic DNA/RNA construct - RAG3A-500-saRNA antisense sequence
ggguugaguggacugucagtt

SEQ ID NO: 1709 - DNA, 21 bp, Artificial Sequence
  Synthetic DNA/RNA construct - RAG3A-1208-saRNA antisense sequence
aaugagagggagauacugutt

SEQ ID NO: 1710 - DNA, 21 bp, Artificial Sequence
  Synthetic DNA/RNA construct - RAG3A-686-saRNA antisense sequence
gacaagaggaauaaccaggtt

SEQ ID NO: 1711 - DNA, 21 bp, Artificial Sequence
  Synthetic DNA/RNA construct - RAG3A-1168-saRNA antisense sequence
agcaagaggggacugucgatt

SEQ ID NO: 1712 - DNA, 21 bp, Artificial Sequence
  Synthetic DNA/RNA construct - RAG3A-218-saRNA antisense sequence
ccaggaggaaucuggcaggtt

SEQ ID NO: 1713 - DNA, 21 bp, Artificial Sequence
  Synthetic DNA/RNA construct - RAG3A-835-saRNA antisense sequence
ucucuguccucaggggcuutt

SEQ ID NO: 1714 - DNA, 21 bp, Artificial Sequence
  Synthetic DNA/RNA construct - RAG3A-291-saRNA antisense sequence
uuucacucccuggcuaucctt

SEQ ID NO: 1715 - DNA, 21 bp, Artificial Sequence
  Synthetic DNA/RNA construct - RAG3A-1222-saRNA antisense sequence
acugucacuccacagaaggtt

SEQ ID NO: 1716 - DNA, 21 bp, Artificial Sequence
  Synthetic DNA/RNA construct - RAG3A-276-saRNA antisense sequence
uaagauuggugggguuuuctt

SEQ ID NO: 1717 - DNA, 21 bp, Artificial Sequence
  Synthetic DNA/RNA construct - RAG3A-156-saRNA antisense sequence
ccaucuuuuccuggacaugtt

SEQ ID NO: 1718 - DNA, 21 bp, Artificial Sequence
  Synthetic DNA/RNA construct - RAG3A-937-saRNA antisense sequence
caugucugcugagaagugutt

SEQ ID NO: 1719 - DNA, 21 bp, Artificial Sequence
  Synthetic DNA/RNA construct - RAG3A-1262-saRNA antisense sequence
cuaggucccuguccugggutt

SEQ ID NO: 1720 - DNA, 21 bp, Artificial Sequence
  Synthetic DNA/RNA construct - RAG3A-1139-saRNA antisense sequence
gucccuugggugaaggaagtt

SEQ ID NO: 1721 - DNA, 21 bp, Artificial Sequence
  Synthetic DNA/RNA construct - RAG3A-535-saRNA antisense sequence
ugugcgcacacgugcacugtt

SEQ ID NO: 1722 - DNA, 21 bp, Artificial Sequence
  Synthetic DNA/RNA construct - RAG3A-719-saRNA antisense sequence
ucuuaggaaagacagcugctt

SEQ ID NO: 1723 - DNA, 21 bp, Artificial Sequence
  Synthetic DNA/RNA construct - RAG3A-726-saRNA antisense sequence
aaagacagcugcuggaccutt

SEQ ID NO: 1724 - DNA, 21 bp, Artificial Sequence
  Synthetic DNA/RNA construct - RAG3A-384-saRNA antisense sequence
uaauccccuagggaguccutt

SEQ ID NO: 1725 - DNA, 21 bp, Artificial Sequence
  Synthetic DNA/RNA construct - RAG3A-834-saRNA antisense sequence
gucucuguccucaggggcutt

SEQ ID NO: 1726 - DNA, 21 bp, Artificial Sequence
  Synthetic DNA/RNA construct - RAG3A-562-saRNA antisense sequence
cugagcuuaggugcucguctt

SEQ ID NO: 1727 - DNA, 21 bp, Artificial Sequence
  Synthetic DNA/RNA construct - RAG3A-940-saRNA antisense sequence
gucugcugagaaguguggctt

SEQ ID NO: 1728 - DNA, 21 bp, Artificial Sequence
  Synthetic DNA/RNA construct - RAG3A-632-saRNA antisense sequence
ggcccuccuuugcuuguuctt

SEQ ID NO: 1729 - DNA, 21 bp, Artificial Sequence
  Synthetic DNA/RNA construct - RAG3A-746-saRNA antisense sequence
ccccgcaaauaccucccugtt

SEQ ID NO: 1730 - DNA, 21 bp, Artificial Sequence
  Synthetic DNA/RNA construct - RAG3A-1309-saRNA antisense sequence
agaagguagggaggcaggctt

SEQ ID NO: 1731 - DNA, 21 bp, Artificial Sequence
  Synthetic DNA/RNA construct - RAG3A-780-saRNA antisense sequence
cuuuggcccuggcucuagctt

SEQ ID NO: 1732 - DNA, 21 bp, Artificial Sequence
  Synthetic DNA/RNA construct - RAG3A-693-saRNA antisense sequence
ggaauaaccaggcccaugutt

SEQ ID NO: 1733 - DNA, 21 bp, Artificial Sequence
  Synthetic DNA/RNA construct - RAG3A-636-saRNA antisense sequence
cuccuuugcuuguucuguctt

SEQ ID NO: 1734 - DNA, 21 bp, Artificial Sequence
  Synthetic DNA/RNA construct - RAG3A-258-saRNA antisense sequence
aagcacagaggucuuguuutt

SEQ ID NO: 1735 - DNA, 21 bp, Artificial Sequence
  Synthetic DNA/RNA construct - RAG3A-1022-saRNA antisense sequence
cugggauagcugggaggautt

SEQ ID NO: 1736 - DNA, 21 bp, Artificial Sequence
  Synthetic DNA/RNA construct - RAG3A-381-saRNA antisense sequence
cuguaauccccuagggagutt

SEQ ID NO: 1737 - DNA, 21 bp, Artificial Sequence
  Synthetic DNA/RNA construct - RAG3A-379-saRNA antisense sequence
uucuguaauccccuagggatt

SEQ ID NO: 1738 - DNA, 21 bp, Artificial Sequence
  Synthetic DNA/RNA construct - RAG3A-663-saRNA antisense sequence
ccaucuccuaccguuccactt

SEQ ID NO: 1739 - DNA, 21 bp, Artificial Sequence
  Synthetic DNA/RNA construct - RAG3A-1099-saRNA antisense sequence
caagagaaaggaggcugggtt

SEQ ID NO: 1740 - DNA, 21 bp, Artificial Sequence
  Synthetic DNA/RNA construct - RAG3A-855-saRNA antisense sequence
cuggccugaccuugauccctt

SEQ ID NO: 1741 - DNA, 21 bp, Artificial Sequence
  Synthetic DNA/RNA construct - RAG3A-1279-saRNA antisense sequence
guaacacaagaaugaggagtt

SEQ ID NO: 1742 - DNA, 21 bp, Artificial Sequence
  Synthetic DNA/RNA construct - RAG3A-34-saRNA antisense sequence
gagccaccagacacuggugtt

SEQ ID NO: 1743 - DNA, 21 bp, Artificial Sequence
  Synthetic DNA/RNA construct - RAG3A-633-saRNA antisense sequence
gcccuccuuugcuuguucutt

SEQ ID NO: 1744 - DNA, 21 bp, Artificial Sequence
  Synthetic DNA/RNA construct - RAG3A-453-saRNA antisense sequence
gagaaaucuccuguuauggtt

SEQ ID NO: 1745 - DNA, 21 bp, Artificial Sequence
  Synthetic DNA/RNA construct - RAG3A-246-saRNA antisense sequence
uugcuggggaagaagcacatt

SEQ ID NO: 1746 - DNA, 21 bp, Artificial Sequence
  Synthetic DNA/RNA construct - RAG3A-592-saRNA antisense sequence
aguggcuuagauggcuacatt

SEQ ID NO: 1747 - DNA, 21 bp, Artificial Sequence
  Synthetic DNA/RNA construct - RAG3A-270-saRNA antisense sequence
cuuguuuaagauuggugggtt

SEQ ID NO: 1748 - DNA, 21 bp, Artificial Sequence
  Synthetic DNA/RNA construct - RAG3A-630-saRNA antisense sequence
agggcccuccuuugcuugutt

SEQ ID NO: 1749 - DNA, 21 bp, Artificial Sequence
  Synthetic DNA/RNA construct - RAG3A-685-saRNA antisense sequence
ugacaagaggaauaaccagtt

SEQ ID NO: 1750 - DNA, 21 bp, Artificial Sequence
  Synthetic DNA/RNA construct - RAG3A-1103-saRNA antisense sequence
agaaaggaggcugggagugtt

SEQ ID NO: 1751 - DNA, 21 bp, Artificial Sequence
  Synthetic DNA/RNA construct - RAG3A-1065-saRNA antisense sequence
acaggagacagggaggcagtt

SEQ ID NO: 1752 - DNA, 21 bp, Artificial Sequence
  Synthetic DNA/RNA construct - RAG3A-523-saRNA antisense sequence
ggggcacauguaugugcgctt

SEQ ID NO: 1753 - DNA, 21 bp, Artificial Sequence
  Synthetic DNA/RNA construct - RAG3A-590-saRNA antisense sequence
ccaguggcuuagauggcuatt

SEQ ID NO: 1754 - DNA, 21 bp, Artificial Sequence
  Synthetic DNA/RNA construct - RAG3A-505-saRNA antisense sequence
gaguggacugucaggugcgtt

SEQ ID NO: 1755 - DNA, 21 bp, Artificial Sequence
  Synthetic DNA/RNA construct - RAG3A-1247-saRNA antisense sequence
gcugccgguuucuggcuagtt

SEQ ID NO: 1756 - DNA, 21 bp, Artificial Sequence
  Synthetic DNA/RNA construct - RAG3A-1281-saRNA antisense sequence
aacacaagaaugaggagcgtt

SEQ ID NO: 1757 - DNA, 21 bp, Artificial Sequence
  Synthetic DNA/RNA construct - RAG3A-747-saRNA antisense sequence
cccgcaaauaccucccugctt

SEQ ID NO: 1758 - DNA, 21 bp, Artificial Sequence
  Synthetic DNA/RNA construct - RAG3A-1220-saRNA antisense sequence
auacugucacuccacagaatt

SEQ ID NO: 1759 - DNA, 21 bp, Artificial Sequence
  Synthetic DNA/RNA construct - RAG3A-1268-saRNA antisense sequence
cccuguccuggguaacacatt

SEQ ID NO: 1760 - DNA, 21 bp, Artificial Sequence
  Synthetic DNA/RNA construct - RAG3A-782-saRNA antisense sequence
uuggcccuggcucuagcuctt

SEQ ID NO: 1761 - DNA, 21 bp, Artificial Sequence
  Synthetic DNA/RNA construct - RAG3A-107-saRNA antisense sequence
uccacuccuccacccacactt

SEQ ID NO: 1762 - DNA, 21 bp, Artificial Sequence
  Synthetic DNA/RNA construct - RAG3A-923-saRNA antisense sequence
uccccaaggaugggcaugutt

SEQ ID NO: 1763 - DNA, 21 bp, Artificial Sequence
  Synthetic DNA/RNA construct - RAG3A-349-saRNA antisense sequence
ccaacaggaugcuugcuuutt

SEQ ID NO: 1764 - DNA, 21 bp, Artificial Sequence
  Synthetic DNA/RNA construct - RAG3A-1085-saRNA antisense sequence
caugaccagggaaccaagatt

SEQ ID NO: 1765 - DNA, 21 bp, Artificial Sequence
  Synthetic DNA/RNA construct - RAG3A-432-saRNA antisense sequence
acacggauauugcccacautt

SEQ ID NO: 1766 - DNA, 21 bp, Artificial Sequence
  Synthetic DNA/RNA construct - RAG3A-129-saRNA antisense sequence
ccacggagaagauuuggautt

SEQ ID NO: 1767 - DNA, 21 bp, Artificial Sequence
  Synthetic DNA/RNA construct - RAG3A-830-saRNA antisense sequence
cacagucucuguccucaggtt

SEQ ID NO: 1768 - DNA, 21 bp, Artificial Sequence
  Synthetic DNA/RNA construct - RAG3A-74-saRNA antisense sequence
uucucuucccuggagcccctt

SEQ ID NO: 1769 - DNA, 21 bp, Artificial Sequence
  Synthetic DNA/RNA construct - RAG3A-856-saRNA antisense sequence
uggccugaccuugaucccatt

SEQ ID NO: 1770 - DNA, 21 bp, Artificial Sequence
  Synthetic DNA/RNA construct - RAG3A-435-saRNA antisense sequence
cggauauugcccacaugagtt

SEQ ID NO: 1771 - DNA, 21 bp, Artificial Sequence
  Synthetic DNA/RNA construct - RAG3A-564-saRNA antisense sequence
gagcuuaggugcucgucugtt

SEQ ID NO: 1772 - DNA, 21 bp, Artificial Sequence
  Synthetic DNA/RNA construct - RAG3A-1251-saRNA antisense sequence
ccgguuucuggcuaggucctt

SEQ ID NO: 1773 - DNA, 21 bp, Artificial Sequence
  Synthetic DNA/RNA construct - RAG3A-1291-saRNA antisense sequence
ugaggagcgaccucagagatt

SEQ ID NO: 1774 - DNA, 21 bp, Artificial Sequence
  Synthetic DNA/RNA construct - RAG3A-781-saRNA antisense sequence
uuuggcccuggcucuagcutt

SEQ ID NO: 1775 - DNA, 21 bp, Artificial Sequence
  Synthetic DNA/RNA construct - RAG3A-261-saRNA antisense sequence
cacagaggucuuguuuaagtt

SEQ ID NO: 1776 - DNA, 21 bp, Artificial Sequence
  Synthetic DNA/RNA construct - RAG3A-1098-saRNA antisense sequence
ccaagagaaaggaggcuggtt

SEQ ID NO: 1777 - DNA, 21 bp, Artificial Sequence
  Synthetic DNA/RNA construct - RAG3A-565-saRNA antisense sequence
agcuuaggugcucgucugctt

SEQ ID NO: 1778 - DNA, 21 bp, Artificial Sequence
  Synthetic DNA/RNA construct - RAG3A-930-saRNA antisense sequence
ggaugggcaugucugcugatt

SEQ ID NO: 1779 - DNA, 21 bp, Artificial Sequence
  Synthetic DNA/RNA construct - RAG3A-422-saRNA antisense sequence
cgaagugggaacacggauatt

SEQ ID NO: 1780 - DNA, 21 bp, Artificial Sequence
  Synthetic DNA/RNA construct - RAG3A-516-saRNA antisense sequence
caggugcggggcacauguatt

SEQ ID NO: 1781 - DNA, 21 bp, Artificial Sequence
  Synthetic DNA/RNA construct - RAG3A-1190-saRNA antisense sequence
cgccugaggauaaguuacatt

SEQ ID NO: 1782 - DNA, 21 bp, Artificial Sequence
  Synthetic DNA/RNA construct - RAG3A-360-saRNA antisense sequence
cuugcuuuccugcuuuucutt

SEQ ID NO: 1783 - DNA, 21 bp, Artificial Sequence
  Synthetic DNA/RNA construct - RAG3A-126-saRNA antisense sequence
acaccacggagaagauuugtt

SEQ ID NO: 1784 - DNA, 21 bp, Artificial Sequence
  Synthetic DNA/RNA construct - RAG3A-938-saRNA antisense sequence
augucugcugagaagugugtt

SEQ ID NO: 1785 - DNA, 21 bp, Artificial Sequence
  Synthetic DNA/RNA construct - RAG3A-853-saRNA antisense sequence
uccuggccugaccuugauctt

SEQ ID NO: 1786 - DNA, 21 bp, Artificial Sequence
  Synthetic DNA/RNA construct - RAG3A-716-saRNA antisense sequence
cugucuuaggaaagacagctt

SEQ ID NO: 1787 - DNA, 21 bp, Artificial Sequence
  Synthetic DNA/RNA construct - RAG3A-1027-saRNA antisense sequence
auagcugggaggauaguggtt

SEQ ID NO: 1788 - DNA, 21 bp, Artificial Sequence
  Synthetic DNA/RNA construct - RAG3A-640-saRNA antisense sequence
uuugcuuguucugucuccgtt

SEQ ID NO: 1789 - DNA, 21 bp, Artificial Sequence
  Synthetic DNA/RNA construct - RAG3A-284-saRNA antisense sequence
gugggguuuucacucccugtt

SEQ ID NO: 1790 - DNA, 21 bp, Artificial Sequence
  Synthetic DNA/RNA construct - RAG3A-705-saRNA antisense sequence
cccaugugucccugucuuatt

SEQ ID NO: 1791 - DNA, 21 bp, Artificial Sequence
  Synthetic DNA/RNA construct - RAG3A-1315-saRNA antisense sequence
uagggaggcaggcuaggcatt

SEQ ID NO: 1792 - DNA, 21 bp, Artificial Sequence
  Synthetic DNA/RNA construct - RAG3A-1381-saRNA antisense sequence
ggggagaucggcugauggatt

SEQ ID NO: 1793 - DNA, 21 bp, Artificial Sequence
  Synthetic DNA/RNA construct - RAG3A-558-saRNA antisense sequence
aagccugagcuuaggugcutt

SEQ ID NO: 1794 - DNA, 21 bp, Artificial Sequence
  Synthetic DNA/RNA construct - RAG3A-498-saRNA antisense sequence
acggguugaguggacuguctt

SEQ ID NO: 1795 - DNA, 21 bp, Artificial Sequence
  Synthetic DNA/RNA construct - RAG3A-581-saRNA antisense sequence
ugcugggguccaguggcuutt

SEQ ID NO: 1796 - DNA, 21 bp, Artificial Sequence
  Synthetic DNA/RNA construct - RAG3A-1135-saRNA antisense sequence
gagggucccuugggugaagtt

SEQ ID NO: 1797 - DNA, 21 bp, Artificial Sequence
  Synthetic DNA/RNA construct - RAG3A-1070-saRNA antisense sequence
agacagggaggcaggcaugtt

SEQ ID NO: 1798 - DNA, 21 bp, Artificial Sequence
  Synthetic DNA/RNA construct - RAG3A-1181-saRNA antisense sequence
ugucgaaugcgccugaggatt

SEQ ID NO: 1799 - DNA, 21 bp, Artificial Sequence
  Synthetic DNA/RNA construct - RAG3A-1206-saRNA antisense sequence
acaaugagagggagauacutt

SEQ ID NO: 1800 - DNA, 21 bp, Artificial Sequence
  Synthetic DNA/RNA construct - RAG3A-461-saRNA antisense sequence
uccuguuauggguuggugutt

SEQ ID NO: 1801 - DNA, 21 bp, Artificial Sequence
  Synthetic DNA/RNA construct - RAG3A-242-saRNA antisense sequence
uguguugcuggggaagaagtt

SEQ ID NO: 1802 - DNA, 21 bp, Artificial Sequence
  Synthetic DNA/RNA construct - RAG3A-1127-saRNA antisense sequence
gagaggcagagggucccuutt

SEQ ID NO: 1803 - DNA, 21 bp, Artificial Sequence
  Synthetic DNA/RNA construct - RAG3A-419-saRNA antisense sequence
uuucgaagugggaacacggtt

SEQ ID NO: 1804 - DNA, 21 bp, Artificial Sequence
  Synthetic DNA/RNA construct - RAG3A-790-saRNA antisense sequence
ggcucuagcuccuuugcuutt

SEQ ID NO: 1805 - DNA, 21 bp, Artificial Sequence
  Synthetic DNA/RNA construct - RAG3A-635-saRNA antisense sequence
ccuccuuugcuuguucugutt

SEQ ID NO: 1806 - DNA, 21 bp, Artificial Sequence
  Synthetic DNA/RNA construct - RAG3A-673-saRNA antisense sequence
ccguuccacaugugacaagtt

SEQ ID NO: 1807 - DNA, 21 bp, Artificial Sequence
  Synthetic DNA/RNA construct - RAG3A-1001-saRNA antisense sequence
ccaagauuaggaaggagggtt

SEQ ID NO: 1808 - DNA, 21 bp, Artificial Sequence
  Synthetic DNA/RNA construct - RAG3A-1005-saRNA antisense sequence
gauuaggaaggagggugcutt

SEQ ID NO: 1809 - DNA, 21 bp, Artificial Sequence
  Synthetic DNA/RNA construct - RAG3A-1064-saRNA antisense sequence
gacaggagacagggaggcatt

SEQ ID NO: 1810 - DNA, 21 bp, Artificial Sequence
  Synthetic DNA/RNA construct - RAG3A-801-saRNA antisense sequence
cuuugcuuucuucccaguctt

SEQ ID NO: 1811 - DNA, 21 bp, Artificial Sequence
  Synthetic DNA/RNA construct - RAG3A-486-saRNA antisense sequence
gaaaggguuuggacggguutt

SEQ ID NO: 1812 - DNA, 21 bp, Artificial Sequence
  Synthetic DNA/RNA construct - RAG3A-555-saRNA antisense sequence
guuaagccugagcuuaggutt

SEQ ID NO: 1813 - DNA, 21 bp, Artificial Sequence
  Synthetic DNA/RNA construct - RAG3A-854-saRNA antisense sequence
ccuggccugaccuugaucctt

SEQ ID NO: 1814 - DNA, 21 bp, Artificial Sequence
  Synthetic DNA/RNA construct - RAG3A-1267-saRNA antisense sequence
ucccuguccuggguaacactt

SEQ ID NO: 1815 - DNA, 21 bp, Artificial Sequence
  Synthetic DNA/RNA construct - RAG3A-1264-saRNA antisense sequence
aggucccuguccuggguaatt

SEQ ID NO: 1816 - DNA, 21 bp, Artificial Sequence
  Synthetic DNA/RNA construct - RAG3A-513-saRNA antisense sequence
ugucaggugcggggcacautt

SEQ ID NO: 1817 - DNA, 21 bp, Artificial Sequence
  Synthetic DNA/RNA construct - RAG3A-1286-saRNA antisense sequence
aagaaugaggagcgaccuctt

SEQ ID NO: 1818 - DNA, 21 bp, Artificial Sequence
  Synthetic DNA/RNA construct - RAG3A-653-saRNA antisense sequence
ucuccgucuuccaucuccutt

SEQ ID NO: 1819 - DNA, 21 bp, Artificial Sequence
  Synthetic DNA/RNA construct - RAG3A-1014-saRNA antisense sequence
ggagggugcugggauagcutt

SEQ ID NO: 1820 - DNA, 21 bp, Artificial Sequence
  Synthetic DNA/RNA construct - RAG3A-61-saRNA antisense sequence
cggaagugacgccuucucutt

SEQ ID NO: 1821 - DNA, 21 bp, Artificial Sequence
  Synthetic DNA/RNA construct - RAG3A-666-saRNA antisense sequence
ucuccuaccguuccacaugtt

SEQ ID NO: 1822 - DNA, 21 bp, Artificial Sequence
  Synthetic DNA/RNA construct - RAG3A-348-saRNA antisense sequence
uccaacaggaugcuugcuutt

SEQ ID NO: 1823 - DNA, 21 bp, Artificial Sequence
  Synthetic DNA/RNA construct - RAG3A-545-saRNA antisense sequence
cgugcacuggguuaagccutt

SEQ ID NO: 1824 - DNA, 21 bp, Artificial Sequence
  Synthetic DNA/RNA construct - RAG3A-584-saRNA antisense sequence
ugggguccaguggcuuagatt

SEQ ID NO: 1825 - DNA, 21 bp, Artificial Sequence
  Synthetic DNA/RNA construct - RAG3A-1162-saRNA antisense sequence
ugagaaagcaagaggggactt

SEQ ID NO: 1826 - DNA, 21 bp, Artificial Sequence
  Synthetic DNA/RNA construct - RAG3A-308-saRNA antisense sequence
ccaaauuucuuuucccuggtt

SEQ ID NO: 1827 - DNA, 21 bp, Artificial Sequence
  Synthetic DNA/RNA construct - RAG3A-158-saRNA antisense sequence
aucuuuuccuggacaugggtt

SEQ ID NO: 1828 - DNA, 21 bp, Artificial Sequence
  Synthetic DNA/RNA construct - RAG3A-161-saRNA antisense sequence
uuuuccuggacaugggcaatt

SEQ ID NO: 1829 - DNA, 21 bp, Artificial Sequence
  Synthetic DNA/RNA construct - RAG3A-1253-saRNA antisense sequence
gguuucuggcuaggucccutt

SEQ ID NO: 1830 - DNA, 21 bp, Artificial Sequence
  Synthetic DNA/RNA construct - RAG3A-926-saRNA antisense sequence
ccaaggaugggcaugucugtt

SEQ ID NO: 1831 - DNA, 21 bp, Artificial Sequence
  Synthetic DNA/RNA construct - RAG3A-1191-saRNA antisense sequence
gccugaggauaaguuacaatt

SEQ ID NO: 1832 - DNA, 21 bp, Artificial Sequence
  Synthetic DNA/RNA construct - RAG3A-697-saRNA antisense sequence
uaaccaggcccaugugucctt

SEQ ID NO: 1833 - DNA, 21 bp, Artificial Sequence
  Synthetic DNA/RNA construct - RAG3A-1224-saRNA antisense sequence
ugucacuccacagaaggggtt

SEQ ID NO: 1834 - DNA, 21 bp, Artificial Sequence
  Synthetic DNA/RNA construct - RAG3A-964-saRNA antisense sequence
uccuggaauuuucccgucctt

SEQ ID NO: 1835 - DNA, 21 bp, Artificial Sequence
  Synthetic DNA/RNA construct - RAG3A-791-saRNA antisense sequence
gcucuagcuccuuugcuuutt

SEQ ID NO: 1836 - DNA, 21 bp, Artificial Sequence
  Synthetic DNA/RNA construct - RAG3A-526-saRNA antisense sequence
gcacauguaugugcgcacatt

SEQ ID NO: 1837 - DNA, 21 bp, Artificial Sequence
  Synthetic DNA/RNA construct - RAG3A-972-saRNA antisense sequence
uuuucccguccagccagactt

SEQ ID NO: 1838 - DNA, 21 bp, Artificial Sequence
  Synthetic DNA/RNA construct - RAG3A-289-saRNA antisense sequence
guuuucacucccuggcuautt

SEQ ID NO: 1839 - DNA, 21 bp, Artificial Sequence
  Synthetic DNA/RNA construct - RAG3A-255-saRNA antisense sequence
aagaagcacagaggucuugtt

SEQ ID NO: 1840 - DNA, 21 bp, Artificial Sequence
  Synthetic DNA/RNA construct - RAG3A-1000-saRNA antisense sequence
cccaagauuaggaaggaggtt

SEQ ID NO: 1841 - DNA, 21 bp, Artificial Sequence
  Synthetic DNA/RNA construct - RAG3A-694-saRNA antisense sequence
gaauaaccaggcccaugugtt

SEQ ID NO: 1842 - DNA, 21 bp, Artificial Sequence
  Synthetic DNA/RNA construct - RAG3A-549-saRNA antisense sequence
cacuggguuaagccugagctt

SEQ ID NO: 1843 - DNA, 21 bp, Artificial Sequence
  Synthetic DNA/RNA construct - RAG3A-1240-saRNA antisense sequence
ggggaaugcugccgguuuctt

SEQ ID NO: 1844 - DNA, 21 bp, Artificial Sequence
  Synthetic DNA/RNA construct - RAG3A-978-saRNA antisense sequence
cguccagccagacgagaugtt

SEQ ID NO: 1845 - DNA, 21 bp, Artificial Sequence
  Synthetic DNA/RNA construct - RAG3A-1290-saRNA antisense sequence
augaggagcgaccucagagtt

SEQ ID NO: 1846 - DNA, 21 bp, Artificial Sequence
  Synthetic DNA/RNA construct - RAG3A-1006-saRNA antisense sequence
auuaggaaggagggugcugtt

SEQ ID NO: 1847 - DNA, 21 bp, Artificial Sequence
  Synthetic DNA/RNA construct - RAG3A-1311-saRNA antisense sequence
aagguagggaggcaggcuatt

SEQ ID NO: 1848 - DNA, 21 bp, Artificial Sequence
  Synthetic DNA/RNA construct - RAG3A-42-saRNA antisense sequence
agacacuggugaaggcccctt

SEQ ID NO: 1849 - DNA, 21 bp, Artificial Sequence
  Synthetic DNA/RNA construct - RAG3A-21-saRNA antisense sequence
caaucagagaagggagccatt

SEQ ID NO: 1850 - DNA, 21 bp, Artificial Sequence
  Synthetic DNA/RNA construct - RAG3A-59-saRNA antisense sequence
cccggaagugacgccuucutt

SEQ ID NO: 1851 - DNA, 21 bp, Artificial Sequence
  Synthetic DNA/RNA construct - RAG3A-1187-saRNA antisense sequence
augcgccugaggauaaguutt

SEQ ID NO: 1852 - DNA, 21 bp, Artificial Sequence
  Synthetic DNA/RNA construct - RAG3A-576-saRNA antisense sequence
ucgucugcugggguccagutt

SEQ ID NO: 1853 - DNA, 21 bp, Artificial Sequence
  Synthetic DNA/RNA construct - RAG3A-639-saRNA antisense sequence
cuuugcuuguucugucucctt

SEQ ID NO: 1854 - DNA, 21 bp, Artificial Sequence
  Synthetic DNA/RNA construct - RAG3A-815-saRNA antisense sequence
cagucccagucuccccacatt

SEQ ID NO: 1855 - DNA, 21 bp, Artificial Sequence
  Synthetic DNA/RNA construct - RAG3A-957-saRNA antisense sequence
gccuagauccuggaauuuutt

SEQ ID NO: 1856 - DNA, 21 bp, Artificial Sequence
  Synthetic DNA/RNA construct - RAG3A-1217-saRNA antisense sequence
gagauacugucacuccacatt

SEQ ID NO: 1857 - DNA, 21 bp, Artificial Sequence
  Synthetic DNA/RNA construct - RAG3A-1165-saRNA antisense sequence
gaaagcaagaggggacugutt

SEQ ID NO: 1858 - DNA, 21 bp, Artificial Sequence
  Synthetic DNA/RNA construct - RAG3A-1134-saRNA antisense sequence
agagggucccuugggugaatt

SEQ ID NO: 1859 - DNA, 21 bp, Artificial Sequence
  Synthetic DNA/RNA construct - RAG3A-667-saRNA antisense sequence
cuccuaccguuccacaugutt

SEQ ID NO: 1860 - DNA, 21 bp, Artificial Sequence
  Synthetic DNA/RNA construct - RAG3A-1339-saRNA antisense sequence
agaggguggugaggguuugtt

SEQ ID NO: 1861 - DNA, 21 bp, Artificial Sequence
  Synthetic DNA/RNA construct - RAG3A-676-saRNA antisense sequence
uuccacaugugacaagaggtt

SEQ ID NO: 1862 - DNA, 21 bp, Artificial Sequence
  Synthetic DNA/RNA construct - RAG3A-153-saRNA antisense sequence
gauccaucuuuuccuggactt

SEQ ID NO: 1863 - DNA, 21 bp, Artificial Sequence
  Synthetic DNA/RNA construct - RAG3A-320-saRNA antisense sequence
ucccuggacaucauaccugtt

SEQ ID NO: 1864 - DNA, 21 bp, Artificial Sequence
  Synthetic DNA/RNA construct - RAG3A-1010-saRNA antisense sequence
ggaaggagggugcugggautt

SEQ ID NO: 1865 - DNA, 21 bp, Artificial Sequence
  Synthetic DNA/RNA construct - RAG3A-999-saRNA antisense sequence
ucccaagauuaggaaggagtt

SEQ ID NO: 1866 - DNA, 21 bp, Artificial Sequence
  Synthetic DNA/RNA construct - RAG3A-567-saRNA antisense sequence
cuuaggugcucgucugcugtt

SEQ ID NO: 1867 - DNA, 21 bp, Artificial Sequence
  Synthetic DNA/RNA construct - RAG3A-862-saRNA antisense sequence
gaccuugaucccaucaucctt

SEQ ID NO: 1868 - DNA, 21 bp, Artificial Sequence
  Synthetic DNA/RNA construct - RAG3A-460-saRNA antisense sequence
cuccuguuauggguuggugtt

SEQ ID NO: 1869 - DNA, 21 bp, Artificial Sequence
  Synthetic DNA/RNA construct - RAG3A-921-saRNA antisense sequence
ccuccccaaggaugggcautt

SEQ ID NO: 1870 - DNA, 21 bp, Artificial Sequence
  Synthetic DNA/RNA construct - RAG3A-704-saRNA antisense sequence
gcccaugugucccugucuutt

SEQ ID NO: 1871 - DNA, 21 bp, Artificial Sequence
  Synthetic DNA/RNA construct - RAG3A-233-saRNA antisense sequence
caggacauuuguguugcugtt

SEQ ID NO: 1872 - DNA, 21 bp, Artificial Sequence
  Synthetic DNA/RNA construct - RAG3A-1203-saRNA antisense sequence
guuacaaugagagggagautt

SEQ ID NO: 1873 - DNA, 21 bp, Artificial Sequence
  Synthetic DNA/RNA construct - RAG3A-247-saRNA antisense sequence
ugcuggggaagaagcacagtt

SEQ ID NO: 1874 - DNA, 21 bp, Artificial Sequence
  Synthetic DNA/RNA construct - RAG3A-721-saRNA antisense sequence
uuaggaaagacagcugcugtt

SEQ ID NO: 1875 - DNA, 21 bp, Artificial Sequence
  Synthetic DNA/RNA construct - RAG3A-391-saRNA antisense sequence
cuagggaguccuaucuugutt

SEQ ID NO: 1876 - DNA, 21 bp, Artificial Sequence
  Synthetic DNA/RNA construct - RAG3A-159-saRNA antisense sequence
ucuuuuccuggacaugggctt

SEQ ID NO: 1877 - DNA, 21 bp, Artificial Sequence
  Synthetic DNA/RNA construct - RAG3A-437-saRNA antisense sequence
gauauugcccacaugagagtt

SEQ ID NO: 1878 - DNA, 21 bp, Artificial Sequence
  Synthetic DNA/RNA construct - RAG3A-29-saRNA antisense sequence
gaagggagccaccagacactt

SEQ ID NO: 1879 - DNA, 21 bp, Artificial Sequence
  Synthetic DNA/RNA construct - RAG3A-63-saRNA antisense sequence
gaagugacgccuucucuuctt

SEQ ID NO: 1880 - DNA, 21 bp, Artificial Sequence
  Synthetic DNA/RNA construct - RAG3A-996-saRNA antisense sequence
gucucccaagauuaggaagtt

SEQ ID NO: 1881 - DNA, 21 bp, Artificial Sequence
  Synthetic DNA/RNA construct - RAG3A-1083-saRNA antisense sequence
ggcaugaccagggaaccaatt

SEQ ID NO: 1882 - DNA, 21 bp, Artificial Sequence
  Synthetic DNA/RNA construct - RAG3A-1128-saRNA antisense sequence
agaggcagagggucccuugtt

SEQ ID NO: 1883 - DNA, 21 bp, Artificial Sequence
  Synthetic DNA/RNA construct - RAG3A-1094-saRNA antisense sequence
ggaaccaagagaaaggaggtt

SEQ ID NO: 1884 - DNA, 21 bp, Artificial Sequence
  Synthetic DNA/RNA construct - RAG3A-771-saRNA antisense sequence
ccccuuuuccuuuggcccutt

SEQ ID NO: 1885 - DNA, 21 bp, Artificial Sequence
  Synthetic DNA/RNA construct - RAG3A-287-saRNA antisense sequence
ggguuuucacucccuggcutt

SEQ ID NO: 1886 - DNA, 21 bp, Artificial Sequence
  Synthetic DNA/RNA construct - RAG3A-981-saRNA antisense sequence
ccagccagacgagaugucutt

SEQ ID NO: 1887 - DNA, 21 bp, Artificial Sequence
  Synthetic DNA/RNA construct - RAG3A-23-saRNA antisense sequence
aucagagaagggagccacctt

SEQ ID NO: 1888 - DNA, 21 bp, Artificial Sequence
  Synthetic DNA/RNA construct - RAG3A-1072-saRNA antisense sequence
acagggaggcaggcaugactt

SEQ ID NO: 1889 - DNA, 21 bp, Artificial Sequence
  Synthetic DNA/RNA construct - RAG3A-415-saRNA antisense sequence
ccccuuucgaagugggaactt

SEQ ID NO: 1890 - DNA, 21 bp, Artificial Sequence
  Synthetic DNA/RNA construct - RAG3A-1287-saRNA antisense sequence
agaaugaggagcgaccucatt

SEQ ID NO: 1891 - DNA, 21 bp, Artificial Sequence
  Synthetic DNA/RNA construct - RAG3A-1201-saRNA antisense sequence
aaguuacaaugagagggagtt

SEQ ID NO: 1892 - DNA, 21 bp, Artificial Sequence
  Synthetic DNA/RNA construct - RAG3A-772-saRNA antisense sequence
cccuuuuccuuuggcccugtt

SEQ ID NO: 1893 - DNA, 21 bp, Artificial Sequence
  Synthetic DNA/RNA construct - RAG3A-1249-saRNA antisense sequence
ugccgguuucuggcuaggutt

SEQ ID NO: 1894 - DNA, 21 bp, Artificial Sequence
  Synthetic DNA/RNA construct - RAG3A-546-saRNA antisense sequence
gugcacuggguuaagccugtt

SEQ ID NO: 1895 - DNA, 21 bp, Artificial Sequence
  Synthetic DNA/RNA construct - RAG3A-160-saRNA antisense sequence
cuuuuccuggacaugggcatt

SEQ ID NO: 1896 - DNA, 21 bp, Artificial Sequence
  Synthetic DNA/RNA construct - RAG3A-64-saRNA antisense sequence
aagugacgccuucucuucctt

SEQ ID NO: 1897 - DNA, 21 bp, Artificial Sequence
  Synthetic DNA/RNA construct - RAG3A-1002-saRNA antisense sequence
caagauuaggaaggagggutt

SEQ ID NO: 1898 - DNA, 21 bp, Artificial Sequence
  Synthetic DNA/RNA construct - RAG3A-1288-saRNA antisense sequence
gaaugaggagcgaccucagtt

SEQ ID NO: 1899 - DNA, 21 bp, Artificial Sequence
  Synthetic DNA/RNA construct - RAG3A-891-saRNA antisense sequence
cagaacuuccucaggcucutt

SEQ ID NO: 1900 - DNA, 21 bp, Artificial Sequence
  Synthetic DNA/RNA construct - RAG3A-231-saRNA antisense sequence
ggcaggacauuuguguugctt

SEQ ID NO: 1901 - DNA, 21 bp, Artificial Sequence
  Synthetic DNA/RNA construct - RAG3A-669-saRNA antisense sequence
ccuaccguuccacaugugatt

SEQ ID NO: 1902 - DNA, 21 bp, Artificial Sequence
  Synthetic DNA/RNA construct - RAG3A-1009-saRNA antisense sequence
aggaaggagggugcugggatt

SEQ ID NO: 1903 - DNA, 21 bp, Artificial Sequence
  Synthetic DNA/RNA construct - RAG3A-656-saRNA antisense sequence
ccgucuuccaucuccuacctt

SEQ ID NO: 1904 - DNA, 21 bp, Artificial Sequence
  Synthetic DNA/RNA construct - RAG3A-229-saRNA antisense sequence
cuggcaggacauuuguguutt

SEQ ID NO: 1905 - DNA, 21 bp, Artificial Sequence
  Synthetic DNA/RNA construct - RAG3A-484-saRNA antisense sequence
gggaaaggguuuggacgggtt

SEQ ID NO: 1906 - DNA, 21 bp, Artificial Sequence
  Synthetic DNA/RNA construct - RAG3A-799-saRNA antisense sequence
uccuuugcuuucuucccagtt

SEQ ID NO: 1907 - DNA, 21 bp, Artificial Sequence
  Synthetic DNA/RNA construct - RAG3A-296-saRNA antisense sequence
cucccuggcuauccaaauutt

SEQ ID NO: 1908 - DNA, 21 bp, Artificial Sequence
  Synthetic DNA/RNA construct - RAG3A-1183-saRNA antisense sequence
ucgaaugcgccugaggauatt

SEQ ID NO: 1909 - DNA, 21 bp, Artificial Sequence
  Synthetic DNA/RNA construct - RAG3A-380-saRNA antisense sequence
ucuguaauccccuagggagtt

SEQ ID NO: 1910 - DNA, 21 bp, Artificial Sequence
  Synthetic DNA/RNA construct - RAG3A-157-saRNA antisense sequence
caucuuuuccuggacauggtt

SEQ ID NO: 1911 - DNA, 21 bp, Artificial Sequence
  Synthetic DNA/RNA construct - RAG3A-1185-saRNA antisense sequence
gaaugcgccugaggauaagtt

SEQ ID NO: 1912 - DNA, 21 bp, Artificial Sequence
  Synthetic DNA/RNA construct - RAG3A-1244-saRNA antisense sequence
aaugcugccgguuucuggctt

SEQ ID NO: 1913 - DNA, 21 bp, Artificial Sequence
  Synthetic DNA/RNA construct - RAG3A-588-saRNA antisense sequence
guccaguggcuuagauggctt

SEQ ID NO: 1914 - DNA, 21 bp, Artificial Sequence
  Synthetic DNA/RNA construct - RAG3A-436-saRNA antisense sequence
ggauauugcccacaugagatt

SEQ ID NO: 1915 - DNA, 21 bp, Artificial Sequence
  Synthetic DNA/RNA construct - RAG3A-1029-saRNA antisense sequence
agcugggaggauagugggutt

SEQ ID NO: 1916 - DNA, 21 bp, Artificial Sequence
  Synthetic DNA/RNA construct - RAG3A-28-saRNA antisense sequence
agaagggagccaccagacatt

SEQ ID NO: 1917 - DNA, 21 bp, Artificial Sequence
  Synthetic DNA/RNA construct - RAG3A-234-saRNA antisense sequence
aggacauuuguguugcuggtt

SEQ ID NO: 1918 - DNA, 21 bp, Artificial Sequence
  Synthetic DNA/RNA construct - RAG3A-249-saRNA antisense sequence
cuggggaagaagcacagagtt

SEQ ID NO: 1919 - DNA, 21 bp, Artificial Sequence
  Synthetic DNA/RNA construct - RAG3A-775-saRNA antisense sequence
uuuuccuuuggcccuggcutt

SEQ ID NO: 1920 - DNA, 21 bp, Artificial Sequence
  Synthetic DNA/RNA construct - RAG3A-951-saRNA antisense sequence
aguguggccuagauccuggtt

SEQ ID NO: 1921 - DNA, 21 bp, Artificial Sequence
  Synthetic DNA/RNA construct - RAG3A-861-saRNA antisense sequence
ugaccuugaucccaucauctt

SEQ ID NO: 1922 - DNA, 21 bp, Artificial Sequence
  Synthetic DNA/RNA construct - RAG3A-259-saRNA antisense sequence
agcacagaggucuuguuuatt

SEQ ID NO: 1923 - DNA, 21 bp, Artificial Sequence
  Synthetic DNA/RNA construct - RAG3A-394-saRNA antisense sequence
gggaguccuaucuugucautt

SEQ ID NO: 1924 - DNA, 21 bp, Artificial Sequence
  Synthetic DNA/RNA construct - RAG3A-534-saRNA antisense sequence
augugcgcacacgugcacutt

SEQ ID NO: 1925 - DNA, 21 bp, Artificial Sequence
  Synthetic DNA/RNA construct - RAG3A-316-saRNA antisense sequence
cuuuucccuggacaucauatt

SEQ ID NO: 1926 - DNA, 21 bp, Artificial Sequence
  Synthetic DNA/RNA construct - RAG3A-560-saRNA antisense sequence
gccugagcuuaggugcucgtt

SEQ ID NO: 1927 - DNA, 21 bp, Artificial Sequence
  Synthetic DNA/RNA construct - RAG3A-1105-saRNA antisense sequence
aaaggaggcugggagugggtt

SEQ ID NO: 1928 - DNA, 21 bp, Artificial Sequence
  Synthetic DNA/RNA construct - RAG3A-709-saRNA antisense sequence
ugugucccugucuuaggaatt

SEQ ID NO: 1929 - DNA, 21 bp, Artificial Sequence
  Synthetic DNA/RNA construct - RAG3A-948-saRNA antisense sequence
agaaguguggccuagaucctt

SEQ ID NO: 1930 - DNA, 21 bp, Artificial Sequence
  Synthetic DNA/RNA construct - RAG3A-998-saRNA antisense sequence
cucccaagauuaggaaggatt

SEQ ID NO: 1931 - DNA, 21 bp, Artificial Sequence
  Synthetic DNA/RNA construct - RAG3A-387-saRNA antisense sequence
uccccuagggaguccuauctt